(19) United States
(12) Patent Application Publication
(10) Pub. No.: US 2025/0197953 A1
(43) Pub. Date: Jun. 19, 2025

(54) METHODS OF DETERMINING SENSITIVITY TO PHOTOPERIOD IN CANNABIS

(71) Applicant: Aurora Cannabis Enterprises Inc., Vancouver (CA)

(72) Inventors: Gregory Baute, Vancouver (CA); Jose Celedon, Vancouver (CA); Keegan Leckie, Vancouver (CA); Jason Sawler, Vancouver (CA); Paul Kapos, Vancouver (CA)

(73) Assignee: Aurora Cannabis Enterprises Inc., Vancouver, BC (CA)

(21) Appl. No.: 18/696,776

(22) PCT Filed: Sep. 30, 2022
(86) PCT No.: PCT/CA2022/051457

§ 371 Date: Mar. 28, 2024

Related U.S. Application Data
(60) Provisional application No. 63/250,936, filed on Sep. 30, 2021.
(60) Provisional application No. 63/250,943, filed on Sep. 30, 2021.

(51) Int. Cl. C12Q 1/6895; A01H 1/04; A01H 5/12; A01H 6/28

(52) U.S. Cl. CPC C12Q 1/6895; A01H 1/04; A01H 5/12; A01H 6/28; C12Q 2600/13; C12Q 2600/156

(57) ABSTRACT

This disclosure pertains to markers and methods useful in identifying Cannabis plants that have a day length neutral phenotype, and methods of breeding plants having a day length neutral phenotype.

### CROSS-REFERENCE TO RELATED APPLICATIONS

This application is a U.S. National Stage Application of PCT/CA2022/051457, filed Sep. 30, 2022, which claims the benefit of U.S. Appln. No. 63/250,936, filed Sep. 30, 2021, and U.S. Appln. No. 63/250,943, filed Sep. 30, 2021, the disclosures of which are incorporated herein in their entirety.

### STATEMENT REGARDING SEQUENCE LISTING

The Sequence Listing XML associated with this application is provided in XML format and is hereby incorporated by reference into the specification. The name of the XML file containing the sequence listing is 1018-P22US.PNP_Seq_List_20241218.xml. The XML file is 298,219 bytes; was created on Dec. 18, 2024; and is being submitted electronically via Patent Center with the filing of the specification.

### FIELD OF THE DISCLOSURE

This disclosure relates to the field of genetic markers for a day length neutral phenotype in Cannabis. More specifically, this disclosure relates to methods and compositions useful for identifying individual plants that display a day length neutral phenotype, or are a carrier for that trait.

### BACKGROUND OF THE DISCLOSURE

Much of the cannabis in production today is day length (photoperiod) sensitive, meaning that it initiates flowering only after a transition from long photoperiod days to short photoperiod days. This has several limitations, especially at more northern (and more southern) latitudes.

### SUMMARY OF THE INVENTION

The present disclosure pertains to the utility of a variation at a polymorphic site in the Cannabis PSEUDO-RESPONSE REGULATOR 7 (PRR7) gene for identifying Cannabis plants that have a day length neutral phenotype (i.e., have an “autoflowering” phenotype). The present disclosure also pertains to the utility of PRR7 in producing Cannabis plants that have a day length neutral phenotype.

The present disclosure also pertains to the utility of a low-recombination region in the Cannabis genome for identifying plants that have the day length neutral phenotype. For example, the present disclosure pertains to the utility of a variation at a polymorphic site within a low-recombination region in the Cannabis genome or an allele in linkage disequilibrium with the polymorphic site within the low-recombination region, the low-recombination region is an about 20 megabase region in chromosome 5 (identified by GenBank Sequence as CM010796.2) between about 40 megabases to about 60 megabases, for identifying Cannabis plants that have a day length neutral phenotype.

Various aspects of the disclosure relate to a method comprising testing nucleic acid from a Cannabis plant to determine the presence or absence of a variation at a polymorphic site in the endogenous PRR7 gene. The method is for identifying whether or not the Cannabis plant has a day length neutral phenotype, or is a carrier of a trait for the day length neutral phenotype. The presence of the variation indicates that the plant has the day length neutral phenotype or is a carrier of the trait for the day length neutral phenotype.

Various aspects of the disclosure relate to a method comprising testing nucleic acid from a Cannabis plant to determine the presence or absence of an allele that is in linkage disequilibrium with a variation in the endogenous PRR7 gene. The allele is located within an about 20 megabase region in Cannabis chromosome CM010796.2 located between about 40 megabases to about 60 megabases.

Various aspects of the disclosure relate to a method comprising testing nucleic acid from a Cannabis plant to determine the presence or absence of a variation at a polymorphic site corresponding to any one or more of SEQ ID NOS: 4 to 9.

Various aspects of the disclosure relate to a method comprising testing nucleic acid from a Cannabis plant to determine the presence or absence of an allele that is in linkage disequilibrium with a variation at a polymorphic site corresponding to any one or more SEQ ID NOS: 4 to 9.

Various aspects of the disclosure relate to a method comprising testing nucleic acid from a Cannabis plant to determine the presence or absence of a variation at a polymorphic site within a low-recombination region in the Cannabis genome or an allele in linkage disequilibrium with the polymorphic site within the low-recombination region. The low-recombination region is an about 20 megabase region in chromosome 5 (CM010796.2) between about 40 megabases to about 60 megabases. The variation at the polymorphic site corresponds to any one of SEQ ID NOs: 69 to 91.

Various aspects of the disclosure relate to a method comprising testing nucleic acid from a Cannabis plant to determine the presence or absence of a variation at a polymorphic site in any one of SEQ ID NOs: 28 to 40 or an allele in linkage disequilibrium with the polymorphic site corresponding to any one of SEQ ID NOs: 28 to 40.

Various aspects of the disclosure relate to a method comprising testing nucleic acid from a Cannabis plant to determine the presence or absence of a variation at a polymorphic site corresponding to any one of SEQ ID NOs: 69 to 115 or an allele in linkage disequilibrium with the polymorphic site corresponding to any one of SEQ ID NOs: 69 to 115.

Various aspects of the disclosure relate to a method comprising testing nucleic acid from a Cannabis plant to determine the presence or absence of a variation at a polymorphic site corresponding to any one of SEQ ID NOs: 69 to 91 or an allele in linkage disequilibrium with the polymorphic site corresponding to any one of SEQ ID NOs: 69 to 91.

Various aspects of the disclosure relate to a method comprising testing nucleic acid from a Cannabis plant to determine the presence or absence of a variation at a polymorphic site corresponding to any one of SEQ ID NOs: 70 to 83 or an allele in linkage disequilibrium with the polymorphic site corresponding to any one of SEQ ID NOs: 70 to 83.

Various aspects of the disclosure relate to a method of producing a Cannabis plant with a day length neutral phenotype. The method comprises performing a first cross of a first parent having at least one allele associated with a day length neutral phenotype at a polymorphic site corresponding to SEQ ID NOs: 4 to 9, with a second parent that has the day length sensitive phenotype. The method further comprises identifying a first progeny plant from the first cross that has the day length neutral phenotype, or that is a carrier of a trait for the day length neutral phenotype, according to the method defined herein.

Various aspects of the disclosure relate to a method of producing a Cannabis plant with a day length neutral phenotype. The method comprising performing a first cross of a first parent that has the day length neutral phenotype and is homozygous for at least one allele associated with the day length neutral phenotype at a polymorphic site corresponding to SEQ ID NOs: 4 to 9, with a second parent that has the day length sensitive phenotype to produce F₁ progeny. The method further comprises selfing the F₁ progeny to produce F₂ progeny. The method further comprises identifying an F₂ progeny plant that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises identifying a first parent homozygous for at least one allele associated with the day length neutral phenotype at a polymorphic site corresponding to SEQ ID NOs: 4 to 9 according to the methods described herein. The method further comprises crossing the first parent with a second parent that has the day length sensitive phenotype to produce F₁. The method further comprises selfing the F₁ progeny to produce F₂ progeny. The method further comprises identifying an F₂ progeny plant that has the day length neutral phenotype.

Various aspects of the disclosure relate to a method of producing a Cannabis plant with a day length neutral phenotype. The method comprises performing a first cross of a first parent identified having at least one loss of function mutation allele of the PRR7 gene with a second parent that has the day length sensitive phenotype. The method further comprises performing a second cross of a first progeny of the first cross with a second progeny of the first cross to produce a plant that is homozygous for the loss of function mutation allele.

Various aspects of the disclosure relate to a method of producing a Cannabis plant with a day length neutral phenotype. The method comprises performing a first cross of a first parent identified having at least one loss of function mutation allele of the PRR5 and/or PRR9 gene with a second parent that has the day length sensitive phenotype. The method further comprises performing a second cross of a first progeny of the first cross with a second progeny of the first cross to produce a plant that is homozygous for the loss of function mutation allele.

Various aspects of the disclosure relate to a method of producing a Cannabis plant with a day length neutral phenotype. The method comprises performing a first cross of a first parent having at least one allele associated with the day length neutral phenotype at a polymorphic site at a polymorphic site corresponding to SEQ ID NOs: 69 to 91 within an about 20 megabase region in Cannabis chromosome CM010796.2 located between about 40 megabases to about 60 megabases, with a second parent that has a day length sensitive phenotype. The method further comprises identifying a first progeny plant from the first cross that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises performing a first cross of a first parent that has the day length neutral phenotype and is homozygous for at least one allele associated with the day length neutral phenotype at a polymorphic site corresponding to SEQ ID NOs: 69 to 91 within an about 20 megabase region in Cannabis chromosome CM010796.2 located between about 40 megabases to about 60 megabases, with a second parent that has a day length sensitive phenotype to produce F₁ progeny. The method further comprises selfing the F₁ progeny to produce F₂ progeny. The method further comprises identifying an F₂ progeny plant that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises identifying a first parent homozygous for at least one allele associated with the day length neutral phenotype at a polymorphic site corresponding to SEQ ID NOs: 69 to 91 within an about 20 megabase region in Cannabis chromosome CM010796.2 located between about 40 megabases to about 60 megabases, according to the methods described herein. The method also comprises crossing the first parent with a second parent that has a day length sensitive phenotype to produce F₁. The method also comprises selfing the F₁ progeny to produce F₂ progeny. The method also comprises identifying an F₂ progeny plant that has the day length neutral phenotype.

Various aspects of the disclosure relate to a method of producing a Cannabis plant with a day length neutral phenotype. The method comprises performing a first cross of a first parent having at least one allele associated with the day length neutral phenotype at a polymorphic site at a polymorphic site corresponding to SEQ ID NOs: 70 to 83 within an about 2.5 megabase region in Cannabis chromosome CM010796.2 located between about 41.09 megabases to about 43.59 megabases, with a second parent that has a day length sensitive phenotype. The method also comprises identifying a first progeny plant from the first cross that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises performing a first cross of a first parent that has the day length neutral phenotype and is homozygous for at least one allele associated with the day length neutral phenotype at a polymorphic site corresponding to SEQ ID NOs: 70 to 83 within an about 2.5 megabase region in Cannabis chromosome CM010796.2 located between about 41.09 megabases to about 43.59 megabases with a second parent that has a day length sensitive phenotype to produce F₁ progeny. The method also comprises selfing the F₁ progeny to produce F₂ progeny. The method also comprises identifying an F₂ progeny plant that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises identifying a first parent homozygous for at least one allele associated with the day length neutral phenotype at a polymorphic site corresponding to SEQ ID NOs: 70 to 83 within an about 2.5 megabase region in Cannabis chromosome CM010796.2 located between about 41.09 megabases to about 43.59 megabases, according to the methods as described herein. The method also comprises crossing the first parent with a second parent that has a day length sensitive phenotype to produce F₁. The method also comprises selfing the F₁ progeny to produce F₂ progeny. The method also comprises identifying an F₂ progeny plant that has the day length neutral phenotype.

Various aspects of the disclosure relate to a method of producing a Cannabis plant with a day length neutral phenotype. The method comprises performing a first cross of a first parent having at least one allele associated with the day length neutral phenotype at a polymorphic site at a polymorphic site corresponding to SEQ ID NOs: 70 to 83 within an about 0.75 megabase region in Cannabis chromosome CM010796.2 located between about 41.09 megabases to about 41.84 megabases, with a second parent that has a day length sensitive phenotype. The method also comprises identifying a first progeny plant from the first cross that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises performing a first cross of a first parent that has the day length neutral phenotype and is homozygous for at least one allele associated with the day length neutral phenotype at a polymorphic site corresponding to SEQ ID NOs: 70 to 83 within an about 0.75 megabase region in Cannabis chromosome CM010796.2 located between about 41.09 megabases to about 41.84 megabases, with a second parent that has a day length sensitive phenotype to produce F₁ progeny. The method also comprises selfing the F₁ progeny to produce F₂ progeny. The method also comprises identifying an F₂ progeny plant that has the day length neutral phenotype, or that is a carrier of a trait for the day length neutral phenotype, according to the methods described herein.

Various aspects of the disclosure relate to a method of producing a Cannabis plant with a day length neutral phenotype. The method comprises identifying a first parent homozygous for at least one allele associated with the day length neutral phenotype at a polymorphic site corresponding to SEQ ID NOs: 70 to 83 within an about 0.75 megabase region in Cannabis chromosome CM010796.2 located between about 41.09 megabases to about 41.84 megabases, according to the method described herein. The method also comprises crossing the first parent with a second parent that has a day length sensitive phenotype to produce F₁. The method also comprises identifying an F₂ progeny plant that has the day length neutral phenotype.

Various aspects of the disclosure relate to a method for producing a day length neutral Cannabis plant. The method comprises decreasing the expression of an endogenous PRR7 gene in the plant.

Various aspects of the disclosure relate to a method for producing a day length neutral Cannabis plant. The method comprises decreasing the expression of an endogenous PRR5 and/or PRR9 gene in the plant.

Various aspects of the disclosure relate to a method of method of generating a Cannabis plant having a day length neutral phenotype. The method comprises i) using a molecular methodology to identify a first plant as comprising a loss of function allele in an endogenous PRR7 gene; ii) performing a first cross of said first plant to a second plant; iii) performing a second cross of progeny from the first cross; and iv) screening progeny of the second cross for a plant that is homozygous for the loss of function allele in the endogenous PRR7 gene.

Various aspects of the disclosure relate to a method of method of generating a Cannabis plant having a day length neutral phenotype. The method comprises i) using a molecular methodology to identify a first plant as comprising a loss of function allele in an endogenous PRR5 and/or PRR9 gene; ii) performing a first cross of said first plant to a second plant; iii) performing a second cross of progeny from the first cross; and iv) screening progeny of the second cross for a plant that is homozygous for the loss of function allele in the endogenous PRR5 and/or PRR9 gene.

Various aspects of the disclosure relate to a Cannabis plant or plant cell generated according to the methods described herein, as well as seed, plant material, or dried flower of such plants.

Various aspects of the disclosure relate to a genetically modified Cannabis plant or plant cell having a day length neutral phenotype, as well as seed, plant material, or dried flower of such plants. The Cannabis plant or plant cell is genetically modified to have reduced expression of an endogenous PRR7 gene.

Various aspects of the disclosure relate to a genetically modified Cannabis plant or plant cell having a day length neutral phenotype, as well as seed, plant material, or dried flower of such plants. The Cannabis plant or plant cell is genetically modified to have reduced expression of an endogenous PRR5 and/or PRR9 gene.

Various aspects of the disclosure relate to an allele-specific polynucleotide for use in the methods described herein.

Various aspects of the disclosure relate to a kit for use in the methods defined herein. The kit comprises at least one allele-specific polynucleotide as descried herein and at least one further component, wherein the at least one further component is a buffer, deoxynucleotide triphosphates (dNTPs), an amplification primer pair, an enzyme, or any combination thereof.

Various aspects of the disclosure relate to a cannabinoid and/or a terpene produced by a plant or plant cell as described herein.

Various aspects of the disclosure relate to a cannabinoid and/or a terpene extracted or isolated from dried flower as described herein.

Various aspects of the disclosure relate to extracts, concentrates, isolates, or oils of a Cannabis plant, plant cell, dried flower, plant material, or seed as described herein. The extracts, concentrates, isolates, and oils comprise a cannabinoid and/or a terpene.

Various aspects of the disclosure relate to a crop comprising a plurality of Cannabis plants as described herein.

Various aspects of the disclosure relate to a method of producing a cannabinoid and/or a terpene, said method comprising extracting or isolating a cannabinoid and/or a terpene from flowers harvested from the plants described herein.

Various aspects of the disclosure relate to an expression vector for generating a day length neutral Cannabis plant, the expression vector comprising a nucleic acid comprising a portion of SEQ ID NO: 1.

Various aspects of the disclosure relate to use of a polynucleotide molecule having a sequence comprising a portion of SEQ ID NO: 1 for generating a Cannabis plant with a day length neutral phenotype.

Various aspects of the disclosure relate to use of a plant or plant cell as described herein for the production of a cannabinoid and/or a terpene.

Various aspects of the disclosure relate to a Cannabis product comprising a Cannabis plant or plant cell, seed, plant material, or dried flower as described herein.

Various aspects of the disclosure relate to a Cannabis product comprising an extract, a concentrate, anisolate, and/or an oil of a Cannabis plant, plant cell, dried flower, plant material, or seed as described herein. The Cannabis product comprises a cannabinoid and/or a terpene. In some embodiments, the Cannabis product comprises a food item, a beverage, a topical, a pharmaceutical composition, or a neutraceutical composition.

Various aspects of the disclosure relate to a method of producing oil. The method comprises extracting oil from a plant, plant cell, seed, plant material or dried flower as described herein.

Various aspects of the disclosure relate to a method of producing an extract comprising a cannabinoids and/or a terpene. The method comprises extracting a cannabinoid and/or a terpene from a plant, plant cell, seed, plant material, or dried flower as described herein.

Various aspects of the disclosure relate to a method of producing a concentrate comprising a cannabinoid and/or a terpene. The method comprises extracting a concentrate comprising a cannabinoid and/or a terpene from a plant, plant cell, seed, plant material, or dried flower as described herein.

Various aspects of the disclosure relate to a method of producing an isolate comprising a cannabinoid and/or a terpene. The method comprises isolating a cannabinoid and/or a terpene from a plant, plant cell, seed, plant material, or dried flower as described herein.

Other aspects and features of the present invention will become apparent to those ordinarily skilled in the art upon review of the following description of specific embodiments of the invention

### BRIEF DESCRIPTION OF THE FIGURES

Exemplary embodiments are illustrated in referenced figures of the drawings. It is intended that the embodiments and figures disclosed herein are to be considered illustrative rather than restrictive.

FIG. 1 is a graph showing the genetic versus physical distance in chromosome 1 (NCBI accession NC_044371.1). The star denotes the location of the UPF2 gene.

FIG. 2 is a diagram showing the structural variants (SVs) in the region of chromosome 1 (NC_044371.1) where the autoflowering marker is located. Sequence similarity is indicated by fill inside each colinear block. Sequence inversions are indicated by boxes on the lower side of the reference line for each chromosome. Translocations are indicated by connecting lines crossing each other.

FIG. 3 are graphs showing Fixation index (F_{ST}) values between 12 autoflowering and 12 photoperiod accessions (top panel), and Nucleotide diversity (Pi) (bottom panel) within 12 autoflowering (bottom line) and 12 photoperiod (top line) accessions.

FIG. 4 is a chart showing a physical genomic map of the 0.75 Mb, 2.5 Mb, and 20 Mb linkage region found in chromosome 5 (Purple Kush Assembly v4). From tracks top to bottom: locations of genetic markers to track recombination for test crosses; proposed 20 Mb linkage region boundaries as determined by WGS fixation index and nucleotide diversity scan; maximum extant of reduced linkage region through test crossing; minimal extant of reduced linkage region though test crossing; location of the auto-flowering associated genes.

FIG. 5A is a graph showing gene expression levels from RNASeq analysis by developmental stage of PRR7 in Cannabis during floral development for autoflowering (dark grey) and photoperiod (light grey) genotypes (N=3).

FIG. 5B is a graph showing gene expression levels from RNASeq analysis by days after first sample of PRR7 during floral development for autoflowering (dark grey) and photoperiod (light grey) genotypes (N=3).

FIG. 6A is a graph showing gene expression levels from RNASeq analysis by developmental stage of TOE1 in Cannabis during floral development for autoflowering (dark grey) and photoperiod (light grey) genotypes.

FIG. 6B is a graph showing gene expression levels from RNASeq analysis by days after first sample of TOE1 during floral development for autoflowering (dark grey) and photoperiod (light grey) genotypes.

FIG. 7A is a graph showing gene expression levels from RNASeq analysis by developmental stage of ELF6 in Cannabis during floral development for autoflowering (dark grey) and photoperiod (light grey) genotypes.

FIG. 7B is a graph showing gene expression levels from RNASeq analysis by days after first sample of ELF6 during floral development for autoflowering (dark grey) and photoperiod (light grey) genotypes.

FIG. 8A is a graph showing gene expression levels from RNASeq analysis by developmental stage of GA2OX8 in Cannabis during floral development for autoflowering (dark grey) and photoperiod (light grey) genotypes.

FIG. 8B is a graph showing gene expression levels from RNASeq analysis by days after first sample of GA2OX8 during floral development for autoflowering (dark grey) and photoperiod (light grey) genotypes.

FIG. 9A is a graph showing gene expression levels from RNASeq analysis by developmental stage of SINAT3 in Cannabis during floral development for autoflowering (dark grey) and photoperiod (light grey) genotypes.

FIG. 9B is a graph showing gene expression levels from RNASeq analysis by days after first sample of SINAT3 during floral development for autoflowering (dark grey) and photoperiod (light grey) genotypes.

FIG. 10A is a graph showing gene expression levels from RNASeq analysis by developmental stage of SKIP5 in Cannabis during floral development for autoflowering (dark grey) and photoperiod (light grey) genotypes.

FIG. 10B is a graph showing gene expression levels from RNASeq analysis by days after first sample of SKIP5 during floral development for autoflowering (dark grey) and photoperiod (light grey) genotypes.

FIG. 11A is a graph showing gene expression levels from RNASeq analysis by developmental stage of LNK2 in Cannabis during floral development for autoflowering (dark grey) and photoperiod (light grey) genotypes.

FIG. 11B is a graph showing gene expression levels from RNASeq analysis by days after first sample of LNK2 during floral development for autoflowering (dark grey) and photoperiod (light grey) genotypes.

FIG. 12A is a graph showing gene expression levels from RNASeq analysis by developmental stage of ARR9 in Cannabis during floral development for autoflowering (dark grey) and photoperiod (light grey) genotypes.

FIG. 12B is a graph showing gene expression levels from RNASeq analysis by days after first sample of ARR9 during floral development for autoflowering (dark grey) and photoperiod (light grey) genotypes.

FIG. 13A is a graph showing gene expression levels from RNASeq analysis by developmental stage of ERF105 in Cannabis during floral development for autoflowering (dark grey) and photoperiod (light grey) genotypes.

FIG. 13B is a graph showing gene expression levels from RNASeq analysis by days after first sample of ER105 during floral development for autoflowering (dark grey) and photoperiod (light grey) genotypes.

FIG. 14A is a graph showing gene expression levels from RNASeq analysis by developmental stage of GRAS in Cannabis during floral development for autoflowering (dark grey) and photoperiod (light grey) genotypes.

FIG. 14B is a graph showing gene expression levels from RNASeq analysis by days after first sample of GRAS during floral development for autoflowering (dark grey) and photoperiod (light grey) genotypes.

FIG. 15A is a graph showing gene expression levels from RNASeq analysis by developmental stage of ELF4 in Cannabis during floral development for autoflowering (dark grey) and photoperiod (light grey) genotypes.

FIG. 15B is a graph showing gene expression levels from RNASeq analysis by days after first sample of ELF4 during floral development for autoflowering (dark grey) and photoperiod (light grey) genotypes.

FIG. 16A is a graph showing gene expression levels from RNASeq analysis by developmental stage of FRIGIDA-like in Cannabis during floral development for autoflowering (dark grey) and photoperiod (light grey) genotypes.

FIG. 16B is a graph showing gene expression levels from RNASeq analysis by days after first sample of FRIGIDA-like during floral development for autoflowering (dark grey) and photoperiod (light grey) genotypes.

FIG. 17A is a graph showing gene expression levels from RNASeq analysis by developmental stage of VRN5 in Cannabis during floral development for autoflowering (dark grey) and photoperiod (light grey) genotypes.

FIG. 17B is a graph showing gene expression levels from RNASeq analysis by days after first sample of VRN5 during floral development for autoflowering (dark grey) and photoperiod (light grey) genotypes.

FIG. 18A is a graph showing gene expression levels from RNASeq analysis by developmental stage of EIN3 in Cannabis during floral development for autoflowering (dark grey) and photoperiod (light grey) genotypes.

FIG. 18B is a graph showing gene expression levels from RNASeq analysis by days after first sample of EIN3 during floral development for autoflowering (dark grey) and photoperiod (light grey) genotypes.

FIG. 19A is a chart showing a protein sequence BLAST alignment between Arabidopsis APRR7 and Cannabis homolog PRR7 (csPRR7).

FIG. 19B is a diagram showing aligned hits to functional domains between Arabidopsis APRR7 and Cannabis homolog PRR7.

FIG. 20 is a schematic showing a gene model of PRR7 with the autoflowering associated SNP at the third intron splice donor site, Cannabis chromosome 5 (identified by GenBank Sequence as CM010796.2) base pair position 41,126,556. Dark grey boxes indicate the coding DNA sequence (CDS) and light grey boxes indicate the untranslated region (UTR). Gene is located on the reverse DNA stand (-).

FIG. 21 is a graph showing hypocotyl length of 11 day old Cannabis seedlings grown under Dark (no light), FarRed (730 nm; 16 h light, 8 h dark), or White (Full spectrum; 16 h light, 8 h dark) for 7 days.

FIG. 22 is a schematic showing circadian clock genes and their regulation of the flowering pathway.

FIG. 23A is a graph showing gene expression levels from RNASeq analysis by developmental stage of FT (12434) homologue in Cannabis for autoflowering (light grey) and photosensitive (dark grey) genotypes.

FIG. 23B is a graph showing qPCR expression levels at specific timepoints after germination of FT (12434) homologue in Cannabis for autoflowering (light grey) and photosensitive (dark grey) genotypes.

FIG. 23C is a graph showing qPCR expression levels at specific timepoints after germination of FT (7085) homologue in Cannabis for autoflowering (light grey) and photosensitive (dark grey) genotypes.

FIG. 23D is a graph showing qPCR expression levels at specific timepoints after germination of FT (4768) homologue in Cannabis for autoflowering (light grey) and photosensitive (dark grey) genotypes.

FIG. 24 is a graph showing qPCR expression levels at specific timepoints after germination of TOE1 in Cannabis for autoflowering (light grey) and photosensitive (dark grey) genotypes.

FIG. 25A-C are graphs showing qPCR expression levels at specific timepoints after germination of three AP2-like homologues in Cannabis for autoflowering (light grey) and photosensitive (dark grey) genotypes.

FIG. 26 is a graph showing qPCR expression levels at specific timepoints after germination under long days (LD) of PRR7 in Cannabis for autoflowering (light grey) and photosensitive (dark grey) genotypes.

FIGS. 27A-C are graphs showing gene expression levels from RNASeq analysis by developmental stage of three identified PRR5 homologues in Cannabis for autoflowering (dark grey) and photosensitive (light grey) genotypes.

FIG. 27D is a graph showing gene expression levels from RNASeq analysis by developmental stage of the PRR9 homologue in autoflowering (dark grey) and photosensitive (light grey) genotypes.

FIG. 28A is a graph showing qPCR expression levels at specific timepoints over a 24-hour period under LD (black line) or short days (SD) (grey line) of PRR7 in Cannabis for photosensitive genotype. Black bars indicate hours without light under LD, grey bars indicate hours without light under SD. X axis indicates hours before or after mid-point of day light cycle.

FIG. 28B is a graph showing qPCR expression levels at specific timepoints over a 24-hour period under LD (black line) or SD (grey line) of PRR7 in Cannabis for autoflowering genotype.

FIG. 29A is a graph showing qPCR expression levels at specific timepoints over a 24-hour period under LD (black line) or SD (grey line) of PRR5 in Cannabis for photosensitive genotype.

FIG. 29B is a graph showing qPCR expression levels at specific timepoints over a 24-hour period under LD (black line) or SD (grey line) of PRR5 in Cannabis for autoflowering genotype.

FIG. 30A is a graph showing qPCR expression levels at specific timepoints over a 24-hour period under LD (black line) or SD (grey line) of PRR9 in Cannabis for photosensitive genotype.

FIG. 30B is a graph showing qPCR expression levels at specific timepoints over a 24-hour period under LD (black line) or SD (grey line) of PRR9 in Cannabis for autoflowering genotype.

### DEFINITIONS

“Cannabis” as used herein is inclusive of all species and varieties falling within the genus Cannabis, whether Cannabis sativa, Cannabis indica, or Cannabis ruderalis, and whether or not the variety is “drug” (i.e., contains appreciable levels of the psychoactive cannabinoid tetrahydrocannabinol, “THC”) or “non-drug”(i.e., contains less than about 0.2% or 0.3% by dry weight of THC, e.g., “hemp”). All Cannabis genomes have 10 chromosomes, including the sex chromosome. However, different cannabis genome assemblies use different chromosome numbering systems. As such, equivalent or homologous chromosomes may be referred to with different numbers. For example, the Purple Kush chromosome 5 (identified by GenBank as CM010796.2) is equivalent or homologous to CBDRx chromosome 1 (identified by NCBI as NC_044371.1) with the only difference that the sequence is inverted. Some of the analysis discussed herein was performed in CM010796.2 and some in NC_044371.1 as the reference. These two chromosomes are homologous and the results and conclusions derived from using one genome as a reference does not affect or change the results and conclusions derived from the other reference genome.

“Photoperiod” as used herein refers to the day length to which a plant is exposed. Many flowering plants, including Cannabis, sense seasonal changes in day length, i.e., photoperiod, which they may take as a signal to flower. For example, most Cannabis varieties in cultivation initiate flowering upon a transition from long days (long day length) to short days (short day length). The skilled person understands that the specific length of the “long days” or the “short days” is not crucial. Rather it is the change in the day length that is important to the initiation of flowering.

An “autoflowering phenotype” or “day length neutral phenotype” as used herein describes Cannabis plants that initiate flowering with developmental age regardless of the photoperiod (or change therein) to which the plants are exposed.

A “photoperiod phenotype”, “day length sensitive phenotype”, “photoperiod sensitive phenotype”, or “photosensitive phenotype” as used herein describes Cannabis plants that initiate flowering with the onset of decreasing day length (i.e., reduced photoperiod).

“Long days” (“LD”) as used herein refers to day lengths of between about 16 h and about 18 h (i.e., between 6 h and 8 h of darkness).

“Short days” (“SD”) as used herein refers to day lengths of about 12 h (i.e., about 12 of darkness).

“Genetic material” as used herein includes any nucleic acid, but particularly deoxyribonucleic acid (DNA) in double-stranded form.

A “polymorphic site” or “polymorphism” as used herein refers to a position within a given genomic region at which variation exists within a population. A “polymorphic site” or “polymorphism” is the occurrence of two or more forms (alleles) at a position in the genome within a population, in such frequencies that the presence of the rarest of the forms cannot be explained by mutation alone. Polymorphic sites have at least two alleles, each occurring at a frequency of greater than 1%. Polymorphic sites may occur in both coding regions and noncoding regions of genes or in intergenic regions. Polymorphic sites may involve a single nucleotide polymorphism (i.e., a SNP), or may involve an insertion or deletion (“indel”), or rearrangement.

A “single nucleotide polymorphism” (“SNP”) as used herein refers to a polymorphism that involves a variation at a single nucleotide position, whether it be an insertion, a deletion, or a substitution.

A “causative polymorphism” as used herein refers to a variation at a polymorphic site that produces an alteration in the expression of a gene product (whether at the transcriptional, translational, or protein product level) to result in or contribute to a relevant phenotype. Thus, a causative polymorphism is the most predictive of a phenotype.

“Causative” as used herein means resulting in or contributing to a relevant phenotype.

“Linkage” as used herein refers to the co-inheritance of alleles at two or more genes or sequences due to the proximity of the genes on the same chromosome.

“Linkage disequilibrium” as used herein refers to any deviation from the expected frequency, i.e., if they were segregating completely independently, of the alleles of two genes in a population. Linkage disequilibrium is defined in the context of the relative frequency of gamete types in a population of many individuals in a single generation, and is discussed extensively, for example, in WO2009/123396. Loci that have a high degree of linkage disequilibrium with an allele of interest are potentially useful in predicting the presence of the allele of interest (i.e., associated with the condition or side effect of interest). Accordingly, two polymorphisms that have a high degree of linkage disequilibrium may be equally useful in determining the identity of the allele of interest. Accordingly, the determination of the allele at a polymorphic site can provide the identity of the allele at any polymorphic site in linkage disequilibrium therewith. The higher the degree of linkage disequilibrium, the more likely that two polymorphisms may reliably function as surrogates for each other. Linkage disequilibrium may be useful for genotype-phenotype association studies. Linkage disequilibrium can be helpful in identifying a range of polymorphisms that may be commercially useful for predicting the day length neutral phenotype of a variety.

“Haplotype” as used herein refers to a set of alleles of closely linked loci on a chromosome that tend to be inherited together. Haplotypes along a given segment of a chromosome are generally transmitted to progeny together unless there has been a recombination event. In the absence of a recombination event, haplotypes can be treated as alleles at a single highly polymorphic site for mapping.

The term “traditional breeding techniques” as used herein includes crossing, selfing, selection, backcrossing, marker assisted breeding/selection, mutation breeding etc. as known to the breeder (i.e., methods other than genetic modification/transformation/transgenic methods), by which, for example, a genetically heritable trait can be transferred from one Cannabis cultivar or variety to another.

“Backcrossing” as used herein is a traditional breeding technique used to introduce a trait into a plant cultivar or variety. A first parent containing a trait of interest is crossed to a second parent to produce progeny plants. Progeny plants which have the trait are then “backcrossed” to one or the other parent, usually the second parent. After several generations of backcrossing and/or selfing, the progeny typically have the genotype of the second parent but with the trait of interest from the first parent.

“Concentrate” as used herein refer to a product derived from Cannabis flowers, whereby excess plant material and other impurities have been removed to leave primarily the cannabinoids and/or terpenes.

“Plant material” as used herein refers to any portion or combination of portions of a Cannabis plant including but not limited to seeds, stems, stalks, leaves, flowers, roots, whether fresh or dry, or whether intact, cut, or comminuted.

A “field” or a “crop” of plants as used herein refers to a plurality of Cannabis plants cultivated together in close proximity.

An “endogenous” gene as referred to herein refers to a gene that is naturally present in a population of Cannabis plants and has not been introduced through genetic modification. An “endogenous” gene may be distinguished from a second copy of the gene that is introduced by, for example, genetic modification, and exists at a separate locus in the genome. For the purposes of this disclosure, unless context dictates otherwise, the terms “endogenous PRR7 gene”, “Cannabis PRR7 gene”, and “PRR7 gene” are used interchangeably.

A “genetic modification” as used herein broadly refers to any novel combination of genetic material obtained with techniques of modern biotechnology. Genetic modifications include, but are not limited to, “transgenes” in which the genetic material has been altered by the insertion of exogenous genetic material. However, genetic modifications also include alterations (e.g., insertions, deletions, or substitutions) in endogenous genes introduced in a targeted manner with techniques such as CRISPR/Cas9, TALENS, etc. as discussed elsewhere herein. However, for the purposes of this disclosure “genetic modification” is not intended to include novel combinations of genetic material resulting from mutations generated by traditional means of random mutagenesis followed by traditional means of breeding. Genetic modifications may be transient or stably inherited.

A “genetically modified” plant or plant cell as used herein broadly refers to any plant or plant cell that possesses a genetic modification as defined herein.

“Nucleotide sequence”, “polynucleotide sequence”, “nucleic acid” or “nucleic acid molecule” as used herein refers to a DNA or RNA polymer which can be single or double stranded and optionally contains synthetic, non-natural, or altered nucleotide bases capable of incorporation into the DNA or RNA polymer.

A “fragment”, a “fragment thereof”, “gene fragment” or a “gene fragment thereof” as used herein refers to a portion of a “nucleotide sequence”, “polynucleotide sequence”, “nucleic acid” or “nucleic acid molecule” e.g., the PRR7 gene sequence. For example, a “fragment” as used herein with respect to a certain gene sequence refers to a portion of the gene sequence that is less than the complete sequence. In various embodiments, the fragment may be useful to reduce expression of a gene of interest, e.g., the PRR7 gene. In various embodiments, the fragment comprises at least 20, at least 40, at least 60, at least 80, at least 100, at least 150, at least 200, at least 250, at least 300, at least 350, at least 400, at least 450 or at least 500 contiguous nucleotides.

“Allele” or “variant” as used herein refers to a nucleotide sequence at a polymorphic site where at least two nucleotide sequences exist at the polymorphic site in a population at appreciable frequencies.

A “non-natural variant” as used herein refers to nucleic acid sequences native to an organism but comprising modifications to one or more of its nucleotides introduced by mutagenesis (including point mutations, insertions, and deletions).

“Identity” as used herein refers to sequence similarity between two polynucleotide molecules. Identity can be determined by comparing each position in the aligned sequences. A degree of identity between nucleic acid sequences is a function of the number of identical or matching amino acids or nucleic acids at positions shared by the sequences over a specified region.

“Heterologous” or “exogenous” as used herein refers to DNA that does not occur naturally as part of the plant's genome or is not normally found in the host genome in an identical context.

“Transgene” as used herein refers to a segment of DNA containing a gene sequence that has been isolated from one organism and introduced into a different organism. In the context of the present disclosure, the nucleic acid molecules may comprise nucleic acid that is heterologous to the plant in which gene expression, e.g., PRR7 gene expression, is reduced.

“Expression” or “expressing” as used herein refers to the process by which information from a gene is used in the synthesis of a functional gene product, and may relate to production of any detectable level of a product, or activity of a product, encoded by a gene. Gene expression may be modulated (i.e., initiated, increased, decreased, terminated, maintained, or precluded) at many levels including transcription, RNA processing, translation, post-translational modification, protein degradation. In the context of the present disclosure, reduced expression of an endogenous Cannabis gene, e.g., PRR7 gene, can be affected by reduced transcription of the endogenous Cannabis gene, by reduced translation of mRNA transcripts, or by the introduction of mutations that either prevent the translation of functional polypeptides or result in the translation of polypeptides with reduced abilities to convert substrate. Such reduced expression of an endogenous Cannabis gene may result from expression of transgenes comprising expression constructs designed to reduce expression of the endogenous genes.

“Decreasing expression”, “decreasing activity”, “reducing expression”, and “reducing activity” as used herein are intended to encompass well known equivalent terms regarding expression and activity such as “inhibiting”, “down-regulating”, “knocking out”, “silencing”, etc.

“Expression construct” as used herein refers to any type of genetic construct containing a nucleic acid coding for a gene product in which part or all the nucleic acid encoding sequence is capable of being transcribed. The transcript may be translated into a protein, but it need not be. An expression construct of the disclosed nucleic acid molecule may further comprise a promoter and other regulatory elements, for example, an enhancer, a silencer, a polyadenylation site, a transcription terminator, a selectable marker or a screenable marker.

“Promoter” as used herein refers to a nucleotide sequence that directs the initiation and rate of transcription of a coding sequence. The skilled person will understand that it would be important to use a promoter that effectively directs the expression of the construct in the tissue in which a Cannabis gene, e.g., PRR7, is usually expressed. For example, endogenous PRR7 gene promoters could be used. Alternatively, constitutive, tissue-specific, or inducible promoters useful under the appropriate conditions to direct high-level expression of the introduced expression construct could be used.

“Constitutive promoter” as used herein refers to a promoter which drives the expression of the downstream-located coding region in all or nearly all tissues regardless of environmental or developmental factors.

### DETAILED DESCRIPTION

### Initiation of Flowering in Response to Day Length

Many Cannabis varieties in production today have a day length sensitive phenotype. Photoperiod sensitivity is especially limiting for crops grown outdoors at more northern and southern latitudes. Near the equator, the variation of temperature and light (e.g., 12 hours of sun and 12 hours of darkness per day) is minimal, such that the plants do not experience a change in the photoperiod, and they flower when the plant reaches maturity. However, at more northerly and southerly latitudes, Cannabis is effectively a seasonal plant that needs favourable temperature conditions to grow, and light conditions to flower. Since flowering can be triggered by a change (i.e., a decrease) in day length (i.e., photoperiod), growing conditions when flowering is triggered may be less than optimal for harvest. For example, a sufficient decrease in day length to initiate flowering may not occur until late summer or early fall when frost may occur impacting a plant's quality and/or yield.

In contrast, day length neutral (i.e., “autoflowering”) plants can be used at more extreme latitudes since they do not depend on photoperiod to flower. Such plants flower automatically with age, typically have short lifecycles, and, therefore, may mature before the end of the outdoor season yielding a better harvest index. In other words, autoflowering plants can be planted and harvested in a manner that takes advantage of the best growing conditions at such extreme latitudes.

Methods for selecting for day length neutral (“autoflowering”) plants are thus needed to develop varieties for more northern/southern latitudes so that plants can be cultivated outdoors and thereby avoid the costs and resources required by indoor cultivation (e.g., in terms of energy consumption, space, and labor that is required for growth in a greenhouse). The suitability of a variety for cultivation in a wide range of conditions may contribute to high productivity.

Day length neutral phenotypes are also valuable for growth in greenhouses at more extreme latitudes. Shade curtains are required in greenhouses to control the amount of light to which a crop is exposed each day in order to avoid initiation of flowering prematurely, or to simulate a decrease in day length if the initiation of flowering is desired. The cultivation of plants with day length neutral phenotypes avoids the need for such shade curtains.

Thus, breeding Cannabis varieties with day length neutral (“autoflowering”) phenotypes has significant economic value. Plants with polymorphisms that are predictive of the day length neutral phenotype can be genotyped early in the life of the plant, thereby allowing for the selection of plants desired for further breeding long before the onset for flowering, and for the culling of photoperiod phenotype plants that do not carry the autoflowering trait.

Thus, there is a need for novel genetic markers that are predictive of the day length neutral phenotype. The identification of such genetic markers associated with autoflowering may also reveal novel mutations that are causative of the day length neutral phenotype, and thus identify novel gene targets for the manipulation of response to photoperiod.

### Polymorphisms

The genomes of all organisms undergo spontaneous mutations in the course of their evolution to generate variant forms of progenitor genetic sequences, whether they be substitutions of one nucleotide for another at the polymorphic site, or insertions or deletions of as few as one nucleotide. In many cases, both progenitor and variant forms survive and co-exist in a species population.

The coexistence of multiple forms of a genetic sequence segregating at appreciable frequencies is defined as a “genetic polymorphism” at a “polymorphic site”, which includes single nucleotide polymorphisms (“SNPs”), i.e., single base positions in DNA at which different alleles, or alternative nucleotides, exist in a population.

Polymorphic sites may have several alleles. However, the vast majority of polymorphic sites are bi-allelic.

Regardless of whether the polymorphism involves a SNP, an insertion, or a deletion, the polymorphic site is usually preceded and followed by highly conserved “context” sequences (e.g., sequences that vary in less than 1/100 or 1/1000 members of the populations). An individual may be homozygous or heterozygous for an allele at each polymorphic site.

Causative polymorphisms may typically be positioned within genes encoding a polypeptide product, for example, SNPs that result in changes in the amino acid sequence that result in a defective product. However, causative polymorphisms do not necessarily have to occur in coding regions, and can occur in any region that affects the expression of the protein encoded by a gene, whether by transcription or translation.

Some polymorphisms that are not causative polymorphisms nevertheless are in linkage disequilibrium with, and therefore segregate with, the phenotype of concern. Thus, such polymorphisms may be commercially useful for predicting the phenotype.

An association study of a polymorphism and a specific phenotype involves determining the presence or frequency of the polymorphic allele in biological samples from individual plants with the phenotype of interest, such as a day length neutral phenotype, and comparing the information to that of controls (e.g., individual plants that have a day length sensitive phenotype).

A polymorphic site may be surveyed in a sample obtained from an individual having a day length neutral phenotype, compared to control (i.e., day length sensitive plant) samples, and selected for its increased (or decreased) prevalence. Once a statistically significant association is established between one or more polymorphisms and the phenotype of interest, e.g., day length neutral, then the genomic region around the polymorphic site(s) can be thoroughly screened to identify the causative polymorphisms or additional polymorphic sites in linkage disequilibrium with the identified polymorphic sites.

### Polymorphism Analysis

Once an individual plant is identified as a candidate for having a day length neutral phenotype, or being a carrier of a day length neutral allele, then genetic sequence information may be obtained from the plant for the purpose of determining the identity of the alleles at one or more polymorphic sites associated with the day length neutral phenotype. Genetic sequence information may be obtained in numerous different ways and may involve the collection of a biological sample that contains genetic material, particularly, genomic DNA containing the sequence or sequences of interest. Any suitable genomic DNA samples from Cannabis may be used, e.g., from seeds, seedlings, tissue cultures or plants of any age, preferably before flowering has been initiated. Many methods are known in the art for collecting biological samples and extracting genetic material from those samples, as surveyed, for example, in WO 2009/124396 and WO2016/197258. As discussed in WO2019/222835, samples may include the leaves of Cannabis seedlings or young plants pressed into a paper matrix, such as a paper matrix (e.g., Whatman™ FTA™ cards) comprising a mixture of chemicals that lyse cells and stabilize nucleic acids on contact for long-term storage at room temperature. The ability to use leaf material pressed into a paper matrix is particularly advantageous because the samples are easily collected, stored at room temperature, and shipped in a format that is not subject to the same restrictions as handling of controlled or regulated substances. Alternatively, the samples may comprise purified DNA isolated from fresh or dry Cannabis plant material.

Once an individual plant's genomic DNA has been obtained, the identity of the allele at one or more of the polymorphic sites associated with the day length neutral phenotype can be determined using any one of several methods known in the art. A variety of methods for determining the sequence at polymorphic sites are discussed in, for example, WO 2009/124396, WO2011/133418, WO2016/197258, and WO2019/222835.

Detection or determination of a nucleotide identity, or the presence of one or more SNP(s), may be accomplished by any one of a number of methods or assays known in the art. Many methodologies are useful for determining the identity of alleles at polymorphic sites, and can be assigned to one of four broad groups (sequence-specific hybridization, primer extension, oligonucleotide ligation and invasive cleavage). Furthermore, there are numerous methods for detecting the products of each type of reaction (e.g., fluorescence, luminescence, etc.).

In one popular method discussed in WO2019/222835, High Resolution Melt (HRM) analysis may be conducted in the presence of a common intercalating fluorescent dye to determine the identity of the alleles present at a polymorphic site. As double stranded DNA denatures during HRM, the loss in fluorescence from the dye over time provides different denaturation curves depending on the which alleles are present at the polymorphic site.

In another favored method, 5′ exonuclease activity or TaqMan™ assay (Applied Biosystems) displaces and cleaves the oligonucleotide probes hybridized to a specific target DNA generating to fluorescent signal.

The skilled person understands that numerous methods for determining the identity of an allele at a polymorphic site are known in the art. The skilled person understands that the particular method of determining the identity of the allele at the polymorphic site is not important, so long as it may be reliably determined.

### Marker Assisted Breeding

Breeding new varieties, lines and hybrids may be achieved using techniques of crossing and selection on a set of parental lines taking advantage of the plant's method of pollination (self-, sib- or cross-pollination). Multiple rounds of crossing and selection may be needed to produce plant varieties with the desired traits. After each round, the breeder selects candidates with the desired traits or markers for the next round of selection.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select for the allele of the desired trait (e.g., day length neutral phenotype) and against the alleles of the undesired trait (e.g., day length sensitive phenotype). The use of this procedure can reduce the number of back crosses to the desired parent in a backcrossing program.

The skilled person understands that other desired traits (e.g., altered height, early or late maturity, disease resistance, cannabinoid and/or terpene content etc.) can be transferred into selected autoflowering lines by cross pollinating plants having a day length neutral (autoflowering) phenotype with a second parent plant having the desired traits, collecting F₁ seed, growing a F₁ plant which is allowed to self-pollinate and collect the F₂ seed. The F₂ seed would then be grown, and individual plants that have the day length neutral phenotype could be selected according to the methods herein. The skilled person will be able to backcross the selected individuals to the second parent.

### Low Recombination Region

F₂ plant populations from F₁ self-pollinated plants resulting from an initial cross between autoflowering and photoperiod sensitive parents were shown to segregate in 1:3 ratio day length neutral phenotype:photoperiod phenotype, which strongly indicates that the day length neutral phenotype is a recessive trait and controlled by a single locus.

A genetic map from an F₂ population segregating the day length neutral phenotype was constructed which revealed a low recombination region of 20 megabases (Mb) in Cannabis chromosome 5 (identified by GenBank Sequence as CM010796.2) associated with the day length neutral phenotype.

An F2 mapping population was made by crossing a photosensitive plant (MOT19P06 (Tangerine Dream or Rex™) and an autoflowering plant (BAT1804 (Blue Dream Auto)) and selfing a single F1 plant. A total of 192 F2 seeds were germinated and flowered for 8 weeks under short days for chemotyping and phenotyping. Flowering photoperiod requirement was not evaluated. Leaf samples were collected from the founding parents and F2 plants for DNA isolation, genome sequencing and genotyping.

The genomes of the two parents (MOT19P06 and BAT1804) were sequenced with a combination of Illumina short read and PacBio long read technologies. Genomic scaffolds were de novo assembled and mapped to the CS10_v2 reference assembly to construct pseudo chromosome molecules. Genotyping of all 192 plants in the F₂ population was done by WGS.

The MOT19P06 assembly was used as a reference genome. All samples were mapped to the reference genome followed by a Variant-Calling pipeline using GATK to process the Skim-Seq data optimally (done by NRGene). Variant filtration included several steps with the following parameters: Step 1: Selection of biallelic SNPs only, SNP is unique to one of the F0 lines (BAT1804 or MOT19P06), and at least 75 bp of clean flanking region with no other variant. The number of SNPs after step one was 142,956. Step 2: Selection of SNPs showing a reasonable segregation in the pedigree (evidence for both homozygous and heterozygous samples). The number of SNPs after Step 2 was 66,728.

After variant filtration, all SNPs from Step 2 were selected for the next stage. The Variant-Calling procedure was followed by a haplotype-inference algorithm that infers the two haplotypes in the F1 generation. The segregating genotypes in the progeny were inferred for each sample at each location along the genome. The three possible genotypes were designated as follows: AA, AB, and BB. The basic genotyping unit is the haplotype-block (HB), defined as a segment between consecutive recombination events in any of the progeny samples. Within haplotype blocks, there are no recombination events, and all markers (SNPs) could be used to measure sample genotypes. Genetic distances were calculated from the observed recombination fractions in the F2 population using the Kosambi function.

A diversity panel of 12 unrelated photosensitive and 12 unrelated autoflowering genotypes was selected from the germplasm and used for whole genome shotgun sequencing at low coverage. Autoflowering versus photoperiod sensitive genotypes were confirmed by keeping plants under long days and monitoring the appearance of flower structures.

WGS data from 12 unrelated photoperiod sensitive genotypes, and 12 autoflowering genotypes was used for comparative population genetics analysis on Chromosome 1. 391K variants were identified from WGS data. Fixation index (F_{ST}) values were calculated to compare allele frequency differences between the two groups. Analyses were performed using a sliding window of 5,000 base pairs and variants contained within, with a step size of 1,000 base pairs across the chromosome. Nucleotide diversity (Pi) was calculated to compare the average number of nucleotide differences between all possible pairs within the 12 autoflowering and 12 photoperiod genotypes.

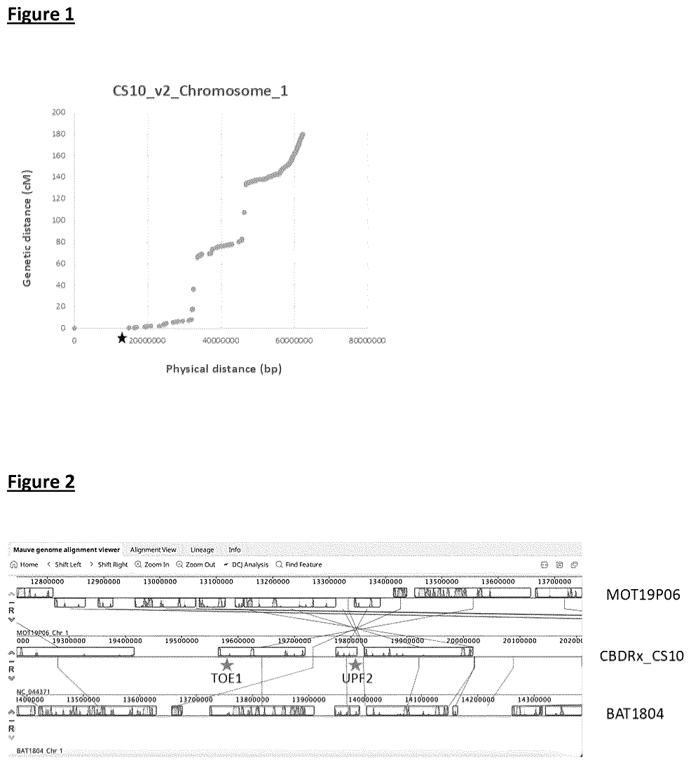

Recombination frequencies in the F2 mapping population showed spikes with abnormally low or high values in 7 out of 10 chromosomes in the cannabis genome (data not shown). Abnormal recombination frequencies could be explained by miss-assembled regions in the genome or, alternatively, by structural variations (SVs) that cause highly divergent regions in the genomes that would drastically reduce homologous recombination. The relationship between genetic and physical distances in chromosome 1 was analyzed, where the UPF2 autoflowering marker is located, and observed a 20 Mb region at the beginning of the chromosome with very low recombination (FIG. 1, see low recombination region between 0-20,000,000 bp, star denotes the UPF2 gene).

The 20 Mb low recombination region in chromosome 1 is composed of 6 haplotype blocks that span only 1.3 cM. Haplotype block 1 was the largest (0-14,915,027 bp) and was characterized by an absolute lack recombination in the F2 population. Haplotype blocks 2-6 covered from 14,915,027 to 20,861,923 bp and also had a very low frequency of recombination.

Using Mauve, the entire sequence of chromosome 1 from MOT19P06 and BAT1804 was aligned to CS10 to investigate possible mechanisms that could be causing the low recombination regions in our F2 population. The alignment revealed several sequence inversions and translocations (FIG. 2, see blocks on the lower side of MOT19P06 chromosome 1 and crossing lines). UPF2 and TOE1 are located in a region highly disrupted by inversions and translocations. Without wishing to be bound by theory, the presence of SVs likely explains the low recombination frequency observed in the first 20 Mb of chromosome 1.

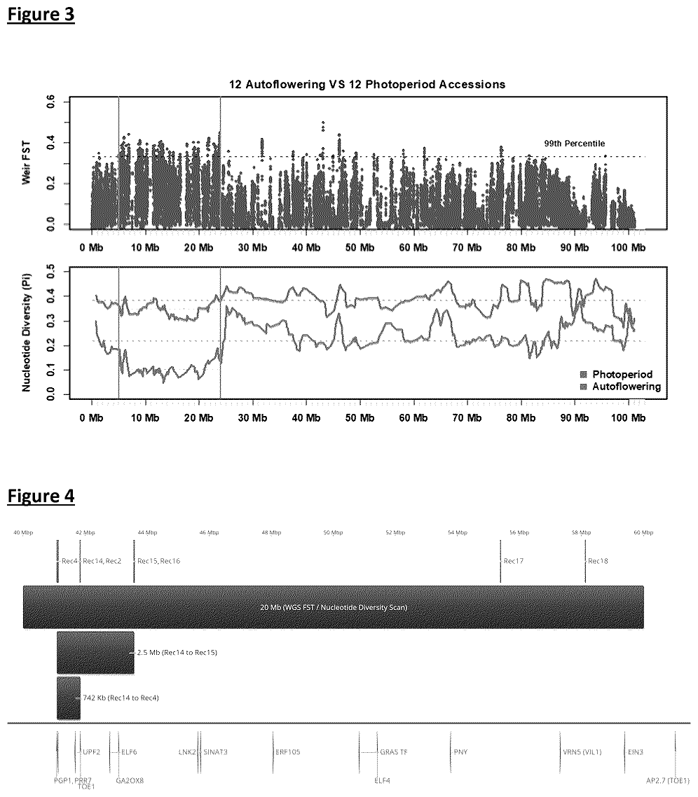

The presence of a low recombination region in chromosome 1 where the autoflowering locus is located should result in fixed alleles in this region for the two sub-populations of autoflowering and photosensitive accessions. The fixation index (F_{ST}) was calculated to compare allele frequency differences (e.g., localized population structure) between 12 unrelated autoflowering and 12 photoperiod accessions. An F_{ST} value of 0 implies no observable difference in allele frequency in each window of variants between the two groups, whereas a value of 1 implies that observed genetic variation is explained entirely by population structure, and that each group is “fixed” for the different observed alleles in that region. The 99th percentile of F_{ST} values observed across all windows (FIG. 3, top panel, F_{ST}=0.36 denoted with a dashed line), suggested that the region between 5 and 24 Mb shows a significant difference in allele frequency between autoflowering and photosensitive genotypes. Of the 395 windows greater than or equal to the 99th percentile of all windows, 313 windows (79.2%) are located between 5 and 24 Mb.

Enrichment of fixed alleles in the 20 Mb region were found in at least 12 unrelated autoflowering genotypes other than BAT1804. This suggests the genetic underpinning of the autoflowering trait is located in a 20 Mb region that has undergone very little recombination with photosensitive genotypes, and that this region is not exclusive to the BAT1804×MOT19P06 mapping population.

Nucleotide diversity (Pi) was calculated to compare the average number of nucleotide differences between all possible pairs within 12 autoflowering and 12 photoperiod accessions within 5K variant windows across the chromosome. The mean nucleotide diversity of all windows spanning the chromosome was 0.385 for Photoperiod accessions and 0.220 for Autoflowering accessions. However, the mean nucleotide diversity of windows located between 5 and 24 Mb was 0.349 for Photoperiod accessions (decrease of 9.35% relative to entire chromosome), and 0.103 for Autoflowering accessions (decrease of 53.18% relative to entire chromosome), strongly supporting a genomic region wherein a lack of admixture between autoflowering and photoperiod germplasm has preserved genetic structure between the two populations.

Further crossing experiments provided evidence for a significantly reduced linkage region with a minimum size of about 0.75 Mb to a maximum size of about 2.5 Mb within the 20 Mb region (FIG. 4). Seven KASP markers were used to test if the 20 Mb linkage region had recombined resulting in a smaller introgression in the progeny of an F₂ population resulting from the cross of an auto-flowering and a photoperiod sensitive parents. A total of 1,106 seeds were germinated and genotyped with KASP markers. The autoflowering phenotype was confirmed by growing the plants under long days and scoring the development of flowers.

In some embodiments, the low recombination region in chromosome CM010796.2 that may be attributable to the autoflowering trait is about 1 megabase, about 2.5 megabases, about 5 megabases, about 7.5 megabases, about 10 megabases, about 12.5 megabases, about 15 megabases, about 17.5 megabases or about 20 megabases located between about 40 megabases to about 60 megabases. In some embodiments, the low recombination region is located within a region in Cannabis chromosome CM010796.2 selected from: about 40 megabases to about 42.5 megabases, about 40 megabases to about 45 megabases, about 40 megabases to about 50 megabases, about 42.5 megabases to about 45 megabases, about 42.5 megabases to about 47.5 megabases, about 42.5 megabases to about 50 megabases, about 42.5 megabases to about 52.5 megabases, about 45 megabases to about 47.5 megabases, about 45 megabases to about 50 megabases, about 45 megabases to about 52.5 megabases, about 45 megabases to about 55 megabases, about 47.5 megabases to about 50 megabases, about 47.5 megabases to about 52.5 megabases, about 47.5 megabases to about 55 megabases, about 47.5 megabases to about 57.5 megabases, about 50 megabases to about 52.5 megabases, about 50 megabases to about 55 megabases, about 50 megabases to about 57.5 megabases, about 50 megabases to about 60 megabases, about 52.5 megabases to about 55 megabases, about 52.5 megabases to about 57.5 megabases, about 52.5 megabases to about 60 megabases, about 55 megabases to about 57.5 megabases, about 55 megabases to about 60 megabases, or about 57.5 megabases and about 60 megabases.

In some embodiments, the low recombination region in chromosome NC_044371.1 that may be attributable to the autoflowering trait is about 1 megabase, about 2.5 megabases, about 5 megabases, about 7.5 megabases, about 10 megabases, about 12.5 megabases, about 15 megabases, about 17.5 megabases or about 20 megabases located between about 0 megabase to about 20 megabases. In some embodiments, the low recombination region is located within a region in Cannabis chromosome NC_044371.1 selected from: about 0 megabase to about 2.5 megabases, about 0 megabase to about 5 megabases, about 0 megabase to about 7.5 megabase, about 0 megabase to about 10 megabases, about 0 megabase to about 12.5 megabases, about 0 megabase to about 15 megabases, about 0 megabase to about 17.5 megabases, about 2.5 megabases to about 5 megabases, about 2.5 megabases to about 7.5 megabases, about 2.5 megabases to about 10 megabases, about 2.5 megabases to about 12.5 megabases, about 2.5 megabases to about 15 megabases, about 2.5 megabases to about 17.5 megabases, about 2.5 megabases to about 20 megabases, about 5 megabases to about 7.5 megabases, about 5 megabases to about 10 megabases, about 5 megabases to about 12.5 megabases, about 5 megabases to about 15 megabases, about 5 megabases to about 17.5 megabases, about 5 megabases to about 20 megabases, about 7.5 megabases to about 10 megabases, about 7.5 megabases to about 12.5 megabases, about 7.5 megabases to about 15 megabases, about 7.5 megabases to about 17.5 megabases, about 7.5 megabases to about 20 megabases, about 10 megabases to about 12.5 megabases, about 10 megabases to about 15 megabases, about 10 megabases to about 17.5 megabases, about 10 megabases to about 20 megabases, about 12.5 megabases to about 15 megabases, about 12.5 megabases to about 17.5 megabases, about 12.5 megabases to about 20 megabases, about 15 megabases to about 17.5 megabases, about 15 megabases to about 20 megabases, or about 17.5 megabases to about 20 megabases.

Identification of Polymorphic Sites in the Cannabis Genome that are Associated with and May be Causing the Autoflowering (“Day Length Neutral”) Phenotype

A plurality of F₂ seeds from F₁ self-pollinated plants resulting from an initial cross between autoflowering and photoperiod sensitive parents were germinated and grown. All plants were kept under long days (16 h light/8 h dark) from sowing to 45 days. Under longs days, autoflowering plants started to flower while photoperiod plants grew vegetatively. After day 45, the light regimen was switched to short days (12 h light/12 h dark) to trigger flowering in photoperiod plants. All autoflowering plants had fully developed flowers before day 45. The skilled person understands that the switch in the light regimen from long days to short days is used to trigger flowering in photoperiod plants and that other parameters with respect to growth conditions (e.g., days under long days vs. short days, temperature, humidity, lumens, pH, solutions etc.) may be varied and/or optimized depending upon a variety of factors, such as the particular plant strain/variety/cultivar of Cannabis grown for example.

To identify plants that were autoflowering, a PCR marker that relies on a deletion in the 3′ UTR of the UPF2 gene in Cannabis chromosome 5 (identified by GenBank Sequence as CM010796.2) of autoflowering plants was used. To validate this UPF2 marker, the following primers amplifying the UPF2 gene and its UTR regions were used: GTACAGTAAACTATCTCAATTTCT (SEQ ID NO: 18) and ACCACACCTTTTCCAATTGGACTO (SEQ ID NO: 19). 1 ul DNA was amplified with 1 uM of each primer, 0.2 mM dNTP, 3 mM MgCl₂ and 2 Units Taq polymerase, using the following cycling program: 5 min at 98° C., then 30 cycles of 10 s at 98° C., 30 s at 58° C., 1 min at 72° C. and final extension 10 min at 72° C. Plants having the day length neutral phenotype showed a deletion at this site. Using the marker, 61 plants were found to be autoflowering and 170 were photoperiod sensitive in line with the expected segregation ratio of 1:3 autoflowering:photosensitive phenotype discussed above (x²<x_{c} and p=0.621).

Leaf tissue from fully expanded leaves from plants growing under long days were collected from autoflowering and photoperiod plants at 17, 20, 22, 25, 27, 30, 33, 35, 38 days after sowing. After switching to short days, leaf tissue was collected from photoperiod plants at 47, 49, 52, 54 and 56 days after sowing. Apical meristem tissue was collected from lateral branches at 20, 25, 27, 30, 45, 47, 49, 52, 54, and 56 days after sowing. Samples representative of vegetative, transition, and flowering stages were selected for RNA isolation using standards RNA isolation techniques known in the art.

Raw RNAseq reads were processed and overall library quality was assessed using Skewer and FastQC. Processed RNAseq reads were mapped to the Purple Kush assembly, NCBI accession ASM23057v4. Mapping was carried out using the Super-Reads pipeline. Super-reads were-de novo assembled using MaSuRCA, an assembly program that utilizes a k-mer index to extend RNAseq reads in both directions so long as the extension is unique. Prior to the construction of the k-mer index table, MaSuRCA uses a modified version of QuorUM to correct errors found in RNAseq reads. Assembled super-reads are subsequently mapped to the Purple Kush genome via HISAT2 v2.1.0 with the following settings: --dta, --mp 1,0, --sp 3,0, --pen-noncansplice. Mapping rates for all libraries ranged between 94 and 96%. The resulting SAM files produced by HISAT2 were converted to BAM format, merged, and sorted using Samtools v1.7.

Mapped reads were assembled into transcripts and quantified for gene expression using Stringtie v2.1.4. Two passes of Stringtie were implemented on mapped libraries. The first used Stringtie's default settings to assemble transcripts and produce gene transfer format annotation files for each library. Annotation files were subsequently merged using Stringtie's merger function. The merged annotation file was compared to the original Purple Kush assembly annotation file using GFFCompare to assess novel loci and transcript discovery. The second Stringtie pass was implemented on each library using the new merged annotation file as the reference guide. Gene expression count tables were generated using the -e and -A switches on the second Stringtie pass.

Read counts estimated with Stringtie were converted to a format suitable for DESeq2 using prepDE.py3 under default settings and an average read length of 150 bp. Counts were normalized using DESeq2 R package v. 1.30.1 and analyzed for differentially expressed (DE) genes using the default pipeline given by the function ‘DESeq’ that uses the Wald test to assess differential expression. FDR was set at 10% cut-off to define DE genes. Genes with an absolute fold-change ≥2 were classified as DE.

Analysis of DE genes was performed between the three different developmental stages (vegetative, transition, and flowering) and between autoflowering and photoperiod sensitive plants. DE analysis of leaf expression libraries identified 8,801 genes that significantly changed their expression between developmental stages or between autoflowering and photoperiod plants. Most DE genes (8,471) were identified in the comparison between autoflowering and photoperiod plants, suggesting that the flowering phenotypes are causing large differences in gene expression in leaves. A subset of 111 genes were DE between autoflowering and photoperiod plants and located in a 20 megabase low recombination region in Cannabis chromosome CM010796.2 located between about 40 megabases to about 60 megabases where the causative polymorphism for the autoflowering phenotype is located.

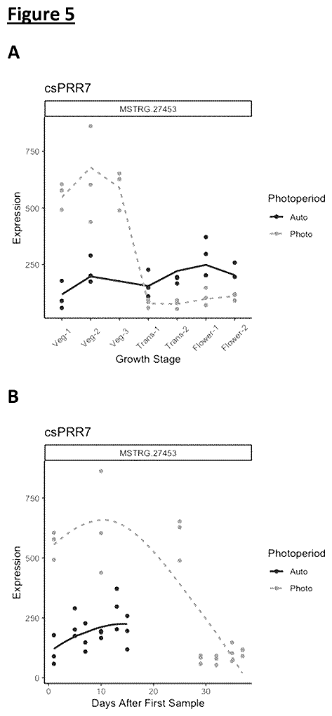

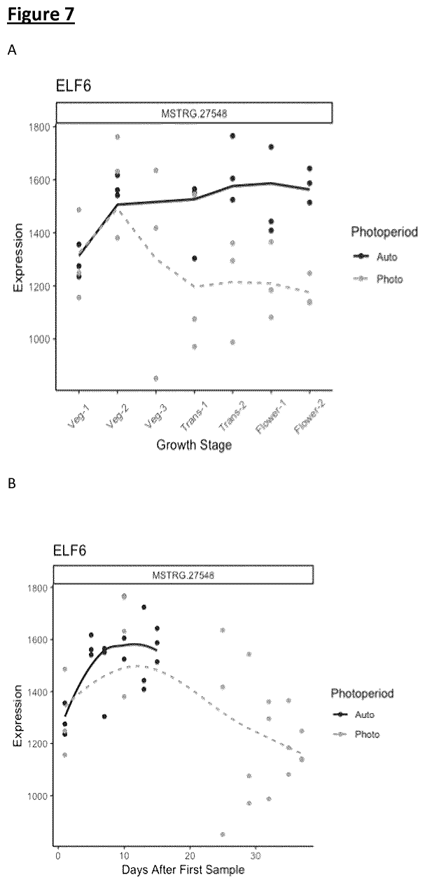

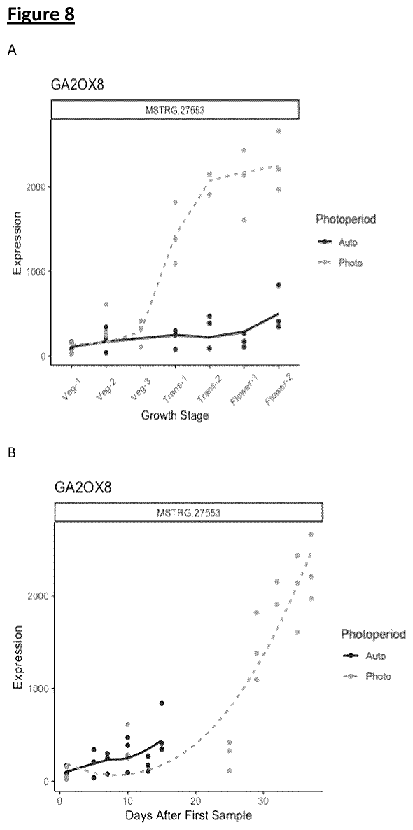

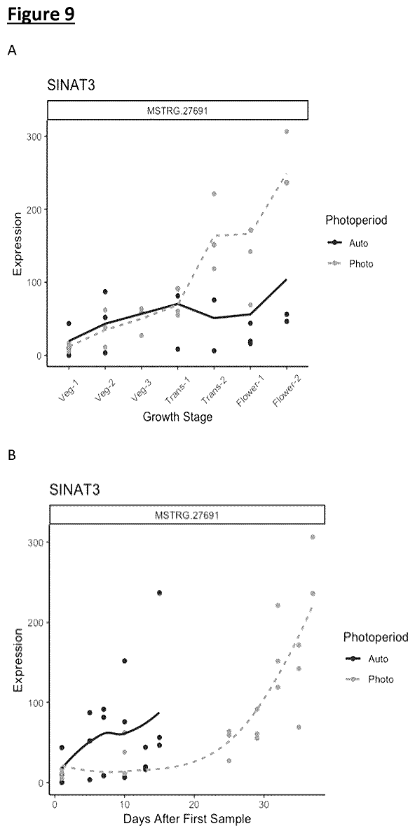

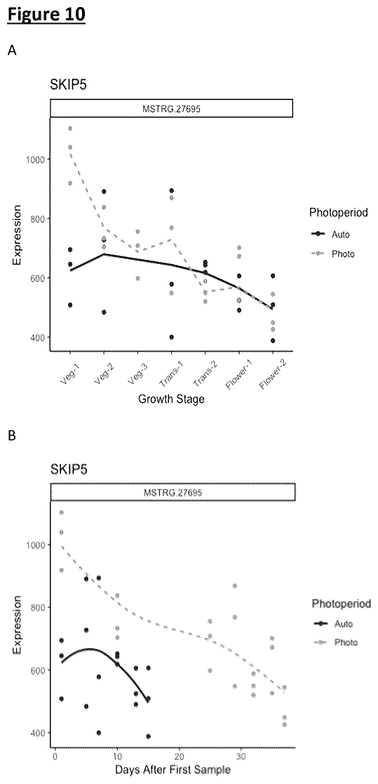

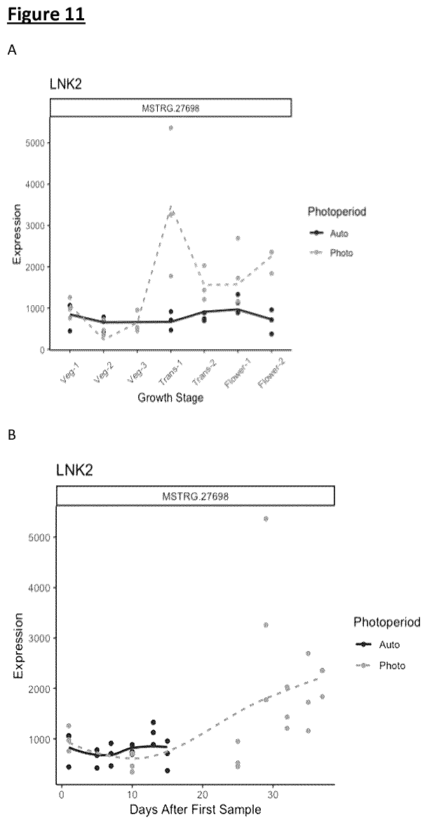

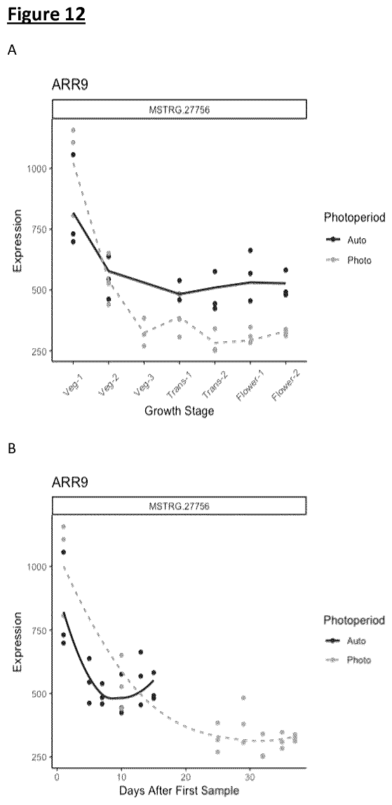

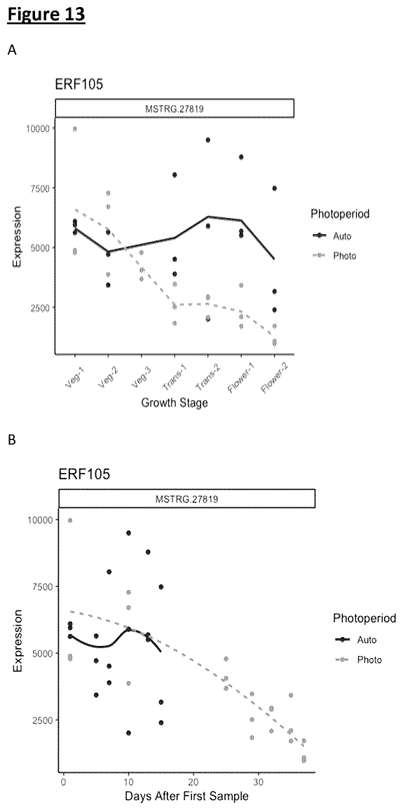

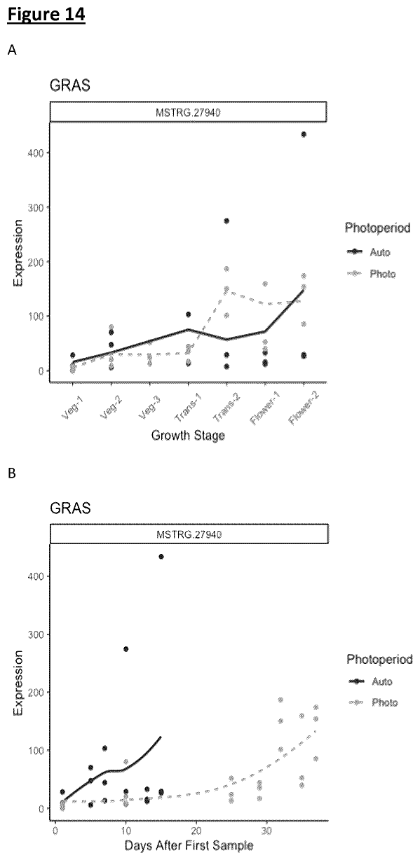

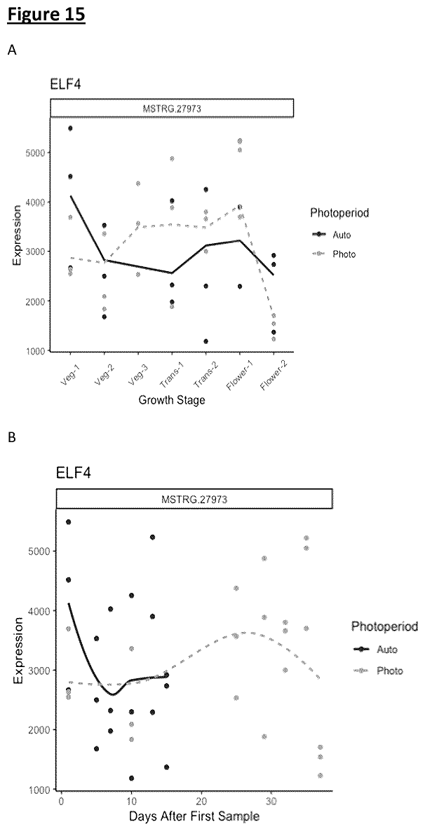

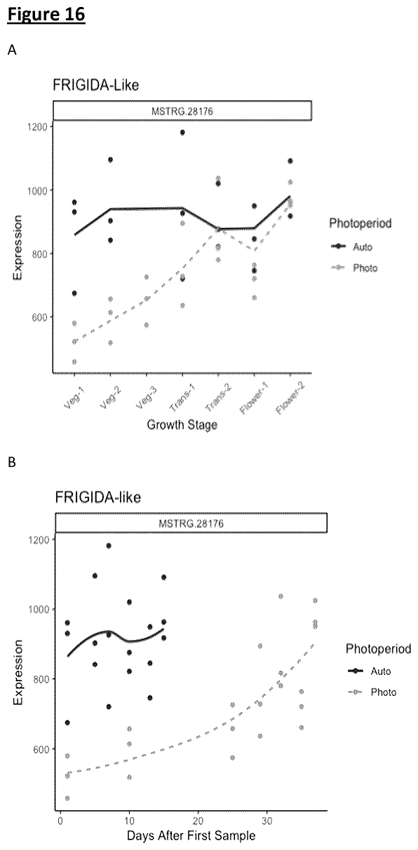

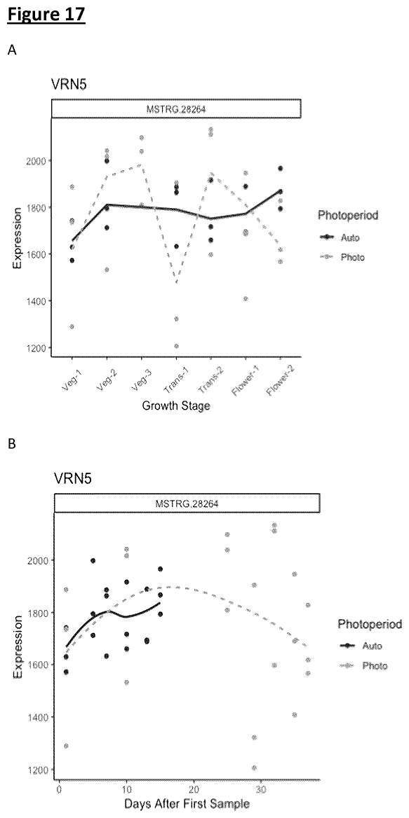

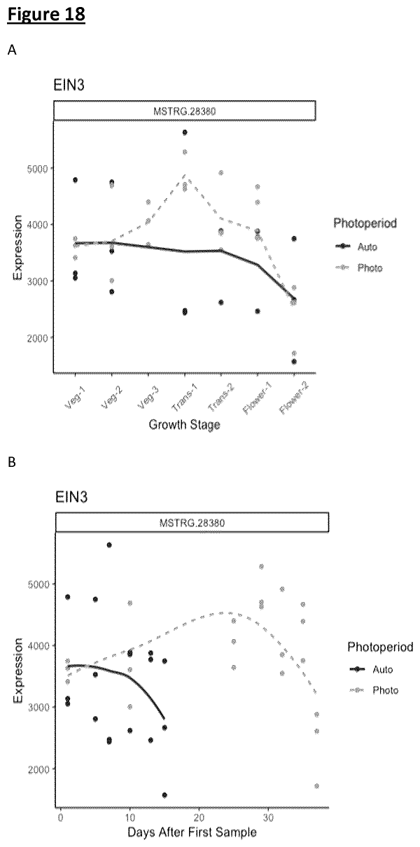

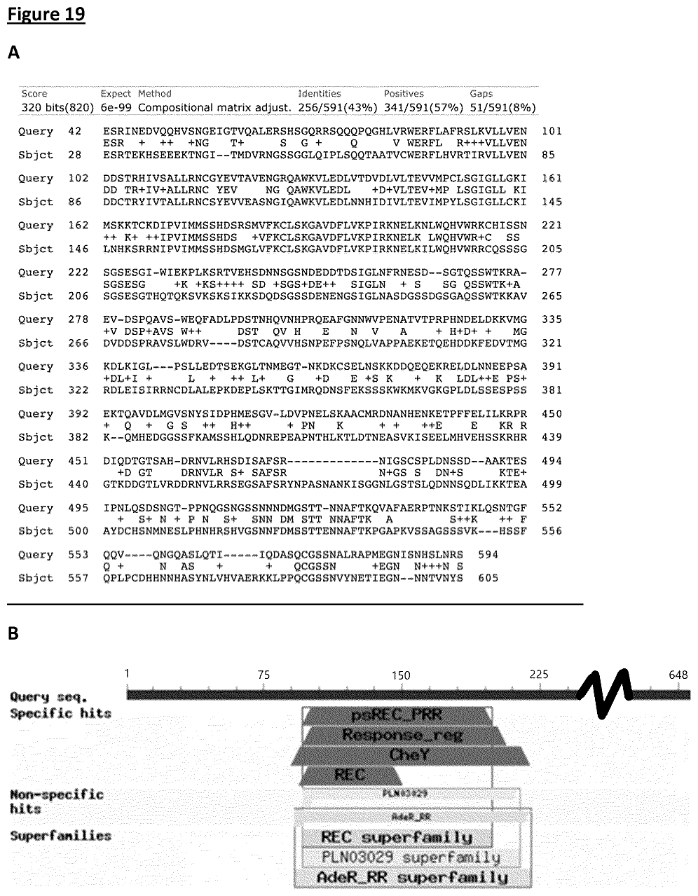

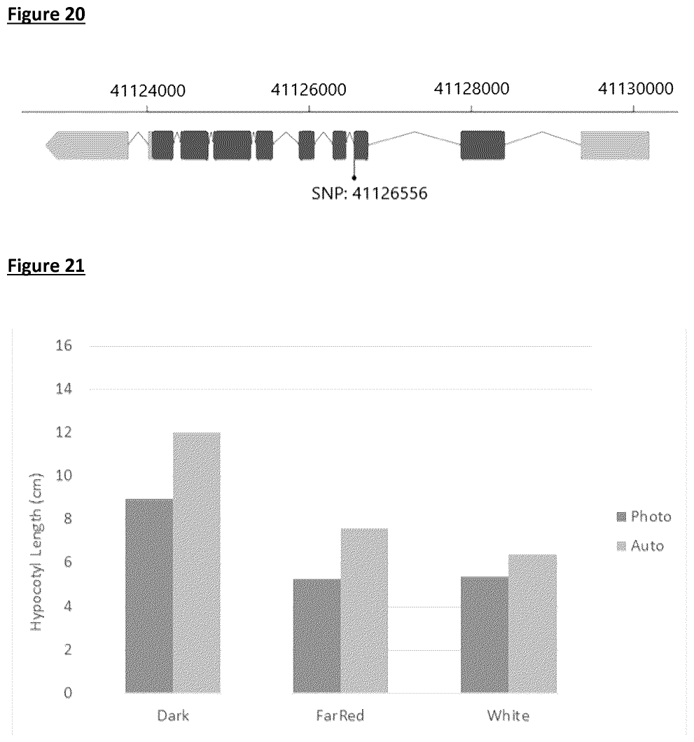

The analysis discussed above with regard to the low recombination 20 Mb linkage region and its role in the autoflowering phenotype, and an analysis of previous literature, highlights several genes associated with the autoflowering phenotype (Table 1). Of the 1172 genes contained in this region a subset of 14 genes were identified as actively expressed in both autoflowering and photoperiod sensitive plants and that have been shown in previous literature to be in some way involved in flowering in other specifics (primary Arabidopsis). These 14 flowering related genes within the 20 Mb linkage region show differential expression at various developmental stages and, in some cases, significantly altered overall expression patterns between autoflowering and photoperiod sensitive genotypes (FIGS. 5 to 18). Within the group of 14 flowering related genes, 4 genes (PRR7, TOE1, ELF6, and GA2OX8) are located within the reduced 0.75 Mb and 2.5 Mb linkage regions.

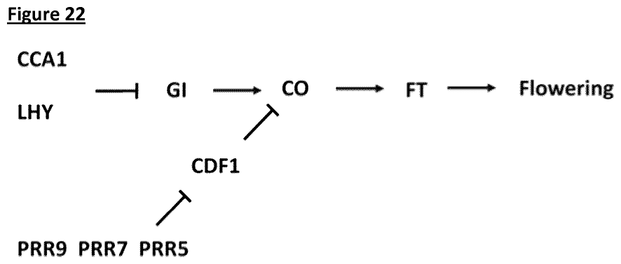

One of the genes within this region, PRR7, is annotated as homologous to Arabidopsis APRR7 (Score 320, E-value 6e-99, FIGS. 19A and B), a gene known to be involved in the circadian clock and with mutant flowering phenotypes. Plant circadian clocks are composed of many highly interconnected genes that collectively compose a self-regulating system. Genes that compose the clock display oscillating expression patterns with varying frequencies and expression amplitudes. These oscillation patterns change with changes in daylength, allowing plants to initiate flowering at appropriate times of the years. In these regards, the circadian clock acts as a mediator between environmental signals and flowering machinery.

In Arabidopsis, the circadian clock is composed of three interlocking feedback loops containing at least a dozen genes. Knock out or overexpression studies on many of these genes have led to altered oscillations of other clock genes highlighting the interconnectedness of the clock. Such disruptions often lead to changes in flowering time as well. It is well known that plant circadian clocks are well conserved among different species, reflecting their biological importance. Several homologues of Pseudo Response Regulator (PRR) genes have been identified in cannabis including PRR5, PRR7, and PRR9.

In further analysis of PRR7, alignment hits to functional domains show that the top hit is a pseudo receiver domain of pseudo-response regulators (FIG. 19B). In Arabidopsis, these domains are present in all PRR genes. Significant differential expression was observed for photoperiod and autoflowering Cannabis plants for PRR7 (FIGS. 5A and B). During vegetative growth, PRR7 was expressed 2 to 3-fold higher in photoperiod genotypes compared to autoflowering genotypes. During transition to flowering, PRR7 expression in photoperiod genotypes dropped to levels below that observed in autoflowering genotypes. In contrast, PRR7 levels in autoflowering genotypes remained constant throughout development from vegetative to flowering stages.

Significant differences in PRR7 expression in autoflowering genotypes compared to photoperiod genotypes and the location of PRR7 in the 20 Mb low recombination linkage region prompted further investigation. Analysis of the RNASeq data uncovered five SNP differences within PRR7 between autoflowering and photoperiod plants represented with genomic context sequence as SEQ ID NOs: 4 to 8, with the predictive variant present in the autoflowering genotype.

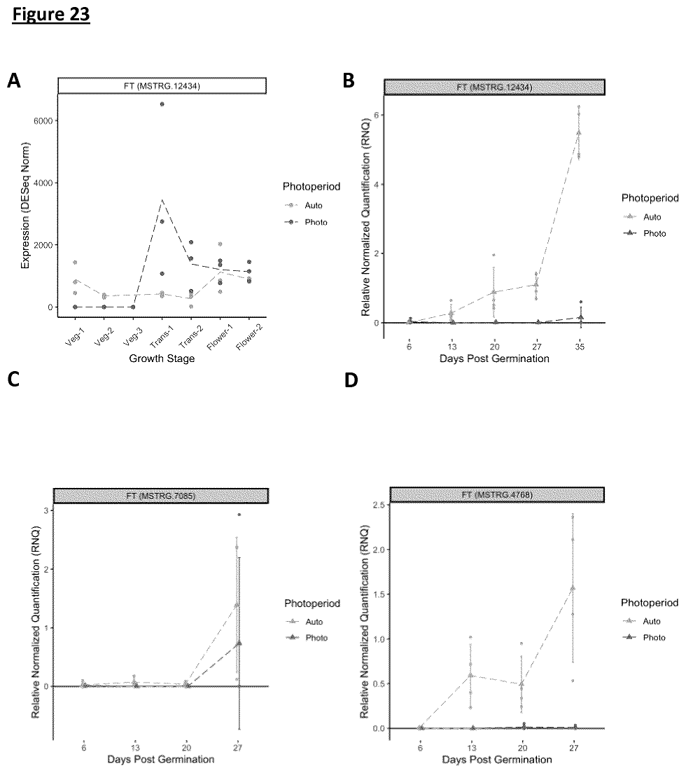

Analysis of whole genome sequencing (WGS) data from twelve autoflowering genotypes and seventeen photosensitive genotypes identified a G to T SNP variant (SEQ ID NO: 9) that exists in all libraries of the autoflowering genotypes and in none of the photosensitive libraries. This SNP is located in the splice donor site immediately after the third exon (FIG. 20). Mapped RNAseq reads spanning the third and fourth exon of PRR7 were isolated for both autoflowering (SEQ ID NO: 12) and photosensitive (SEQ ID NO: 13) genotypes and selected reads were aligned to the PRR7 genomic sequence from the Purple Kush assembly (SEQ ID NO: 11). Reads from photosensitive genotypes all showed proper splicing with no extended third exon. In contrast, reads from autoflowering genotypes all contained a third exon that incorporated 4 bp of the proceeding intron along with the T SNP variant at the intron splice donor site (Table 2). The resulting 4 bp intron retention causes a frame shift in the expressed transcript. Nucleic acid amplification primers designed to verify the presence or absence of this SNP are listed in Table 3 below. PCR derived amplification products confirming this PRR7 mutation in autoflowering plants were also verified by Sanger sequencing.

To further confirm the splice defect observed in autoflowering PRR7, cDNA from both autoflowering and photoperiod sensitive varieties was sequenced to determine potential variations in transcript splicing. The cDNA used for these autoflowering and photoperiod samples came from different developmental stages, specifically samples from 13 days, 27 days and 48 days post germination. For sequencing, PRR7 was PCR amplified using primers based on the predicted transcript, either from start codon (SEQ ID NO: 138) to coding exon 8 (SEQ ID NO: 141) or from start codon (SEQ ID NO: 138) to stop codon (SEQ ID NO: 142). Initial Sanger sequencing of PCR amplified PRR7 was carried out using primers represented by SEQ ID NOs: 138 and 140. Initial sequencing showed a single photoperiod splice variant which was as predicted. For the autoflowering sequences, there was a breakdown of sequencing surrounding the mutated splice junction, indicating the presence of multiple splice forms.

To identify the specific splice variants present in autoflowering plants the pJET1.2/blunt cloning vector was used to isolate independent transcripts. The autoflowering cDNA samples used represented two individual plants and three time points (13 days post germination, 27 days post germination, and 48 days post germination). The photoperiod samples used represented two plants and two time points (13 days post germination and 48 days post germination). Predicted full length PRR7 sequences were PCR amplified from each of these cDNA samples. PCR reactions were run on an agarose gel and individual bands approximately 2 Kb in size were recovered; no other bands of significant intensity were observed. These products were then blunt ligated into the pJET1.2/blunt cloning vector and transformed into competent E. coli via heat shock treatment. Transformed cells were plated on LB agar media with ampicillin selection and individual transformed colonies were identified using colony PCR (SEQ ID NOs: 143 and 144). Colonies with a single band of approximately 2 kb in size were selected for analysis. Individual colonies were selected for overnight culture in LB media with ampicillin: 8 colonies from each autoflowering plant (24 total) and 4 from each photoperiod plant (8 total). Plasmids were recovered from overnight cultures and Sanger sequencing was done using four primers (SEQ ID NO: 143, SEQ ID NO: 144, SEQ ID NO: 139, and SEQ ID NO: 140) which provide a complete coverage of the transcript.

The sequenced photoperiod transcripts were consistent in sequence. A representative sequence for Sanger sequenced photoperiod transcripts is provided as SEQ ID NO: 145. Of the 24 autoflowering sequences 17 were usable for analysis (5 indicated the presence of multiple splice form contamination and 2 were of poor quality). None of the autoflowering transcripts showed a canonical splicing pattern. Four different noncanonical splicing patterns were observed in the autoflowering sequences. A summary of the splice form variants, the rates of occurrence, and protein effects is provided in Table 4. Of the 17 high quality sequences, 13 had frameshifts (Forms 1, 2, and 4) which resulted in early stop codons shortly after the splice mutation. Form 3 represents 4/17 sequences and results in a 7 amino acid deletion in the middle of the highly conserved PRR domain. All the splice variants in the autoflowering plants resulted from the utilization of cryptic splice sites near the mutation site. The predicted mutational effects on the resulting PRR7 protein for each splice form is severe which strongly indicates a disrupted or non-functional PRR7 protein that is causative of the day length neutral phenotype.

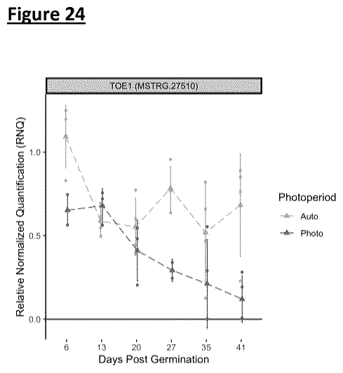

In Arabidopsis, PRR7 mutants display longer hypocotyls under red and far-red light compared to wildtype PRR7 seedlings. Accordingly, to further verify a non-functional PRR7 gene in Cannabis autoflowering genotypes as a determinant of the day length neutral phenotype, hypocotyl length in 11 day old autoflowering and photoperiod sensitive Cannabis seedlings grown under distinct light conditions was measured. Seedlings were germinated in dark for four days prior to being transplanted to soil and placed in either Dark (no light), FarRed (730 nm; 16 h light, 8 h dark), or White (Full spectrum; 16 h light, 8 h dark) for 7 days (FIG. 21). Hypocotyl measurements were made from soil to the top of cotyledons. Data shown is for inbred photoperiod (Photo) and autoflowering (Auto) varieties (N=5). Similar to Arabidopsis PRR7 mutants, increased hypocotyl length was observed in Cannabis seedlings in PRR7 mutants, indicating a non-functional PRR7 gene in autoflowering genotypes as a determinant of the day length neutral phenotype.

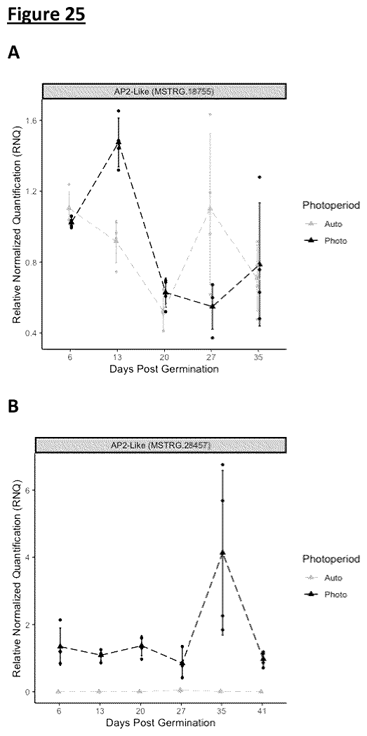

PRR7 is one of a few circadian clock genes involved in the flowering pathway (FIG. 22). Further investigation of FLOWERING LOCUS T (FT), which is the main florigen signal whose expression promotes the conversion of vegetative shoot apical meristem (SAM) to reproductive floral SAM, showed elevated expression in auto-flowering genotypes under long days (LD) as early as 20 days post sowing of seeds (FIG. 23a). In contrast, FT expression in photoperiod sensitive genotypes was not detected until after daylength was changed to short days (SD). These results appear to indicate that auto-flowering genotypes may be under a constant signal to flower beginning in juvenescence that results in early expression of FT under LD.

To determine how early FT is expressed in auto-flowering genotypes, leaf tissue samples were collected weekly from auto-flowering and photoperiod sensitive genotypes up to 6 weeks, starting 6 days post germination. Quantitative reverse transcription PCR (q-RT PCR) was used to measure the expression of three FT homologues identified in Cannabis. Primers were designed such that each pair spanned an exon-exon junction to prevent amplification of genomic DNA (SEQ ID NOs: 118 to 123). Primers were also designed for two genes (SEQ ID NOs: 128 to 131) that were selected to use as reference genes for qRT-PCR: CM010792.2.g1061/MSTRG.15101 (CUL3A) located on chromosome CM010792.2 between 26863617-26867132 bp, Purple Kush Assembly version 4, with sequence homology to Arabidopsis CUL3A (AT1G26830.1); CM010795.2.g29/MSTRG.22065 (AGC) located on chromosome CM010795.2 between 318929-335325 bp, Purple Kush Assembly version 4, with sequence homology to Arabidopsis AGC (AT3G23310.1). These genes were selected based on their stable gene expression throughout development as determined from previous RNAseq studies. Both genes were further tested for their suitability as reference genes with CFX's qPCR software Maestro using its Reference Gene Selection Tool. This tool graded both reference genes as ideal.

FT expression was detected as early as 13 days post germination (in some instances as early as 6 days post germination albeit detected at 37 amplification cycles or later) (FIG. 23b-d). FT expression continued to increase in auto-flowering genotypes while being either low expressed or not detected at all in photoperiod sensitive genotypes. First morphological signs of flowering were observed in auto-flowering genotypes at week 5 (35 days post germination) with the emergence of axillary pistils. Given the remarkably early detection of FT expression in auto-flowering genotypes, indicating that a premature signal to flower may be present as early as germination. This further suggests that the cause of auto-flowering in Cannabis may be acting upstream of FT.

Current evidence surrounding FT and PRR7 expression point to the possibility that auto-flowering genotypes may be under a signal to flower as soon as germination. However, first signs of flowering were not observed until week five with the emergence of pistils. Similar flowering times are observed in photoperiod sensitive genotypes that are grown under SD from germination. These observations suggest that there is a minimum age for flowering. While this period may be in part explained by the need for FT to accumulate to levels needed to induce flowering, Cannabis likely needs to transition out of its juvenile phase to be competent to flower. In Arabidopsis, the transition from juvenile to mature development is controlled by the age-dependency pathway. Briefly, this pathway involves AP2-like genes regulated by micro RNAs (miRNA) which repress flowering by directly interacting with the FT promoter. A similar pathway likely controls flowering in Cannabis, and indeed several genes have been identified with significant homology to known Arabidopsis age-dependency genes, including AP2-like genes TOE½, AP2, and regulatory micro RNAs miRNA156 and miRNA172.

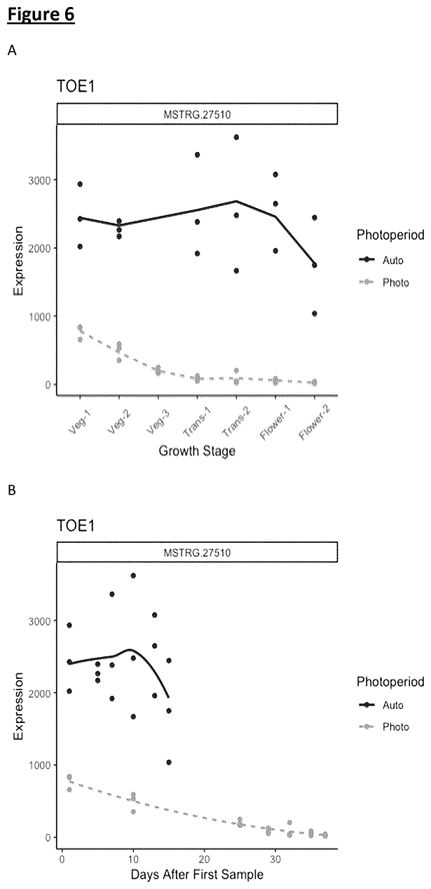

In Arabidopsis, TOE1 represses FT to prevent flowering until the plant has grown out of its juvenile phase whereby TOE1 expression begins to wane. Along with PRR7, TOE1 is positioned within the linkage region on chromosome 5 that segregates with the auto-flowering phenotype. RNAseq results showed that within auto-flowering genotypes, TOE1 expression was upregulated during development compared to photosensitive genotypes (FIG. 6a). This upregulation remained constant in auto-flowering plants, while in photoperiod sensitive genotypes TOE1 expression slowly continued to decrease throughout development. Quantitative RT-PCR was performed to measure TOE1 expression (SEQ ID NOs: 126 and 127) during early development and to validate results from RNAseq. FIG. 24 depicts TOE1 expression levels during the first 6 weeks of growth. TOE1 levels in photosensitive genotypes began to decrease after 13 days post germination. These results mirror those observed in RNAseq and are expected given TOE1's role in repressing flowering during early development. In auto-flowering genotypes, again TOE1 was upregulated compared to photoperiod sensitive genotypes.

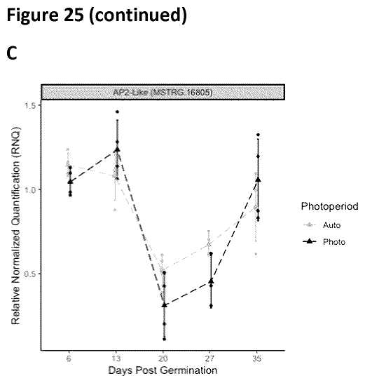

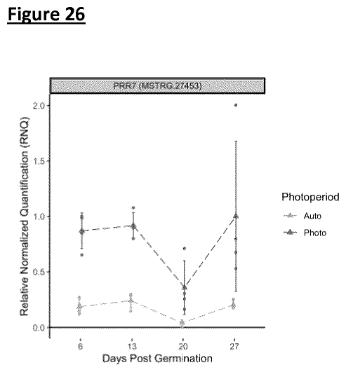

TOE1 expression in auto-flowering genotypes during development showed continued upregulation. If TOE1 in cannabis behaves as a repressor of FT, as it does in Arabidopsis, low levels of TOE1 expression would be expected at time of flowering. This was observed in photoperiod sensitive genotypes, suggesting TOE1 may indeed act as a repressor, but its relatively higher and more stable gene expression in auto-flowering genotypes suggested a different explanation. Without wishing to be bound by theory, it is possible that the upregulation in TOE1 is a response to a constitutive flowering signal in auto-flowering plants (e.g. PRR7). However, auto-flowering plants will still require 5 weeks to flower, similarly as photoperiod sensitive ones when grown under SDs from germination, indicating a functional age-dependency pathway. The level of gene redundancy within the age-dependent pathway may offer an explanation. In Arabidopsis, both TOE1 and AP2-like gene SMZ repress FT in young plants. In Cannabis, three other AP2-like genes were identified. Data from qRT-PCR from these genes (FIG. 25) revealed one to be differentially expressed, while the other two showed no significant expression differences between auto-flowering and photosensitive genotypes. It may be that while TOE1 remains upregulated in auto-flowering genotypes as a response to an early flowering signal, additional AP2-like repressors remain unaffected which ultimately maintains a functional age-dependency pathway whereby flowering is repressed before maturity (roughly five weeks). From an evolutionary perspective, it would make sense for selective pressures to maintain a working age-dependency pathway in auto-flowering genotypes as to increase fitness while adapting to higher latitudes.

As discussed above, several homologues of Pseudo Response Regulator (PRR) genes have been identified in Cannabis including PRR5, PRR7, and PRR9. Further investigation of these genes was conducted following the studies and analysis of PRR7 discussed above.

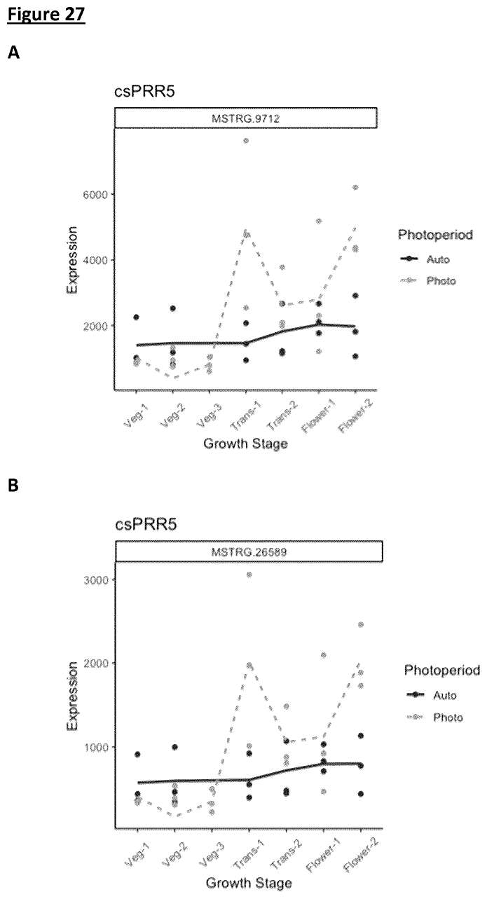

PRR7 expression within the first 4 weeks of development was also investigated using qRT-PCR with primers identified in SEQ ID NOs: 124 and 125. PRR7 expression during the first 4 weeks of development was downregulated in auto-flowering genotypes compared to photosensitive genotypes (FIG. 26). These results complement those observed in the RNAseq study, where the first vegetative timepoint corresponds to approximately 3 weeks post germination of the qRT-PCR results. PRR7 differential expression between auto-flowering and photoperiod sensitive genotypes is detected as early as 6 days post germination. Given that the circadian clocks functions upstream of FT, PRR7's early downregulation supports it as a likely candidate for controlling flowering time. This is further supported by RNAseq data showing PRR7 expression in photosensitive genotypes drops and stabilizes at levels observed in auto-flowering genotypes after switching to SD. It appears that abnormal circadian clock function surrounding PRR7 in auto-flowering genotypes may lead plants to perceive SDs thereby triggering flowering regardless of actual photoperiod.

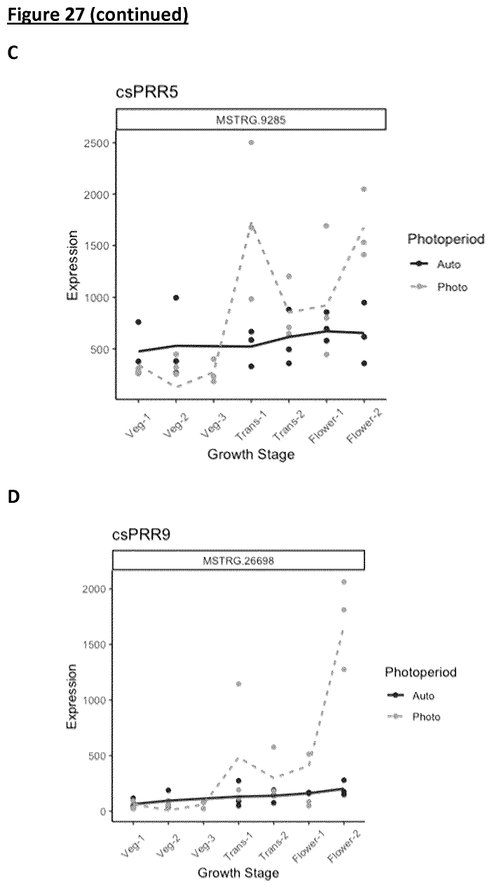

Following the analysis of PRR7, expression of both PRR5 and PRR9 was investigated. Although both PRR5 and PRR9 are not in the 20 Mb low recombination region associated with the autoflowering genotype, PRR5, PRR7, and PRR9 are interconnected. Both PRR5 and PRR9 homologues in Arabidopsis, APRR5 and APRR9, have been shown to be partially redundant with APRR7. Three putative homologues for PRR5 (PRR5 homologue #1 genomic sequence-SEQ ID NO: 14, PRR5 homologue #2 genomic sequence-SEQ ID NO: 15, PRR5 homologue #3 genomic sequence-SEQ ID NO: 16) and one homologue for PRR9 (PRR9 homologue genomic sequence-SEQ ID NO: 17) were identified from assembled RNA transcripts. PRR5 expression remained relatively constant in autoflowering genotypes, while in photoperiod genotypes a sharp increase in expression at the first transition time-point and last flowering time-point was observed (FIGS. 27A-C). Similarly, PRR9 expression remained relatively constant in autoflowering genotypes, while in photoperiod genotypes a sharp increase during the last flowering time-point and a small spike during the first transition time-point was observed (FIG. 27D).

Following the above observations with respect to PRR5, PRR7, and PRR9, an investigation of their 24-hour oscillation patterns between auto-flowering and photoperiod sensitive genotypes under both long days (LD) (18 h light) and short days (SD) (12 h light) using qRT-PCR was conducted (see qRT-PCR methods above). Primer pairs used in the 24-hour qRT-PCR analysis include sequences represented in SEQ ID NOs: 132 and 133 (PRR7), SEQ ID NOS: 134 and 135 (PRR5), and SEQ ID NOs: 136 and 137 (PRR9).

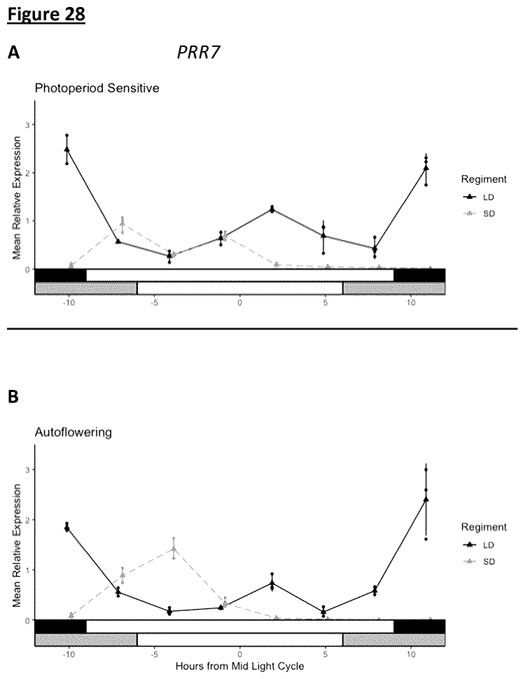

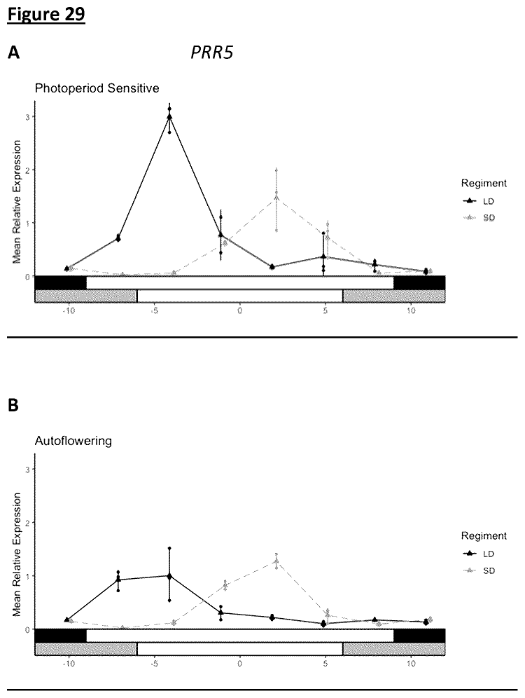

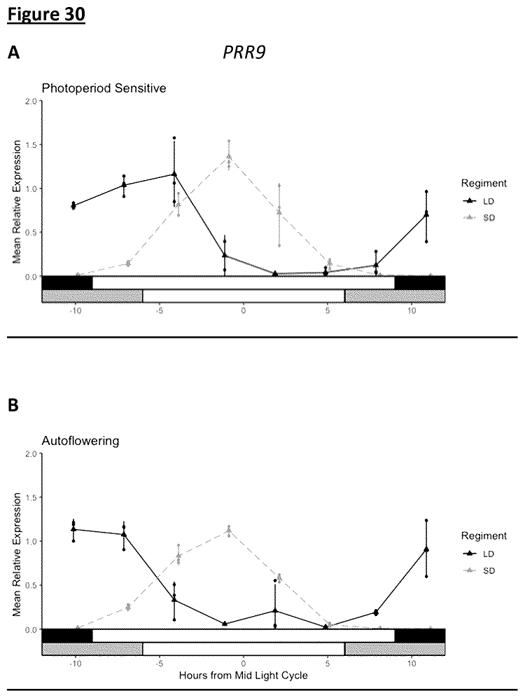

Oscillating expression patterns of PRR5, PRR7, and PRR9 showed significant differences between auto-flowering and photoperiod sensitive genotypes under LD. Under SD, other than in PRR7, significant differences were not observed. PRR7 displays a biphasic oscillation pattern peaking 1 hour before lights turn on and again 11 hours after lights-on (mid-day) (FIG. 28). Time of expression peaks between auto-flowering and photoperiod sensitive genotypes did not vary, however the mid-day expression peak in auto-flowering genotypes were significantly lower compared to photoperiod genotypes (FIG. 28A-B). Expression of PRR5 peaks approximately 5 hours after lights turn on under LD. Peak expression is 3-fold higher in photoperiod sensitive genotypes compared to auto-flowering (FIG. 29A-B). For PRR9 expression under LD, peak expression was also observed 5 hours post lights-on for photoperiod sensitive genotypes (FIG. 30A). In auto-flowering genotypes, peak expression was observed 1 hour before lights-on, indicating a 6-hour phase shift between auto-flowering and photoperiod sensitive genotypes (FIG. 30B).

Under SD light regiments, lesser differences were observed in circadian clock genes between auto-flowering and photoperiod sensitive genotypes. PRR7 expression in photoperiod sensitive genotypes under SD continued to show a biphasic expression pattern (FIG. 28A). While the first expression peak was still 1 hour before lights-on, the mid-day peak was observed 5 hours after lights-on, 6 hours earlier than LD. A significant difference was observed in PRR7 expression under SD for auto-flowering genotypes. The biphasic expression pattern observed under LD changed to a single expression peak occurring 2 hours after lights-on (FIG. 28B). No differences in expression patterns were observed for PRR5 and PRR9 between auto-flowering and photo-sensitive genotypes. PRR5 peak expression was observed 8 hours after lights-on (FIG. 29A-B). For PRR9, peak expression was observed 5 hours after lights-on (FIG. 30A-B). It is interesting to note that peak expression for PRR9 was also 5 hours after lights-on in LD for photoperiod sensitive genotypes, indicating the that 6-hour phase shift observed between phenotypes under LD does not persist under SD.

These results indicate that under LD (18 h light) there are significant expression differences in PRR5, PRR7, and PRR9, indicating a disruption in the auto-flowering LD clock. However, under SD, expression differences are only observed in PRR7, with PRR5 and PRR9 showing identical expression peaks between auto-flowering and photoperiod genotypes which indicates a less disrupted circadian clock. This supports the observation that both auto-flowering and photoperiod sensitive genotypes flower approximately the same time when grown from seed under SD.

The identified splice site mutation in auto-flowering PRR7 (discussed above) appears to be causing a disrupted circadian clock under LD, which, in turn, fails to repress flowering until daylength changes to SD. In addition to identification of the splice site mutation in PRR7, the location of PRR7 within all the linkage regions discussed above (0.75 Mb, 2.5 Mb, and 20 Mb) supports the mutation in this gene as the polymorphism causative of the autoflowering phenotype. No other clock genes have been identified in the linkage region and no mutations have been identified in other clock genes. PRR7 likely regulates other clock genes explaining the expression differences observed in PRR5 and PRR9 under LD. The observation that PRR5 and PRR9 expression is not altered between auto-flowering and photoperiod sensitive genotypes under SD indicates PRR7's important role in repressing flowering under LD specifically.

Knockout mutants for APRR7 in Arabidopsis have been shown to result in altered flowering times. The phenotype has also been shown to be further exacerbated with double or triple mutants involving APRR5 and/or APRR9, indicating their partial redundancy and collective repression of CDF1 (FIG. 22). Accordingly, a knockout in PRR5 and/or PRR9 may similarly affect flowering time in Cannabis.

Now that an allele of the PRR7 gene has been established as associated with the day length neutral phenotype, the skilled person understands that any allele at additional polymorphic sites on the chromosome that is in linkage disequilibrium with this allele of PRR7 will also be associated with the day length neutral phenotype.

Moreover, the skilled person understands that it may be possible to use any one of polymorphisms that are in linkage disequilibrium with the PRR7 gene as a surrogate marker for the variation in the PRR7 gene for the identification of plants that have the day length neutral phenotype or are carriers of the trait for the day length neutral phenotype.

Identification of Plants that have a Day Length Neutral (Autoflowering) Phenotype

The polymorphisms represented in any one of SEQ ID NOs: 4 to 9, or combinations thereof (Table 5), are useful in methods of identifying whether or not a Cannabis plant has a day length neutral phenotype, or is a carrier of a trait for a day length neutral phenotype.

In one aspect, this disclosure provides a method comprising testing nucleic acid from a Cannabis plant to determine the presence or absence of a point mutation at position 101 of SEQ ID NO: 9. In one aspect, this disclosure provides a method comprising testing nucleic acid from a Cannabis plant to determine the presence or absence of a point mutation at position 101 of SEQ ID NO: 4. In one aspect, this disclosure provides a method comprising testing nucleic acid from a Cannabis plant to determine the presence or absence of a point mutation at position 101 of SEQ ID NO: 5. In one aspect, this disclosure provides a method comprising testing nucleic acid from a Cannabis plant to determine the presence or absence of a point mutation at position 101 of SEQ ID NO: 6. In one aspect, this disclosure provides a method comprising testing nucleic acid from a Cannabis plant to determine the presence or absence of a point mutation at position 101 of SEQ ID NO: 7. In one aspect, this disclosure provides a method comprising testing nucleic acid from a Cannabis plant to determine the presence or absence of a point mutation at position 101 of SEQ ID NO: 8. In one aspect, this disclosure provides a method comprising testing nucleic acid from a Cannabis plant to determine the presence or absence of an insertion starting at position 163 of SEQ ID NO: 12. In particular, the method is for identifying whether or not the Cannabis plant has a day length neutral phenotype, or is a carrier of a trait for a day length neutral phenotype. The presence of the variation indicates that the plant has a day length neutral phenotype or is a carrier of the trait for the day length neutral phenotype. In such embodiments, testing nucleic acid from the Cannabis plant to determine the presence or absence of the variation comprises testing for the presence or absence of the point mutation at position 101 of SEQ ID NO: 9, wherein the presence of A at position 101 of SEQ ID NO: 9 or T in the complement in both copies of the endogenous PRR7 gene indicates that the plant has a day length neutral phenotype. The presence of A at position 101 of SEQ ID NO: 9 or T in the complement in only one copy of the PRR7 gene indicates that the plant is a carrier of the trait for the day length neutral phenotype. In other embodiments, testing nucleic acid from the Cannabis plant to determine the presence or absence of the variation comprises testing for the presence or absence of the point mutation at position 101 of SEQ ID NO: 4, wherein the presence of T at position 101 of SEQ ID NO: 4 or A in the complement in both copies of the endogenous PRR7 gene indicates that the plant has a day length neutral phenotype. The presence of T at position 101 of SEQ ID NO: 4 or A in the complement in only one copy of the PRR7 gene indicates that the plant is a carrier of the trait for the day length neutral phenotype. In some embodiments, testing nucleic acid from the Cannabis plant to determine the presence or absence of the variation comprises testing for the presence or absence of the point mutation at position 101 of SEQ ID NO: 5, wherein the presence of C at position 101 of SEQ ID NO: 5 or G in the complement in both copies of the endogenous PRR7 gene indicates that the plant has a day length neutral phenotype. The presence of C at position 101 of SEQ ID NO: 5 or G in the complement in only one copy of the PRR7 gene indicates that the plant is a carrier of the trait for the day length neutral phenotype. In some embodiments, testing nucleic acid from the Cannabis plant to determine the presence or absence of the variation comprises testing for the presence or absence of the point mutation at position 101 of SEQ ID NO: 6, wherein the presence of C at position 101 of SEQ ID NO: 6 or G in the complement in both copies of the endogenous PRR7 gene indicates that the plant has a day length neutral phenotype. In some embodiments, testing nucleic acid from the Cannabis plant to determine the presence or absence of the variation comprises testing for the presence or absence of the point mutation at position 101 of SEQ ID NO: 7, wherein the presence of T at position 101 of SEQ ID NO: 7 or A in the complement in both copies of the endogenous PRR7 gene indicates that the plant has a day length neutral phenotype. The presence of T at position 101 of SEQ ID NO: 7 or A in the complement in only one copy of the PRR7 gene indicates that the plant is a carrier of the trait for the day length neutral phenotype. In some embodiments, testing nucleic acid from the Cannabis plant to determine the presence or absence of the variation comprises testing for the presence or absence of the point mutation at position 101 of SEQ ID NO: 8, wherein the presence of G at position 101 of SEQ ID NO: 8 or C in the complement in both copies of the endogenous PRR7 gene indicates that the plant has a day length neutral phenotype. The presence of G at position 101 of SEQ ID NO: 8 or C in the complement in only one copy of the PRR7 gene indicates that the plant is a carrier of the trait for the day length neutral phenotype. In some embodiments, testing nucleic acid from the Cannabis plant to determine the presence or absence of the variation comprises testing for the presence or absence of the insertion at position 163 of SEQ ID NO: 12, wherein the presence of TTGA at position 163 of SEQ ID NO: 12 or TCAA in the complement in both copies of the PRR7 cDNA transcript indicates that the plant has a day length neutral phenotype. The presence of TTGA at position 163 of SEQ ID NO: 12 or TCAA in the complement in only one copy of the PRR7 cDNA transcript indicates that the plant is a carrier of the trait for the day length neutral phenotype.

However, the skilled person with the understanding that the PRR7 allele comprising the point mutation of SEQ ID NO: 9 is causative of the day length neutral phenotype, i.e., if the day length neutral phenotype results from a loss of PRR7 gene expression/activity, will recognize that any loss of function allele of the PRR7 gene may function as a marker of and/or cause the day length neutral phenotype. Thus, in another aspect, this disclosure more generally provides a method comprising testing nucleic acid from a Cannabis plant to determine the presence or absence of a variation in the endogenous PRR7 gene. The method is for identifying whether or not the Cannabis plant has a day length neutral phenotype, or is a carrier of a trait for a day length neutral phenotype. The presence of the variation indicates that the plant has a day length neutral phenotype or is a carrier of the trait for the day length neutral phenotype. The variation is a loss-of-function mutation in the PRR7 gene, which may be a point mutation (e.g., a substitution, a missense mutation, or a nonsense mutation), a deletion, or an insertion. The presence of the variation in both copies of the endogenous PRR7 gene indicates that the plant has a day length neutral phenotype. The presence of the variation in only a single copy of the endogenous PRR7 gene indicates that the plant is a carrier of the trait for the day length neutral phenotype.

Similarly, the skilled person understands that if the PRR5 and/or PRR9 genes function in at least a partially redundant manner with PRR7 in Cannabis, then any loss of function allele of the PRR5 and/or PRR9 gene may also cause or enhance the day length neutral phenotype. Thus, in another aspect, this disclosure more generally provides a method comprising testing nucleic acid from a Cannabis plant to determine the presence or absence of a variation in the endogenous PRR5 and/or PRR9 gene. The method is for identifying whether or not the Cannabis plant has a day length neutral phenotype, or is a carrier of a trait for a day length neutral phenotype. The presence of the variation indicates that the plant has a day length neutral phenotype or is a carrier of the trait for the day length neutral phenotype. The variation is a loss-of-function mutation in the PRR5 and/or PRR9 gene, which may be a point mutation (e.g., a substitution, a missense mutation, or a nonsense mutation), a deletion, or an insertion. The presence of the variation in both copies of the endogenous PRR5 and/or PRR9 gene indicates that the plant has a day length neutral phenotype. The presence of the variation in only a single copy of the endogenous PRR5 and/or PRR9 gene indicates that the plant is a carrier of the trait for the day length neutral phenotype.

The skilled person further understands that any polymorphism in linkage disequilibrium with an allele of the PRR7 gene (i.e., SEQ ID NOs: 4 to 9) can be used as a surrogate marker for the day length neutral phenotype. Thus, in another aspect, this disclosure provides a method comprising testing nucleic acid from a Cannabis plant to determine the presence or absence of one or more variation (i.e., allele at a polymorphic site) that is in linkage disequilibrium (i.e., where the coefficient of correlation of the alleles, r2, is 0.6 to 1, 0.7 to 1, 0.8 to 1, or 0.9 to 1) with the PRR7 allele comprising the polymorphisms of any one of SEQ ID NOs: 4 to 9. The method is for identifying whether a Cannabis plant has a day length neutral phenotype, or is a carrier of a trait for a day length neutral phenotype. The presence of the variation indicates that the plant has a day length neutral phenotype or is a carrier of the trait for the day length neutral phenotype. The presence of the allele indicates that the plant has a day length neutral phenotype or is a carrier of the trait for the day length neutral phenotype. In some embodiments, the allele is located within an about 20 megabase region in Cannabis chromosome CM010796.2 located between about 40 megabases to about 60 megabases. In some embodiments, the allele located within the region in Cannabis chromosome CM010796.2 is selected from: about 40 megabases to about 42.5 megabases, about 40 megabases to about 45 megabases, about 40 megabases to about 47.5 megabases, about 40 megabases to about 50 megabases, about 40 megabases to about 52.5 megabases, about 40 megabases to about 55 megabases, about 40 megabases to about 57.5 megabases, about 42.5 megabases to about 45 megabases, about 42.5 megabases to about 47.5 megabases, about 42.5 megabases to about 50 megabases, about 42.5 megabases to about 52.5 megabases, about 42.5 megabases to about 55 megabases, about 42.5 megabases to about 57.5 megabases, about 42.5 megabases to about 60 megabases, about 45 megabases to about 47.5 megabases, about 45 megabases to about 50 megabases, about 45 megabases to about 52.5 megabases, about 45 megabases to about 55 megabases, about 45 megabases to about 57.5 megabases, about 45 megabases to about 60 megabases, about 47.5 megabases to about 50 megabases, about 47.5 megabases to about 52.5 megabases, about 47.5 megabases to about 55 megabases, about 47.5 megabases to about 57.5 megabases, about 47.5 megabases to about 60 megabases, about 50 megabases to about 52.5 megabases, about 50 megabases to about 55 megabases, about 50 megabases to about 57.5 megabases, about 50 megabases to about 60 megabases, about 52.5 megabases to about 55 megabases, about 52.5 megabases to about 57.5 megabases, about 52.5 megabases to about 60 megabases, about 55 megabases to about 57.5 megabases, about 55 megabases to about 60 megabases, or about 57.5 megabases to about 60 megabases. In some embodiments, the allele is located within an about 2.5 megabase region in Cannabis chromosome CM010796.2 located between about 41 megabases to about 43.5 megabases. In some embodiments, the allele is located within an about 0.75 megabase region in Cannabis chromosome CM010796.2 located between about 41.09 megabases to about 41.84 megabases.

Again, the skilled person understanding that this point mutation of SEQ ID NO: 9 is causative of the day length neutral phenotype, will recognize that the presence or absence of one or more variation (i.e., allele at a polymorphic site) that is in linkage disequilibrium with any loss of function variation in the endogenous PRR7 gene could be used as a marker for the day length neutral phenotype.

Due to the low recombination of the about 0.75, 2.5, or 20 megabase region in Cannabis (e.g., chromosome CM010796.2 or NC_044371.1 any polymorphism in this low recombination region would be in strong linkage disequilibrium with the PRR7 gene and could be used as a marker to select individuals with the day length neutral phenotype in a segregating population. For example, polymorphic sites within genes identified in the about 0.75, 2.5, or 20 megabase region could also be used as a marker to select individuals with the day length neutral phenotype.

Polymorphic sites in genes associated with the day length neutral phenotype, and within the about 20 megabase region, are presented in Table 6. The skilled person understands that additional polymorphic sites not identified in Table 6 but that are within the about 20 megabase region, and the alleles present at these sites, may be used for the identification of plants that have the day length neutral phenotype or that are carriers of the trait for day length neutral phenotype.

Polymorphic sites in genes associated with the day length neutral phenotype, and outside the about 20 megabase region, are presented in Table 7. The skilled person understands that polymorphic sites that are outside the about 20 Mb region but are nonetheless within genes that are within the about 20 megabase region, may be used for the identification of plants that have the day length neutral phenotype or that are carriers of the trait for day length neutral phenotype.

Moreover, the skilled person understands that any allele at additional polymorphic sites on the chromosome that is in linkage disequilibrium with an allele at a polymorphic site in any one of SEQ ID NOs: 28 to 40 will also be associated with the day length neutral phenotype. For example, once an allele at a polymorphic site of any one of SEQ ID NOs: 28 to 40 is established as associated with the day length neutral phenotype, the skilled person understands that any allele at additional polymorphic sites on the chromosome that is in linkage disequilibrium with this allele of any one of SEQ ID NOs: 28 to 40 will also be associated with the day length neutral phenotype.

The skilled person understands that it may be possible to use any one of the polymorphisms presented in Tables 6 and 7 as a marker for the identification of plants that have the day length neutral phenotype or that are carriers of the trait for the day length neutral phenotype. The skilled person also understands that additional polymorphic sites not identified in any of Tables 6 and 7 but that are in linkage disequilibrium with any one of SEQ ID NOs: 28 to 40, and the alleles present at these sites, may be used for the identification of plants that have the day length neutral phenotype or that are carriers of the trait for day length neutral phenotype.

Thus, the skilled person understands that the genes disclosed in Table 1 and the polymorphisms disclosed in Tables 6, and 7 are useful in methods of identifying whether or not a Cannabis plant has a day length neutral phenotype, or is a carrier of a trait for a day length neutral phenotype, but that other polymorphisms within the about 20 megabase region may be useful in such methods.

In one aspect, this disclosure provides a method comprising testing nucleic acid from a Cannabis plant to determine the presence or absence of a polymorphic site in an about 20 megabase region in chromosome CM010796.2 located between about 40 megabases and about 60 megabases (or in chromosome NC_044371.1 located between about 0 megabase to about 20 megabases). For example, the disclosure provides a method comprising testing nucleic acid from a Cannabis plant to determine the presence or absence of a variation at a polymorphic site within a low-recombination region in the Cannabis genome or an allele in linkage disequilibrium with the polymorphic site within the low-recombination region. In particular, the methods are for identifying whether or not the Cannabis plant has a day length neutral phenotype, or is a carrier of a trait for a day length neutral phenotype. The presence of a variation indicates that the plant has a day length neutral phenotype or is a carrier of the trait for the day length neutral phenotype.

In another aspect, this disclosure provides a method comprising testing nucleic acid from a Cannabis plant to determine the presence or absence of a polymorphic site in an about 2.5 megabase region in chromosome CM010796.2 located between about 41.09 megabases and about 43.59 megabases (or in chromosome NC_044371.1 located between about 1.09 megabase to about 3.59 megabases).

In another aspect, this disclosure provides a method comprising testing nucleic acid from a Cannabis plant to determine the presence or absence of a polymorphic site in an about 0.75 megabase region in chromosome CM010796.2 located between about 41.09 megabases and about 41.84 megabases (or in chromosome NC_044371.1 located between about 1.09 megabase to about 1.84 megabases).

The skilled person further understands that any polymorphism in linkage disequilibrium with a variation at a polymorphic site of the about 20 megabase region can be used as a surrogate marker for the day length neutral phenotype. For example, any polymorphism in linkage disequilibrium with an allele of any one of the endogenous flowering related genes (i.e., SEQ ID NOs: 28 to 40) can be used as a surrogate marker for the day length neutral phenotype.

In another aspect, this disclosure provides a method comprising testing nucleic acid from a Cannabis plant to determine the presence or absence of a variation at a polymorphic site corresponding to any one of SEQ ID NOs: 69 to 115 or an allele in linkage disequilibrium with the polymorphic site corresponding to any one of SEQ ID NOs: 69 to 115.

In another aspect, this disclosure provides a method comprising testing nucleic acid from a Cannabis plant to determine the presence or absence of a variation at a polymorphic site corresponding to any one of SEQ ID NOs: 69 to 91 or an allele in linkage disequilibrium with the polymorphic site corresponding to any one of SEQ ID NOs: 69 to 91.

In another aspect, this disclosure provides a method comprising testing nucleic acid from a Cannabis plant to determine the presence or absence of a variation at a polymorphic site corresponding to any one of SEQ ID NOs: 70 to 83 or an allele in linkage disequilibrium with the polymorphic site corresponding to any one of SEQ ID NOs: 70 to 83.

The skilled person understands that while the polymorphisms of SEQ ID NOS: 92 to 115 fall outside of the 20 Mb low recombination region, these markers are useful due to their close proximity to markers within the 20 Mb region (i.e., SEQ ID NOs: 69 to 91). The skilled person further understands that any polymorphism in linkage disequilibrium with the polymorphisms of SEQ ID NOs: 69 to 115 can also be used as a surrogate marker for the day length neutral phenotype.

In various embodiments of the methods described above, “testing” comprises nucleic acid amplification, e.g., amplification carried out by polymerase chain reaction (PCR).

The skilled person understands that testing may also involve sequencing, 5′ nuclease digestion, molecular beacon assay, oligonucleotide ligation assay, size analysis, single-stranded conformation n polymorphism analysis, denaturing gradient gel electrophoresis (DGGE), or any other appropriate methodology as described elsewhere herein and in references cited herein.

In preferred embodiments, testing is performed using an allele-specific method, e.g., allele-specific probe hybridization, allele-specific primer extension, or allele-specific amplification. Testing may be carried out using an allele-specific primer that comprises a sequence selected from the group consisting of SEQ ID NOS: 20 to 26, and sequences fully complementary thereto.

### Marker-Assisted Breeding

The methods for identifying plants that have a day length neutral phenotype, or are a carrier of the trait, are particularly useful in methods of marker-assisted breeding to efficiently introduce the day length neutral trait into Cannabis cultivars/varieties having desirable traits but a photoperiod phenotype.

Thus, another aspect of the disclosure relates to methods of producing a Cannabis plant with a day length neutral phenotype. The method comprises initially performing a first cross of a first parent having at least one allele associated with a day length neutral phenotype at a polymorphic site in the PRR7 gene, with a second parent that has a day length sensitive phenotype. The skilled person understands that it is not necessary that the first parent have the day length sensitive phenotype so long as the first parent is at least a carrier of the trait, such that the methods described above can be used to screen progeny of the cross for the presence of the allele associated with the day length neutral phenotype. The method further comprises identifying a first progeny plant from the first cross that has a day length neutral phenotype, or that is a carrier of a trait for a day length neutral phenotype, according to a method as described above under the section entitled “Identification of Plants that have a Day Length Neutral (Autoflowering) Phenotype”.

Thus, another aspect of the disclosure relates to methods of producing a Cannabis plant with a day length neutral phenotype. The method comprises initially performing a first cross of a first parent having at least one allele associated with a day length neutral phenotype at a polymorphic site corresponding to SEQ ID NOs: 69 to 91 within an about 20 megabase region in Cannabis chromosome CM010796.2 located between about 40 megabases to about 60 megabases with a second parent that has a day length sensitive phenotype. The skilled person understands that it is not necessary that the first parent have the day length sensitive phenotype so long as the first parent is at least a carrier of the trait, such that the methods described above can be used to screen progeny of the cross for the presence of the allele associated with the day length neutral phenotype. The method further comprises identifying a first progeny plant from the first cross that has a day length neutral phenotype, or that is a carrier of a trait for a day length neutral phenotype, according to a method as described above.

In another aspect of the disclosure relates to methods of producing a Cannabis plant with a day length neutral phenotype. The method comprises initially performing a first cross of a first parent having at least one allele associated with a day length neutral phenotype at a polymorphic site corresponding to SEQ ID NOs: 69 to 115 with a second parent that has a day length sensitive phenotype. The skilled person understands that it is not necessary that the first parent have the day length sensitive phenotype so long as the first parent is at least a carrier of the trait, such that the methods described above can be used to screen progeny of the cross for the presence of the allele associated with the day length neutral phenotype. The method further comprises identifying a first progeny plant from the first cross that has a day length neutral phenotype, or that is a carrier of a trait for a day length neutral phenotype, according to a method as described above.

In another aspect of the disclosure relates to methods of producing a Cannabis plant with a day length neutral phenotype. The method comprises initially performing a first cross of a first parent having at least one allele associated with a day length neutral phenotype at a polymorphic site corresponding to SEQ ID NOs: 70 to 83 within an about 2.5 megabase region in Cannabis chromosome CM010796.2 located between about 41.09 megabases to about 43.59 megabases with a second parent that has a day length sensitive phenotype. The skilled person understands that it is not necessary that the first parent have the day length sensitive phenotype so long as the first parent is at least a carrier of the trait, such that the methods described above can be used to screen progeny of the cross for the presence of the allele associated with the day length neutral phenotype. The method further comprises identifying a first progeny plant from the first cross that has a day length neutral phenotype, or that is a carrier of a trait for a day length neutral phenotype, according to a method as described above.

In another aspect of the disclosure relates to methods of producing a Cannabis plant with a day length neutral phenotype. The method comprises initially performing a first cross of a first parent having at least one allele associated with a day length neutral phenotype at a polymorphic site corresponding to SEQ ID NOs: 70 to 83 within an about 0.75 megabase region in Cannabis chromosome CM010796.2 located between about 41.09 megabases to about 41.84 megabases with a second parent that has a day length sensitive phenotype. The skilled person understands that it is not necessary that the first parent have the day length sensitive phenotype so long as the first parent is at least a carrier of the trait, such that the methods described above can be used to screen progeny of the cross for the presence of the allele associated with the day length neutral phenotype. The method further comprises identifying a first progeny plant from the first cross that has a day length neutral phenotype, or that is a carrier of a trait for a day length neutral phenotype, according to a method as described above.

After a first progeny plant that is a carrier of a trait for a day length neutral phenotype is identified, then the first progeny plant may be self-pollinated to produce F₂ progeny. The method may then further include identifying an F₂ progeny plant that has a day length neutral phenotype according to the method described above. However, the skilled person would understand that 25% of the resulting F₂ progeny should have the day length neutral phenotype, such that the skilled person may choose not to screen for plants carrying two copies of the allele associated with the day length neutral phenotype. Alternatively, the skilled person may be interested in selecting F₂ progeny that are carriers of the trait and thus may choose an F₂ progeny plant that is a carrier of the trait for the day length neutral phenotype according to the method described above.

Alternatively, after a first progeny plant that is a carrier of a trait for a day length neutral phenotype is identified, the method may comprise backcrossing the first progeny plant to the first parent, and then identifying a progeny plant from the backcross that has a day length neutral phenotype according to a method described above. Such strategy may be of interest where the interest is in introducing a particular trait from the photoperiod sensitive variety to the variety with the day length neutral phenotype.

Alternatively, the method may comprise backcrossing the first progeny plant to the second parent, and then identifying a progeny plant from the backcross that is a carrier of the trait for the day length neutral phenotype according to the method as described above. Such strategy may be useful after a first progeny plant that is a carrier of a trait for a day length neutral phenotype is identified where the goal is to introduce only the autoflowering trait to the variety of the second parent.

In another embodiment, after a first progeny plant that is a carrier of a trait for a day length neutral phenotype is identified, the method further comprises performing a second cross of the first progeny plant with a second progeny plant identified as a carrier of a trait for a day length neutral phenotype according to a method as described above. The method then comprises identifying a progeny plant from the second cross that has a day length neutral phenotype, or is a carrier of the trait, according to a method as described above.

In another aspect, the disclosure provides methods of producing a Cannabis plant with a day length neutral phenotype comprising initially performing a first cross of a first parent that has a day length neutral phenotype and is homozygous for at least one allele associated with a day length neutral phenotype at a polymorphic site in the PRR7 gene, with a second parent that has a day length sensitive phenotype to produce F₁ progeny. The skilled person understands that each of the F₁ progeny will be a carrier of the trait for the day length neutral phenotype such that it is not necessary to screen for a carrier using any of the methods described above. Rather, the method further comprises selfing the F₁ progeny to produce F₂ progeny and then identifying an F₂ progeny plant that has a day length neutral phenotype, or that is a carrier of a trait for a day length neutral phenotype, according to a method as described above.

In yet another aspect, the disclosure provides methods of producing a Cannabis plant with a day length neutral phenotype comprising initially identifying a first parent homozygous for at least one allele associated with a day length neutral phenotype at a polymorphic site in the PRR7 gene according to a method described above. The method further comprises crossing the first parent to a second parent that has a day length sensitive phenotype to produce F₁ progeny. The skilled person understands that each of the F₁ progeny will be a carrier of the trait for the day length neutral phenotype such that it is not necessary to screen for a carrier using any of the methods described above. The method thus further comprises selfing the F₁ progeny to produce F₂ progeny, and identifying an F₂ progeny plant that has a day length neutral phenotype. While it is not necessary to screen the F₂ progeny according to the methods described above to determine whether or not a plant has a day length neutral phenotype (i.e., because 25% of the plants should have a day length neutral phenotype), the skilled person could do so if it were desirable to know the phenotype before flowering is initiated. Alternatively, the skilled person may want to screen the F₂ progeny for carriers of the trait.

In another aspect, the disclosure provides methods of producing a Cannabis plant with a day length neutral phenotype comprising initially performing a first cross of a first parent that has a day length neutral phenotype and is homozygous for at least one allele associated with a day length neutral phenotype at a polymorphic site corresponding to SEQ ID NOs: 69 to 91 within an about 20 megabase region in Cannabis chromosome CM010796.2 located between about 40 megabases to about 60 megabases, with a second parent that has a day length sensitive phenotype to produce F₁ progeny. The skilled person understands that each of the F₁ progeny will be a carrier of the trait for the day length neutral phenotype such that it is not necessary to screen for a carrier using any of the methods described above. Rather, the method further comprises selfing the F₁ progeny to produce F₂ progeny and then identifying an F₂ progeny plant that has a day length neutral phenotype, or that is a carrier of a trait for a day length neutral phenotype, according to a method as described above.

In another aspect, the disclosure provides methods of producing a Cannabis plant with a day length neutral phenotype comprising initially performing a first cross of a first parent that has a day length neutral phenotype and is homozygous for at least one allele associated with a day length neutral phenotype at a polymorphic site corresponding to SEQ ID NOs: 69 to 115 with a second parent that has a day length sensitive phenotype to produce F₁ progeny. The skilled person understands that each of the F₁ progeny will be a carrier of the trait for the day length neutral phenotype such that it is not necessary to screen for a carrier using any of the methods described above. Rather, the method further comprises selfing the F₁ progeny to produce F₂ progeny and then identifying an F₂ progeny plant that has a day length neutral phenotype, or that is a carrier of a trait for a day length neutral phenotype, according to a method as described above.

In another aspect, the disclosure provides methods of producing a Cannabis plant with a day length neutral phenotype comprising initially performing a first cross of a first parent that has a day length neutral phenotype and is homozygous for at least one allele associated with a day length neutral phenotype at a polymorphic site corresponding to SEQ ID NOs: 70 to 83 within an about 2.5 megabase region in Cannabis chromosome CM010796.2 located between about 41.09 megabases to about 43.59 megabases, with a second parent that has a day length sensitive phenotype to produce F₁ progeny. The skilled person understands that each of the F₁ progeny will be a carrier of the trait for the day length neutral phenotype such that it is not necessary to screen for a carrier using any of the methods described above. Rather, the method further comprises selfing the F₁ progeny to produce F₂ progeny and then identifying an F₂ progeny plant that has a day length neutral phenotype, or that is a carrier of a trait for a day length neutral phenotype, according to a method as described above.

In another aspect, the disclosure provides methods of producing a Cannabis plant with a day length neutral phenotype comprising initially performing a first cross of a first parent that has a day length neutral phenotype and is homozygous for at least one allele associated with a day length neutral phenotype at a polymorphic site corresponding to SEQ ID NOs: 70 to 83 within an about 0.75 megabase region in Cannabis chromosome CM010796.2 located between about 41.09 megabases to about 41.84 megabases, with a second parent that has a day length sensitive phenotype to produce F₁ progeny. The skilled person understands that each of the F₁ progeny will be a carrier of the trait for the day length neutral phenotype such that it is not necessary to screen for a carrier using any of the methods described above. Rather, the method further comprises selfing the F₁ progeny to produce F₂ progeny and then identifying an F₂ progeny plant that has a day length neutral phenotype, or that is a carrier of a trait for a day length neutral phenotype, according to a method as described above.

In yet another aspect, the disclosure provides methods of producing a Cannabis plant with a day length neutral phenotype comprising initially identifying a first parent homozygous for at least one allele associated with a day length neutral phenotype at a polymorphic site corresponding to SEQ ID NOs: 69 to 91 within an about 20 megabase region in Cannabis chromosome CM010796.2 located between about 40 megabases to about 60 megabases according to a method described above. The method further comprises crossing the first parent to a second parent that has a day length sensitive phenotype to produce F₁ progeny. Again, the skilled person understands that each of the F₁ progeny will be a carrier of the trait for the day length neutral phenotype such that it is not necessary to screen for a carrier using any of the methods described above. The method thus further comprises selfing the F₁ progeny to produce F₂ progeny, and identifying an F₂ progeny plant that has a day length neutral phenotype. While it is not necessary to screen the F₂ progeny according to the methods described above to determine whether or not a plant has a day length neutral phenotype (i.e., because 25% of the plants should have a day length neutral phenotype), the skilled person could do so if it were desirable to know the phenotype before flowering is initiated. Alternatively, the skilled person may want to screen the F₂ progeny for carriers of the trait.

In another aspect, the disclosure provides methods of producing a Cannabis plant with a day length neutral phenotype comprising initially identifying a first parent homozygous for at least one allele associated with a day length neutral phenotype at a polymorphic site corresponding to SEQ ID NOs: 69 to 115 according to a method described above. The method further comprises crossing the first parent to a second parent that has a day length sensitive phenotype to produce F₁ progeny. Again, the skilled person understands that each of the F₁ progeny will be a carrier of the trait for the day length neutral phenotype such that it is not necessary to screen for a carrier using any of the methods described above. The method thus further comprises selfing the F₁ progeny to produce F₂ progeny, and identifying an F₂ progeny plant that has a day length neutral phenotype. While it is not necessary to screen the F₂ progeny according to the methods described above to determine whether or not a plant has a day length neutral phenotype (i.e., because 25% of the plants should have a day length neutral phenotype), the skilled person could do so if it were desirable to know the phenotype before flowering is initiated. Alternatively, the skilled person may want to screen the F₂ progeny for carriers of the trait.

In another aspect, the disclosure provides methods of producing a Cannabis plant with a day length neutral phenotype comprising initially identifying a first parent homozygous for at least one allele associated with a day length neutral phenotype at a polymorphic site corresponding to SEQ ID NOs: 70 to 83 within an about 2.5 megabase region in Cannabis chromosome CM010796.2 located between about 41.09 megabases to about 43.59 megabases according to a method described above. The method further comprises crossing the first parent to a second parent that has a day length sensitive phenotype to produce F₁ progeny. Again, the skilled person understands that each of the F₁ progeny will be a carrier of the trait for the day length neutral phenotype such that it is not necessary to screen for a carrier using any of the methods described above. The method thus further comprises selfing the F₁ progeny to produce F₂ progeny, and identifying an F₂ progeny plant that has a day length neutral phenotype. While it is not necessary to screen the F₂ progeny according to the methods described above to determine whether or not a plant has a day length neutral phenotype (i.e., because 25% of the plants should have a day length neutral phenotype), the skilled person could do so if it were desirable to know the phenotype before flowering is initiated. Alternatively, the skilled person may want to screen the F₂ progeny for carriers of the trait.

In another aspect, the disclosure provides methods of producing a Cannabis plant with a day length neutral phenotype comprising initially identifying a first parent homozygous for at least one allele associated with a day length neutral phenotype at a polymorphic site corresponding to SEQ ID NOs: 70 to 83 within an about 0.75 megabase region in Cannabis chromosome CM010796.2 located between about 41.09 megabases to about 41.84 megabases according to a method described above. The method further comprises crossing the first parent to a second parent that has a day length sensitive phenotype to produce F₁ progeny. Again, the skilled person understands that each of the F₁ progeny will be a carrier of the trait for the day length neutral phenotype such that it is not necessary to screen for a carrier using any of the methods described above. The method thus further comprises selfing the F₁ progeny to produce F₂ progeny, and identifying an F₂ progeny plant that has a day length neutral phenotype. While it is not necessary to screen the F₂ progeny according to the methods described above to determine whether or not a plant has a day length neutral phenotype (i.e., because 25% of the plants should have a day length neutral phenotype), the skilled person could do so if it were desirable to know the phenotype before flowering is initiated. Alternatively, the skilled person may want to screen the F₂ progeny for carriers of the trait.

The skilled person also understands that there may be many reasons as to why it may be beneficial to maintain lines that are heterozygous for the trait that causes the day length neutral phenotype. First, the phenotype is not completely recessive, such that plants heterozygous for the allele are somewhat sensitive to day length and thus initiate flowering later than plants having the day length neutral phenotype when grown under long days. Thus, there may be situations where it would be advantageous to select for and cultivate plants that had such an intermediate phenotype. Second, vegetative propagation of plants having a day length neutral phenotype may induce flowering. Accordingly, it may be advantageous in some circumstances to maintain the autoflowering trait in heterozygous plants and then perform the cross (or self-pollinate) to produce plants having the day length neutral phenotype when it is desired.

With the understanding that the point mutation of SEQ ID NO: 9 from the PRR7 gene is causative of the day length neutral phenotype, then the skilled person will understand that any plant having a loss of function mutation in the PRR7 gene could be used in breeding programs to introduce the day length neutral phenotype into a day length sensitive variety. Thus, further aspects of the disclosure pertain to methods of producing a Cannabis plant with a day length neutral phenotype comprising initially performing a first cross of a first parent identified as having at least one loss of function mutation allele of the PRR7 gene according to a method as described above with a second parent that has a day length sensitive phenotype. The method further comprises performing a second cross of a first progeny of the first cross with a second progeny of the first cross to produce a plant that is homozygous for the loss of function mutation allele, which plant would have a day length sensitive phenotype.

Similarly, the skilled person understands that if the PRR5 and/or PRR9 genes function in at least a partially redundant manner with PRR7 in Cannabis, then any plant having a loss of function mutation in the PRR5 and/or PRR9 gene could be used in breeding programs to introduce the day length neutral phenotype into a day length sensitive variety. Thus, further aspects of the disclosure pertain to methods of producing a Cannabis plant with a day length neutral phenotype comprising initially performing a first cross of a first parent identified as having at least one loss of function mutation allele of the PRR5 and/or PRR9 gene according to a method as described above with a second parent that has a day length sensitive phenotype. The method further comprises performing a second cross of a first progeny of the first cross with a second progeny of the first cross to produce a plant that is homozygous for the loss of function mutation allele, which plant would have a day length sensitive phenotype.

### Polynucleotides and Kits

The skilled person further understands that the disclosure pertains to reagents and kits that are useful or necessary for performing the methods described above for identifying plants that have a day length neutral phenotype or are carriers of the trait for the day length neutral phenotype, and for marker assisted breeding of the day length neutral phenotype.

Thus, the disclosure further pertains to an allele-specific polynucleotide for use in the methods described herein. In particular embodiments, the polynucleotide is specific for a variation in the endogenous PRR7 gene. In particular embodiments, the polynucleotide is specific for the allele of PRR7 comprising the point mutation of SEQ ID NO: 9. In particular embodiments, the allele-specific polynucleotide is specific for an allele at a polymorphic site in the PRR7 gene, or an allele that is in linkage disequilibrium therewith. In various embodiments, the polynucleotide is detectably labeled, e.g., with a fluorescent dye.

The disclosure further pertains to an allele-specific polynucleotide for use in the methods described herein. In some embodiments, the polynucleotide is specific for a variation (e.g., a point mutation, an insertion, or a deletion) in any one or more of the 13 endogenous flowering related genes within the about 20 megabase region. In particular embodiments, the allele-specific polynucleotide is specific for variation at a polymorphic site as indicated for any one or more of SEQ ID NOs: 69 to 115, or an allele that is in linkage disequilibrium therewith. In other embodiments, the allele-specific polynucleotide is specific for a variation at a polymorphic site as indicated for any one or more of SEQ ID NOs: 69 to 91, or an allele that is in linkage disequilibrium therewith. In other embodiments, the allele-specific polynucleotide is specific for a variation at a polymorphic site as indicated for any one or more of SEQ ID NOs: 70 to 83, or an allele that is in linkage disequilibrium therewith. The allele-specific polynucleotide may be at least 16 nucleotides in length. In various embodiments, the polynucleotide is detectably labeled, e.g., with a fluorescent dye.

The disclosure further pertains to kits for use in the methods described above. The kits comprise at least one allele-specific polynucleotide as described above and at least one further component, e.g., a buffer, deoxynucleotide triphosphates (dNTPs), an amplification primer pair, an enzyme, or any combination thereof. The enzyme may be a polymerase or a ligase. In some embodiments, the kit comprises an amplification primer pair that comprises one or more of: i) a forward primer of SEQ ID NO: 20 and a reverse primer of SEQ ID NO: 22, ii) the forward primer of SEQ ID NO: 20 and a reverse primer of SEQ ID NO: 23, iii) a forward primer of SEQ ID NO: 21 and the reverse primer of SEQ ID NO: 22, iv) the forward primer of SEQ ID NO: 21 and the reverse primer of SEQ ID NO: 23, v) a forward primer of SEQ ID NO: 24 and a reverse primer of SEQ ID NO: 26, or vi) a forward primer of SEQ ID NO: 25 and the reverse primer of SEQ ID NO: 26.

In certain embodiments, the kits are provided to laboratories, whereby a breeder collects biological samples from individual plants and submits the samples to the laboratory for testing using the kits to determine the genotype at one or more polymorphic sites disclosed herein, and the plant's likelihood of having a day length neutral phenotype, and to provide the results to the breeder e.g., in the form of a report as described below. Alternatively, the kits include reagents for performing the testing of the samples by the breeders themselves.

The skilled person further understands that the methods disclosed herein could be automated and implemented with the use of computers. The results of a test to determine the identity of an allele at a polymorphic site may be presented as a “report”, that can be generated as part of a testing process, which can be provided to the breeder or any other intended recipient in physical or electronic form. Reports may include the alleles that the individual plant carries at various polymorphic sites and/or an individual plant's likelihood of having a day length neutral phenotype, or being a carrier of the trait.

### Molecular Modification

Presuming that the point mutation of SEQ ID NO: 9 from the PRR7 gene is causative of the day length neutral phenotype, then the skilled person would understand that a Cannabis plant could be genetically modified to decrease expression of PRR7 gene to result in the day length neutral phenotype.

Accordingly, a further aspect of the disclosure relates to methods for producing a day length neutral Cannabis plant comprising decreasing the expression of the endogenous PRR7 gene in the plant. Decreasing the expression of the endogenous PRR7 gene may include introducing or producing a loss of function allele in the endogenous gene. The loss of function allele may include a point mutation, an insertion, or a deletion. The point mutation may be a substitution. The point mutation may be a nonsense mutation. An insertion could include a T-DNA or a transposable element. Decreasing the expression of the endogenous PRR7 gene may comprise expressing a heterologous nucleic acid molecule homologous to a portion of the endogenous gene, wherein the heterologous nucleic acid molecule decreases expression of the endogenous PRR7 gene. The heterologous nucleic acid molecule may decrease expression of the PRR7 gene by RNA interference. The heterologous nucleic acid molecule may comprise a portion of SEQ ID NO: 1 (genomic sequence of PRR7).

A further aspect of the disclosure relates to methods of generating a Cannabis plant having a day length neutral phenotype comprising initially using a molecular methodology to identify a first plant as comprising a loss of function allele in the PRR7 gene. The method further comprises performing a first cross of said first plant to a second plant followed by performing a second cross of progeny from the first cross. Prior to performing the second cross, progeny from the first cross may be screened to identify plants comprising the loss of function allele in the PRR7 gene for use in the second cross. The method further comprises screening progeny of the second cross for a plant that is homozygous for the loss of function allele in the endogenous PRR7 gene. The loss of function allele in the endogenous PRR7 gene may be generated by genetic modification of the first plant or an ancestor thereof. The molecular methodology may include targeting induced local lesions in genomes (TILLING) methodology.

### Reduction of PRR7 Gene Expression

PRR7 gene expression and/or activity in genetically modified plants of the present invention may be reduced by any method that results in reduced activity of the PRR7 protein in the plant. This may be achieved, for example, by altering PRR7 gene activity at the DNA, mRNA and/or protein levels.

### Mutating the Endogenous PRR7 Gene

In one aspect, the present disclosure relates to genetic modifications targeting the endogenous PRR7 gene to alter PRR7 gene expression and/or activity. The endogenous PRR7 genes may be altered by, without limitation, knocking-out the PRR7 gene; or knocking-in a heterologous DNA to disrupt the PRR7 gene. The skilled person would understand that these approaches may be applied to the coding sequences, the promoter, or other regulatory elements necessary for gene transcription. For example, technologies such as CRISPR/Cas9 and TALENS can be used to introduce loss of function mutations in the endogenous PRR7 gene. Plants having at least one mutagenized allele comprising a loss of function mutation can then be self-fertilized to produce progeny homozygous for the loss of function alleles in the PRR7 gene.

Deletions lack one or more nucleotides of the endogenous PRR7 gene or residues of the endogenous PRR7 protein. For the purposes of this disclosure, a deletion variant includes embodiments in which no amino acids of the endogenous protein are translated, e.g., where the initial “start” methionine is substituted or deleted.

Insertional mutations typically involve the addition of material at a non-terminal point in the gene or polypeptide but may include fusion proteins comprising amino terminal and carboxy terminal additions. Substitutional variants typically involve a substitution of one amino acid for another at one or more sites within the protein.

### Expression of Transgenes Targeting the Endogenous Genes

In another aspect, the present disclosure relates to reducing the expression and/or activity of the endogenous PRR7 gene by targeting its mRNA transcripts. In this regard, levels of PRR7 mRNA transcripts may be reduced by methods known in the art including, but not limited to, co-suppression, antisense expression, short hairpin (shRNA) expression, interfering RNA (RNAi) expression, double stranded (dsRNA) expression, inverted repeat dsRNA expression, micro interfering RNA (miRNA), simultaneous expression of sense and antisense sequences, or a combination thereof. Various such methods are described in WO2018/027324.

In one embodiment, the present disclosure relates to the use of nucleic acid molecules that are complementary, or essentially complementary, to at least a portion of the molecule set forth in SEQ ID NO: 1. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO: 1 under physiological conditions.

The phenomenon of co-suppression in plants relates to the introduction of transgenic copies of a gene resulting in reduced expression of the transgene as well as the endogenous gene. The observed effect depends on sequence identity between the transgene and the endogenous gene.

The term “RNA interference” (RNAi) refers to well-known methods for down regulating or silencing expression of a naturally occurring gene in a host plant. RNAi employs a double-stranded RNA molecule or a short hairpin RNA to change the expression of a nucleic acid sequence with which they share substantial or total homology.

Antisense suppression of gene expression does not involve the catalysis of mRNA degradation, but instead involves single-stranded RNA fragments binding to mRNA and blocking protein translation.

PRR7 gene expression may be suppressed using a synthetic gene(s) or an unrelated gene(s) that contains about 20 bp regions or longer of high homology (preferably 100% homology) to the endogenous coding sequences of the PRR7 gene

Nucleic acid molecules that are substantially identical to portions of the PRR7 gene may also be used in the context of the disclosure. As used herein, one nucleic acid molecule may be “substantially identical” to another if the two molecules have at least 60%, at least 70%, at least 80%, at least 82.5%, at least 85%, at least 87.5%, at least 90%, at least 92.5%, at least 95%, at least 96%, at least 97%, at least 98%, at least 99%, or at least 99.5% sequence identity. Thus, a nucleic acid sequence comprising a nucleic acid sequence that is at least 60%, at least 65%, at least 70%, at least 75%, at least 80%, at least 85%, at least 90%, at least 92.5%, at least 95%, at least 96%, at least 97%, at least 98%, at least 99%, or at least 99.5% identical to SEQ ID NO: 1 may be suitable for use in the context of this disclosure.

Fragments of nucleic acid sequences of the PRR7 gene may be used. Such fragments may have lengths of at least 20, at least 30, at least 40, at least 50, at least 60, at least 70, at least 80, at least 90, at least 100, at least 150, at least 200, at least 250, at least 300, at least 350, or at least 400 contiguous nucleotides of a nucleic acid sequence of the PRR7 gene as the case may be.

In one embodiment, a genetically modified Cannabis plant of the disclosure comprises, stably integrated into its genome a nucleic acid molecule heterologous to the plant. The nucleic acid molecule encodes an RNA, e.g., a hairpin RNA, for reducing expression of the PRR7 gene. The nucleic acid molecule may be arranged in the sense orientation relative to a promoter. In another embodiment, the nucleic acid molecule may be arranged in the anti-sense orientation relative to a promoter. In a further embodiment, a genetic construct may comprise at least two nucleic acid molecules in both the sense and anti-sense orientations, relative to a promoter. A genetic construct comprising nucleic acids in both the sense and anti-sense orientations may result in mRNA transcripts capable of forming stem-loop (hairpin) structures.

In various instances, the nucleic acid molecule comprises at least 20, at least 30, at least 40, at least 50, at least 60, at least 70, at least 80, at least 90, at least 100, at least 150, at least 200, at least 250, at least 300, at least 350 or at least 400 contiguous nucleotides of a nucleic acid sequence possessing at least 80%, at least 85%, at least 90%, at least 92.5%, at least 95%, at least 96%, at least 97%, at least 98%, at least 99%, at least 99.5%, or 100% sequence identity to the nucleic acid sequence set forth in SEQ ID NO: 1.

The skilled person will also readily understand that although in the foregoing illustrative examples partial PRR7 gene sequences were suggested for constructing the constructs, complete PRR7 coding sequences, alternative PRR7 coding sequences, 5′UTR and/or 3′UTR, or mutated derivatives of these sequences can also be used. The maximum number of nucleic acid molecules that may be used in the context of the invention may be limited only by the maximum size of the construct that may be delivered to a target plant or plant cell using a given transformation method.

The skilled person would also appreciate that a nucleic acid molecule comprising the sequence of a PRR7 gene promoter and/or other regulatory elements may be used in the context of the invention. In an embodiment, a heterologous nucleic acid molecule comprising sequences of a PRR7 gene promoter and/or regulatory element may be used to bias the cellular machinery away from an endogenous PRR7 gene promoter thus resulting in reduced PRR7 expression.

### Plant Transformation

The introduction of DNA into plant cells by Agrobacterium mediated transfer is well known to those skilled in the art. Use of Agrobacterium transformation to transform Cannabis plants has been described, for example, in US patent application publication no. 2019/0085347A1.

Nevertheless, the present invention is not limited to any method for transforming plant cells, and the skilled person will readily understand that any other suitable method of DNA transfer into plant cells may be used. Methods for introducing nucleic acids into cells (also referred to herein as “transformation”) are known in the art as described in WO2018/027324.

Another method for introducing DNA into plant cells is by biolistics, which involves the bombardment of plant cells with microscopic particles (such as gold or tungsten particles) coated with DNA. The particles are rapidly accelerated, typically by gas or electrical discharge, through the cell wall and membranes, whereby the DNA is released into the cell and incorporated into the genome of the cell. This method is used for transformation of many crops, including corn, wheat, barley, rice, woody tree species and others.

Another method for introducing DNA into plant cells is by electroporation. This method involves a pulse of high voltage applied to protoplasts/cells/tissues resulting in transient pores in the plasma membrane which facilitates the uptake of foreign DNA.

Plant cells may also be transformed by liposome mediated gene transfer.

The nucleic acid constructs of the present invention may be introduced into plant protoplasts, which are cells in which its cell wall is completely or partially removed and then transformed with known methods.

A nucleic acid molecule of the present invention may also be targeted into the genome of a plant cell by a number of methods including, but not limited to, targeting recombination, homologous recombination, and site-specific recombination. Methods of homologous recombination and gene targeting in plants are known in the art.

As used herein, “targeted recombination” refers to integration of a nucleic acid construct into a site on the genome, where the integration is facilitated by a construct comprising sequences corresponding to the site of integration.

Homologous recombination relies on sequence identity between a piece of DNA that is introduced into a cell and the cell's genome. Homologous recombination is an extremely rare event in higher eukaryotes. However, the frequency of homologous recombination may be increased with strategies involving the introduction of DNA double-strand breaks, triplex forming oligonucleotides or adeno-associated virus.

“Site-specific recombination” as used herein refers to the enzymatic recombination that occurs when at least two discrete DNA sequences interact to combine into a single nucleic acid sequence in the presence of an enzyme. Enzymes and systems that have been developed to induce targeted mutagenesis and targeted deletions include Zinc Finger Nucleases (ZFNs), Meganucleases, Transcription Activator-Like Effector Nucleases (TALENS), and Clustered Regularly Interspaced Short Palindromic Repeats (CRISPR)-associated nucleases (e.g., CRISPR/Cas9).

In one embodiment, the polynucleotide encodes a zinc finger protein that binds to the PRR7 gene, resulting in reduced expression of the gene. In particular embodiments, the zinc finger protein binds to a regulatory region of the PRR7 gene. In other embodiments, the zinc finger protein binds to PRR7 mRNA to prevent its translation. Methods of selecting sites for targeting by zinc finger proteins have been described, for example, in U.S. Pat. No. 6,453,242, and methods for using zinc finger proteins to inhibit the expression of genes in plants are described, for example, in US2003/0037355.

The nucleic acid molecule becomes stably integrated into the plant genome such that it is heritable to daughter cells in order that successive generations of plant cells have reduced PRR7 gene expression. This may involve the nucleic acid molecules of the present invention integrating, for instance randomly, into the plant cell genome. Alternatively, the nucleic acid molecules of the present invention may remain as exogenous, self-replicating DNA that is heritable to daughter cells. As used herein, heterologous, self-replicating DNA that is heritable to daughter cells is also considered to be “stably integrated into the plant genome”.

Disruption of the endogenous PRR7 gene may be confirmed by methods known in the art of molecular biology. For example, disruption of endogenous genes may be assessed by PCR followed by Southern blot analysis. PRR7 mRNA levels may, for example, be measured by real time PCR, RT-PCR, Northern blot analysis, micro-array gene analysis, and RNAse protection.

### Targeted Screening for Loss of Function Mutations in the PRR7 Gene

This disclosure further relates to methods of generating Cannabis plants with day length neutral phenotype that involve targeted screening for loss of function mutations in the endogenous PRR7 gene and subsequent breeding of plants to obtain plants homozygous for the loss of function mutation at the locus. Cannabis breeders have used a variety of selection techniques in the development of improved cultivars. However, the most successful breeding method involves the hybridization of parents with a variety of different desired characteristics.

The term “T-DNA insertion” refers to methods utilizing transfer-DNA (T-DNA) for disrupting genes via insertional mutagenesis. Down-regulating or silencing expression of the endogenous PRR7 gene could be achieved by T-DNA mutagenesis, wherein the T-DNA is randomly inserted in the plant genome to introduce mutations. Subsequently, plants can be screened for T-DNA insertions in the PRR7 gene.

Mutations (including deletions, insertions, and point mutations) can also be introduced randomly into the genome of a plant cell by various forms of mutagenesis to produce non-natural variants. Methods for mutagenesis of plant material, including seeds, and subsequent screening or selection for desired phenotypes are well known, as described in WO2009/109012. Mutagenized plants and plant cells can also be specifically screened for mutations in the PRR7 gene, for example, by TILLING (Targeting Induced Local Lesions in Genomes). Loss of function mutations present in natural plant populations can be identified by EcoTILLING.

Once the loss of function mutation in the endogenous PRR7 gene has been identified, traditional breeding processes can be used to produce plants homozygous for the loss of function mutations at the endogenous PRR7 gene locus.

The skilled person understands that if the PRR5 and/or PRR9 gene functions in at least a partially redundant manner with the PRR7 gene in Cannabis, then the methods described in the sections above with respect to PRR7 pertaining to molecular modification, reduction in gene expression, gene mutation, transgene expression, plant transformation, screening for loss of function, and plants having a day length neutral phenotype would also apply to PRR5 and/or PRR9.

### Plants Having a Day Length Neutral Phenotype

The skilled person further understands that aspects of this disclosure pertain to novel plants having a day length neutral phenotype, or that are carriers of the day length neutral phenotype.

In one aspect, the disclosure pertains to a Cannabis plant or plant cell generated according to a method as described above in the sections entitled “Marker-assisted Breeding” and “Molecular Modification”.

In another aspect, the disclosure pertains to a genetically modified Cannabis plant or plant cell having a day length neutral phenotype, wherein the Cannabis plant or plant cell is genetically modified to have reduced expression of the endogenous PRR7 gene. In various embodiments, the plant or plant cell comprises an expression construct for reducing the expression of the endogenous PRR7 gene. The expression construct comprises a nucleic acid molecule encoding a hairpin RNA for reducing expression of the endogenous PRR7 gene. The endogenous PRR7 gene may have the sequence of SEQ ID NO: 1 or be at least 95% identical to SEQ ID NO: 1. The nucleic acid molecule encoding the hairpin RNA may comprises a portion of SEQ ID NO: 1. In some embodiments, the plant or plant cell is a seed or seed cell.

In various aspects, the disclosure pertains to seed produced by a Cannabis plant or plant cell as described above. In various aspects, the disclosure pertains to a method of producing seeds comprising growing a Cannabis plant as described above, allowing the Cannabis plant to flower, be pollinated, and set seed, and harvesting the seed from the plant.

In various aspects, the disclosure pertains to dried flower from a Cannabis plant having a day length neutral phenotype as described above, or comprising a Cannabis plant cell as described above.

In various aspects, the disclosure relates to a crop comprising a plurality of Cannabis plants as described above.

In various aspects, the disclosure relates to expression vectors for generating a day length neutral Cannabis plant, the expression vector comprising a nucleic acid comprising a portion of SEQ ID NO. 1.

In various aspects, the disclosure relates to use of a polynucleotide molecule having a sequence comprising a portion of SEQ ID NO: 1 for generating a Cannabis plant with a day length neutral phenotype.

In various aspects, the disclosure relates to use of a plant or plant cell as described above for the production of a cannabinoid and/or a terpene.

Various aspects of the disclosure relate to a method of producing a Cannabis plant with a day length neutral phenotype. The method comprises performing a first cross of a first parent identified having at least one loss of function mutation allele of the PRR5 and/or PRR9 gene according to the methods described herein with a second parent that has the day length sensitive phenotype. The method further comprises performing a second cross of a first progeny of the first cross with a second progeny of the first cross to produce a plant that is homozygous for the loss of function mutation allele.

Various aspects of the disclosure relate to a method for producing a day length neutral Cannabis plant. The method comprises decreasing the expression of an endogenous PRR5 and/or PRR9 gene in the plant.

Various aspects of the disclosure relate to a method of method of generating a Cannabis plant having a day length neutral phenotype. The method comprises i) using a molecular methodology to identify a first plant as comprising a loss of function allele in an endogenous PRR5 and/or PRR9 gene; ii) performing a first cross of said first plant to a second plant; iii) performing a second cross of progeny from the first cross; and iv) screening progeny of the second cross for a plant that is homozygous for the loss of function allele in the endogenous PRR5 and/or PRR9 gene.

Various aspects of the disclosure relate to a genetically modified Cannabis plant or plant cell having a day length neutral phenotype, as well as seed, plant material, or dried flower of such plants. The Cannabis plant or plant cell is genetically modified to have reduced expression of an endogenous PRR5 and/or PRR9 gene.

Various aspects of the disclosure relate to an expression vector for generating a day length neutral Cannabis plant, the expression vector comprising a nucleic acid comprising a portion of one or more of SEQ ID NOs: 14 to 17.

Various aspects of the disclosure relate to use of a polynucleotide molecule having a sequence comprising a portion of one or more of SEQ ID NOs: 14 to 17, for generating a Cannabis plant with a day length neutral phenotype.

In another aspect, the disclosure pertains to a genetically modified Cannabis plant or plant cell having a day length neutral phenotype, wherein the Cannabis plant or plant cell is genetically modified to have reduced expression of the endogenous PRR5 and/or PRR9 gene. In various embodiments, the plant or plant cell comprises an expression construct for reducing the expression of the endogenous PRR5 and/or PRR9 gene. The expression construct comprises a nucleic acid molecule encoding a hairpin RNA for reducing expression of the endogenous PRR5 and/or PRR9 gene. The endogenous PRR5 and/or PRR9 gene may have the sequence of one or more of SEQ ID NOs: 14 to 17, or be at least 95% identical to one or more of SEQ ID NOs: 14 to 17. The nucleic acid molecule encoding the hairpin RNA may comprises a portion of any one of SEQ ID NOs: 14 to 17. In some embodiments, the plant or plant cell is a seed or seed cell.

In various aspects, the disclosure relates to use of a plant or plant cell as described above for the production of cannabinoids and/or terpenes.

### Value Added Products

The skilled person understands that the disclosure further pertains to a cannabinoid and/or a terpene derived from a plant, plant cell, plant material, or seed as described above, or extracted from dried flower as described above. Thus, the disclosure further pertains to methods of producing a cannabinoid and/or a terpene comprising extracting the cannabinoid and/or the terpene from flowers harvested from Cannabis plants as described above.

The skilled person further understands that the Cannabis plants and plant cells described above could further be used to derive extracts, concentrates, isolates, and oils containing a cannabinoid and/or a terpene, which may be consumed or optionally used in the production of a variety of Cannabis products. The Cannabis products may be edible oils. The Cannabis products may be food items. The food items may be snack foods, e.g., candy. The candy may be chocolate, gummies, mints, lozenges, lollipops, or chewing gums. The food items may be baked goods, including, but not limited to, cookies, brownies, cakes, or breads. The Cannabis products may be beverages including, but not limited to, soft drinks, energy drinks, teas, coffees, juices, or waters.

The skilled person further understands that the Cannabis plants, plant cells, plant material, and seed described above could further be used to derive extracts, concentrates, isolates, and oils containing a cannabinoid and/or a terpene, which may be used in the production of topicals. The topicals may include, but are not limited to, cosmetics, massage creams, massage oils, bath oils, body oils, cosmetic oils, oils for toiletry purposes, skin creams, skin lotions, lip care preparations, face and body lotions, bath additives, soaps for personal use, beauty creams for body care, non-medicated skin preparations, medicated skin preparations.

The disclosure further pertains to extracts, concentrates, isolates, and oils containing a cannabinoid and/or a terpene derived from a Cannabis plant, plant cell, plant material, seed, or dried flower, as described above, and pharmaceutical compositions comprising such extracts, concentrates, isolates, and oils

The disclosure further pertains to methods of producing oils, extracts, and concentrates containing a cannabinoid and/or a terpene, the methods comprising extracting the oils, extracts, and concentrates from a Cannabis plant, plant cell, plant material, seed, or dried flower, as described above.

The disclosure further pertains to methods of producing isolates comprising a cannabinoid or terpene, the methods comprising isolating the cannabinoid or terpene from a Cannabis plant, plant cell, plant material, seed, or dried flower, as described above.

While specific embodiments of the invention have been described and illustrated, such embodiments should be considered illustrative of the invention only and not as limiting the invention as construed in accordance with the accompanying claims.

All applications and publications referred to herein are incorporated by reference in their entirety.

TABLE 1
List of autoflowering-associated genes within the low recombination 20 megabase region

| | Annotation in | SEQ ID NO for Genomic | SEQ ID NO for cDNA | SEQ ID NO for Protein |
|---|---|---|---|---|
| Gene Name | Arabidopsis | Sequence | Sequence | Sequence |
| PRR7 | AT5G02810.1 | 1 | 2 | 3 |
| RAP2.7, | AT2G28550.3 | 28 | 41 | 54 |
| TOE1 | | | | |
| ELF6 | AT5G04240.1 | 29 | 42 | 55 |
| GA2OX8 | BLASTx Results: | 30 | 43 | 56 |
| | gibberellin 2- | | | |
| | beta-dioxygenase | | | |
| | 8 | | | |
| SINAT3 | AT3G61790.1 | 31 | 44 | 57 |
| SKIP5 | AT3G54480.1 | 32 | 45 | 58 |
| LNK2 | AT3G54500.3 | 33 | 46 | 59 |
| RR9 | AT3G57040.1 | 34 | 47 | 60 |
| ERF105 | AT5G51190.1 | 35 | 48 | 61 |
| GRAS | AT3G49950.1 | 36 | 49 | 62, 63, and 64 |
| Family TF | | | | |
| ELF4 | AT2G40080.1 | 37 | 50 | 65 |
| FRIGIDA- | AT4G14900.1 | 38 | 51 | 66 |
| like | | | | |
| VRN5, VIL1 | AT3G24440.1 | 39 | 52 | 67 |
| EIN3 | AT3G20770.1 | 40 | 53 | 68 |

TABLE 2
Sequence alignment of partially represented PRR7 CDS transcripts
of autoflowering and photoperiod sensitive genotypes against the
Purple Kush genomic sequence (forward slashes indicate a break
in sequence for purposes of brevity; donor and acceptor splice
sites for the third intron of PRR7 and the G to T substitution
in the autoflowering assembled transcripts are in bold)

| | Sequence |
|---|---|
| CM010796.2: | AAAACATGCAAGGACATCCCTGTAATTAGTGAGTATATTTCCT/--/ |
| 41126584- | TTTGTGTTTTCACAGTGATGTCTTCTCATGATTCTAGGAGTATGG |
| 41126409 | |
| (Purple Kush | |
| genomic seq.) | |
| Photoperiod | AAAACATGCAAGGACATCCCTGTAATTA--------------/--/ |
| sensitive | ----------------TGATGTCTTCACATGATTCTAGGAGTATGG |
| PRR7 | |
| consensus | |
| CDS | |
| Autoflowering | AAAACATGCAAGGACATCCCTGTAATTATTGA---------/--/ |
| PRR7 | ----------------TGATGTCTTCACATGATTCTAGGAGTATGG |
| consensus | |
| CDS | |

TABLE 3
Nucleic acid amplification primers for verifying presence
or absence of 4 bp frameshift mutation in PRR7

| | | SEQ |
|---|---|---|
| | | ID |
| Primer Sequence | Description | NO: |
| ATCCCTGTAATTATTGATGATGTC | cDNA specific forward primer | 20 |
| | spanning exons 2 and 3 with | |
| | the inclusion of the | |
| | Autoflowering specific | |
| | splice inclusion mutation | |
| CATCCCTGTAATTATGATGTCTTC | cDNA specific forward primer | 21 |
| | spanning exons 2 and 3 | |
| | matching the Photoperiod | |
| | sensitive background | |
| GCTGCCACTGTTGTTGTCAG | Common reverse primer in | 22 |
| | exon 4 for amplification | |
| | of cDNA | |
| TAGGCCTCTCTGCAAAAGCC | Common reverse primer in | 23 |
| | exon 7 for amplification | |
| | of cDNA | |
| TGTGGCTACGAAGTTACAGCT | cDNA specific forward primer | 24 |
| | for sequencing spanning exons | |
| | 1 and 2 | |
| CTGAGGTAGTCATGCCCTGT | Forward primer for sequencing | 25 |
| | located in exon 2 | |
| AGTCAACGGCACCTTTCGAT | Reverse primer for sequencing | 26 |
| | located in exon 3 | |
| Primer Pair Combinations | | |
| (SEQ ID NOs) | Purpose | |

| | |
|---|---|
| Forward-SEQ ID NO: 20 | Designed to specifically amplify the |
| Reverse-SEQ ID NO: 22 | Autoflowering variant cDNA transcript |
| or 23 | of PRR7 with the inclusion of four |
| | additional base pairs that are included |
| | due to the predicted alternate splice |
| | site |
| Forward-SEQ ID NO: 21 | Designed to specifically amplify the |
| Reverse-SEQ ID NO: 22 | Photoperiod sensitive variant cDNA |
| or 23 | transcript of PRR7 based upon the |
| | original splice site |
| Forward-SEQ ID NO: 24 | Designed for the amplification |
| Reverse-SEQ ID NO: 26 | of PRR7 cDNA for sequencing |
| Forward-SEQ ID NO: 25 | Designed for the amplification |
| Reverse-SEQ ID NO: 26 | of PRR7 genomic DNA for sequencing |

TABLE 4
Autoflowering PRR7 splice variants and protein effects

| Splice Form | Count | Percentage | RNA Splice Effect | Protein Effect |
|---|---|---|---|---|
| Form 1 (SEQ ID NO: 146) | 7 | 41.2% | 82 bp exclusion | Frameshift/Early Stop |
| Form 2 (SEQ ID NO: 147) | 5 | 29.4% | 4 bp inclusion | Frameshift/Early Stop |
| Form 3 (SEQ ID NO: 148) | 4 | 23.5% | 21 bp exclusion | Deletion (7 Amino Acids) |
| Form 4 (SEQ ID NO: 149) | 1 | 5.9% | 7 bp exclusion | Frameshift/Early Stop |

TABLE 5
Polymorphic sites located within the Cannabis PRR7 gene.

| | | | | | SEQ |
|---|---|---|---|---|---|
| Polymorphism | | | Vari- | | ID |
| ID | Chromosome | Haplotype | ant | Genomic Context Sequence | NO: |
| SCM010796.2_ | CM010796.2 | C/T | T | TGATATTTCAAGAAACAACacaagaagaaggaaagaagaAAGGTAAA | 4 |
| 41123402 | | | | CAGGAAAAGAGTTTCTTCAACCATCCATTCAAGTTAAAATGATCATC | |
| | | | | TAGtcacgaaataataatattacaccCACTTAAAGTGATTTATTGAG | |
| | | | | AGAAGGCTCTATTCTTTTTGAGGGTTTATCACCTTCTATTTAGTAGA | |
| | | | | ATTGTAGTACTTA | |
| SCM010796.2_ | CM010796.2 | A/C | C | GGGGTGATTTTCTGTTTACCTTTTTCTCAAAGCATCTTTCTTGCCTC | 5 |
| 41124102 | | | | TTCTGGCGGAATCTTTTCAAAGCAGCTTCTCTTTGTGCAAATCGATT | |
| | | | | TCCATCAGATTCACTTCCTTCAATGGTTTTCCCTCTTCCAGTAACCA | |
| | | | | CACTCTCACTTTCTGTCTTTTTCCCCTGGAGTTTGAAGCATTGGTGC | |
| | | | | TTCTCTTTTGTCC | |
| SCM010796.2_ | CM010796.2 | T/C | C | CTTCATTGATCCTAGATTCATGTTCCTCTGAGAGCCCTTGGCGTTC | 6 |
| 41128199 | | | | TCTCGTAACCCTCtcccttctttcttttttcccaTCTTGAATCCGG | |
| | | | | TGATTCAGTTCAGTTAGCTCATCATTGGTTACGGGAGCTTTATTAT | |
| | | | | TCATCTGGACCATTTTCATCAACCAGATTCAATAAGATCAATGATG | |
| | | | | AGAAGGGTAATGGATAG | |
| SCM010796.2_ | CM010796.2 | G/T | T | TTGTGACAAAAATACAGGCTCAAGTAAAACAGAAAGCCCCTTCA | 7 |
| 41128443 | | | | CAGATCCTCTACAAAAGACAAATAATAAGTACAAATATATTGCTA | |
| | | | | TCAGAAACCAAGAAGAAGAATAAGGTGGATAGCATATGTGTGTG | |
| | | | | TCTGTGTGTTCTGATCAAAGTCCCAATAGCATAAAATGAGGAAAA | |
| | | | | GTTGAATTAGACACAACAAATAA | |
| SCM010796.2_ | CM010796.2 | T/G | G | AAGTACAAATATATTGCTATCAGAAACCAATAAGAAGAATAAGG | 8 |
| 41128513 | | | | TGGATAGCATATGTGTGTGTCTGTGTGTTCTGATCAAAGTCCCAA | |
| | | | | TAGCATAAAATTAGGAAAAGTTGAATTAGACACAACAAATAAAA | |
| | | | | GGGAAGGAAATGATTATGATCCTTTTCTATTCCCATATCTCTACTA | |
| | | | | CTTTCCCACTATCAATCTATAT | |
| SCM010796.2_ | CM010796.2 | C/A | A | CAAACACACACAATAAATGTATTAGTTAAAAGTATAAATTACAAA | 9 |
| 41126556 | | | | AAGATACTAGAGAAAAAAGTATTATTTTAAATCCAAAATAAAGG | |
| | | | | AAATATACTCACTAATTACAGGGATGTCCTTGCATGTTTTTTTGCT | |
| | | | | CATGATCTTGCCTAGAAGACCAATACCAGATAAACAGGGCATGA | |
| | | | | CTACCTCAGTCAAAACGAGATC | |

TABLE 6
Polymorphic sites located in autoflowering-associated genes
within a 20 megabase region of the Cannabis chromosome
CM010796.2 (polymorphic sites located between about
40 megabases to about 60 megabases).

| | | | | | SEQ |
|---|---|---|---|---|---|
| Chromosome_ | | | | | ID |
| Position | Gene | Haplotype | Variant | Genomic Context Sequence | NO: |
| CM010796.2_ | ELF4 | A/T | T | ACATCCCAGACCCAGAATaaaaaacaaacacaaaagaagagtaaagaaaaaa | 69 |
| 30981079 | | | | aaaacagagagcTTTATGAGATCAATGTCTTCACACACACACCAAAAATATA | |
| | | | | CCcataaactaaaacaaaaactcAAACCAAAAAAACGAACCGACCCCAAAAG | |
| | | | | ATTTCCCATTTACAATCATGTTTTCaagatgaatatatatatatt | |
| CM010796.2_ | TOE1/ | T/C | C | ttaatataattttccatctgtcctctctttctctcttccatATTTATGTCTG | 70 |
| 41829089 | AP2.7 | | | TGATCCATTTTTCTTCAGACCCAACTGCCGTTTTtctcttttcattttttca | |
| | | | | CACAACTTTACAGGAAAacctctgttttttttttttttttttttcccttctt | |
| | | | | gggtttttttttttgatctgAACTGGGTTTGGATTTTCTAGAGct | |
| CM010796.2_ | TOE1/ | TTTA/T | T | tcccttcttgggtttttttttttgatctgAACTGGGTTTGGATTTTCTAGAG | 71 |
| 41829236 | AP2.7 | | | ctttttttctgaaaaataatcCATTCAGGAAGTgggaattttttattttttt | |
| | | | | ttttttcccatttCAACCTCTTCAAAACAAATTTACCATCTGAAAATTTTGA | |
| | | | | TAAAGAGATCGAGAGagaaaatatataaagaaagaAACTGAGGAA | |
| CM010796.2_ | TOE1/ | T/TTA | TTA | cccttcttgggtttttttttttgatctgAACTGGGTTTGGATTTTCTAGAGC | 72 |
| 41829237 | AP2.7 | | | tttttttctgaaaaataatcCATTCAGGAAGTgggaattttttatttttttt | |
| | | | | tttttcccatttCAACCTCTTCAAAACAAATTTACCATCTGAAAATTTTGAT | |
| | | | | AAAGAGATCGAGAGagaaaatatataaagaaagaAACTGAGGAAT | |
| CM010796.2_ | TOE1/ | C/G | G | GGAAGATTCTGGTTCCTCTGGTTCTTCGGTTGTTAATGTTGAAATCGATGCT | 73 |
| 41829569 | AP2.7 | | | CTGAGTAGTACTACAACAATTACTACCTCTAATAGCGTCTTTAGAGAAGAAG | |
| | | | | ACTCTTCCATCAATGTCACGAACACAACATCTTCGACATTTTTCTTTGACAT | |
| | | | | AATGAAGAGAGAAAAGGACTGTAACAACGGTGCCACTGCCGggaa | |
| CM010796.2_ | TOE1/ | A/T | T | AGGACTGTAACAACGGTGCCACTGCCGggaaagaaactaataacatcTCTCC | 74 |
| 41829739 | AP2.7 | | | GCCGGGATTTCTAACCAGGTCTTTTTTCCCGGTGGCCGGAGAAAAGGTAGGG | |
| | | | | AATCAGTTCGTTGAAGCCGGTTCTGGGTCGTCTTCCTCGCGACCTCAGTGGT | |
| | | | | TGAACTTATCTTTTGCGGATTCCGGTGGAGGAGGCGCCGCCGTCC | |
| CM010796.2_ | TOE1/ | T/C | C | GAGAAAAGGTAGGGAATCAGTTCGTTGAAGCCGGTTCTGGGTCGTCTTCCTC | 75 |
| 41829829 | AP2.7 | | | GCGACCTCAGTGGTTGAACTTATCTTTTGCGGATTCCGGTGGAGGAGGCGCC | |
| | | | | GCCGTCCAACCGCCGGCGGATGTTAAAGTTTTGCAGCAGAAACAACAGATAA | |
| | | | | AGAAAAGCCGGCGGGACCAAGGTCCCGGAGCTCTCAGTACCGTGG | |
| CM010796.2_ | TOE1/ | T/C | C | GCAAAAATGCAGGCAATGACCACAACCTTGATCTGAACTTGGGAATTTCTCC | 76 |
| 41831468 | AP2.7 | | | ACCATGTGATGGTCCAAAAGGGCGTGAGTACTCTTTTGGTCTTGGGGACACT | |
| | | | | CGTGTTCATTGGAACCCTCGTGAAGGACCTCACAGAATAAGACCGATAATgg | |
| | | | | tacattgattttaataataGGCACAGTTTAGAAAGTGCAGTTTTC | |
| CM010796.2_ | TOE1/ | C/A | A | CATCCCCACATATTCTACCTCCCAACCATGCTGCTTCAGCTACATGTTCAGG | 77 |
| 41831731 | AP2.7 | | | AGTGTATCCAGCCTTCTTACCAAAACATGAGGTACTTTCTTTCGCATCCTCA | |
| | | | | CAAACTTGCCAAATACTTAAAGTTGATTATCTGAAACACTTTTTTGGGAGAT | |
| | | | | ACAGGAGATGAGAGCAATGGCAGATGATCATCAAAAGAGGATCGA | |
| CM010796.2_ | TOE1/ | T/C | C | TCTACCTCCCAACCATGCTGCTTCAGCTACATGTTCAGGAGTGTATCCAGCC | 78 |
| 41831744 | AP2.7 | | | TTCTTACCAAAACATGAGGTACTTTCTTTCGCATCCTCACAAACTTGCCAAA | |
| | | | | TACTTAAAGTTGATTATCTGAAACACTTTTTTGGGAGATACAGGAGATGAGA | |
| | | | | GCAATGGCAGATGATCATCAAAAGAGGATCGAAGCCGCCAATTCT | |
| CM010796.2_ | TOE1/ | G/A | A | ACCTCCCAACCATGCTGCTTCAGCTACATGTTCAGGAGTGTATCCAGCCTTC | 79 |
| 41831747 | AP2.7 | | | TTACCAAAACATGAGGTACTTTCTTTCGCATCCTCACAAACTTGCCAAATAC | |
| | | | | TTAAAGTTGATTATCTGAAACACTTTTTTGGGAGATACAGGAGATGAGAGCA | |
| | | | | ATGGCAGATGATCATCAAAAGAGGATCGAAGCCGCCAATTCTTCG | |
| CM010796.2_ | TOE1/ | C/A | A | ACCAAAACATGAGGTACTTTCTTTCGCATCCTCACAAACTTGCCAAATACTT | 80 |
| 41831801 | AP2.7 | | | AAAGTTGATTATCTGAAACACTTTTTTGGGAGATACAGGAGATGAGAGCAAT | |
| | | | | GGCAGATGATCATCAAAAGAGGATCGAAGCCGCCAATTCTTCGCAGGGTTTT | |
| | | | | TTGAACTGGGCATGGAAAATTCATGGCAATGGTAGCaacactact | |
| CM010796.2_ | TOE1/ | G/A | A | AAACATGAGGTACTTTCTTTCGCATCCTCACAAACTTGCCAAATACTTAAAG | 81 |
| 41831805 | AP2.7 | | | TTGATTATCTGAAACACTTTTTTGGGAGATACAGGAGATGAGAGCAATGGCA | |
| | | | | GATGATCATCAAAAGAGGATCGAAGCCGCCAATTCTTCGCAGGGTTTTTTGA | |
| | | | | ACTGGGCATGGAAAATTCATGGCAATGGTAGCaacactactacta | |
| CM010796.2_ | TOE1/ | A/G | G | ATACTTAAAGTTGATTATCTGAAACACTTTTTTGGGAGATACAGGAGATGAG | 82 |
| 41831847 | AP2.7 | | | AGCAATGGCAGATGATCATCAAAAGAGGATCGAAGCCGCCAATTCTTCGCAG | |
| | | | | GGTTTTTTGAACTGGGCATGGAAAATTCATGGCAATGGTAGCaacactacta | |
| | | | | ctactactactacatcTGGTTGTGTAACAGCCATGCCTACTTTCT | |
| CM010796.2_ | TOE1/ | A/G | G | AAACACTTTTTTGGGAGATACAGGAGATGAGAGCAATGGCAGATGATCATCA | 83 |
| 41831868 | AP2.7 | | | AAAGAGGATCGAAGCCGCCAATTCTTCGCAGGGTTTTTTGAACTGGGCATGG | |
| | | | | AAAATTCATGGCAATGGTAGCaacactactactactactactacatcTGGTT | |
| | | | | GTGTAACAGCCATGCCTACTTTCTCCATTGCAGCATCATCAGGAT | |
| CM010796.2_ | ELF4 | G/C | C | AACATTTCGAAGGTCCGTTCGCTCTACTCCGACCTCGGATCCAACTTCACAA | 84 |
| 51406087 | | | | CCTACTTCCACAACAACAAATCGCAGAATGGCAAGCCCGATAACCTTCGTCA | |
| | | | | TCATCATTGATATTCTTCGTTTAAAAAGTTTTCAAAATCATTGCTTTTAGTA | |
| | | | | TCttctttttaaataatcatGTTGTCTTTTAATAAAGTGAGGCTT | |
| CM010796.2_ | FRIGIDA | T/A | A | TCTTCTTCATCACCAAAAATAGGGTTTAGGGTTTTTCATATTCTTCCATTCA | 85 |
| 55539930 | | | | GTGACCTTTAAACTCCACCatttatcatcatcatcaatggCGACCGAATTGG | |
| | | | | TTGTCAACACGGAGAGAGTCCAGAAATTCTTCAACGACCTGGAGGAGCAAAA | |
| | | | | GACCATCCTTTCTACCTGCACTCAGCTATTCACAACCATATCCAC | |
| CM010796.2_ | FRIGIDA | T/C | C | GAAAGATTCTCTCCTGTTACTTTGCTCAAGTCTCATCTCAAGAACTTCAAAA | 86 |
| 55541565 | | | | AGAATGTTGCCAACATATTAAAGAATGGAAACAATAGCTTGGTTGCAATGGT | |
| | | | | TTGTCTATATTCTGACATATTTTACAAATGTCTTTTTGCATGTAGTCACATG | |
| | | | | ACTGcactttttaagattttataatCCAAGTCGTATCATCCACAG | |
| CM010796.2_ | FRIGIDA | C/T | T | AGAGAAAGAAGAGTTCAGGAGCCTCCAGTAAACCTCAAAATAAGCGAGGATA | 87 |
| 55541899 | | | | CAGTGCCAGCAGCGGccgaggtggtggtggtggatCATCTTCTTTTCGCCAA | |
| | | | | TCCAAAGCTGCCAAGTATTCCAACACTTATTCCTCTTTTAGCAGGAGGAATG | |
| | | | | CGAATCCTCCTCCGCCGGTTCATCACAGCACTGCTGCAAGATATT | |
| CM010796.2_ | FRIGIDA | G/A | A | TAAATTGCTACTGGAAACCTGTCTATTTTCTAGTGCAATGAAGTATAATAT | 88 |
| 5554113 | | | | GGACAATGAGGCAAGGCAATGTAAGTAGGTTAAGAGATAAAAGTTTCAAGG | |
| | | | | CACTGGAAAAGGAATGGAAAGTATAATCATAGTTAGGCAGAGAAAGATTT | |
| | | | | GAACTGTTATACGGTTCTTTTATGGGATGGGAGTTTATTATGAATTTCA | |
| CM010796.2_ | FRIGIDA | GGAAAAG | GAGAA | GCTACTGGAAACCTGTCTATTTTCTAGTGCAATGAAGTATAATATGGACAA | 89 |
| 5554113 | | GAA/GAG | AAGAA | TGAGGCAAGGCAATGTAAGTAGGTTAAGAGATAAAAGTTTCAAGGCACTGG | |
| | | AAAAGAA | | AAAAGGAATGGAAAGTATAATCATAGTTAGGCAGAGAAAGATTTGAACTG | |
| | | | | TTATACGGTTCTTTTATGGGATGGGAGTTTATTATGAATTTCAAGTGGA | |
| CM010796.2_ | ERF105 | G/A | A | TTTTCTCTCTTCTACCTCAATTTTCAAAATCAATCTCTCTCTACTCAATTT | 90 |
| 48050858 | | | | TCAAAATACTCTTATATATTATTACAACTTAATGGCTTTGGAAGGAGAAGT | |
| | | | | TACCACTTTGGAGCTCATAAGGCAACATCTTCTTAATGATTTTGGTTCAAT | |
| | | | | GGACAGTTTTATCAACAGCCTTAACAACTGTACGGCCAATATTAATAC | |
| CM010796.2_ | ERF105 | T/G | G | AGCTAGGCTAGCTATTCACAATGGCTAGAAAAAGTCAATGGGTAGTTAGGGT | 91 |
| 48051933 | | | | TTTATGTGTAAATATACGTATATGTATGTATGAGTGGTATTAATTATATGTT | |
| | | | | CAAATTAAGTAGTATTTTTTTTAGACAGCTTGCTTAAGTATGCAAAATGGCT | |
| | | | | GATGTACTATGATGTAAGCACCACTATTTATATGGTTGAGAATTC | |

TABLE 7
Polymorphic sites located in autoflowering-associated genes
within a 20 megabase region of the Cannabis chromosome
CM010796.2 (polymorphic sites located just outside the
about 40 megabases to about 60 megabases region).

| | | | | | SEQ |
|---|---|---|---|---|---|
| Chromosome_ | | | | | ID |
| Position | Gene | Haplotype | Variant | Genomic Context Sequence | NO: |
| AGQN03003328.1_ | ELF4 | G/C | C | AACATTTCGAAGGTCCGTTCGCTCTACTCCGACCTCGGAT | 92 |
| 15084 | | | | CCAACTTCACAACCTACTTCCACAACAACAAATCGCAGAA | |
| | | | | TGGCAAGCCCGATAACCTTCGTCATCATCATTGATATTCT | |
| | | | | TCGTTTAAAAAGTTTTCAAAATCATTGCTTTTAGTATCtt | |
| | | | | ctttttaaataatcatGTTGTCTTTTAATAAAGTGAGGCT | |
| | | | | T | |
| AGQN03003328.1_ | ELF4 | A/AT | AT | ATTGCTTTTAGTATCttctttttaaataatcatGTTGTCT | 93 |
| 15227 | | | | TTTAATAAAGTGAGGCTTCTGGGTGTAAGTCCTTGAGCTA | |
| | | | | ATTAGAGAGAGCCCACatggattatatatatatatatata | |
| | | | | tatatatattgtataagtATGAACTGTGTTTGTTGTGATG | |
| | | | | TCTGCTTGTCCCCTGTTTGTACTAATATGTGTAACACTTG | |
| | | | | T | |
| AGQN03003328.1_ | ELF4 | T/TA | TA | TGCTTTTAGTATCttctttttaaataatcatGTTGTCTTT | 94 |
| 15229 | | | | TAATAAAGTGAGGCTTCTGGGTGTAAGTCCTTGAGCTAAT | |
| | | | | TAGAGAGAGCCCACatggattatatatatatatatatata | |
| | | | | tatatattgtataagtATGAACTGTGTTTGTTGTGATGTC | |
| | | | | TGCTTGTCCCCTGTTTGTACTAATATGTGTAACACTTGTT | |
| | | | | GT | |
| AGQN03003328.1_ | ELF4 | A/ATT | ATT | TATCttctttttaaataatcatGTTGTCTTTTAATAAAGT | 95 |
| 15238 | | | | GAGGCTTCTGGGTGTAAGTCCTTGAGCTAATTAGAGAGAG | |
| | | | | CCCACatggattatatatatatatatatatatatatattg | |
| | | | | tataagtATGAACTGTGTTTGTTGTGATGTCTGCTTGTCC | |
| | | | | CCTGTTTGTACTAATATGTGTAACACTTGTTGTGTTTTAA | |
| | | | | A | |
| CM010796.2_ | ELF7 | C/CT | CT | ATCTAATGATGAGATCGAACATTTCCCTGCACCTTCAAGA | 96 |
| 9191898 | | | | GTAACTGTAAGGAAGAGACCAACTACTTCTGTTATAGAAT | |
| | | | | TGAAAGAACCAGAGGTACGGCCCTACTTGATGTTTTAGCT | |
| | | | | TTAACTGGTAGAGTGGGATTCAGTTGACTtgatatggttt | |
| | | | | tttttttcttgttttaggTTTATTCAAATTCAAGGGGAAG | |
| | | | | T | |
| CM010796.2_ | ELF7 | C/ | CTAAAT | TCTAATGATGAGATCGAACATTTCCCTGCACCTTCAAGAG | 97 |
| 9191899 | | CTAAAT | | TAACTGTAAGGAAGAGACCAACTACTTCTGTTATAGAATT | |
| | | | | GAAAGAACCAGAGGTACGGCCCTACTTGATGTTTTAGCTT | |
| | | | | TAACTGGTAGAGTGGGATTCAGTTGACTtgatatggtttt | |
| | | | | ttttttcttgttttaggTTTATTCAAATTCAAGGGGAAGT | |
| | | | | C | |
| CM010796.2_ | EMF2 | C/T | T | AAACCAAATAACATAAGTAGGAAACTCATCCGATTTTCTT | 98 |
| 11680304 | | | | TTTCACATTACTTTGGGACatagttattaatttttattaa | |
| | | | | ggTGACACAACTGATATGGACATTTAAGAAATGATTGGAC | |
| | | | | GAATAACCAGTTCAAGTGAACTTATAAAACTTAaaccttt | |
| | | | | ctttttctttttttcattaaaagaaAAGGCAGATGGTTAG | |
| | | | | A | |
| CM010796.2_ | EMF2 | C/G | G | TAGCTTTCTTAGTGGTGATTCTGTAGAACCGGTTCTAAAG | 99 |
| 11687670 | | | | CATGTATATCCCTTAGATTCATTACAATAATTTTAGTTGT | |
| | | | | TTTTCGTTtgctttttttttcctctttttctGTTTTAGTC | |
| | | | | CTATTTTAGTGTGGCTTGGGTCTCATTTAGACAAAATCAT | |
| | | | | TTAAATTGTCATATTGGCCATTTTTCTAGTTTGATTAAAT | |
| | | | | G | |
| CM010796.2_ | FLD | C/T | T | CTACTGATGCAGTAACGAGAGTTCTTCAAATTCTCAAGGG | 100 |
| 17880360 | | | | TAATGTATtatcaaaaataatttataaagctttattttca | |
| | | | | ttttaatttaatacccAGTTTTCTGACTTACTAAATCTGG | |
| | | | | AAGAAGATAATGCAATGAATCCGACACAATTCCATCATTC | |
| | | | | ACTATCCAATATATCATGGATGAttagaaaataagaaaag | |
| | | | | a | |
| CM010796.2_ | TOE1/A | TA/T | T | ttcttgggttttttatttttgatctGAACTGGGTTTGGAT | 101 |
| 61021736 | P2.7 | | | TTTCTAGAgcttttttttctgaaaaataatcCATTCAGGA | |
| | | | | AGTgggaattttttattttattttttttttcccattTCAA | |
| | | | | CCTCTTCAAAACAAATTCACCATCTGAAAATTTTGATAGA | |
| | | | | GATCGAGAGAGAAGATAGATAAAGAAAATTTTTGATATCA | |
| | | | | T | |
| CM010796.2_ | TOE1/A | T/A | A | tcttgggttttttatttttgatctGAACTGGGTTTGGATT | 102 |
| 61021737 | P2.7 | | | TTCTAGAgcttttttttctgaaaaataatcCATTCAGGAA | |
| | | | | GTgggaattttttattttattttttttttcccattTCAAC | |
| | | | | CTCTTCAAAACAAATTCACCATCTGAAAATTTTGATAGAG | |
| | | | | ATCGAGAGAGAAGATAGATAAAGAAAATTTTTGATATCAT | |
| | | | | G | |
| CM010796.2_ | TOE1/A | C/G | G | GGAAGATTCTGGTTCCTCTGGCTCTTCCGTTGTTAATGTT | 103 |
| 61022027 | P2.7 | | | GAAATCGATGCTCTGAGTAGTACTACAACTATTACTACCT | |
| | | | | CTAATGGCGTCTTTAGAGAAGAAGACTCTTCCATCAATGT | |
| | | | | CACGAACACAACATCTTCGACATTTTTCTTTGACATAATG | |
| | | | | AAGAGAGAAAAGGACTGTAACAACGGTGCCACTGCCGgga | |
| | | | | a | |
| CM010796.2_ | TOE1/A | A/T | T | AGGACTGTAACAACGGTGCCACTGCCGggaaagaaactaa | 104 |
| 61022197 | P2.7 | | | taacatcTCTCCGCCGGGATTTCTAACCAGGTCTTTTTTC | |
| | | | | CCGGTGGCCGGAGAAAAGGTAGGGAATCAGTTCGTTGAAG | |
| | | | | CCGGTTCTGGGTCGTCTTCCTCGCGACCTCAGTGGTTGAA | |
| | | | | CTTATCTTTTGCGGATTCCGGTGGAGGAGGCGCCGCCGTC | |
| | | | | C | |
| CM010796.2_ | TOE1/A | T/C | C | GAGAAAAGGTAGGGAATCAGTTCGTTGAAGCCGGTTCTGG | 105 |
| 61022287 | P2.7 | | | GTCGTCTTCCTCGCGACCTCAGTGGTTGAACTTATCTTTT | |
| | | | | GCGGATTCCGGTGGAGGAGGCGCCGCCGTCCAACCGCCGG | |
| | | | | CGGATGTTAAAGTTTTGCAGCAGAAACAACAGATAAAGAA | |
| | | | | AAGCCGGCGGGGACCAAGGTCCCGGAGCTCTCAGTACCGC | |
| | | | | G | |
| CM010796.2_ | TOE1/A | CT/C | C | CTTGGATTATTCGACAGCGAACTAGAGGCTGCAAGGTCCT | 106 |
| 61023460 | P2.7 | | | CATTTGTTCATGAATATATTTGAAGAATTCAATATTGTCC | |
| | | | | TCTAATATTGACaacattttctttttaactCGATAGAATA | |
| | | | | AAACACATTATGCCTTTGGCTTGGCTTTTGTTTGGTGGGG | |
| | | | | ATTTAATCCAACACaagttttttcttttatctttgTTTAA | |
| | | | | A | |
| CM010796.2_ | TOE1/A | C/T | T | ATATTGACaacattttctttttaactCGATAGAATAAAAC | 107 |
| 61023544 | P2.7 | | | ACATTATGCCTTTGGCTTGGCTTTTGTTTGGTGGGGATTT | |
| | | | | AATCCAACACaagttttttcttttatctttgTTTAAATTC | |
| | | | | AGGGCTTATGACAAAGCAGCCATCAAATGTAATGGAAGGG | |
| | | | | ATGCAATTACCAACTTTGAGCATAGTACTTATCAGGGTGA | |
| | | | | G | |
| CM010796.2_ | TOE1/A | T/A | A | AGTACTCTTTTGGTCTTGGGGACACTCGTGTTCATTGGAA | 108 |
| 61023923 | P2.7 | | | CCCTCGTGAAGGACCTCACAGAATAAGACCAATAATggta | |
| | | | | cattgattttaataataGGCACAGTTTAGAAAGTGCAGTT | |
| | | | | TTCTTTCCTTTCTgatataaaaactaaaattagtATCCAC | |
| | | | | TACTCCCAACAGATTGATGGGCAGTCATCCCCACATATTC | |
| | | | | T | |
| CM010796.2_ | TOE1/A | C/T | T | CTCGTGTTCATTGGAACCCTCGTGAAGGACCTCACAGAAT | 109 |
| 61023947 | P2.7 | | | AAGACCAATAATggtacattgattttaataataGGCACAG | |
| | | | | TTTAGAAAGTGCAGTTTTCTTTCCTTTCTgatataaaaac | |
| | | | | taaaattagtATCCACTACTCCCAACAGATTGATGGGCAG | |
| | | | | TCATCCCCACATATTCTACCTCCCAACCATGCTGCTTCAG | |
| | | | | C | |
| CM010796.2_ | TOE1/A | A/T | T | gtacattgattttaataataGGCACAGTTTAGAAAGTGCA | 110 |
| 61024000 | P2.7 | | | GTTTTCTTTCCTTTCTgatataaaaactaaaattagtATC | |
| | | | | CACTACTCCCAACAGATTGATGGGCAGTCATCCCCACATA | |
| | | | | TTCTACCTCCCAACCATGCTGCTTCAGCTACATGTTCAGG | |
| | | | | AGTGTATCCATCCTTCTTACCAAAACATGAGGTACTTTCT | |
| | | | | T | |
| CM010796.2_ | TOE1/A | C/A | A | ACCAAAACATGAGGTACTTTCTTTCGCATCATCACAAACT | 111 |
| 61024178 | P2.7 | | | TGCCAAATACTTAAAGTTGATTATCTGAAACACTTTTTTG | |
| | | | | GGAGATACAGGAGATGAGAGCAATGGCAGATGATCATCAA | |
| | | | | AAGAGGATCGAAGCCGCCAATTCTTCGCAGGGTTTTTTGA | |
| | | | | ACTGGGCATGGAAAATTCATGGCAATGGTAGCaacactac | |
| | | | | t | |
| SCM010796.2_ | TOE1/A | G/A | A | AAACATGAGGTACTTTCTTTCGCATCATCACAAACTTGCC | 112 |
| 61024182 | P2.7 | | | AAATACTTAAAGTTGATTATCTGAAACACTTTTTTGGGAG | |
| | | | | ATACAGGAGATGAGAGCAATGGCAGATGATCATCAAAAGA | |
| | | | | GGATCGAAGCCGCCAATTCTTCGCAGGGTTTTTTGAACTG | |
| | | | | GGCATGGAAAATTCATGGCAATGGTAGCaacactactact | |
| | | | | a | |
| SCM010796.2_ | TOE1/A | A/G | G | ATACTTAAAGTTGATTATCTGAAACACTTTTTTGGGAGAT | 113 |
| 61024224 | P2.7 | | | ACAGGAGATGAGAGCAATGGCAGATGATCATCAAAAGAGG | |
| | | | | ATCGAAGCCGCCAATTCTTCGCAGGGTTTTTTGAACTGGG | |
| | | | | CATGGAAAATTCATGGCAATGGTAGCaacactactactac | |
| | | | | tacatcTAGTTGTGTAACAGCCATGCCTACTTTCTCCATT | |
| | | | | G | |
| SCM010796.2_ | TOE1/A | A/G | G | AAACACTTTTTTGGGAGATACAGGAGATGAGAGCAATGGC | 114 |
| 61024245 | P2.7 | | | AGATGATCATCAAAAGAGGATCGAAGCCGCCAATTCTTCG | |
| | | | | CAGGGTTTTTTGAACTGGGCATGGAAAATTCATGGCAATG | |
| | | | | GTAGCaacactactactactacatcTAGTTGTGTAACAGC | |
| | | | | CATGCCTACTTTCTCCATTGCAGCATCATCAGGATTCTCA | |
| | | | | T | |
| SCM010796.2_ | TOE1/A | TG/T | T | CCAATTCTTCGCAGGGTTTTTTGAACTGGGCATGGAAAAT | 115 |
| 61024314 | P2.7 | | | TCATGGCAATGGTAGCaacactactactactacatcTAGT | |
| | | | | TGTGTAACAGCCATGCCTACTTTCTCCATTGCAGCATCAT | |
| | | | | CAGGATTCTCATCTTCCACATCTTTGGCTGCTCTATCAGC | |
| | | | | TACCAATAATATAAATCCCCAAGCCAATTttgttcaaaat | |
| | | | | a | |

1-201. (canceled)

202. A method comprising testing nucleic acid from a Cannabis plant to determine the presence or absence of a variation at a polymorphic site in an endogenous PRR7 gene.

203. The method of claim 202, wherein the variation is a point mutation, a deletion, or an insertion.

204. The method of claim 203, wherein the variation is a point mutation at position 101 of SEQ ID NO: 9 or its complement.

205. The method of claim 204, wherein the presence of A at position 101 of SEQ ID NO: 9 or T in the complement in both copies of the endogenous PRR7 gene indicates that the plant has a day length neutral phenotype.

206. The method of claim 205, wherein the point mutation causes a loss of function.

207. The method of claim 203, wherein the variation is a point mutation at position 101 of SEQ ID NO: 4 or its complement.

208. The method of claim 203, wherein the variation is a point mutation at position 101 of SEQ ID NO: 5 or its complement.

209. The method of claim 203, wherein the variation is a point mutation at position 101 of SEQ ID NO: 6 or its complement.

210. The method of claim 203, wherein the variation is a point mutation at position 101 of SEQ ID NO: 7 or its complement.

211. The method of claim 203, wherein the variation is a point mutation at position 101 of SEQ ID NO: 8 or its complement.

212. The method of claim 203, wherein the variation is an insertion starting at position 164 of SEQ ID NO: 12 or its complement.

213. A method comprising testing nucleic acid from a Cannabis plant to determine the presence or absence of a variation at a polymorphic site within a low-recombination region in the Cannabis genome or an allele in linkage disequilibrium with the polymorphic site within the low-recombination region.

214. The method of claim 213, wherein the low-recombination region is an about 20 megabase region in chromosome 5 (CM010796.2) between about 40 megabases to about 60 megabases.

215. The method of claim 214, wherein the variation at the polymorphic site corresponds to any one of SEQ ID NOs: 69 to 91.

216. The method of claim 213, wherein the low recombination region in Cannabis chromosome CM010796.2 is selected from: about 40 megabases to about 42.5 megabases, about 40 megabases to about 45 megabases, about 40 megabases to about 47.5 megabases, about 40 megabases to about 50 megabases, about 40 megabases to about 52.5 megabases, about 40 megabases to about 55 megabases, about 40 megabases to about 57.5 megabases, about 42.5 megabases to about 45 megabases, about 42.5 megabases to about 47.5 megabases, about 42.5 megabases to about 50 megabases, about 42.5 megabases to about 52.5 megabases, about 42.5 megabases to about 55 megabases, about 42.5 megabases to about 57.5 megabases, about 42.5 megabases to about 60 megabases, about 45 megabases to about 47.5 megabases, about 45 megabases to about 50 megabases, about 45 megabases to about 52.5 megabases, about 45 megabases to about 55 megabases, about 45 megabases to about 57.5 megabases, about 45 megabases to about 60 megabases, about 47.5 megabases to about 50 megabases, about 47.5 megabases to about 52.5 megabases, about 47.5 megabases to about 55 megabases, about 47.5 megabases to about 57.5 megabases, about 47.5 megabases to about 60 megabases, about 50 megabases to about 52.5 megabases, about 50 megabases to about 55 megabases, about 50 megabases to about 57.5 megabases, about 50 megabases to about 60 megabases, about 52.5 megabases to about 55 megabases, about 52.5 megabases to about 57.5 megabases, about 52.5 megabases to about 60 megabases, about 55 megabases to about 57.5 megabases, about 55 megabases to about 60 megabases, or about 57.5 megabases to about 60 megabases.

217. A method of producing a Cannabis plant with a day length neutral phenotype, the method comprising:
performing a first cross of a first parent having at least one allele associated with a day length neutral phenotype at a polymorphic site corresponding to SEQ ID NOS: 4 to 9, with a second parent that has a day length sensitive phenotype; and
identifying a first progeny plant from the first cross that has the day length neutral phenotype, or that is a carrier of a trait for the day length neutral phenotype, by testing nucleic acid from the first progeny plant to determine the presence or absence of a variation at a polymorphic site in an endogenous PRR7 gene.

218. The method of claim 217, wherein the first progeny plant is a carrier of the trait for the day length neutral phenotype, wherein the method further comprises self-pollinating the first progeny plant to produce F₂ progeny, and identifying an F₂ progeny plant that has the day length neutral phenotype by testing nucleic acid from the F₂ progeny plant to determine the presence or absence of a variation at a polymorphic site in the endogenous PRR7 gene.

219. The method of claim 217, wherein the first progeny plant is a carrier of the trait for a day length neutral phenotype, wherein the method further comprises:
backcrossing the first progeny plant to the first parent; and
identifying a progeny plant from the backcross that has the day length neutral phenotype by testing nucleic acid from the progeny plant to determine the presence or absence of a variation at a polymorphic site in the endogenous PRR7 gene.

220. The method of claim 217, wherein the first progeny plant is a carrier of the trait for the day length neutral phenotype, wherein the method further comprises:
backcrossing the first progeny plant to the second parent; and
identifying a progeny plant from the backcross that is a carrier of the trait for the day length neutral phenotype by testing nucleic acid from the progeny plant from the backcross to determine the presence or absence of a variation at a polymorphic site in the endogenous PRR7 gene.

221. The method of claim 217, wherein the first progeny plant is a carrier of the trait for the day length neutral phenotype, wherein the method further comprises:
performing a second cross of the first progeny plant with a second progeny plant identified as a carrier of the trait for the day length neutral phenotype by testing nucleic acid from the second progeny plant to determine the presence or absence of a variation at a polymorphic site in the endogenous PRR7 gene; and
identifying a progeny plant from the second cross that has the day length neutral phenotype by testing nucleic acid from the progeny plant from the second cross to determine the presence or absence of a variation at a polymorphic site in the endogenous PRR7 gene.


SEQUENCE LISTING (149 sequences)

SEQ ID NO: 1 - DNA, 6183 bp, Cannabis sativa
  source 1..6183: mol_type = genomic DNA
ctcttacgtggcgtcctacgtggcaacctttattcgctccatttagaactttgtagggacaattctaatatcttttttctcatttatatcatatatccatcgcttccatttacctttccaaccttccttctttctctaccaataatttttcttttcaattatttttttttaaatataatttggaaaaaaaaaatggtacaaaactcactgagatttctcaaactctacagaaaaaaataaataattttttttaaaaaaaaaaattaacttcataggaaaaaaaaaaaaagaaaaacctctggtttctaacttttgaactttattccaccattggcgattatcatccaagttcgaatgaagttttgagagagaggtattctcactttgtataaagtagcaacttgttgctcaatgttgattttcttttccctatttgtttcttcatcttttgtttggctgttaagaaaattttaaaggaacacaacgggaaaaatgttataatgaaggaacgagagtatttgatttttcacagtatagtgtttccgagtactactcactcattcactgaaaaatgaaaaaaaaatatgtatatatatgtagtatatttgtatttactgaattatttggctttctttctcttcccgctctctaaattgtttgcatgtattctataaattcataaattaatatatattttatctcggataattgaagtgttttttaacgtcttgatgaaatgaaaaggaaaatgacctttggaattaagtgttctgaaatcttccaaagaacctctttctcaccatatattagcaaaccctataagttttaattgagttgaatatgcatgattaaagtcttacacacttgttaattgtttgaagattcatgagtttggtatatcatgatgctgttattatctctatatcaaaagatagtttgatatgaactttatattcaggaacttttttattcttatttaaccatataggtgtgcttctgttattttttatgtccattatctgggttcagtttttgttaattatactgttatatgcatcaaaaaaagctacggtcaaatttatatgtgctacataccttgtatactttcgatcgcaatatagattgatagtgggaaagtagtagagatatgggaatagaaaaggatcataatcatttccttcccttttatttgttgtgtctaattcaacttttcctcattttatgctattgggactttgatcagaacacacagacacacacatatgctatccaccttattcttcttattggtttctgatagcaatatatttgtacttattatttgtcttttgtagaggatctgtgaaggggctttctgttttacttgagcctgtatttttgtcacaacgggatgtgcaagaactcttgtggatcagctttctcgccagctctatccattacccttctcatcattgatcttattgaatctggttgatgaaaatggtccagatgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaaggtcagtacgtttcagcaacatgaaatgatcattgcatttctctaatgcagaagtatacactctatgttaatggctttatgtttgattattgacggagatgacttttaaagtaaacactgtagtagatcttatgaataaacttgatgttatatgatatgaaaggaaaccatgtttatttgctccactacgagtaataatatccatggtctaatagttgttgtcattaaccataacctagaaagagtggtcttaggattagcctcgctgatccacactcacctcttgtagagaaggtcaagggttcaatccctcccccgccctccaaagaaagatataacaaacagaaaacgaatcaaaagaataaaaaacaaacaagtaaggtgagcatggcactattattagactaaagcttgttttagtttaatataagtagatcttatttctgaaatgttattttccaccatctaattacacacatttctagactgatcccctgatcttgtggcagcgctctttttctgagttcctatcagaaatgctgaaaactattaaactagtttttgttacttatttattttcttttgttttaacactacattttaacaacagttcctatactctggtgcttcacgtgtctatttgtgctattttgatgttcatatttatagtctagcgggaagttttttagtcatttcgttcatgaagggtcaagtacgatttcttgactagcttagattttgacatagaaccattcttgaggatactacagtgggttacttagtttgtagagtatgtttatgtgtaccttctaaagataacctggttataagtatgatattctcgaaaagaaaaacatgttgctccagacctgttggcaattgacatcttactttctagctatgataattagacattcagtcgctatatttatgtcatgctttcttcatcatcatttttcatatatgtgttcaagttatggtaagctatttaaatattgttattttatttgaccatgttttatttccaacgcaaactgcttggtataatattacataaatgttagccagaagctttgatttagtttacatcctaaaagtcaaaactagaattttattggttccctcaataaactaacaattattcaatgtattctcatattggcagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattagtgagtatatttcctttattttggatttaaaataatacttttttctctagtatctttttgtaatttatacttttaactaatacatttattgtgtgtgtttgtgtttgtgttttcacagtgatgtcttctcatgattctaggagtatggtctttaagtgtttatcgaaaggtgccgttgactttttagtgaaacctattcgaaagaatgagctgaaaaacctttggcaacatgtttggagaaaatgccacattgtgagttgcaatctatttgatttattatatcatggaccaattcaccttgaggttcaggtttcttctcatttatgcatttttgtttttcattaagcaccaattgtcgaactataccaaagaaaacaaacaaaaaaatggcttcaatctttgcttgataaaaaatatgttttatgtaatgcaaatatcacttaattgacataataccattaaacactgaaattgctcgagctgttcattaatttttcatattttcttcagtctagtaatagtggaagtgaaagtggaatatggattgaaaagcctttaaagtcaagaactgtggaacattctgacaacaacagtggcagcaatgatgaggatgatactgacagcattggtctaaatttcaggaatgaaagtgacagtggaacacaggtatttcactaaatttcatgaaagagtttttttttttggtatgaatgaagttttatgtctttgtatttacagaaatattagcgaaatgttagtttccacatgaagttttatgttttatgtatcaagaaactactataaatatgttagtttccacacacctagtttggaatttgtctatcacagcacccatatatctatttaccatgtttgcatattactcatttgatcttggtggaggaatattcatgtaagagtttttaatacttttatgtatatgtaagtggagaaggaatgataattagcaagataagaaaacaagaaaaagaatgaaaacttactactgagcttttacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttggtaggcaaattttcctaacatcttttttttttttaactttcttccattctcgatcattctctaagttatgatttatttaaattgtttagacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtatagaaatgttttgtgttgatataaacatgctcaatacaattaactctaaatttaagaatataactaagtcccattaccatgcaggtataacattgtttcaactgcaaacaagctccaacaaggaacataggaagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcctcccttcagactattattcaaggtaaatactcgaatatgatgcctcagtaattaattcaaataagacacaagcaaaggccacataccacttcataatatcttttcatcgatgttgatttgttttacaaaaactatggctgcagatgcttcgcagtgtggttcatccaatgcattgagagcacccatggaaggcaatattagtaatcacagtctcaacaggagtgggtcaggtagtaaccatggtagcaacggacaaaagagaagcaccaatgcttcaaactccaggggaaaaagacagaaagtgagagtgtggttactggaagagggaaaaccattgaaggaagtgaatcggatggaaatcgatttgcacaaagagaagctgctttgaaaagattccgccagaagaggcaagaaagatgctttgagaaaaaggtaaacagaaaatcaccccccttattttctctaagaagtaaatcatggaaacaaacaagtaggcgtctgaaagaaggaagtttcattttcaacactacacatttgaaccatcatctttgggatccagttagtatgaagctttttgggaaaaaaaaaaggaaaagaacagaaaattgtttccctgaaatataaataattgatctgtttcctttgaagttccatattgctgacctgaagcattaatttttactttttcaggtgagatatcagagtagaaaaaaactggcagaacaaagaccccgaattcgaggacaatttattagaaagggaatgaatgaaaacaagggaaaaggcataaattacgaacctgaaccaatttcataacaggagccattagaatccttgatgcagatgtggtgcgtttgcagtagatagttacagagcttttatgattgaatatatgggactgtattaa

SEQ ID NO: 2 - DNA, 1947 bp, synthetic construct
  misc_feature 1..1947: note = cDNA sequence of PRR7
  source 1..1947: mol_type = other DNA
atgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattatgatgtcttctcatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggaatatggattgaaaagcctttaaagtcaagaactgtggaacattctgacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggaacataggaagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcctcccttcagactattattcaagatgcttcgcagtgtggttcatccaatgcattgagagcacccatggaaggcaatattagtaatcacagtctcaacaggagtgggtcaggtagtaaccatggtagcaacggacaaaagagaagcaccaatgcttcaaactccaggggaaaaagacagaaagtgagagtgtggttactggaagagggaaaaccattgaaggaagtgaatcggatggaaatcgatttgcacaaagagaagctgctttga

SEQ ID NO: 3 - protein, 648 aa, Cannabis sativa
MNNKAPVTNDELTELNHRIQDGKKERRERVTRERQGLSEEHESRINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTEVVMPCLSGIGLLGKIMSKKTCKDIPVIMMSSHDSRSMVFKCLSKGAVDFLVKPIRKNELKNLWQHVWRKCHISSNSGSESGIWIEKPLKSRTVEHSDNNSGSNDEDDTDSIGLNFRNESDSGTQSSWTKRAEVDSPQAVSWEQFADLPDSTNHQVNHPRQEAFGNNWVPENATVTPRPHNDELDKKVMGKDLKIGLPSLLEDTSEKGLTNMEGTNKDKCSELNSKKDDQEQEKRELDLNNEEPSAEKTQAVDLMGVSNYSIDPHMESGVLDVPNELSKAACMRDNANHENKETPFFELILKRPRDIQDTGTSAHDRNVLRHSDISAFSRNIGSCSPLDNSSDAAKTESIPNLQSDSNGTPPNQGSNGSSNNNDMGSTTNNAFTKQVAFAERPTNKSTIKLQSNTGFQQVQNGQASLQTIIQDASQCGSSNALRAPMEGNISNHSLNRSGSGSNHGSNGQKRSTNASNSRGKRQKVRVWLLEEGKPLKEVNRMEIDLHKEKLL

SEQ ID NO: 4 - DNA, 201 bp, Cannabis sativa
  variation 101: note = "y" is t or c, wherein t is the predictive variant
  source 1..201: mol_type = genomic DNA
tgatatttcaagaaacaacacaagaagaaggaaagaagaaaggtaaacaggaaaagagtttcttcaaccatccattcaagttaaaatgatcatctagtcaygaaataataatattacacccacttaaagtgatttattgagagaaggctctattctttttgagggtttatcaccttctatttagtagaattgtagtactta

SEQ ID NO: 5 - DNA, 201 bp, Cannabis sativa
  variation 101: note = "m" is a or c, wherein c is the predictive variant
  source 1..201: mol_type = genomic DNA
ggggtgattttctgtttacctttttctcaaagcatctttcttgcctcttctggcggaatcttttcaaagcagcttctctttgtgcaaatcgatttccatcmgattcacttccttcaatggttttccctcttccagtaaccacactctcactttctgtctttttcccctggagtttgaagcattggtgcttctcttttgtcc

SEQ ID NO: 6 - DNA, 201 bp, Cannabis sativa
  variation 101: note = "y" is t or c, wherein c is the predictive variant
  source 1..201: mol_type = genomic DNA
cttcattgatcctagattcatgttcctctgagagcccttggcgttctctcgtaaccctctcccttctttcttttttcccatcttgaatccggtgattcagytcagttagctcatcattggttacgggagctttattattcatctggaccattttcatcaaccagattcaataagatcaatgatgagaagggtaatggatag

SEQ ID NO: 7 - DNA, 201 bp, Cannabis sativa
  variation 101: note = "k" is g or t, wherein t is the predictive variant
  source 1..201: mol_type = genomic DNA
ttgtgacaaaaatacaggctcaagtaaaacagaaagccccttcacagatcctctacaaaagacaaataataagtacaaatatattgctatcagaaaccaakaagaagaataaggtggatagcatatgtgtgtgtctgtgtgttctgatcaaagtcccaatagcataaaatgaggaaaagttgaattagacacaacaaataa

SEQ ID NO: 8 - DNA, 201 bp, Cannabis sativa
  variation 101: note = "k" is g or t, wherein g is the predictive variant
  source 1..201: mol_type = genomic DNA
aagtacaaatatattgctatcagaaaccaataagaagaataaggtggatagcatatgtgtgtgtctgtgtgttctgatcaaagtcccaatagcataaaatkaggaaaagttgaattagacacaacaaataaaagggaaggaaatgattatgatccttttctattcccatatctctactactttcccactatcaatctatat

SEQ ID NO: 9 - DNA, 201 bp, Cannabis sativa
  variation 101: note = "m" is a or c, wherein a is the predictive variant
  source 1..201: mol_type = genomic DNA
caaacacacacaataaatgtattagttaaaagtataaattacaaaaagatactagagaaaaaagtattattttaaatccaaaataaaggaaatatactcamtaattacagggatgtccttgcatgtttttttgctcatgatcttgcctagaagaccaataccagataaacagggcatgactacctcagtcaaaacgagatc

SEQ ID NO: 10 - protein, 179 aa, Cannabis sativa
MNNKAPVTNDELTELNHRIQDGKKERRERVTRERQGLSEEHESRINEDVQQHVSNGEIGTVQALERSHSGQRRSQQQPQGHLVRWERFLAFRSLKVLLVENDDSTRHIVSALLRNCGYEVTAVENGRQAWKVLEDLVTDVDLVLTEVVMPCLSGIGLLGKIMSKKTCKDIPVIIDDVFS

SEQ ID NO: 11 - DNA, 401 bp, Cannabis sativa
  source 1..401: mol_type = genomic DNA
ttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattagtgagtatatttcctttattttggatttaaaataatacttttttctctagtatctttttgtaatttatacttttaactaatacatttattgtgtgtgtttgtgtttgtgttttcacagtgatgtcttctcatgattctaggagtatggtctttaagtgtttatcgaaaggtgccgttgactttttagtgaaacctattcgaaagaatgagctgaaaaacctttggcaacatgtttggag

SEQ ID NO: 12 - DNA, 287 bp, Cannabis sativa
  source 1..287: mol_type = other DNA
ttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattattgatgatgtcttcacatgattctaggagtatggtctttaagtgtttatcgaaaggtgccgttgactttttagtgaaacctattcgaaagaatgagctgaaaaacctttggcaacatgtttggag

SEQ ID NO: 13 - DNA, 283 bp, Cannabis sativa
  source 1..283: mol_type = other DNA
ttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattatgatgtcttcacatgattctaggagtatggtctttaagtgtttatcgaaaggtgccgttgactttttagtgaaacctattcgaaagaatgagctgaaaaacctttggcaacatgtttggag

SEQ ID NO: 14 - DNA, 4062 bp, Cannabis sativa
  source 1..4062: mol_type = genomic DNA
aaaaatatctaccaaatatagttttctatttacaactcccacaactctaagttttttttctgagctttcttcctcaagcttttcccagcttatcaacaattcaagactggattttttcggtttaggaagaaaaaagactggatttttacagatttagagagagaatttgaagatgggggaggctgttttgagtagtagtgaggattgggcagtgaaaggagatagtgaagttgagaaaaggaggaagaagaagatggaagaaatggagaataataataaaattgataatgattgtggtgatgattgtgatgatggtgatgatgatgaagttgtggagattaagatgaaagatgggtgtagctcaagtggagctattactacctgggaaagatttttgcccagaatgatgatgagggttttgttggttgaagctgatgattcaacaaggcagattattactgctcttctcagaaaatgtagctatagaggtatgtatgttgtattgtgtttcttttaatttgattttgcttttatgttttttttagggggtttgcaaaattgaaatggggtttcttccaattttgtgtttttggtcaagtttatgtaattggggtgaaatttcatgtttttgttgctttaatgtttgttgtaaattggaaaaaaaaaaaccaattgaaggaaggatatggtttgacttttactctatttagaactcagattttagggttttaaaaaaatgaaacaagaatatgtgaatcaaatttagaatctgagatccaaacaaagcccaagtgaattgagggggttgttttctaatggcttagcttccttatgtttatgctgtttattatgattagtaactgaaaatgttttctttggtttgggcagttgctgctgtggctgatggcttaaaagcatgggaattactgaaaggaagaccccataacatagatcttatattgacagaagtggatcttccatcagtatccggttttcggttacttaccttaatcatggaacatgatgtttgcaaaaacattcctgttataagtatgtttttcaaatatggaaaaatgatgtatttgttaagtgtttagtgtttttcgattttcatgtacttatgtattttgtggatctttgcagtgatgtcttcacaagattcgatcggtatagtttacagatgcatgatgagaggtgctgctgattatcttgttaagcctattagaggaatgaactgagtaatctatggcaacatgtttggagaagaaaatctgtaagcacaggcttttaagatttcgtgttgtatcgaatattattgtcttgttcttacaatgttctaattttgtttgtttcgtgattctattaggcctcgagaaatggtcttcgatatgaaggtgttgcacaaaataagggtgaagccacttctgaaaatgaccctgcaaccaatcaatccaatggctatattccttccattcaaagaaatgaagaaaacatcgataaagggagcgattctcaggtatctcatattttctgtatgagcctcccaagaatttgtagcctttattgtttgacagaaatgtaattgctagtatctgaatgttgaagatattaacaagtgggttttagtttagaataaatttagactggtcataagcgatatagagtatgttcttcgccgaggaatatgttagtattaattcttaaaaaaggaatttccttccagtctaggtttcatatagagactgcatgtttgctgtctcctgcagagctcttgtacaaggccagaaatggaggttgagaatgtccacgtcgatcagatgcaggaatgctcacagccaacacggggcgagtcttttccaagtgactcgagaacacagaagagtgaagggcacatcaatacaggacagagattcatgcatggaaatgatgcaagaggtgaataatattacaacttttttttaagtattattcttgaatcagtgttcaagaatctaacggtgtcacaatcgaaataggttcaatgatgggaatctatcaggatggtgaaaccgaaatttcaccaggcaagtgttccaagccagaaagtattagtaggagggatgctattatgtctagtgaggcttgtgagaacaataaattcttgtttaatcattctagaggcaccattgatttgaatggagctttcgatgaaaatcaaaattgcaattatcaaaattgttcatctagtaatggaactagcaagtttgattctggtccaaaattggatcttaccttgagatcaagttctggtgactttgagagccatatggttgagaagaggcacaaccttggtcattctaatgcttcggcctttacacggtgagttctatttcgatcatctcactttttgagttttgtggtttcttcacatgatccaagtaatttggaccaagtgagacatgtcatgatgatatggaaagtttcatcaattggattatctttcttaacttgtatttgctaaacatttcggtgattattcatgcagatacatcaataaaccgttgcagcccacaaaccctacatcagccggtgtatgcgatcaagataaagaaagcggatcaaacaccgaaaagcatatgttcggttttgctgctggacttagttttaactctgataccccccctggtccctcaaaaagctcaccgaaaagtcatattacattggctacaggtcaatctaaccattctgaagttgcaactcattcccctcaaccgagattatttcctctaccgattcctgtgaaaggtgtaagggttaataatccagtggcaggctatggttctgtagtagtaccttcagggttctgttcacaatcaggttcttcatccgggatcagtccaagctcggttgcccaacaagaatcttcattgctaatcaatgcgttttaccaaacgactcctgaaaagaacaaatctgagcagtcaaatggcctttgtcgaaacaacagcatcgccaaagatcaatccttgcaaaatcaagaacataaattggattttttcgaagatcgtgggcgtatctctcctacaaccgatcaaagtgctagtagtagtttgtgtaatggaaatgcaagccatctaaatagtattgggtatggaagtgcttgtggaagtaatagcaatgttgatcagactgccatgattagaactgtttccgagaacaagaatgatgaaagttactttaataacagcgggaattctcaccgagccattcaaagagaagcagctcttacaaaatttcgaatgaagaggaaagatagatgctttgaaaagaaggtataacattgtttaccattctcaattccattactttttttaagtgttcttcgtttctcttacgaattcatgtctattccaggttcgttacgagagtagaaagaaactcgctgagcaacgtccccgagtcaagggacagtttgttcgccaagcgcaaacagatccttcgacagcagaaaccgaaggaaattcttaggagttaataactatttagagctttgaataacattaatgattgaagaaaacgctcataatgatgatcaatagtagtttatttcgaaatcttttacttcttttgttgtagtatgagagtgaactttgggggccataagcacaagagaaaatcacttagtgtggtttatgtatagctttgtaatttctactcaattctttttttctttttgggtagctgtgaattttgttgattaacaatctcataggtttatattggtatatacactcttttagtattgtgatttgtatccatagtaacaccatcattaccaatcaccagcttgtatactactatatatgaaagacttcattttttttagtagttcttacaatttttgtatttgtataaatacacaaagtggtattgagtattgatttgacaagtttcaataaagggtttgttctttacaaaatactttttgaaataaagataagtgtggttatgctatgaaggtgtgccattgaaatttgttagagctgaaaagaacaaaaaccataatataaattgactaccatt

SEQ ID NO: 15 - DNA, 4187 bp, Cannabis sativa
  source 1..4187: mol_type = genomic DNA
aagtacatcacttcaaacaattgaatcggttacctattttcttaaaaactttacctttgcacaattatgatccatgggatttaactttttcacaattagaatccaatctaacagctcaaattgaattgagacatctcacacctcaaattgaaaagcgaggtactgcataaaaatatctaccaaatatagttttctatttacaactcccacaactctaagttttttttctgagctttcttcctcaagcttttcccagcttatcaacaattcaagactggattttttcggtttaggaagaaaaaagactggatttttacagatttagagagagaatttgaagatggggaggctgttttgagtagtagtgaggattgggcagtgaaaggagatagtgaagttgagaaaaggaggaagaagaagatggaagaaatggagaataataataaaattgataatgattgtggtgatgattgtgatgatggtgatgatgatgaagttgtggagattaagatgaaagatgggtgtagctcaagtggagctattactacctgggaaagatttttgcccagaatgatgatgagggttttgttggttgaagctgatgattcaacaaggcagattattactgctcttctcagaaaatgtagctatagaggtatgtatgttgtattgtgtttcttttaatttgattttgcttttatgttttttttagggggtttgcaaaattgaaatggggtttcttccaattttgtgtttttggtcaagtttatgtaattggggtgaaatttcatgtttttgttgctttaatgtttgttgtaaattggaaaaaaaaaaaccaattgaaggaaggatatggtttgacttttactctatttagaactcagattttagggttttaaaaaaatgaaacaagaatatgtgaatcaaatttagaatctgagatccaaacaaagcccaagtgaattgagggggttgttttctaatggcttagcttccttatgtttatgctgtttattatgattagtaactgaaatgtttttctttggtttgggcagttgctgctgtggctgatggcttaaaagcatgggaattactgaaaggaagaccccataacatagatcttatattgacagaagtggatcttccatcagtatccggttttcggttacttaccttaatcatggaacatgatgtttgcaaaaacattcctgttataagtatgtttttcaaatatggaaaaatgatgtatttgttaagtgtttagtgtttttcgattttcatgtacttatgtattttgtggatctttgcagtgatgtcttcacaagattcgatcggtatagtttacagatgcatgatgagaggtgctgctgattatcttgttaagcctattagaggaatgaactgagtaatctatggcaacatgtttggagaagaaaatctgtaagcacaggcttttaagatttcgtgttgtatcgaatattattgtcttgttcttacaatgttctaattttgtttgtttcgtgattctattaggcctcgagaaatggtcttcgatatgaaggtgttgcacaaaataagggtgaagccacttctgaaaatgaccctgcaaccaatcaatccaatggctatattccttccattcaaagaaatgaagaaaacatcgataaagggagcgattctcaggtatctcatattttctgtatgagcctccaaagaatttgtaacctttattgtttgacagaaatgtaattgctagtatctcaatgttgaagatattaatgaacggttttagtttagaataaatttagactggtcataagcgatatagagtatgttcttcgccgaggaatatgttagtattaattcttaaaaaaggaatttccttccagtctaggtttcatatagagactgcatgtttgctgtctcctgcagagctcttgtacaaggccagaaatggaggttgagaatgtccacgtcgatcagatgcaggaatgctcacagccaacacggggcgagtcttttccaagtgactcgagaacacagaagagtgaagggcacatcaatacaggacagagattcatgcatggaaatgatgcaagaggtgaataatattacaacttttttttaagtattattcttgaatcagtgttcaagaatctaacggtgtcacaatcgaaataggttcaatgatgggaatctatcaggatggtgaaaccgaaatttcaccaggcaagtgttccaagccagaaagtattagtaggagggatgctattatgtctagtgaggcttgtgagaacaataaattcctgttcaatcattctagaggcaccattgatttgaatggagctttcgatgaaaatcaaaattgcaattatcaaaattgttcatctagtaatggaactagcaagtttgattctggtccgaaattggatcttaccttgagatcaagttctggtgactttgagagccatatggttgagaagaggcacaaccttggtcattctaatggttcggcctttacacggtgagttctatttcgatcatctcactttttgaccagtgagatatgtcatgatgatatggaaagtttcatcaattggattatctttcttaacttgtatttgctacatttcggtgattattcatgcagatacatcaataaaccgttgcagcccacaaaccctacatcagccggtgtatgcgatcaagataaagaaagcggatcaaacaccgaaaagcatatgttcggttttgctgctggacttagttttaactctgataccccccctggtccctcaaaaagctcaccgaaaagtcatattacattggctacaggtcaatctaaccattctgaagttgcaactcattcccctcaaccgagattatttcctctaccgattcctgtgaaaggtgtaagggttaataatccagtggcaggctatggttctgtagtagtaccttcagggttctgttcacaatcaggttcttcctccgggatcagtccaagctcggttgcccaacaagaatcttcattgctaatcaatgcgttttaccaaacgactcctgaaaagaacaaatctgagcagtcaaatggcctttgtcgaaacaacagcatcgccaaagatcaatccttgctaaatcaagaacataaattggattttttcgaagatcgtgggcgtatctctcctacaaccgatcaaagcgctagtagtagtttgtgtaatggaaatgcaagtcatcttaatagtattgggtatggaagtgcttgtggaagtaatagcaatgttgatcagactgccatgattagaactgtttccgagaacaagaatgatgaaagttactttaataacagcgggaattctcaccgagccattcaaagagaagcagctcttacaaaatttcgaatgaagaggaaagatagatgctttgaaaagaaggtataacattattaccattctcaatttcattactttttttaagtgttcttcatttttctaacgaattcatgtctattccaggtccgttacgagagcagaaagaaactcgctgagcaacgtccccgagtcaagggacagtttgttcgccaagcgcaaacagatccttcgatagcagaaaccgaaggaaattcttaggagttaataactatttagagctttgaataacattaatgattgaagaaaacactcataatgatgatcaatagtagtttatttcgaaatcttttacttcttttgttgtagtatgagagtgaactttgggggccataagcacaagagaaaatcacttagtgtggtttatgtatagctttgtaatttctactcaattctttttttctttttgggtagctgtgaattttgttgattaacaatctcataggtttatattggtatatacactcttttagtattgtgatttgtatccatagtaacaccatcattaccaatcaccagcttgtatactactatatatgaaagacttcattttttttagtagttcttacaatttttgtatttgtataaatacacaaagtggtattgagtattgatttgacaagtttcaataaagggtttgttctttacaaaatactttttgaaataaagataagtgtggttatgctatgaaggtgtgccattgaaatttgttagagctgaaaagaacaaaaaccataatataaattgactaccatt

SEQ ID NO: 16 - DNA, 3155 bp, Cannabis sativa
  source 1..3155: mol_type = genomic DNA
atgtttttctttggtttgggcagttgctgctgtggctgatggcttaaaagcatgggaattactgaaaggaagaccccataacatagatcttatattgacagaagtggatcttccatcagtatccggttttcggttacttaccttaatcatggaacatgatgtttgcaaaaacattcctgttataagtatgtttttcaaatatggaaaaatgatgtatttgttaagtgtttagtgtttttcgattttcatgtacttatgtattttgtggatctttgcagtgatgtcttcacaagattcgatcggtatagtttacagatgcatgatgagaggtgctgctgattatcttgttaagcctattagaggaatgaactgagtaatctatggcaacatgtttggagaagaaaatctgtaagcacaggcttttaagatttcgtgttgtatcgaatattattgtcttgttcttacaatgttctaattttgtttgtttcgtgattctattaggcctcgagaaatggtcttcgatatgaaggtgttgcacaaaataagggtgaagccacttctgaaaatgaccctgcaaccaatcaatccaatggctatattccttccattcaaagaaatgaagaaaacatcgataaagggagcgattctcaggtatctcatattttctgtatgagcctccaaagaatttgtaacctttattgtttgacagaaatgtaattgctagtatctcaatgttgaagatattaatgaacggttttagtttagaataaatttagactggtcataagcgatatagagtatgttcttcgccgaggaatatgttagtattaattcttaaaaaaggaatttccttccagtctaggtttcatatagagactgcatgtttgctgtctcctgcagagctcttgtacaaggccagaaatggaggttgagaatgtccacgtcgatcagatgcaggaatgctcacagccaacacggggcgagtcttttccaagtgactcgagaacacagaagagtgaagggcacatcaatacaggacagagattcatgcatggaaatgatgcaagaggtgaataatattacaacttttttttaagtattattcttgaatcagtgttcaagaatctaacggtgtcacaatcgaaataggttcaatgatgggaatctatcaggatggtgaaaccgaaatttcaccaggcaagtgttccaagccagaaagtattagtaggagggatgctattatgtctagtgaggcttgtgagaacaataaattcctgttcaatcattctagaggcaccattgatttgaatggagctttcgatgaaaatcaaaattgcaattatcaaaattgttcatctagtaatggaactagcaagtttgattctggtccgaaattggatcttaccttgagatcaagttctggtgactttgagagccatatggttgagaagaggcacaaccttggtcattctaatggttcggcctttacacggtgagttctatttcgatcatctcactttttgaccagtgagatatgtcatgatgatatggaaagtttcatcaattggattatctttcttaacttgtatttgctacatttcggtgattattcatgcagatacatcaataaaccgttgcagcccacaaaccctacatcagccggtgtatgcgatcaagataaagaaagcggatcaaacaccgaaaagcatatgttcggttttgctgctggacttagttttaactctgataccccccctggtccctcaaaaagctcaccgaaaagtcatattacattggctacaggtcaatctaaccattctgaagttgcaactcattcccctcaaccgagattatttcctctaccgattcctgtgaaaggtgtaagggttaataatccagtggcaggctatggttctgtagtagtaccttcagggttctgttcacaatcaggttcttcctccgggatcagtccaagctcggttgcccaacaagaatcttcattgctaatcaatgcgttttaccaaacgactcctgaaaagaacaaatctgagcagtcaaatggcctttgtcgaaacaacagcatcgccaaagatcaatccttgctaaatcaagaacataaattggattttttcgaagatcgtgggcgtatctctcctacaaccgatcaaagcgctagtagtagtttgtgtaatggaaatgcaagtcatcttaatagtattgggtatggaagtgcttgtggaagtaatagcaatgttgatcagactgccatgattagaactgtttccgagaacaagaatgatgaaagttactttaataacagcgggaattctcaccgagccattcaaagagaagcagctcttacaaaatttcgaatgaagaggaaagatagatgctttgaaaagaaggtataacattattaccattctcaatttcattactttttttaagtgttcttcatttttctaacgaattcatgtctattccaggtccgttacgagagcagaaagaaactcgctgagcaacgtccccgagtcaagggacagtttgttcgccaagcgcaaacagatccttcgatagcagaaaccgaaggaaattcttaggagttaataactatttagagctttgaataacattaatgattgaagaaaacactcataatgatgatcaatagtagtttatttcgaaatcttttacttcttttgttgtagtatgagagtgaactttgggggccataagcacaagagaaaatcacttagtgtggtttatgtatagctttgtaatttctactcaattctttttttctttttgggtagctgtgaattttgttgattaacaatctcataggtttatattggtatatacactcttttagtattgtgatttgtatccatagtaacaccatcattaccaatcaccagcttgtatactactatatatgaaagacttcattttttttagtagttcttacaatttttgtatttgtataaatacacaaagtggtattgagtattgatttgacaagtttcaataaagggtttgttctttacaaaatactttttgaaataaagataagtgtggttatgctatgaaggtgtgccattgaaatttgttagagctgaaaagaacaaaaaccataatataaattgactaccatt

SEQ ID NO: 17 - DNA, 11400 bp, Cannabis sativa
  source 1..11400: mol_type = genomic DNA
ctcgcttgtcttctcacgaaaccgactgaactaagacctaagttacactcacttctggagccaacatcgtcttcataccctcaaccgactagaatcgaagaaaaaaaaggaatcgaagctaggacccacacgaattgcaaaccccccatttgtgaaccaaaaactcgataaatcgaagaaaccactgaaacccttttcgctattgttgtttagatgttcgatatgcggtttctttgtgttgtttttgcttattttttgttaaaaaatcgagttttggtacgataaggtacgataatagtgtaatctgaaagttagggatgaaggtttgataggaaacgatagtgatatgatggggtacaatagggtatgataattaataaaatacgatagtggtacaatgcggtacgataaggtatgatatggtataaatttttatgcacatatcgtactactatcggaccactattagactatatcgtttctttatcaaccatcgcaccaccatcatatctcatcgaacatttatccctaacctttcatcatattacactaccctattgtataataaccttaatattatcatgccattaatttatcatggcataaattttaagtcactatcgttcatatcatactctaaaaatttacagaaaatataaatttcacaaaaatttgaattttaccaaaaaaataaataaagaaatacaaagaaaccacatatcgtttgtgtgtctattcgacggtggtggtcgaaggttgtgggtcgttggccactcaccagagagaactatagagagagggagggtcgtaggttgtcaagagttgtgggtggaggtcatcgatgttcaacggtagtttcgatggtggagtttggtgagaatcaaagagagagatagagagaacaagatggtgaaaaagagagaagggctaattttttttggataatttttaaactattgagagaagtaatttagagtatacaataaaatatacctatactattaatattgagctaacttaaatttaagttcttatttttgacctaaaatatgaataaaaaaagacataaaatatataaggataataataaggcttatagaccattgaatttggtgcacaaacgtcttattccatacgaagaatagtttttaatttgtttaaagtttgatgggtgtgtgtatcaattgagattcacaaattctggaacagctaaaagtttgaagatttttttaggaatatggaaaaagctagttataaaagaaagaggttattttacattttatatctttcttatccaataatatattgccaattaaatatgcaaataacttctagaaagttctttcttgatttaaattaatatagaacgatgattaattttataaaccttctcaatctcaaccattggaaacattattaattataactaccacttaccacccaattggcttttacatataattataatcaaggctgttactgttagtataataaagtccttaattgttaggtgttttttttttcttttttttaaaaaaattttagaaatgttatgatacgtaattatcagtacaatttaagatttagttctatttattttattttaataaaattgtaaaaaaatactaattaattataaagtatcacttaataataatattaagcaccttaattttttataacaatgctttttttttttttttttttttttttttgcaaaagaaaaattgttaattgatgttgtttggccggcctcttatgctgttaggagatgttttcttgcttcccttattgtagaagtctgatacttaatagccaattgttacaaattacaataaaatcttaagtatgtcttttgactggacaaataattatgaggtttttacaaaaatattagattttaagtaaaaatttataattttattgtcatgcggattttttttataaaaaatactatctttttataaaacaaccgtaaaaacaaaacagaacaactaaaaaataattatagagcaactatgaaaacttaatataatacatagtataaaccttgcacattattttttttttttaaaaaaaaaatatagtatttttgaaaaaaattcttctccataaaaaaaagtaaaaaattaaaaatttcaatatgtgatgtaaaaacctaaaaaattatttttaattattgtattaaattataaatttttattttagttattattattattattattttgtaaagtgatatagatcactttaagaattcacgatgtagacatcaacgatcgtcaaagtagtttgcatttctgttccacaagcttaaagttatttttgtcaatgttaaagtcatgacagatgaccgtgaggtcgattccagtcacttccttatgcgaccagacggatacatcttggtttcgccagagacaagccacgagaagtgttctcaaggttggccccaggtttttgccgattttgacagtcttagtcgagtatgcttcgttgagagggacctcttctagttcttcggtcaaaccaactcctaagtcattatctccaaagcatgggcagatatcttccaagcccagggtgcacccttaggcagttgcgggtgcagtctccatcttctccttagcgagattcaacacctcaaaagtcttcttcttctcattagatgcttttgcagtcaatgaggtcttttttgccacaactagcaaagtattataacactcacgagctgcacgctagttacccaacgagcatccaatgcttgtcgacattgagaacttgatgaagaagtaaaaaatggatgagatggctcgaaggtcataaagactaggcctccctgtcatcgcattatacaaagattttgtatcgaccactattaaagtgaccattatagtgttagtatgtggggattttcccacagtcagtggcaatttgatcttgccacacggagcaatgctcgcttcactgaaatcatacattagctacttgcatggctccaagtccttcaaagacaaactcattcgttccagggtttattcgaacatgatgttggatgactctccatcgtcaaccatcactcttgctaccatcatgttggcaatttgtatctccacgaccaaggggtcgttgtgagggtaccatacatgagtagtgtcagactctgcgaatgtgatatgttcacacaagcatttagtcttgggaggtctctcctcaacttccaacatgttgacacaatgctcatggcgcatcgaccgtgcgtacttctccaatgcttttctcaagtttccagctatatgtgggcctccaacaatcacatctaaccgtcctgcaatggttgcaggttggagaggtgcgacatgctcttgagggttgttagtcgtacatacctaggggtgttcatcaaactacctgcaccgtaccatactgcataaaaaatgcaatttgaaatcttttgcagtgcagctgcgatttacattttccccaaatcgtagagtgtggtgcagtacgattttaaatttagtaattacggttcaaatcgcaccgcactatacattgtaaagttaccaatttttataatatgatttttattcttttttcatataagaacaaatatatgtaatacatgtatatcttacattattttttagagacaatttaatataataacttgatgattaatatatatgttataatgttaaaattttattaagattttttttttggattgctaaaactttatagattgatttttaaatttttattataatgttcgataatgatgatagtgtaaattgctcaaactgcaccactatactgtattttcactgtgtggttttaagatctcgtagtgcagttgcggtttgagaaattacaaaaaccacatatggagtttggtttgaaaaaaaagttacaacccgcaccattcgcaccgcgaataaccccatagatattgctggagggcatcatgtttttgacagataatgaactcgatctcgtcccacaactgattacacttattagtatcatgaccgactgtccgtaatcattgtgaatctgaaaaagattagtttattctttgactttggagaagttctcttggtaagtttacttggtatgcacgggtgatgcctgatgaataactaatgacgactaggtgacactcaggtgatcactagtgtacatggatatcgatgactttgcacatgacacatgtacatgagatattgacttacgaaaacatccaaatcttattttaggggcaaaataagactgatacctagaacataatctttatttgaaggacagagcagtatgccccaggccaatgccaacctaggcgcttagtgtgtttgcacactggcgcccggtgattccttgaggataactcactatttgaacaatatttctcctgatttcaagtctcatatcttgtttttggaacaaaacaagaaaaattctcaagaaaaaaatacttcgaactgactcaggttgacaaaacttcaacttgggcactaggactaggcattaggtcaacttaggcgctgagtcctagttttaccgatagatgcttatatttgattcctaaaatcacttgtatcttaattgttggtgaacaagagaatatgccgaaaaaacttatcgtgaaaaatgtttagcaatggcccaagctgacaatatgtcaacctgggcactagaccttagcactaggtcaacttaatattaatatatctaatacttatactcaataatatttgttgaagaaaaaagaataataattttttttaccaatatacgccaaaagttattgagataataaaaatttaagagactcttagttgggttaagagccaaaactggcattatggttttggtccaagtcatttgtagtaatcttgcaatgtgtttaacttttggctttgttgttgggaactgccaatttcttttatttatgatatttattgtttaattattttttataagacaatcaataaattgagtagcacattatgttgttaatactttcggtatcaaccctgttgtagtatttttgaatttctaatttcaattaatcttcaatatcttttgtatttagaacgtgaattcattttaatgaattatattttaattaaaaaaatatttttttttactaaaaatatatattccaacttttatcattttatatccaccaagcatcacattaacattttttttttcccattttccttagaagattttttcctttcgatcaattataggtgtagttgtagaaaagaatataaaaccagagagagagagatagaaagaaatagaagaaggtttaggaaataggaagagtgaaaaagaaaagaaacttctcaaagaagctgccacgtgtgatgctcccaaccatgaaatccaaattcgtggtggcacaaggtgtacgtggcagttcctcaagagaaatatcaaacaaagaatcaaataaaaattaataaaaattacacgtacctttattttaagaaatatgtagtaccaagtgggccccttcaccaccttggtcgatcatcaccaccacaccatgtgacaaaacttaaaaaatcaaatacggtggctcatatttggccacgtgtactgtaagaaaccattgcccaatcacggcccacgcccacacctctgtcttctccagatatttttctttctcaaaactttgtttcgcggctgtgatacagagcttaacattttgtaattttctttgttttttttttttttttattaactaaaatagagaataaatctgctttttgagttttgttttcgaatttggtttcagatacttttttttttttttttgtcccgggttttatatgaagaggatgaggtagagttttttttaagtttattaaagttgtagagtagacctgatttttcttttaatagtatttttatttgcattttctcgtggtttcttttttaatctcaactgggtttctggaattttgggtttgggttgtgtttacagggtgtttgaaaatgggtgaagtagctgagagtattgaagagcttatggcggcggaagttgaagttgagaaacgagagcagaatgaagaagaagaagaaaaagacggatccaaggaggtagttaggtgggagagttatcttcccagaatggtgcttagagttcttttggttgaggctgatgattccacgcggcagattgtgacagcccttcttcgaaaatgcggttataaaggtcaaatactttgctcctttatgtattactctcattttatttgctattattggcttatgtatagtaatatatgtgtatatgtattactatatttgctgtgcgttcttggattagtggcattctttttatttaattttttgtagaggctagtacagttacagttttgttttttaacagaaccattctttgtactttgttgtatatgaaaatgagatggaacattagaaaaactttgcgtttgaattctgtcgattggtgattttggtttttgggttggatatggaattttatattttgtaggattttatgctgttattgatattcattataggttatgagatgttaagcttgttcccaactgaaccccttctttgcctctttagtttttatagcaattactcgatgtttcttgtcctttcgtttcaatttcatgctaagtatttgaagttttatctttttggattgttaattcaaagagagatgttagacacatttcatacataagatttatactcttcagaccgatgcatcattttttattaacctcacttatttttatggggcttaaaaataagccacatcactgatgagtgttaaaaagtttgtgtttaatattaagtctgaaccaagttatgaagtggatgaaaacattatacagatgcaaatgtatacccaatagaattatttaaagtacatcaataattttcaataactttattgtaggctttctaaattaggaaaggggggggggggggggggggtgttgcatataattttactataattgacaaaaaatttctcttatgataaaaatttgagggggcaggcatatgcccaccattagcactgtttgttgttgttgttaaaccatagggaataatatatttcacagtttagttatgctagtcattgattttctctatgaagatcagacatttcttatgactaaatgtttcacaatttttcttttatgcttctgtgttagatagttaattggattgcttagattcccatggctaatttgcagtttcttgcatctttatgtaaaattctatcatttttgtatgatattttttttcgaatgggtttgtaacataaaaacaattattggtctgtaatccgaccaagttaactcaattcggttatcatttcctattcttaataagcatttgaaccattatcgataactgttttcttaataatgtatgagcaacatcgaaattcatttttttttttaaaatgttggtaaaagtttgtaattaaaagtgaatactttgtttggtcaaccagtttctgcagtacctgatggactgaaagcatgggaggttttaaaggagagggctcgaaacatagatctcatacttactgaaatggagttgccatcaatatcaggattcacacttctttcgttagtcatggaacatgaagtttgcaaaaacatacctgtcataagtatgttttctgtagaaattgctgtttcttttgctaacttgtggtcctcaaaattagatgtgagactaatataatacatattctttcattgtagtgatgtcctctcaagattcagttagcatggttttcaagtgtatgctaaaaggagctgctgactatctaatcaagcctattaggaagaatgagctaaagaatctttggcagcatgtgtggagaaagcgcaccgtatgttccttttctcttcggcgtgtattggattattttgttcattccatattcctgtaaccataaatgatatatttttcgttccatggggttctgtttttcagttttctaagaacatgacagctgctcaacatggagcggagcttgcttctgaaattaatacagcaagcaatcagtcaagcgactgtgtggtttcaataaagaaaattaaggaatgcagtgagaaggagagtgattcccaagtaagtttatttaatcatccagtcaattctttgtcaattgagaaatgatagggtaatctatgttttcttttcccaaccataattgtatgcttcgattacatataatttatgtgcccattgttagagttaattttatgcccatttctcatatttttgactgtcttctacctgtctatgatgtaacaagggattagaattgttattaacaactaaaacaaataagaagagtttatcaagtttcttcattacattttatttttttgaaactatgatccccgtctaaccttataaggggaccagaaaactattgaaattgctatgtaggtatttagaacagcctcgaagctctgaccttgtgggagcaaaccacatgtctgaccatttgagttattccacttgggtatcaagtttcttcattattagagtatcaaatgttttccctcttgtttgctaattactgcacaactttgaatgtatcttcttgattgctagtggacctaaaagggacacaaattaatggttagaacatagatattgaaatccatgtcgaagttaatatgaaagcggggctacttagcttgagagaaattttcttattccactgaaggctgatttcatgaagtttttgctaagagactgcatgttttcccatgttgtgcagagctcttgtacaatgcctcacttggaagctaagagtgagcacgtccaaaatatgcaagatatttcacagataaggagtgggaattcttctaaaattgacaggaacaatgtaaagagtcacgaagattgtactcctttaaataaagaagcagtaatgcatgaaaacaagacagaaggaggtatgccataaccttttgtttcccactctgtgtctgtgctcatgtattttcttctttttttttttttgagtggagcagctaatagctatttaactgcagataaatcaaacctttttaaaccagaggttgtatcttgcaatgaagcttttaggtcatctctcttgggattaaaggaagagtacattggcaataagagaatgaagctagtcgaaacttcccaacctgaaaattgtgaagtcaatgctaatgccaatattcagagttgttatgacgaactagtgaaaccctcttgtggagctattgacttgatcggtacatttgatgatcacccaaggtgcactgataagtcggatgatcagtgcataagcatctctgagtatcctccacagctggaactttctttgaaaggaatttgtccaaggaactcgaatagccaaggtactggtgaaaggccaaaactcaaccattctaatgcctctgcattttcatggtgcgttctctatcacttttagacgccttatgatttgcttagttagcttaattgaggttcaagagagcctcaccttccttccattgttatgttctcaatactttatcagaggaacaagaatttggtattttcttttgtgtgccatttaattataaaaaagctttttaagaatttaccacagaagaatctgtataattaagcttggaaggtttccttgtctctgatgaattgtatggaagaaaggcctaagtggaactgtaagagtactgaattatatatacatatatatctataaaaaatgagggaattcgtggtatgtttcctgaatatgtaatcattgtatcccttctgcaggtatagtaatagcaatacactgcaacccctttttcccactcttcccaacactgaaattaccccaaaggagggtccaaagaaatctcctgagttaattttcactccatcgtttagctccaccggttcttctccacagcatgctgccacaaggagcggtagtcactacaatttgccggcttcggttgctgggcaatccgggcataccactgtgtccttccccagtcctcaacttggaatgagtccagttaagggtctaaagtatgaaaattctggaggcaaccagttattaccatccttttatacacaatcaagtgtatcctctacgtgggttcccatttcggaaagccagcgagagaagtctccatttgcagcaaatacctcaagtcaacactcggaacaaggttaccaactttctgaagaagcaactcataaatcttctgatcaaaccaagcaagagaagaaagagtttgatcttgtggaggaagtgaagcctagctcttttgctgctgctgatcagagccatggtggtagtttttgcaatggagtgggagatcaaattagtagtgctgaacatggcagctcttgtggtagaaacgatggaaacaccaattcttcttccggggctgatgagaaaaccaccacttctcgtggtctgaacgacaatggacattttgtttatgaaagttttagagggatggatttgcatcgcatgagtcagagagaagcagctctaaccaaatttcgaatgaaacgtaaagatcgttgctttgagaaaaaggtactactactacatagtacttactattgtttttaattgtcattatcagtgttcattagttcactgttgtgctattgaaaattttcaaatgatcacaaactaagtcatctctgtaaagggtcgagattggaacctaaacattggctcattcttttacacaggttcgatatcagagcaggaaaagactggcagaacagaggccccgggtgaaaggacaatttgttcgtcaaggacaaactgacactccaattggtaatagtacgtgaagatcaattgctctcgtttgctttatgacacaatagcgttgaagttgacgtagggttcttgtgctctcaaagcaatcggtaactataggactaggagtaggatgtagagtgacgagaatgactatcaaagatggttgtaaccttgcaaatcactttcggaattgctagttggtttggctgtgttttacgacgtggttttctttttttttttttttttttttatatagtttggcttgaaaaggtttaaaggttgtgtcataactacttagtaactgaatttagatgactgatcttagatttatttgattggtggattatttgtatttataggccttgtttagtaccagatactagtaattactgtaatgctacatatataatgtatgataatatattatatatcagttcataattcatagctacatatttaatgctacgtgtacatgagatgtactcatatatgtacatgctgtcatcagatcatcgtgcttaactttatcatggaaagaaatatataccgaaagt

SEQ ID NO: 18 - DNA, 24 bp, synthetic construct
  misc_feature 1..24: note = Primer 1 for UPF2 gene
  source 1..24: mol_type = other DNA
gtacagtaaactatctcaatttct

SEQ ID NO: 19 - DNA, 24 bp, synthetic construct
  misc_feature 1..24: note = Primer 2 for UPF2 gene
  source 1..24: mol_type = other DNA
accacaccttttccaattggactc

SEQ ID NO: 20 - DNA, 24 bp, synthetic construct
  misc_feature 1..24: note = cDNA specific forward primer spanning exons 2 and 3 of PRR7 withthe inclusion of the Auto specific splice inclusion mutation
  source 1..24: mol_type = other DNA
atccctgtaattattgatgatgtc

SEQ ID NO: 21 - DNA, 24 bp, synthetic construct
  misc_feature 1..24: note = cDNA specific forward primer spanning exons 2 and 3 of PRR7matching the Photo background
  source 1..24: mol_type = other DNA
catccctgtaattatgatgtcttc

SEQ ID NO: 22 - DNA, 20 bp, synthetic construct
  misc_feature 1..20: note = Common reverse primer in exon 4 of PRR7 for amplification of cDNA
  source 1..20: mol_type = other DNA
gctgccactgttgttgtcag

SEQ ID NO: 23 - DNA, 20 bp, synthetic construct
  misc_feature 1..20: note = Common reverse primer in exon 7 of PRR7 for amplification of cDNA
  source 1..20: mol_type = other DNA
taggcctctctgcaaaagcc

SEQ ID NO: 24 - DNA, 21 bp, synthetic construct
  misc_feature 1..21: note = cDNA specific forward primer for sequencing spanning exons 1 and2 of PRR7
  source 1..21: mol_type = other DNA
tgtggctacgaagttacagct

SEQ ID NO: 25 - DNA, 20 bp, synthetic construct
  misc_feature 1..20: note = Forward primer for sequencing located in exon 2 of PRR7
  source 1..20: mol_type = other DNA
ctgaggtagtcatgccctgt

SEQ ID NO: 26 - DNA, 20 bp, synthetic construct
  misc_feature 1..20: note = Reverse primer for sequencing located in exon 3 of PRR7
  source 1..20: mol_type = other DNA
agtcaacggcacctttcgat

SEQ ID NO: 27 - protein, 727 aa, Arabidopsis thaliana
MNANEEGEGSRYPITDRKTGETKFDRVESRTEKHSEEEKTNGITMDVRNGSSGGLQIPLSQQTAATVCWERFLHVRTIRVLLVENDDCTRYIVTALLRNCSYEVVEASNGIQAWKVLEDLNNHIDIVLTEVIMPYLSGIGLLCKILNHKSRRNIPVIMMSSHDSMGLVFKCLSKGAVDFLVKPIRKNELKILWQHVWRRCQSSSGSGSESGTHQTQKSVKSKSIKKSDQDSGSSDENENGSIGLNASDGSSDGSGAQSSWTKKAVDVDDSPRAVSLWDRVDSTCAQVVHSNPEFPSNQLVAPPAEKETQEHDDKFEDVTMGRDLEISIRRNCDLALEPKDEPLSKTTGIMRQDNSFEKSSSKWKMKVGKGPLDLSSESPSSKQMHEDGGSSFKAMSSHLQDNREPEAPNTHLKTLDTNEASVKISEELMHVEHSSKRHRGTKDDGTLVRDDRNVLRRSEGSAFSRYNPASNANKISGGNLGSTSLQDNNSQDLIKKTEAAYDCHSNMNESLPHNHRSHVGSNNFDMSSTTENNAFTKPGAPKVSSAGSSSVKHSSFQPLPCDHHNNHASYNLVHVAERKKLPPQCGSSNVYNETIEGNNNTVNYSVNGSVSGSGHGSNGPYGSSNGMNAGGMNMGSDNGAGKNGNGDGSGSGSGSGSGNLADENKISQREAALTKFRQKRKERCFRKKVRYQSRKKLAEQRPRVRGQFVRKTAAATDDNDIKNIEDS

SEQ ID NO: 28 - DNA, 3425 bp, Cannabis sativa
  source 1..3425: mol_type = genomic DNA
attcttctgccttttatttcttactcttaagctacactttgtttttcttcttctttctttctttatttatttatttctacaaatttttaatataattttccatctgtcctctctttctctcttccatatttatgtctgtgatccatttttcttcagacccaactgccgtttttctcttttcattttttcacacaactttacaggaaaacctctgttttttttttttttttttttcccttcttgggtttttttttttgatctgaactgggtttggattttctagagctttttttctgaaaaataatccattcaggaagtgggaattttttatttttttttttttcccatttcaacctcttcaaaacaaatttaccatctgaaaattttgataaagagatcgagagagaaaatatataaagaaagaaactgaggaattttgatatgatgttggatcttaatgttaatatcaacaacggtgccgattcaacctacgagaagactaaggaaagaggagcagagttgatgataatggaggtagaagagaaaggttccacaacccaaataatggaagattctggttcctctggttcttcggttgttaatgttgaaatcgatgctctgagtagtactacaacaattactacctctaatagcgtctttagagaagaagactcttccatcaatgtcacgaacacaacatcttcgacatttttctttgacataatgaagagagaaaaggactgtaacaacggtgccactgccgggaaagaaactaataacatctctccgccgggatttctaaccaggtcttttttcccggtggccggagaaaaggtagggaatcagttcgttgaagccggttctgggtcgtcttcctcgcgacctcagtggttgaacttatcttttgcggattccggtggaggaggcgccgccgtccaaccgccggcggatgttaaagttttgcagcagaaacaacagataaagaaaagccggcgggaccaaggtcccggagctctcagtaccgtggcgttacgttttaccggagaaccggccggtgggaatcccatatttggtgagtttgtttccatggaaaatcaacaaacatgtatatatatataaaaatataatatataattatattatatttactaacttcatttttcttcttctttactttttggaatacttagggattgtgggaagcaagtatatttaggtacgtaatttggctaattattagtttctttttaattataatgtaactaatttcatggcaagaggattttgttgatttgtttgtttttttttttgtttattcatttatttatttatttattttaggtggattcgacacagctcatgctgctgccaggtatgcttaattatttactttttttgtcttttttttttttttttctgataaaaaaataaacatttgtgtaaaatatacatatatatttgtgtgtatataatgttagctaaattgggttattgatcatcattattttttctaacttcagagcttacgatcgagctgcaatcaagtttcgtggagttgatgctgatatcaatttcaatgtaaccgattacgatgaagatatgatgcaggtgccacatataaataaatattttttttttttgagataacacatataaataaatatgtatagtatatgatttatatttgtttatagttaaagttttgtattaacaatttctctaattttgaaaatttttgctcttatttgagatgaaaaacctgagcaaagaagaatttgttcaaatacttcgacgacaaagcactggattttctcgtgggagctcaaaatatagaggagtaactttgcataaatgtggtcgatgggaagctcgaatgggacaattccttggaaaaaagtgagaaccatttctcatctaattacacaattatgtattattatgagagtcaaaacattgagaaagttggtagtcatcacatatgtggttgatggtagtgttatgcatacaaaagaagaaatgccttaatgggcactccacttttgtatctataaaatcatgatccttctttgcaggtatatttatcttggattattcgacagcgaactagaggctgcaaggtcctcatttgttcatgaatatatttgaagaattcaatattgtcctctaatattgacaacattttctttttaactcgatagaataaaacacattatgcctttggcttggcttgtttggtggggatttaatccaacacaagttttttcttttatctttgtttaaattcagggcttatgacaaagcagccatcaaatgtaatggaagggatgcaattaccaactttgagcatagtacttatcagggtgagataattttagacaccaatgcacaaggtactaaatttaatgactttatagctaaaggagttattatctagtactacaaatataacaattaattcttctgttgcaaaaatgcaggcaatgaccacaaccttgatctgaacttgggaatttctccaccatgtgatggtccaaaagggcgtgagtactcttttggtcttggggacactcgtgttcattggaaccctcgtgaaggacctcacagaataagaccgataatggtacattgattttaataataggcacagtttagaaagtgcagttttctttcctttctgatataaaaactaaaattagtatccactactctcaatagattgatgggcagccatccccacatattctacctcccaaccatgctgcttcagctacatgttcaggagtgtatccagccttcttaccaaaacatgaggtactttctttcgcatcctcacaaacttgccaaatacttaaagttgattatctgaaacacttttttgggagatacaggagatgagagcaatggcagatgatcatcaaaagaggatcgaagccgccaattcttcgcagggttttttgaactgggcatggaaaattcatggcaatggtagcaacactactactactactactacatctggttgtgtaacagccatgcctactttctccattgcagcatcatcaggattctcatcttccacatctttggctgctctatcagctaccaataatataaatccccaagccaattttgttcaaaataatatttgccttagtcctagtatgcccattaccactacaaattctgtcacaaacaattttcacaactcacaaattcatagaggctaaacatcaaacaccttgaagaaaagcaagatgtgctaaaaaattaatattttatattagatgtagttctgtattatttgtactacgtgtaagagattttttctttctttctttctctgaatgcctcccttgtaatatgccaactattttattgatcactgattttgctaaattgtaatgtaataatatttcttcattgta

SEQ ID NO: 29 - DNA, 5496 bp, Cannabis sativa
  misc_feature 1: note = "n" is a, g, c, or t
  source 1..5496: mol_type = genomic DNA
naggttttttaattgtggggaagctgctaactttgggacaccacagtggctcaaagtggctaaagaagctgcagtgcgtagagctgctatgaattatcttcccatgctttctcatcagcagttgctatacctgttgacaatgtcatttatgtcaaggtcagtttatgctctaaatttggaatcaaaacttatatgtttttataatggccatataaatatcaatttcaggatattctcatagtcttcttgatttttgtgtactcgatatatttgcatattctctttagaaaagaactcaactgcccatctaatacctatttattatttaattgaattgtttgtatccagagtgccgagatccttgcttcctggtgtgcggagttctcgtctgaaggatcgcctgaaagaaggaagagagtttttagtaaagaaggcatttattgaagacatattgaatgaaaacaataccttatctattgttcttcaaaaagattcaagatatcgtgctgtattatggaatcctgatatactaacatatccaagcaaagagactcttgcacccactgcaggtgccattgttagtacaagttcaacagcaacagaaaacactcccctcgattatagaaaacatgacaatgagcaaaacaatcttcttgatgagatgaatttgtatatggagaatttgaatgatttatatttgggtagcgatgatttgtcatgtgatcttcaagttgactcaggggcattggcatgtgtagcttgtggtatccttggttttccatttatgtctgtggtacaaccatccgaaaaggcctcactagaacttcttcatgataatcatcttctgtttcaagaatgccctggaggatatttgaactgtttttcctctctggacctcgatgtctctaataatgcttctattacaggtactggttacttcactggtcttgtatattgtccttgatttttttttttctatcagcattcttttgtatggaaaagattagtagtatatatgtttattttttcggctcattgataacaaccacaacagacatattacggaataatgagatttacatgagtagtgttgatgtagttgcaagtggatagctacaattttgaatgtctaagtgtccacatagttatcaatataataagacattggctgcccatttggctttgttaaggaattctatgtgattccgttctctgatctatatagggaacagcaatattttatgtaaaaaaaattagccctgtttgaatttgttttctgcgggttttttctaacccactttttttttatcatcaatctaatacaagcttggggttttgtttcaccaggaaaaagaaaaaagttagtggagtgaaaataaatccattttcatttcatcctttgtttggttgttacataaaacatgagtcagtcatttttatatttctcattgtatacttggttatttttttattaccttttttttctcattcaatttaatttcattttactcctattcatattcccattttcatttttctgttttcattaattccaaccaaacctacccttaattgtgaatgttttaccgtcgatatagtagtttaagtgaatcactgttttttttttttttttcttttttaatgtttttgtttaaacacaagacaagtttgcttgcattggaaacatttcattggatcttttaaacaccaaactatatgcaattcctcttttgttctgtagagaatgtttctacggtttccaatcctccttcaatggatcttccagtaccttcagttaacaagtttgatggtggtcaaaatgcttacaataaatttctgagaccacgtagcttctgcctggaacatgctgttgaaattatagacattttacagggtataggtggagcagaggtgcttgtaatttgtcattctggtaagattgtcctgctacttcattgtcatttgtacttgcattttccattttcttgagttgctcagtttctcttataaaaggtgcctgatggctcataaaatttaatgttcatcgttgaaaattgcatcggtaactggtaaatgatatgtttcataatttttttttaactcgtagttgattatttttttatttttattttgggatgagtttgtgcagactatcagaagataaaggcacatgctgctgccattgcagaggaaattggtagcccttttaattataaagaggttcctttagatattgcatcgaaagatgatctgaacttgatagatattgctattgatgacgaacacgatgaatgtggtaaagactggacatcaaaattggggatcaacttgcagcactgtatcaaggtcagaagaaactcctcctctaagcaagttcgacatgctcttacattgggtgggttgttctctgataaaccgtcctctcaagatttctcgcctctcaagtggcagttgagaagatctcgttcaaaaaaaatttatgttaaaccatttgatagtgctcaagtgaagaaagtagtgggtgaaacatcttccaccattgagaaggaaaaaaaactgattcagtattcaagaaggaagttcaatagaaaaccaggtggtttaacaggagcaggtaaattttgtggacaacatgctaaaggggagatcttgatgaacttgccaaaagagcatctgaaactggtaatgatgttgggaattccacaagctgtaaattctcagctttaattaagatatcggaaacacacaatgaaaaccagacatctgaagcaacttggaacaatagcatgaaagctagctgtggaagtgttgagaatcaagttttgaaggaaatgaatttggatagtcaagcttgtcttgtagcaagttctgatgcttctaagatgcaagatgagagcagtgttccaacccaaaaagtcagcataaccgataagcttgttgaaaaactagaggcttgtccattttcccatgatactaaggaaagctccggacttctaggagatcagcttaaagtaggacaatctaacaaaactgatggggctaatgttgattctgaagggcactccaatgtcaaggctggcaaggagggtgttttgactgatttgaattctgatctttctaacatgacaaactcacatgctattgaatcccaggatgacaacttgatgggacaaataaaaaatgctgtagtggagaatgtttccatgactgttgaagtttgtgattctgtttctgtggataaagaagtgacgctagtagtgcatgctaccagaattgatgacggggaaccagtttcttgcaactctacagtcataatagataatattggtacttcaaaggaagaatgctttaattgttcaggagagatctgttctgtagaataccccagcagcaatggtaaagctcttaaagttgaatcagaagaagaaattaaaactagaataaacaaataccctcttgcctcagttgaagaaacttctaaagctcggagcgagactcattctacaaaggattttagtgagaccattcctgtcttaattgaagaaacttctgaatttttaagtgatccttcttctgcagctaaattatgctgtgctgtggcttcggaacagattctaaagcaaaaagttgggacttcagatgggagtagtgaggaacttgcttctagttctgtgacacaaatggaagtccaccaccgtcattgtgtttcaatagaggaacattctgatgttctcagggaaagctcagtggaagacgacttgggaacggatgtgacttcagataatggatttcataaggaagcctcgatgaaaaatggagagcagagatcagtttctcctgttacacaaacagttgatcagcctacacccataagtgttggagaatctgaaactccatggaatccttccaaagaacatcccggtgatggcttgactttagatgataaaaagaggaaaagtgagaccactgctttgccaaatagcatttccttccaaaaatgttcaagggttagaagagagactcatactaatgaagacttgtgcaacggtagtgaagtttgttcgtcgcaggatgatagagcattggatagcgagtctaaagatatagacacttgttcaaacccagtaaaaggaagaaagaggaagaaagagatagaactactaacagaaaaccagttcagttgcaatggctttacgaggagtccatgtgaagggttaagaccaaggactttgaaggatgcaacaagttgtgaaactgatacccttagagaagtcgagagaaagcctgcagtgaaggtaaagaaaacttcaaatgttagaggtcctcctaaggataagaaagaaaattcaaggaagcctcacaaatgtgacttagaaagctgtcgcatgagttttgacactaaggcagagctaatgttgcacaaacgtaaccgctgcccacatgaagggtgtggaaagaggtttagctctcataagtatgcgatgattcaccaccgtgttcatgatgaccagaggcccctcaagtgcccctgggaaggttgttcaatgtctttcaagtgggcatgggcaaggactgagcatatacgcgtgcacaccggagagcgaccataccagtgcaagattgatggatgtggcctctctttcaggtttgtatcagatttcagccggcatagacggaaaacaggacattatgtgaactagccagcctttctgggggtgactgggaggccctagattcgcacatcatttggtctacttttccaattcttgttgatgagaattaggttagggacatttcttttagcagttctcattcactgatacttgaatggcccttttttggtgggttccccttgttagcttttcatttgagcattctgaaattgtataatcttctggcatagccacaacggtttgtataagtcgaggcttgaaaggaagtctgagagttgtggctcctgaattcttggctggatgtctagatgtagataatgatgctcctagtttctgtaaatcatatcttttttcaagtaaaatttctacaaaattctcttgtgagattggattaatatttatttttctcattattttgaaacaaaattcgtacaaacaacaaaacaaccaaaagtgtaaccaaagcttacacaaatctttgtcttaaacaagtagcggagttgatgcatagattcaagtggtggcagagctgtgcagtgggcatggatggcagagaagcacgatcgaaggaaatggtgtagccaggttagggccatcagagggaatgcggcttgccaccagaactggtggagttcctcgatagagctgggttgatgaataatagcaaggagtaagaaaaagagagaaaggggttttgagttcttttattttatttccatttttcagttataaactttttattactttttccatatgaaagtataattgatgtggcaatgacattttttgaagctttgagaccagataaagttctttttatttt

SEQ ID NO: 30 - DNA, 1051 bp, Cannabis sativa
  source 1..1051: mol_type = genomic DNA
ttttgatagaggaatatggaaagcacctaggaaggttagcaagatccatattcgaaagaatggcaaagaatctgaatttggataaaactgaatccgaatccgacctatccgaactaaccggattcattcgggcataccggtacccgatatgctccgagttcagtaaggaacaccctgcatggggcatggactctcacacagacagctcagtcttatccatactgagttgtgaagaccaagttggtggacttcaagtcctcaaaaacgacaattggttaacggtcgaacctatctccaacactctaatagtcaaccttggagacatgatgcaggtacgtactataattaattaattaattaattaatattaattctaattttaataaaacgacgtcgtacgtgataatgatgatggttttgtgtttgttgctttgttgatatatgtatatataaaggcgattagtaacgatgagtacaagagtgtgaagcatagagtgaaagtgaacagatataaagagagaatttctatatgttacttcgtttttccaaaagaagggagtgttatacgaagcatcaactataagccttttacttatactgaatttcagaagcaagttcagcaagatgttaaggcccttggattcaaagttggcttagaccgtttcaagcatactactactgctgctgctgctattactgaggcgtgttaaacgttttcttttatataatatataatgtcgttttattgggtcctatatactgctactactactagtactttgtttgaattttgcttttgttttttttgaatgggtttgacttttactttaattctcagttatatgtgggttttattttgtactcgttgtagtattatataaataatattaattagacgttgttgtgtacgtgtgtgttgttaataacttttggtgtttgaggttagaagagtaattagacgttgttgtgtacgtgtgtgttaattaataacttttaaaaacgtatcgaatgccatttgtttttttttttttaataaaaaaagtcaagtctt

SEQ ID NO: 31 - DNA, 2850 bp, Cannabis sativa
  source 1..2850: mol_type = genomic DNA
attttgtataaaacacttaagtatatttccaaaagtagttttgtaatttgcatttttcttttgtgtatattgtttggtcgtgtgtatcattagactctccatttcaactcttctaaatttcttttggtcatattgtttttggattaatcagtgtaatctcatatcattttcattctgtaacaagttttatttatttttttgaataataacaagttttatttatttgttattttcaacttggtctttttccaccttattaaaatttatgttttctacattaagttaagacctctcaatcccatttgccccaaaccaaatgagtaatttccttgttttttttttgaaaagtgtatcccaatgataaagacagaaaagttcagtgattatgtacttgtaagttgtaacatatgttagttagtgccatcaaatgaagtccatattaatctgctttaccataatatgcatctttttcttgctttaattaatgcattgaaacaaatacactgcacatttcttgatcttagttctcttttagcaaaagtactccaattttgaattttggtactagttgtattaaacagtgaacttggggcttgtttagtatattgtattaggtcgtattgtactatattgaattagattatatataatactatgttaaattttaatttatactaaaatattatatatttaggtgtcgataaaagtcaataccatttatagttttacataaaatataattcaatgtaatactatacaatacaatatgatgtgatatggcattaccaaacgaaccctccttgatgtttatcagattatattttcctttgcagtgaaaagattgagccagctcccctttacatcatggcatctgtgaatccatttttcgaagatatgcgaacaaaatccgaggttattgatcctcctcaaagtgaagatatgcttgatgttggtgaaattctgaatgattctccacaaaatataggaaagcctaatgtagctgttacaagcagtgtccgcgatcttttggagtgccctgtttgcttaaatgcaatgtaccctccaattcatcaggtctggtttatcctagaacatgttaacaagtgttcatgctctctttgcatctacaaaatcatattaaacataatatttacgtgcgatttcttctaagctttgcagctcatggttttttttctgtgttgcagtgttcaaatggtcacacattgtgttctggttgcaagccaagagttcataaccggtgtcccacctgcaggcatgaactcgggaacattcgatgtcttgctttagagaaagttgctgcatctcttgagcttccatgtaaatatcagagttatggttgcattggtatatacccttattacagcaagttgaagcatgagtctcaatgcatttacagaccatacaattgcccatatgctggatcagaatgcactattgttggtgatatcccttctcttgtagcccatttaaaagatgatcacaaagttgatatgcataatggctgcacttttaatcatcgctatgtcaaatcaaacccgcaggaggttgaaaatgcaacctggatgttaacggtatttctcttctttccattctcatcttgctaaaacattcatgaaattgtcctcttgtttatctttcattgtgatcttttcatttgtttatcttaaggctatgttggaagtggattttagtgagaaaagagaaaggtagggaatatataagaaaaaaagtggaaaaaaaataataaattggaaagaaaaattggatagataaacactcaatttaacaaaattttccatatcaaacttgaaaattgatttagtttttttgttgtccccactttttttttcttacattttcctactatttctttctcaccaaaatctaacttccaacttctaattttggacagccacttgggtctttaaaaccaagcactttgaggctttttctttctagacttaagtaggaaatggagtgtttcgttgtaaaactcaggacttttcgtttgttgggtcacttttagttctatttacactgtatttgttttttcattttgtgtaccacggttttcctgcgccataagtgagactataatctattcgggggagacctgtcaagtggtctccttctccttttcaaaaaaaaaaaagaacttagtatatttgatattgattgcagtagaaaagctaattacaagtgagtactcaatacattgcatatcccaattcctcccttataatttttcactcaatactaccctctcaaataaagactcatatacctttattatttactataagctagcacaccctaatcctagggcctaacaaagattaatattgtgctgctttattcttcagaggattgagcctccacttttgtttttgttttttaatctacttgcaggttttcagctgttttggccagtatttttgtctgcattttgaagctttccagctcggtacagctcccgtctacatagctttccttcggtttatgggtgatgatgttgaggcaaagagttataactacagtcttgaagtggtggacatgggaggaagatggtatggcaggggattccaagaagcattagggacagtcaccgaaaagttcgtgacagttttgatggtctcattatccaacgaaatatggctctctccttctctgggggagaccggaaagagttgaagcttagggtgactggtagaatttggaaagaacagtaactgaaatttctcatttcatcttagttagttactagttagtaggttagaaacaagtgtttgtactagtcatgtgtttttagaattaagaagcaaactctct

SEQ ID NO: 32 - DNA, 1637 bp, Cannabis sativa
  source 1..1637: mol_type = genomic DNA
tggaggaggagagaaagagagggaaattgaggaggaaaccctgttcagctgccatatccaatctcgatgatggtactctcatgcacatcttcagctttctctctcctattccaggtgggtattattctttttcaactcccaaattagggtttttcaatttgctgattttgacctaaatttggggaatgaaagaaatgaaaatgaagaagttttgatagtaattatacataaataaaagaatagagattagattagatgatgtgtggttgcagatcggtataacacagccctggtttgcgacagatggcggtacttggcatgtcatcctcgactatggcttcgagttgatcgttctcttaaagaatccgaagctggcgttttcccaaacattgactcagctgtagctgctgcaaggttggttttttatatacatgtaaagaaaaaaaaacaaaaataagcaaagtccccattgattttgcagactatcattagttcggtgtaaatttagtttatggttgcttgagctactgtgaaggtctttcatggctgcttagtgtaattccatgaatttatgatgggaatgttgcacagtgcacagacccatattatgtagaggaacagaaagagaaaccctttgataatatcttaaatttgtttgaaaatttgttttgcaaatgtaactttttcattggagtcattctataccatatatgtgtgtttctcttcaggcctggtgacactattctaattgcagctggtggtatgcatctggcttcaaatattcaaattaaaaaaccactttgcctggtaaatttcaatccataaaacttgcttgagtcttttttattttacttgtttaaataagtagagtcataaatcatttggaagatttatgaaaagtatgtaaatattggtattgttatttgttactacttttgttgattcgaattctttacagattggtggaggtgagcttcctgatgaaactaccctcatatgttctcgaggttcagacaggtttgattaatttagattatgttttattccttttgattgatatatgttcattgagtctcagccgtatgtatcgatggggatgtgatttttgtctaaggtgaaattgaagatgcaaaacattagccatatttactggcatattatgatatgcgtctttaagtgagaaaattagtgaaagaattgattagttctatcactgctattctctatagtctatagtctcagtatggtaggataatatgatcttttgttgggttctcttaactctagagttcaatccgatccggctcttacagtagtctttactttcctgatttttctgttctttgttactccttttctgtctacctttcattcactgtaagaaatcatggtgcatatgttcgaatctatttgggcagcgcattggaattattgtccacctgcaaactggcaaacctaactgttagggcagaactcggctgctgcttgcttcatagaagtgggaggcttacaattgagaggtgtgtgcttcaatgcgaatcaaaccccttagattttttatcctgccccattgtaagcacagccactgcagacaacttctcttcctcagttaagtactataaagacagtgtttcagt

SEQ ID NO: 33 - DNA, 12116 bp, Cannabis sativa
  source 1..12116: mol_type = genomic DNA
agacatttcagttaaatcttctaagagctggaaaatcatgccgccttcaatgtgctccttttcacaaaggtataatacaaatgtctgtacaggaaggtgagacaggtaaggcgcgtgttgagaagaaacaatgcatactaagtgaagataaagtgattccaaagaccatcttttcacgcatatttaagcaccacaacatccaccgtaagcaaaaaaacttataggaagtctaaattttggtgtttttaaatagataggtgtgtaatgtatactcatagttaatactaatatttcagctctacaaatttcagattatttgttttacaattggttagctttaagatttgtactagttgtagttcaagtttacgaggcaagtcctaaagggactatcatttcagtaaagaattaagtatcaaagcttcaacaaatggtacaagcataacctgacgtgatacgtttacatttttgctattaaaggaaccaaggattttactacatctataaaatagaacaaagtataaaaagtgaaattatactcttccattataaatgtttaaagagttgaagttgcaacaaatctagagcactaataattggaaacacacataacaagactaacaaatcaaacatgcacataccaagaagacgcggaatatggccagggaactctccccctcatctatcgattgcccatattgcattattggctcccctgactgtactgcacatgactgtatgatacaagcaccaaaaataaagtgaggctgactgcctacagtatattaaaacaaacaaagtgaaggtttctccagttctataaaagaaaacaatcctcaacaccaaattcaatttacagaaaagggcatagaacgttaataaatactataaaaatgtagctcgcatacttatatatatatatatatagcaaaacataccaccagttcctgaagcaaagttcggagaatattttctaataactgattctcttgcgctaactttgtttgtttctgtaataaaggattccaataattaaggataaccaagtcaatgccttcaaattaaaaatatcctattccataacctaaaattcaaagacaagaaaagattgagatattatatacctgaggtttaatgacttgttgaactgtttgtaaagcaaaagtttcgggtggcccaggttgcagtatggccctcctaaaaatttcctgggaaatggtagaaagatgggttatttatatgtcaattactcaaataagtattaaatacaaattttaagaagatacggatacaattcaactgactgaccatttggttgttggaggagccaatgatacacaacctgcagttgaaatcaaaacacatttgattattttaatgatatcaaagcaggattatggattggtcaatagtctgatgcatgaacaatttgttctacttcttagtaaaacactaaaactacattgcttataagcataaccaatagtataacatttgctttggacaaaaaaaaaacctcaaatttgaaaactaaatcttatctacatataatatatatatgtgcctatatacaaaattttaacagaatattttgaaaaattctgctagtttacattagcagcctcaaaaccctaagtagagtacctgttgtgttgtgaattgtgagtgaagtaattgtgttatccaaccaatgaatgaaacggaaaaaacctactctcgatctccgatctccgatgagtaagcgaacgaggttgaatatatttctagggtaccgctgatgagttagagtttagtgacagaaccgaagaaagccatacaagccaatagagagagagagagccaagaagttgtgttgtgactggaacccgccacttcggtttatatgggcctgggcctgggcctgggcttctcatattgggctaactcaaacaatacaaacatatccacccttttatgaaaattaccacaattatattcatcttttttaattattaaaactaatttctatatatttagccaaaaaaaaaaagtaataaagacaatagttcaattattagaactaatttttgtaggactttgaagaaaaaaaaaaagataagttttgatatatatggcattaaaatttaattgtaataaatatggcattttttttaaaaaaacttaaaagtaagaaaaaaaatagggaaattatccgatatactatttttttaatttttttttttttaaatttacggtttgagtttctaaaatggttgcagcgctagttgtaataggggtttctatacgattttttgttgcaatttaggttgcagcgctatagttgcaataggaatttttatgtagaatttcgttaaaaaaaattattttgaggtgtaaaaataaaaaagttaaaaaaataatctgagttgtaactagcttattattctttttacagatgctaaagcaaatactaaaacaattataaaatgttgacggtgactattaattctatcactagtactaaactaaaatctctaatgtctaatcttaaattatagtaacaattagatttaaaaaataaattaattatagtaacaattagcattcatcatagatattcgataccacatttaaaatttaataaatttatgtttttaacaatatttttatagttttttcccttctaacaaaaaataaaataataataataaaagaacctgagatccacgtttaatcgaatgttaggtgtacaaggtgaaggtggggtgggggcccacctgaaaacgagagctcaaatattctggaccataattacttattaaaataaattaaaataaaaatcttaaatcactcaccaataagaatcaaggatttgattgggcttaacccgaccacaatttcgatacgacccctccacgtgtcacttaattccaccaccatttttcttttccatgacgtcacgtgacgtgggtcccacttacgaccactcccttagctctctctagcgctttctttctctctctctctctggtttttccttgggattttgctgtttggctacctggaaactttgatgttttctttctgaatctttatctgaaatttgtcggcgatgtgtgaaataccttacctgtgtttcttcgattttggttttggattcttcgtcagaaatgtttgattgggacgaggaggaggtcagttttcttcttcttcttcttactgttattttcatacttgtatgaagttttggatacatgtgttaaatagctgaatagttttgaggaatttttttagttgaatagttgtttacgagttctatttatttcttaatttatgtaatttcttttgtttttaatttgcttaaaagttttaacttaattgttcaacgaaaatttacattcataaacatgtaaagattcatctctacatattggatagtttataaaaacagttaaattttgtataggtggggatgcatatgtatgctttgtggttttcatttaatgttttttgttttgttttgggggtaattgggtttacaggaacatacaagttatcaaagtttaagtttcttttaatttcgtttccgagcagggttgaatccatgttgggaatattgaatattttttatgtgattaactctgaaattagtgatgttggtattgttgtagtggatttgttgattttattaggttagtgaaataagtaaataagtaattgatgcatggtttttcttaggggttgtttggcaatctattgactgtagaaagcagaatttgagtgacttcacatggtgagaaacatcgaagagaattatgttggcttcagtattctagttaaggaatcaacatgccatgttaataaaaactaagttaaatggtcatattttgacaagtaaagcatatttggtctttttctcaaaatagaggaaaaagaaactaatgaggtgagtggttattctgtgacaattacaaaatggtttaaactttctgaaatagctatgagaaatgcaaagaactattgtattagagagtgggggaatgtatcgaattagatgttttcttttcatattttccacagactatgagtgtctagtttcagttataattgtagaaattttgcttttgtcctttgttattcgtctgccttatgtgagggttctaaatgcaaccaagcaaagtaatggaaggaaaattgattcattttcatttcattaccccaaaccgtagtgcttttccagaccaatcgcacctgtagtgcttttccaggagtccttgtaaattaatatgctttagtttctattctcgtggtttcctaaagtgtgcaccgaattctcttgtgctgcttgtgatgtttgaatttgtctttgttccatttacttgaatatattgagctagagatggtgttgtaattgtttttgtgttctgtcttttctatgaacatgtcttttggttgatttctgcaattgttgaaaactctgttgctctttcagctagcaaatataatatgggatgaggctgctgagaatggtgaccatattgttccttatcctgccaaaagtgaagattgtcgcaataagaaggaatggaaacaagaagccaatataaataagcctactgagcagaacaattctaaagttgatttacgtaaagaacaggtggaaagcagtaccaatttgatcaaccatgataaaagttctgattcaagaatgggggtggagtcctggcctgatttgcctttatctaatagtgaaagggaatctatgggtaccaacgtatctaacgccttaagtgagattacgaaatttgactcgtcaacaggtaacatagttaaaaataccatttgttaataacacacatggttgtaagtacacaagactacaaaaaggtaacaaaattgcttagcccttctaagccacaagaaaagctactcttactgaagtaattctatgaaatgttagtttgtttacaggtccttctttacacaaatactcttactagttacacatccaggcctattttatgtattacttgaaattgctaagtgtaatttaaagtactgtgggaactacaacatatattggagtggaaaattgatttttggtctgttattcaacaaattctgtagatcaactaagatccttgagccttcaatattttcatggtgatagtgacccatgattttcaattttgtaaacagtgataatatcttgttggtgtccttaaaattgtaatgggtattggatttaataaaaagatgtaaaggattttccacatttgaatcttcaaataagagccttgaattatcaatatattaattgtgatagtaacccgtgattttcagtatactgtaagaagtggtaatatcttgttggagtccttaaaatcgtaaagggtatgctcagttggtggaattaataaaagatgaaaaagatttccctcatttgtcattttgtataggctctttgaaattctagaacagtagcacctgaaacaacaaaatctttctttttaaattactgaaaatttctaatggcattaaacagataaggaagtaaaataacactattgtctggtaagtgtagcgtttctacctgcatatgtacatgtattagttggcgaagccatgggagtaagtttgagttattgattgcggctccctttcatttatacttgaaattctaatttcagggactgatcagcttgataaagatgctgaaatatatccaagttcccacgaaggcaaagaacaaggtgattttgactatggctgggctaacattggaagttttgatgattttgagcgtatattcaggtaatgcacaatatatagttgtctaatctatacaattaatatacataaacacactcattcataactttctctattctctctcacctcttgtgaaagaattctgaatgttgaaagttctaaagtgcattttccaagttagctttcttattatgatgtccctgtgataatgccgtgtaatatgaattttccttgcctgtttaactgcaaagaaaaaaaagacacgcagaaattttaatcattttcaaacatttccaaatgtttctgttctactaggcattgatcttttatgttcatgaatttttttctcaattagatctgagagtgaacggaggtagtcatataagtagtaagtccccactcctaagtgtgtctgttgatattgcccttctacacttccattcataacgtttatttacccatctgttttctctctttttaatccttttgatatgcccccgctcccaagtgtatgtgttgataaatttctgctacacttcctttcatagcatttatttaccctccttttcatctgttttttcttgcttttttctcatatattttatgcataatggtagccatctgactaaatatccttacaagtgatgattttgcagcaacgatgatgctgtatttggtcaaactagtctgggtaatgctgatgagctttggtgttcctccaaagatattactaacagcccagacaaatccttccccatttcttctgagttgctcagtctgagcccaaaaacagtaagtaatacatctcagcagttggaaataaagacaaaagttgtgcaagaagatgacctgtgtagcctccgctatggggaaacagatgattccacatctcacagtttgcacagtgttaatgcaattctggagcatgcagaagatgcagtaggaaaaagtgagcctcttgccaaggagaaggtattgcgattttgtttctctgatctgtccctggcatgtacattctttacatgatggaactaaaattgtgagaacttttaattggctttcagataaatttggacagagtggtgaagacaaccttatcaacatctaatccagctattgaaaatgcttctacccaaaataaaaatgctgagaaggttttctctccaactgtatgatctgaatactttttcagtcattatgaaacatttgaagagctttcagcctttgcaagctcttccaaagaacacacgccttcatggcttactatgacgaattactagaaaaacactaagacactgaaacttttttgaggtttagaatgaatgaaatgtttgtatatccaatatcaagcagatctaaatgcttataactatgaagagtcaatttccatttgcaaaagagcagtcaagagaatgaactcttcagaattaaaatcctatatacaatctaaagtctcaattggattcaaagaaatcttatagagagaaaattatagagttcatgtagctcttagttagtgaagaattttttatcctgtgtaaagcttggtagattaaagattgtatggattcaagataatattttcttgttgttattgttcttgtcctagttgcaaagaaaattttaaactagtttgtcttgtttcattatttcttttgttaggaatattttgtgcacctaatgtatatgacttgtcttttacattgttgttttcacttctcctgtctctgcatggtattgctatgcttatatttataaatattacatatatgtacattctctacaggtgactagacagaagaagctatttaaatctcgtaaaaagtcggatgtcaagagtgagggtaaacagttacaagaattatatggtgcctggccttcaccaagaaaccattctggtcaactagaaaatcgtatacaacaccctgcaattcaaccatctccctctacagttcagcagatgcagcttcaatgtcctgaaatttttcatttccagcatattccaaatcaatttgtggtgcatcctatgtatggaaacctggcaaatacatttcctgccatgcctgtgctatcgcgtattcagcctgggaagtataagcagcagcctttgctttctagccgtgatgtttcaccagctaacagcaatcttgtaaacaattcggcagaagctcctgtgaaaactacaatcatgacgcctcaggaaaaaatcgagaagttaaggcgacgacaacaatttcaggcgatgcttgctattcagaaacagcagcaacaacttagccaacaagtctcttcttccaaccattccaccactcaaacttgtcctcaacaatttgggggaagtgatgttcgacttgaagatttgagcactttctcttctttggaacctatctctcctatggaagaagatgattcaagttctacatctgcagctatcgatgattactctgtcaatgaatctatacttcacagacttcaagatgtgatatcaaaggtattgctagtctgctattgaattttactgttatgaagtaaaatggatttagttacaatcctattcttgctgcagttggacatgacagtgagaatctgtatacgagatagcttgttccgtctggctcaaagtgcaagagaaaggcagaatctcagtagtacaaacaaaggtagcaaggacgaacgtgaaacacttgcagtagatgtaaccaatacctctaacaggtttacacacttccttttcattttagctgccttacataactagttgtgattgttctgctgcattcattccttaaatggctgctcttccaattcttcttagtgcaattttcacttccaagatgagttgataggagaaaataattatgataggtggataattttaattttctaatacataaatttggaaagaatgggtgggttcaattcacattttttttaaagaaaaggaatacttaacaacaatgaattacttcttttctattattcttcatttgataccaggtttaaaatcaaatacaacagttgacctgatgattgaaaacaaatagagaaacttgcctatttacaattatgacttctagttgtaaatataagattctccacagttgtatgaagatgtgtatgaagatgtaattgtttgaaacttactaagagatgaaaggaaaatatttcttgccaaatctttggtcaaagcatctgctaattgttttccaattatgacaaaagttgttcaagaggcctctttaatgaggcatatggtgtaagccttcatctttcttgaaaagcactatattgttaattacccaactattattaatgtgaatggtgataggagaatgtgtattctacctttattctgcgcttgcttcctatcagcttactcaccatcgtttctgaaacaaaataaggcaaaacctattaatattagtattagtctcattagtataattgattaggattttagaggttgagttgtcaaattcttagtggctttaagtgagctatcctttagtcctaaggtgttaaaatagttaagggatacatagtataaatagagggttgatcaattgtaaaagataggggaatcgttttgtaacaaagtgagtaagagatttcttgacttcattaagaaggcgcttcagccgcattccttcacacatgtgctaaggcgcgaaattctgcttcagcactgctctgagcaattacttgttgcttcttgctacaataggatacaagatttccccaaatataggaacaatacccagaagtggatctccggttagtgagggaactgatccattctgcatttgtatagattttaagatatctgtgcaatgtttttctaaacattagccctttttctgttgttcccttgaggtacttaagaattcggtagacaacatccatgtgttcctctaagggattattaaggaattggctaacaactcttacagtaaagtttatatcaggttgagtgtgagtcaagtaaattagttttcccacaaggtgttggtaccttcctctatcaactttttattccatctgtgtgtgctccaagtttagtattggggtccattgatttgtcaacaagtttgcatccaatcatctctctttcctttaagagtttaaggacatatttttgctgattcactaagattccttggctggatcttactactcccatccctaggacatatcttaattgccctagatccttgatatcaaattcattggaaacgagttgcttgagatgagtaatttcctccataaaatttcctgtaaccacaattcatcgacacaaactattaacactgttaacttttgactagttgagtgctttataaacaaagtgtgattagattggcattgagtgtagccatcgcccttgagaaccttagtaaacctgtcaaaccaagctcgaggggattgcttgagaccgtagagagattttttgagtttgcacacctttccttttggatatttattctcaaatcctcgaggaatgtccatgtcaacttcttccacaaggtcgccattaagaaatgcgttctttacatcaagttggagagaggccattcctcatttacaacaatagacaagagaacctggttagtgtttaacttagcaacaagagcaaatgtctcttgataatcaatgccaaaggattgggtgaatcctttggcatctaagcaggccttaaatcgttcaattgttccatcagccttaaatctttcaattgttccatcaaccttaaatcgttcaattgttccatcaaccttaaatcgttcaattgttccattaaccttatgtttgactgaaaagatccattttcacccaactgtgcgttttccaagaggaagatccgtgaagatccatgtttcattcttttcaagtgcttgaagctcctccattgaggcatccttccattgaagaatctgaagggcttcttggatcaaaccaaggaatctgaacttagtccaaagaggtgataaaagctctatatgtaagagataagcttaagtaacttagatagttgcaaagggggtgttgggtgcaagagcgaaccccttttccatgagcaatgagaattctaagatcgtctgacacattttcttccataataggctcaggtctaacatcattctgtgtttgttcaaggatgaatgggatcagattagattcttgaccttgctgagttcgcgtagtgacttgtgcaatggttgctagttgcttctttcttcttgagtaaaaatagatctttttgcttggattatggacatgacttgaagggattgaggcaaagttccttatttgactcgattgaatggtgaggttggacctctttgcttggaatatggataggatttggagagattggatgaggaatttgataggcattgggatattaaataaaaagggcataagtgtaattgtaataggggtattgttattatatataggactcaatagtgaaggaaaaaggagggaatgatttagttaagtgagcttttagctcttgggagagagctgggtctctcgaatactcagcaaacttgttctctttgtcactctaatatcaatatactagctattttctattgttgctactgtttggttctattttcttctattttgcttctgtttgtagcaaaatgataggggattcctatcaaaattctcagctcatgtgagtcagctgaatagtttggagctgttgggtcccgctcttccatcttgttgggtcaccacctccccttaactcccatattagtatgatataagctctcaatgtgttccacagatattaagaattttagatggttctcacatgtctccatgaatgagagagaagggttgagaagtttgcaaggccttggggcgaaagaatgacaagtgtttagaaaattgacaaatatcaaagagagattgagggttattattgacaaatagggatgaaattaagcgtttgaggtacataaaatgtgaatgaccaagacggtaatgccataatgtgattgtgttgattttattggattggtttactgatgtaagaaattgacattgaaggagagaacaatgatttcttgtagtgctgttgtacaattcctgtttgttttcttaataggattttgttttctttgtatggtgggtggtttaatatttgtacacactttggccttttgtattattctacttattaataatagagtattggcagcagtggtaactagacgtgttaccttgtaattggttagtgatactcttttaaggttattatattaaattatatagaacttgatacttaattacactaataacgatattgacacgtagaaggacatttctcctaataataaggaagtactttt

SEQ ID NO: 34 - DNA, 7248 bp, Cannabis sativa
  misc_feature 5882..5981: note = "n" is a, g, c, or t
  source 1..7248: mol_type = genomic DNA
gtgttcaccaaatattaaaaaaaaaagacaaggtagaagatccggatgattgttgagcaaatctgtgggatcttactttccaaactgtcaatatttggtatcctatcttcattctcatcatgtctcctctcctctctcttctattttatattctcacttttcaccattctttctcttcaattcttttctctctcctttctctcttctcaattttctctctaaaacattattatcatcatgggtttggctactaacttagaattggatcactttcatgtgttggctgtggatgatagcatcatagatagaaaattgattgaaactctcctcaagacttcttcctatcaaggtatatatatacacattaatctccaaaaagaattgatgtattttgtttaatgatgcattgttgttatttatgaaattttgtagttactgcagtagattctggaactaaagcactagaatttcttggtctaaatactattcaagaagaagaagaaaatgaagaagtaacaaatttcccatctttttcttcaacaactcatcatgatgatgatcatgatcatgatattatggaagttaatttgatcattacagattactttatgcctggaatgactggctatgatctcctcaagaaagttaaggtaacataatttatataatattatcagggtcagtcctgatctgagacgaaggaggcggtggcctcaagcctcgcaccgcttaaatataattagtaataaaataaataataatactcttaatattataatttaattagtttaacaattcactattcaaatttcaattaatttagatattgtttttttttatatggtattgcaggaatcaaaatctcttaaagatataccagttgttatcatgtcatctgagaatgaaccatccagaatcaacaggtaattttatctttaatacaaattaattaattacatataatctgaaattttgtttgaatgtataaattataatatatataatgattactttataaaggtgtttggaagatggagctgaggaattcttcttgaaaccagttcagttatcagatgtgaagaaactaagatctcatttggtaaagggaataagatctgatcatcatataattgaaaatcaacccaacattatggaaaaaacccaatctgctgttgaattggaatttcaatctctatgaaaataatcatatgaatttcatatacatatgttgttttaattatttcataggtatttattagatcagatttagtggagagaaataacattattgatctatgctagtattattatattattcaaatatcaacaacattttttacaattatttatttttttaagtcattaaatttttgcctctattgatcatattaattgaacagggcaataagatttgaattgttttttgaagtcaaagatcttatgaaagagaaatgcatgatttcatttcaatcaagttgattggaaatattgaataataaataaaaattaaaaaaaaatagtgattggagaataataataattgaaattgttagctttgccacctcattcatagctggttccaactttattagtgtggtttttgtaaaatagggaaaagtagaaaaggtacaattttttatttattttataatactaataataataggaaaatttatgtcaaaagttctaaaaaaaattagtttgatacaaataaattcaaacttaaaaagtaataatatttttttttataggtaatcacttttttaaagttgtttacttattaaaaaccactattattatctaaattttaattttgatttaatttatttaaaataaaaaaaaaactaataagtaaaccattcaattaaaaaaaggattaatgaacttacggaaaatatcacttaaaaattattatcgttattttttaagattaaaaatttatttgtattaaactaaattttttgaaaacttttatcgtaaatttaaaagtaaaataagtttatggtaatatatctattagtctacttttctcatttgacacttattctttcaattgagtttatgtatcttttacatattccattcttgaataattacaattaggatctttttagttaaaataaaaaatattggaaatcatatatgttatattttataaattgtatgcttccttacaaacaaaaaagaaacaataataaattgtatgaaaaaatacggttttatattgaaattcttaattatggcctatttgtttttttttaataaaaaataattagtttaagtcaagagtattcataaaataatccatggtataaaaaaaatttagtttaaaattttttgtggcacaattatagtttgtgtttattttttaaaattgtaaggtttaaaatttaaataagaacaatgatatgtacatatgttaatcataaacaaattgtggcatatatattgaagacaattttacatgtcctatggaaagataataatttttttacaaaaatattaatttatatatatttgtttttgctcaaaataatatttatatttattaaagtatatatatgtgcaaaagtctaacaacttaatttaattttatgcaccaacatcgacttgctagattgacaagataaaatatgagttaactaattaataagaaagacacataataccatacaaatcacaggttaaaaccttgtgatttttagctacggctctctcaatattacaattgtgagaatgcatgttatagatggaaatatgagaataacttagaagaacagagagacttagagaggcttaagctcttaggtgcgtgtgagaggacttgtggagaccctttagtgtgtgtagaatgacttagccttgagagagtgatctaatgccttttatattgtgtagggcttacaaaatatcctaattcaaataggcaaagatatccctttaaaatagaaaagaaaacatattttatatcttgtatttgaattacaaaaaaggataattaaatcctaaaatataagactaatatcttgagggaatatctctatcttcatggattgttggatctatctgtccgatatagtaggtcagcttgagaatcctctagaaggttgttgtggaccccataattcgttcagccttttttgaggctgtttgcatgtgttggccaaccaactctatttttagggtctggccgacatgtaaggcttcattggccggccaggtatggtttctatacagttttgttgttcagcttctttgtacaactttatatacatcttcttcgtacaactttgctgcttaacttggccggacaaacctccttggttggtcagcaatggtctggccagggtttaaaaacttgtcttgttttacttggaagctgcacggagtgtacctgttcggttgttgacatgttgtatttcacgtgtcttttgttttgactatgtagacatggcacgtcatgtgaaaaaaaaatgggataactataggtgttcatccgatccaattcgtattattttactcatagtgcatctgatctgcaccaagtgcagattttatattttgaatccaattcaatccatatagtaactctaatctaatccaatctaatccgtaaaagtgcggatttaatctgttattttagattggttacttttttaatattgaattatttaatatttattatcaaaaatatttttgtttacataactaataacaaaataaaataaattattgttattttatgtatcaaataaaaaattaaaataaataaaataaaaaataacaaaaatttttttgaggaaaacaaaaaaaaatattcaaagcaagaaaataattatatgtataaaaaaatatttaatattgttttatataatattttccttgtaagtagggatgaaatatacatttgatatactatattttaaaatatatttgtataatatgtattatattttaaattattatttttatattaaaatattaattgtatatatataatttttttaaatagatatataaatatgtgtagattggattggatcagttacttttacatatcaatcaacatctaattcgcacaagtacagattttaaattttccaatccaatctaatccgcacaagtgtagatatccgtattttgctgattggattaggtattttatgaacacatctagggataacaccaacagagatcaaatttcttttaaaatcatgtactagtcggacgtccttcaacaccatcgtgaagtttcaaccttacagttccaattcccaccatcttacaagacttgttatctccaagatacatagtctcataattttcagatgtaaattcttctaaccaagatttattcggtgttatatgaaatgaacaccgaaagcccaagatccattcagaatatgaattctcaaaggttaccagcaaagtgccagatgagtcataactgtcactacttattgttgcatttccttcagatcttgaattacctgtcttatgccgctttgggcacgcctttctgagatgccatgttttcttataaatccaacatctaaaattcttctggttatcagattgtgctttagatttaaattttttaaaaaaatacttgttctttagatctagattttacttttgaatctttgtgactttatcttttctcacttttgcccttacaatccatcaccacttttaaaaaattttgactcactcctttgattcagctctttcgaattcaaggttgattgcacatcatcgattgtgatcgattctcttctatacaacattgtatctttaaagtgtacataaaactttggtaaagaactgagcaaccacaaggctagatctttgtcttcaatagtgacaccaatattttcaagatcgagaatcaacttgttaaactcatcaatttgttctccaacagatttgtcttctgtcatcttaaatgaatacagggcatgtttcaaatataatctattcactaatgatttagccatatacaaactttcaagttttagccacacacctgcgattgcatcttctttagcaacctttctcataatcttatttctaagattcaaaatgatcacactatgagcctttgtaggagtctttttcttgtctttttcagatattaaatcaggaagagacttcgtattttgcagcaaccatgattacaaaccaagctttgataccaatttattgtgcgaaacagaaataatcaatgacttatgatttagtttacagtatcaacaaacacgcaacgcaatcaagaaataatataccaagacatttcaagattcaagtaatgaattaagcaataaaccagaaaagattaacaccaggtttaacgagttcgagccatgtacaatgctttacatctcgatcggaacaacaacaaccatatctttattgatattagtgtttacaaggttacaagaatcctcataaactctcagaaattacgagaacaagtttatctctctagtttagactctctctcttaaccttacattatcacgattttcaatcaaacaaatctggtctaattctcacataggttataaaaatttacaactatttatagatttgcattattgcctaaaatatatcctaaatcaccgtgacacatttacaccaccttaaatgatctagctcatcactagcaataataatccacgctaacttaaaaaacacactttcaattcgaacaatctggaagctttataaaacaagcctttcggatacgtacatagcttccttcccggatagatcttcaagactgacaataagttgtctgggaatgtatttccggattaataacaagctatccaagagagacattccatacacggtacaagcttatctagaagtaccttccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaacattttagaagtctttcgctggtgcaaatctcattagcttaaaaatttgtttcttggtgtcaatctcattaacaaagtttacattctactaataatagaccatcaaatagaaaagttcactacacttttcagattgtttttttgggaaaagagcacttgtttcctttaccaactatagattagttggttgttagtgagttcttctttgtaccaagtttaactcaaacctctttttactgggaaacttggaatactattaacaaagtgaacatcttacaaatctttcactggtgccaatctcattgacttatagtttatttcttggtgtttcatgtgtttgttgtttacaatttcaaattatcaactatttttatcttgtgatcacattcagacactcatacaatcttttcatggtcatttttcttttgtggatgaactaatatatctcggctgaaattctttttatcatttcttgtagggaggaggctaaagatatccagtcacgttcttcaaagcctagaatacccttagcttctgaggacattcctgagcgtattaaaaggtataaccattgtagaacagaatttttatatttgtcagtgtgcacattgtgaaatctaactaaactcactatttagattcttaattagttcttacctattattctccaaacatcacctattattttcatgtctcatgctattttatctttcatggacaggaaaacagcagcttcctacagagaactagaagcaagaaagaaaagggtcaatgagttggagaaaatctacatggatatgtcaatgcagcaggaattgcaggtaatgacatacaaaaacttttccatgtttttctttctctatatggtactaaattagagtttctagaaataaaccattctttataaacttgtagaaaaagggtcggaagcgcaaactacgcgaggatgagattgtcacgccaacatccaaaccggttttcaaatggagggcagagcggaagcgttgaaaagaatagtgagacagtgtggttggattggatcaaaacaattttgtttatataaaacaagtttgcttctacattttaggcatgccaattttcaaagacatctactttgggaaaactaggctctagttaatccagattattgtataacggttatctctttatttatataacaattttgtatggtgtattaccaactatgattattagtcaaaattttgatttatcaactcttcctttataaatttccaaataaatt

SEQ ID NO: 35 - DNA, 1206 bp, Cannabis sativa
  source 1..1206: mol_type = genomic DNA
ctcaattttctctcttctacctcaattttcaaaatcaatctctctctactcaattttcaaaatactcttatatattattacaacttaatggctttggaaggagaagttaccactttggagctcataaggcaacatcttcttaatgattttggttcaatggacagttttatcaacagccttaacaactgtacggccaatattaatacccaaaaccaaactaatcatattgatcatgattattctttctcatcgcaaaataaccaatcattcaaagttccaaagaaagaaacaactaataacgatgactattgtgaggaggcaaatcacagttttgagtttcaaaccaaactaaacacatcagttaatacccgcggccactactctaaaccctccagtttaagccagcgccgcccttctattaacgtagccataccaccggcaaattctccggttccggtgaaggttccggccatgtcatcgtcggtttctgacaacgctcctgacagcagacactaccggggtgtaaggaagaggccgtgggggaagtacgccgccgagattcgtgaccccaatcgccgcggctcgcgcgtgtggctaggaacctttgaaaccgccatagaagctgccagagcttacgaccgcgctgccttcaggatgagaggtagcaaagccatactgaattttcctctagaggccggcaggtcgccgatgccggagagcactgaggttcacgcgccggccaattgtactgttttgaaaaggaggagaggaagtgatgaggataaggaggttattagagaagcgaaggagttgaagaaggaagaaacgacgtcgtcgacattaacggagagtactacatcgtcaaatgatcatcaggaggtgaactcatcagggagtagtaacggtaacagtgcagttgtaccgttaacgccgtcaaattggacgtcggtatgggacagtcaagacgttaaggggatttttactgtgcctccgttatcacccttatctcctcacttttctattggatattctcaacttatggttatctgaatcaaataataataataataatatgttaatagctaggctagctattcacaatggctagaaaaagtcaatgggtagttagggttttatgtgtaaatatacgtatatgtatgtatgagtggtattaattatatgttcaaattaagtagtatttttttt

SEQ ID NO: 36 - DNA, 2304 bp, Cannabis sativa
  source 1..2304: mol_type = genomic DNA
acgctcttatattttcttcttccttttttctttttcttctcttttcgggataagagttttttcttttttttttttgaaaaaatatttgtttctctctatttttctgagcatgaccagtcctttttttctcccatgagaaaagaaaacaatacaaacaaaatcctcacataaagctgaactgtctatttctctttcgattcatttgcagaattctctcaaactcttctgctcttctttaatttctttcctccattataatttttcacctcggttggtttcccaatattacccagagacgtacccactactagtctcgtctctttctctcttaactagtttctttctttttacttaattttagatatttatatatacataaatatattctcaaatgatgcaattcactactaccagtactactcctactcctactactacaacaattacacctccaaatattcctccaatattattgtgtaataatcttcctattatgaacaaaacccaattaaaccgaggtcatcatcatcatcgtccatggccaggaggtttcccaacctccaaggccttaggaacctcctttggggacgccaattgcatggagcaacttctagttcattgcgccaacgccattgaaaccaacgacgtcactttggcccaacagatcttgtgggtcctcaacaacatagctcaaccagacggagattcaaaccagagactcacctcagctttcctccgagccctaatcacacgcgccgcacgcatcggcaccagctgcatcaagatgctcgcctcagccgttgcagttcaaacccaaaacgacgataatctcaacgtcgtttttgacgttccacacagattctcagtcattgacttcgctaccttcatcgacttgacaccctggcaccgcttcggcttcacagccgctaacgcagccatcctagaagccgtcgagggcttttcggtaattcacttggtcgatttgagctcaactcactgcatgcagatcccaaccttactcgacgccattgcctcccgacactacgaggtaatgtaagaaaaattaaataaaataaaaaatatttttgtttaaattttaaaatttagtttaattaattaattaattaacaggtgacacctgtaatcaaactgacggtagtggtggcagcagaagcaaacgacgtcgttccaccaatccttgacatctcatacgacgaactcggtttaaagttggtcaacttcgctcggtcaagaaacataattctagaatttcggttagttcattcaacttactcagacggtttcgcttcactaatcgaacatctccgactccaacaacaacagtacttagttaataataatcataattacgaaataacccccgaagctctcgttattaactgtcacatgatgcttcattacatcccagaagaaaccctcataaacagtaataataataataatgataatgatattaactgttcatcatcgtcatcctctctgagatcaatgtttttgaaatcgattcggagtttggatccaaacgtggtcgttttagtggatgaagacgcagacttaacgtctaataacttggtgtgtagattaaggtctgcgtttaactttctttggataccttatgacacggtggagacttttcttcctaaggggagtaaacaaaggcagtggtacgaagctgatattggttggaagattgagaacgtaattgctaatgaaggagttcagagagtggagaggcttgagccgaagagtaggtgggttcagaggatgagaaatgcgagttttcggagcgttggattcggagaagaagctgtttcggaggttaagactatgcttgatgaacatgctgctggttggggacttaagaaggaagatgaagatcatcttgttcttacatggaaaggacataatgttgtttttgctactctttggatgcctgcttctgttgcactaactactactaattaattaatgcttcttacttaattattaattagatacttaataattaatttaagtagcatattattattattatctaattttatatattataattatctgattggttgatgagtgttttcttctgttatattttttctataatttgtagtttaaacattatgtatatatatggtgctctttctaattatatcgtaatcgtaattaattattataaaggtaataagctgtaaaaaaatgaatatgaaaaggtcctctcgtgtttattttcaatgccaattttatactgctatgtgtgtgttgttataaataagttttataata

SEQ ID NO: 37 - DNA, 934 bp, Cannabis sativa
  source 1..934: mol_type = genomic DNA
accaactctaacttgaccaaaattacgaaaaaggacaaaagaagctcaaaacaatgtgttcccatggctaagccgattctgctccggcaaccctagcttatcctcttcttctttctctctcacatgaacacgtttgattccttctctgccgtagaatcgagcatggaagacgaatcgaaccgccggaatcggagtaggatgcgggcccgaagccgatccaaacccgccccagcctccattgccgtctccgatggagaagaggaagatggagatcctgaattgtggaagaacttcgaccagaatttcggacaggtacagacagttctggatcgaaacagagcgttgattcaacaggtgaacgagaatcaccagtctcgaattccagacaacatggtgaagaacgtggctctaatccaggagatcaacggcaacatttcgaaggtccgttcgctctactccgacctcggatccaacttcacaacctacttccacaacaacaaatcgcagaatggcaagcccgataaccttcgtcatcatcattgatattcttcgtttaaaaagttttcaaaatcattgcttttagtatcttctttttaaataatcatgttgtcttttaataaagtgaggcttctgggtgtaagtccttgagctaattagagagagcccacatggatatatatatatatatatatttgtataagtatgaactgtgtttgttgtgatgtctgcttgtcccctgtttgtactaatatgtgtaacacttgttgtgttttaaaggaggatgaagtacaagaccctctttgggtttcctcatcattccatagacgtaataaggtattatttgaactttgtgtttattttgttttagtttcctttgtttgattatttactatctctctctgttatcggtactaaataaaggtgctatgactat

SEQ ID NO: 38 - DNA, 2675 bp, Cannabis sativa
  source 1..2675: mol_type = genomic DNA
catcctcatttagtgaacaccgaaaccggaacaagagaaaaggagacaagaaaaccaccgccaccagttcttgatttctcagaatcatacataatcccttttatcttcttcatcaccaaaaatagggtttagggtttttcatattcttccattcagtgacctttaaactccaccatttatcatcatcatcaatggcgaccgaattggttgtcaacacggagagagtccagaaattcttcaacgacctggaggagcaaaagaccatcctttctacctgcactcagctattcacaaccatatccacccacttcaattctctccaacattccctttcccaaaagtaccaatccctcgaatcaagaatccaagctcttgaatcccattcccgcgacaccctccagtccctcgacacccgcgaggcttccattcccgaacgagaatccgcctccctcgcccgcatccaggagcagaaggaagctgctttgtccgagttcgaaaagcaaattcccgaaaacttggaactttcagaggcccttaaatcattttcccggaaaatggattcggcgggattggtgaaattcatcgtttccaagaggaaggagtccgtatctttgagggcagagatcgttcagtccttggcggaggctgtggacccgcctagactggttcttgattcattggaggattttctgaattccaagtcggccaaagctggggttactgataagcgatgggcttgtggattgcttgttcaggctttgtttcctgagagccattcggagaagataggacctcaatttgcgagaactgtcgtggagagagcagtcatgattttggagcagtggaaggggcagatggatggtggtgaatcagaaggcttggctttaggggctgccgaagctgttatgtttgtgcagatggtagttgggttcgggttgaatgcgaagttcgataaggctttcttgagaaagctaatgctgcagcatgcttcgaggagggatatggcaaagctcgcagctgttttagaagataaagactcaatgcaaggtaaggttggtttttaatttagagaactacattaagtagcttaataaaccttttgcttagcagtgatagtagatatgtgtagttacatttgattgactcctagcatggtgaaattgtagaatttataacattacatgagaattcacacgtttaaatgattgatatacatacaagtgaatcctgtatatgttacaaaggtactgtgaacagtaccattacagtgagtataacagaaaacttcttgaaacttattgtctaaattgctactggaaacctgtctattttctagtgcaatgaagtataatatggacaatgaggcaaggcaatgtaagtaggttaagagataaaagtttcaaggcactggaaaaggaatggaaagtataatcatagttaggcagagaaagatttgaactgttatacggttcttttatgggatgggagtttattatgaatttcaagtggagaaaaaaaataaaatgaaggacaactcttatatattcaaaagtgacattggttgctcacatctgaacttgtttttgtttaagtaaaagcttttgaaagttccaaatgtttactttgaatgcatataatcgagacatttttttctgactttgcagagattattgatgagttggtgaagaataacaaagaagtggaagctgtctattttgcttgtgaatctgggttggctgaaagattctctcctgttactttgctcaagtctcatctcaagaacttcaaaaagaatgttgccaacatattaaagaatggaaacaatagcttggttgcaatggtttgtctatattctgacatattttacaaatgtctttttgcatgtagtcacatgactgcactttttaagattttataatccaagtcgtatcatccacaggaggaatctagtaatgtggagttaaattccatcagagcaataatcaaatgtgtagaagaccacaaattagagtcagaattttctgttgaaagcttaagaaagcgggctactcatctggagaaagccaaggcagagagaaagaagagttcaggagcctccagtaaacctcaaaataagcgaggatacagtgccagcagcggccgaggtggtggtggtggatcatcttcttttcgccaatccaaagctgccaagtattccaacacttattcctcttttagcaggaggaatgcgaatcctcctccgccggttcatcacagcactgctgcaagatattctggagcatacaactaccctgttcaagctgtgtacgacacaccagctccagccccagccccatatccgtcctcatacggggttccccatgctcaaagccctgtcgcaattcctcaacactatctctcggccgacaacttgagtagtactgcctttcggagcagtggctcttacagtggtctgaccaactacagaccatatgattatgccgaagcagctccacatacttaccaaccgtcctcatatccaaaatagattaggagcggaaatggcctaatgataagagccttgtaatattacttttcttttctcttttctggccaacagatgagagaattttgcttaaaatggccgactttttaaggttaaacccaaaagttggtaataagccctgtttttatga

SEQ ID NO: 39 - DNA, 4435 bp, Cannabis sativa
  source 1..4435: mol_type = genomic DNA
aactcgtataagcctcttctttctcttctctttcttgcataaaccaactaacacatacaacggagagaggtttctctctccttctctcttagcgatttcattctttctggtttcgaattctgagaatttgggggcgcatcgaaaccctagattcgctcccattgttaaccggtgagtttctcagattcgttttcttttttcttttattttatttttatttttgtttcgatttttttttcggcatttttagaaactatgttatgagcgcagatccgttcgagctttcatgttttatgtttgtgtttttgggtttttgtattcgtttcctcgaaccctttctccacggtgtcctaaatttagagtcttggtttggtttgctgcgttttgaaccccaatatttggattacgtttgtgaccttcaaaatagtttctgatttgtttgtggttgattcccacaatgtatttacggtagattagtggtgtgtatataattgtacgacccttgttcttctcgctttgagttttagtttgtgatggctatattatatttgtttctatgtattaaatatctctctatagatattatccttttgtacttcttaatatgacggagaaaaatatatctgagtctttcgtcgtggctgatttgcatggtactatctgttttctttctttttctaatggaagcaaaaagtagcctttttgtgggtttaaatgtttatttgcttctctttcatgttgtctgtttattttatgatttcttttttacctctggttttgttctctgttttattacgagtttctttttacgggtcattttgttaaattgatcaactgcattgtggtgagaaagtagaaggaatagagaacaggggatgcggttttagagcttgatttgttcatcagagttaattgtttttatataatacttcttagctaagagaggacgtaggagaaaaaatcagagttttactcgattgtggtggatatgatggagatttcatgaatagccaattaataatctttggacaactagaccagtattagcagctgaaggaaaaaaaaacatatatgatatgatgcttgtgctaaaaaatttttttgcatctatacttttttggttttatgataattaccagtctgaaatgtgattatgttgtcacagggaggactgcggaattgtttgtaactaacgtttgtaaaatggatttagaagataaattgcttgctaaaggtatgacctttatgtttcctattgtgcttgtgtctttgtgtgtgttttccactcttatgtgtttgaaaacttcagtagaacttaagaacaaattgttcgagtactgtacattctagtaggtgacacttataatcaatttcgtgtactgtacattttagtgagaacactcttacactcttacactctttggtttttataatcaatttcttaaagttttaatcacttagtagagcatgatgctgttttatcctgacttaacaaactacacttttgatgctgtgttggtttttgctatttatattattacgcttgtatagaaattgtattggttttcattttctcttatcttttattgtttttaattttaagtgatggagaatttttcaatagcttcgttttaagtccagttcttgaaaagacatgctgagctcagttttgtgttttccatctccagtttctggtgttcaaagcctgtcttccagtgtgcaaagcactcctgagaagaatggtaattctgatgatgcttcaagaagtccagagctacttcaagaatttttgagatctggtcccaagaaggaactgttaaggacttgttttgataaggaaaaaagaacttagttacatcaaaaaacaaaaccatggagatagcaaaggtgacgagcaagactaataagaaggtggattcaagaaaagcttcttctagtcctagcaatcagccttctagaaagcaaaacaggaagggagaaaatccagtgcgactccctccatctcttgaacaatcttccgaatttggattgtcaaactcgtggatatgtaaaaattctgcatgtagagctgtcctgtcaatagatgacacattttgcaggcggtgctcttgctgtatctgccacttgtttgatgacaacaaggaccctagtctttggctggtttgctcatcggaatctggggagggagactcttgtggattgtcttgccacattgagtgtgcccttcaacgtgaaaaggttggggtggttgatcttgggcagctgatgcagctagatggaagttattgttgtgcttcttgtggtaaagtgtctgggatacttgggtgagtaacaaattaatgcatcttaaagattaaatataggcactgataatgctttttatatgttaagaaattcttgttaactttaaacctgtactgtgtgaaagaccatccagttttaatattgtactaaagatctatttgattttattttataatggcagatgctggaaaaagcaactacttgtggcaaaagatgcacgtcgtgttgatgtcctttgttacagaatatacttgagttacagactcttagatgggacttcaggtttcgagaggtgcatgatatagttaaagaagccaaggctaaattggaaacagaagttggcccggtgaatggtgtttcttctaaaatggcacgtggaatcgtcagcagactctctattgctggtgacgtgcagaagctctgctcccttgcaattgggaaagcagatgaatggctggccaatgtttctagtctcaatccaaatagcagaggttggaacatcttatgtcttttctttttaattactctattttttcaattgaagttgacatgaatacatatgtttcttgtttctcttgcagaggattcacttccttctgcttgcaagtttctttttgaagaagtaacaccatcatctgttgtgattattttaattgagctgtctaaagcatcttcgaatgagattaagggctacaagctttggtattacaatagtagagaagatggatattcaaaagagcccaattgtgtctttccaagaatcagagaaggattttgatatccaatttaaagccttgcactgagtatacttttcggatcatatcttatacagaaactggtgatttgggtcactctgaggcaaagtgtttcaccaagagcattgagatacttcacaagaatcctaatttacaagctgtgaaaatcgataagaaagagaatacctccactgaggaaagtcctagtgggaagggggaatcgaaaaatgcagtggcagctggttctgaattcaaggttcgagacctgggaaagatactacatctagcatgggctcaagagaaaggctacctagaagggttttgcagtgcggatgtagaaaagtgttgtgcagctgacaaagtagtaaaaacagaaatcatacaagaacgggtgccttctgtctcacgtgggcttgacttgaatgttgtttctgtccctgatctaaatgaagaactaactccaccatttgagtcttccagagatgaagacaatggctgcagcttgcagctaactgttgaagcagatgacgctgcttctcatgacatattgaagaacggtttagcaagatcgcatggtagtggtgactcccagacctggactcatggagtgacaggagatgtgccagccgttgattcaaggaccgatttttgtcggaaaagggcattggtgaacacaattgaggaggcacatgagtgtgacagtacactgattaatggttctccattccgaatatccaatggatctagttccttggatgaaaactttgagtactgtgtgaaaataatccggtggcttgaatgtgaaagtcatataacacaggaatttaggttgaaattgctaacttggtttagccttagatcaactgagcaggagcggagagttgtgaatacgttcattcaaaccatgattgatgatccaagtagcttggctggccagctggtagactctttctcagatattatctccagcaagaggtcaaagaacggtttctgtagtaagctgtggcattaaaatgagaggagggtcatcatacttatgatgaggattgctatttgctctcctccctcaacagttttagtttacgctcttctttcgataaacgacctttgacctgatcaatttcgccaccatctttgtacaggcaaatattttttgttaatcactgtctgtatctgtagtgtttctcattagtatcacgggcaagatttgttgcggctaaatgatttggttgctgatcaaggagacaaaatatctgaattttctctggagggaggatttggatgaaaggatatatttcacatctacttctacttagagaggaagcatttcttatatttcttaattgttctaattacaatgaagcagtagtagcatt

SEQ ID NO: 40 - DNA, 2515 bp, Cannabis sativa
  source 1..2515: mol_type = genomic DNA
agatttgtgatttgttgaataaagattggtctttccttcttgagggtctgtgcgaaatgggaatttttgaagagatgggattctgtggtaattttgactttatttcatgcccttctcgggatggagaggaggttcaagagtgcgaaccagaggcaactgtggaagaggattatagcgatgaagagatggatgtggatgagcttgagaggaggatgtggcgggacaggatgttattgaggcggctgaaagagcaaagcaaggggaagcaaggagttgacaatgcaaagcagcgtcaatcccaggagcaagcgcggaggaagaagatgtctagagcacaagatgggatcttgaagtacatgctaaaaatgatggaggtttgtaaagctcaaggttttgtgtatggtattattcctgagaagggaaagcctgtgactggcgcttctgacaatctccgtgcttggtggaaagaaaaggttagatttgatcggaatggtccagctgcaattgctaggtaccaggcagaacactccatccctggtcagaatgaggactgcagtaccatggcatcgactcctcataccctgcaggagcttcaagacactactcttggctcgctcttgtctgctttgatgcagcactgtgatccaccccaaaggcgtttcccattggaaaagggtattgccccaccatggtggcctactggtagtgaggagtggtggcctcaattgggtatgcctaaggatcaggggcctccaccttataagaaacctcatgacttaaaaaaggcttggaaggttagtgtcctcacagctgtgatcaagcacatgtctcctgacattgcaaagattcgcaagcttgttcgtcaatccaaatgcttgcaggacaaaatgactgctaaggagagtgcaacttggttggccattataaaccaggaggaagctttggcccgaaagctgtatcctgataaatgtccacctatgtcagctactgggaatgggtctcttgtaatcagtgagaccagtgattatgatgttgatggtgtggatgatgaacctaattttgaagtggaggaatgcaagccccgtgatattggtcgcttcaacatcgggactattgttccttcaagggataggattcttatgcctcaagtggcttctcgaattaagggagaaatcattgaaaccaaccctgattttgttcaaaagaggaagcaagtgggtgatgagccacagatgatgatggatcagaagatattcacctgtgagtactctcagtgtccatatcatgattaccgtcttggctttctcgataggacctccagaaacaatcaccagatgaattgtgcataccgtcccaattcttctcaagcttctcagcaaacttttggaatgccaagctttcagattaacaacgataaaccagctgtcttctccttaccattttcccaacctaagccaccattggctcctcaacaaataaaccaatcatctcctttcactggccttgatattccggaagatgggcagaagatgatctctgaccttatgtcactttatgacacaaatgcacagcagaggaacaaaggtttgaatcctgggaaccattctgttcccaccaacatggagaaccatactcaaccacaacaaaaataccaatttcagatggatgatagcttcttcagccagggtgttatgggaggaaacatgtctgagcaagcaaataatatctcatcacaacaagtggttttcccatccgcagaggttcagtttgatcagtgcaaggcgttcgattctccattcgacaataatcccaatgataatattgcagacttaagattttcttctccatttaacatggcaccagttgaatttgctgtggactcactgccaaagcaggatgtctccctctggtacctctgaaaccagaacaatatcacaagacttttactgcttcagtaatttgggtaactgcagggtaattaagtgggaggctcatcttaagactttggagaaaccaggttcttcaaactttattttctagagtttaagctgtctaatgtttttctttttcttttttgctgttttgaagtagggtataataagggactggaaagaatccaacccagtatttgctgctgctgaaaagttgggtttttttttttctttttcttttttcttgtttctttagttgatgttggggctgctatatatctttattatcattattattattattattattattattattattatctacacatactttttatgtatgtaatgatctcatctttgagctacatttagactggtagagtccattttaattacttgtgctgaaatgcctagttcttgtaaatataaccagttatcatattcaagtgctctgttatcctctctgtgctgggatttttttttttttaaaggaaattgctgaagagaatattctcatggtagtgaatttcttcagctatagattttcatgagattctgatcaatatactatgttattcgtt

SEQ ID NO: 41 - DNA, 1686 bp, Cannabis sativa
  source 1..1686: mol_type = other DNA
atgatgttggatcttaatgttaatatcaacaacggtgccgattcaacctacgagaagactaaggaaagaggagcagagttgatgataatggaggtagaagagaaaggttccacaacccaaataatggaagattctggttcctctggttcttcggttgttaatgttgaaatcgatgctctgagtagtactacaacaattactacctctaatagcgtctttagagaagaagactcttccatcaatgtcacgaacacaacatcttcgacatttttctttgacataatgaagagagaaaaggactgtaacaacggtgccactgccgggaaagaaactaataacatctctccgccgggatttctaaccaggtcttttttcccggtggccggagaaaaggtagggaatcagttcgttgaagccggttctgggtcgtcttcctcgcgacctcagtggttgaacttatcttttgcggattccggtggaggaggcgccgccgtccaaccgccggcggatgttaaagttttgcagcagaaacaacagataaagaaaagccggcgggaccaaggtcccggagctctcagtaccgtggcgttacgttttaccggagaaccggccggtgggaatcccatatttgaggattttgttgatttgtttgtttttttttttgtttattcatttatttatttatttattttaggtggattcgacacagctcatgctgctgccagagcttacgatcgagctgcaatcaagtttcgtggagttgatgctgatatcaatttcaatgtaaccgattacgatgaagatatgatgcagatgaaaaacctgagcaaagaagaatttgttcaaatacttcgacgacaaagcactggattttctcgtgggagctcaaaatatagaggagtaactttgcataaatgtggtcgatgggaagctcgaatgggacaattccttggaaaaaagtatatttatcttggattattcgacagcgaactagaggctgcaagggcttatgacaaagcagccatcaaatgtaatggaagggatgcaattaccaactttgagcatagtacttatcagggtgagataattttagacaccaatgcacaaggcaatgaccacaaccttgatctgaacttgggaatttctccaccatgtgatggtccaaaagggcgtgagtactcttttggtcttggggacactcgtgttcattggaaccctcgtgaaggacctcacagaataagaccgataatgattgatgggcagccatccccacatattctacctcccaaccatgctgcttcagctacatgttcaggagtgtatccagccttcttaccaaaacatgaggagatgagagcaatggcagatgatcatcaaaagaggatcgaagccgccaattcttcgcagggttttttgaactgggcatggaaaattcatggcaatggtagcaacactactactactactactacatctggttgtgtaacagccatgcctactttctccattgcagcatcatcaggattctcatcttccacatctttggctgctctatcagctaccaataatataaatccccaagccaattttgttcaaaataatatttgccttagtcctagtatgcccattaccactacaaattctgtcacaaacaattttcacaactcacaaattcatagaggctaa

SEQ ID NO: 42 - DNA, 3447 bp, Cannabis sativa
  source 1..3447: mol_type = other DNA
atgaattatcttcccatgctttctcatcagcagttgctatacctgttgacaatgtcatttatgtcaagagtgccgagatccttgcttcctggtgtgcggagttctcgtctgaaggatcgcctgaaagaaggaagagagtttttagtaaagaaggcatttattgaagacatattgaatgaaaacaataccttatctattgttcttcaaaaagattcaagatatcgtgctgtattatggaatcctgatatactaacatatccaagcaaagagactcttgcacccactgcaggtgccattgttagtacaagttcaacagcaacagaaaacactcccctcgattatagaaaacatgacaatgagcaaaacaatcttcttgatgagatgaatttgtatatggagaatttgaatgatttatatttgggtagcgatgatttgtcatgtgatcttcaagttgactcaggggcattggcatgtgtagcttgtggtatccttggttttccatttatgtctgtggtacaaccatccgaaaaggcctcactagaacttcttcatgataatcatcttctgtttcaagaatgccctggaggatatttgaactgtttttcctctctggacctcgatgtctctaataatgcttctattacagagaatgtttctacggtttccaatcctccttcaatggatcttccagtaccttcagttaacaagtttgatggtggtcaaaatgcttacaataaatttctgagaccacgtagcttctgcctggaacatgctgttgaaattatagacattttacaggactatcagaagataaaggcacatgctgctgccattgcagaggaaattggtagcccttttaattataaagaggttcctttagatattgcatcgaaagatgatctgaacttgatagatattgctattgatgacgaacacgatgaatgtggtaaagactggacatcaaaattggggatcaacttgcagcactgtatcaaggtcagaagaaactcctcctctaagcaagttcgacatgctcttacattgggtgggttgttctctgataaaccgtcctctcaagatttctcgcctctcaagtggcagttgagaagatctcgttcaaaaaaaatttatgttaaaccatttgatagtgctcaagtgaagaaagtagtgggtgaaacatcttccaccattgagaaggaaaaaaaactgattcagtattcaagaaggaagttcaatagaaaaccaggtggtttaacaggagcaggtaaattttgtggacaacatgctaaaggggagatcttgatgaacttgccaaaagagcatctgaaactgacatctgaagcaacttggaacaatagcatgaaagctagctgtggaagtgttgagaatcaagttttgaaggaaatgaatttggatagtcaagcttgtcttgtagcaagttctgatgcttctaagatgcaagatgagagcagtgttccaacccaaaaagtcagcataaccgataagcttgttgaaaaactagaggcttgtccattttcccatgatactaaggaaagctccggacttctaggagatcagcttaaagtaggacaatctaacaaaactgatggggctaatgttgattctgaagggcactccaatgtcaaggctggcaaggagggtgttttgactgatttgaattctgatctttctaacatgacaaactcacatgctattgaatcccaggatgacaacttgatgggacaaataaaaaatgctgtagtggagaatgtttccatgactgttgaagtttgtgattctgtttctgtggataaagaagtgacgctagtagtgcatgctaccagaattgatgacggggaaccagtttcttgcaactctacagtcataatagataatattggtacttcaaaggaagaatgctttaattgttcaggagagatctgttctgtagaataccccagcagcaatggtaaagctcttaaagttgaatcagaagaagaaattaaaactagaataaacaaataccctcttgcctcagttgaagaaacttctaaagctcggagcgagactcattctacaaaggattttagtgagaccattcctgtcttaattgaagaaacttctgaatttttaagtgatccttcttctgcagctaaattatgctgtgctgtggcttcggaacagattctaaagcaaaaagttgggacttcagatgggagtagtgaggaacttgcttctagttctgtgacacaaatggaagtccaccaccgtcattgtgtttcaatagaggaacattctgatgttctcagggaaagctcagtggaagacgacttgggaacggatgtgacttcagataatggatttcataaggaagcctcgatgaaaaatggagagcagagatcagtttctcctgttacacaaacagttgatcagcctacacccataagtgttggagaatctgaaactccatggaatccttccaaagaacatcccggtgatggcttgactttagatgataaaaagaggaaaagtgagaccactgctttgccaaatagcatttccttccaaaaatgttcaagggttagaagagagactcatactaatgaagacttgtgcaacggtagtgaagtttgttcgtcgcaggatgatagagcattggatagcgagtctaaagatatagacacttgttcaaacccagtaaaaggaagaaagaggaagaaagagatagaactactaacagaaaaccagttcagttgcaatggctttacgaggagtccatgtgaagggttaagaccaaggactttgaaggatgcaacaagttgtgaaactgatacccttagagaagtcgagagaaagcctgcagtgaaggtaaagaaaacttcaaatgttagaggtcctcctaaggataagaaagaaaattcaaggaagcctcacaaatgtgacttagaaagctgtcgcatgagttttgacactaaggcagagctaatgttgcacaaacgtaaccgctgcccacatgaagggtgtggaaagaggtttagctctcataagtatgcgatgattcaccaccgtgttcatgatgaccagaggcccctcaagtgcccctgggaaggttgttcaatgtctttcaagtgggcatgggcaaggactgagcatatacgcgtgcacaccggagagcgaccataccagtgcaagattgatggatgtggcctctctttcagtcgaggcttgaaaggaagtctgagagttgtggctcctgaattcttggctggatgtctagatgtagataatgatgctcctagtttctattcaagtggtggcagagctgtgcagtgggcatggatggcagagaagcacgatcgaaggaaatggtgtagccaggttagggccatcagagggaatgcggcttgccaccagaactggtggagttcctcgatagagctgggttga

SEQ ID NO: 43 - DNA, 528 bp, Cannabis sativa
  source 1..528: mol_type = other DNA
atggcaaagaatctgaatttggataaaactgaatccgaatccgacctatccgaactaaccggattcattcgggcataccggtacccgatatgctccgagttcagtaaggaacaccctgcatggggcatggactctcacacagacagctcagtcttatccatactgagttgtgaagaccaagttggtggacttcaagtcctcaaaaacgacaattggttaacggtcgaacctatctccaacactctaatagtcaaccttggagacatgatgcaggcgattagtaacgatgagtacaagagtgtgaagcatagagtgaaagtgaacagatataaagagagaatttctatatgttacttcgtttttccaaaagaagggagtgttatacgaagcatcaactataagccttttacttatactgaatttcagaagcaagttcagcaagatgttaaggcccttggattcaaagttggcttagaccgtttcaagcatactactactgctgctgctgctattactgaggcgtgttaa

SEQ ID NO: 44 - DNA, 879 bp, Cannabis sativa
  source 1..879: mol_type = other DNA
atggcatctgtgaatccatttttcgaagatatgcgaacaaaatccgaggttattgatcctcctcaaagtgaagatatgcttgatgttggtgaaattctgaatgattctccacaaaatataggaaagcctaatgtagctgttacaagcagtgtccgcgatcttttggagtgccctgtttgcttaaatgcaatgtaccctccaattcatcagtgttcaaatggtcacacattgtgttctggttgcaagccaagagttcataaccggtgtcccacctgcaggcatgaactcgggaacattcgatgtcttgctttagagaaagttgctgcatctcttgagcttccatgtaaatatcagagttatggttgcattggtatatacccttattacagcaagttgaagcatgagtctcaatgcatttacagaccatacaattgcccatatgctggatcagaatgcactattgttggtgatatcccttctcttgtagcccatttaaaagatgatcacaaagttgatatgcataatggctgcacttttaatcatcgctatgtcaaatcaaacccgcaggaggttgaaaatgcaacctggatgttaacggttttcagctgttttggccagtatttttgtctgcattttgaagctttccagctcggtacagctcccgtctacatagctttccttcggtttatgggtgatgatgttgaggcaaagagttataactacagtcttgaagtggtggacatgggaggaagatggtatggcaggggattccaagaagcattagggacagtcaccgaaaagttcgtgacagttttgatggtctcattatccaacgaaatatggctctctccttctctgggggagaccggaaagagttga

SEQ ID NO: 45 - DNA, 324 bp, Cannabis sativa
  source 1..324: mol_type = other DNA
atgcacatcttcagctttctctctcctattccagatcggtataacacagccctggtttgcgacagatggcggtacttggcatgtcatcctcgactatggcttcgagttgatcgttctcttaaagaatccgaagctggcgttttcccaaacattgactcagctgtagctgctgcaaggcctggtgacactattctaattgcagctggtggtatgcatctggcttcaaatattcaaattaaaaaaccactttgcctgattggtggaggtgagcttcctgatgaaactaccctcatatgttctcgaggttcagacaggtttgattaa

SEQ ID NO: 46 - DNA, 1806 bp, Cannabis sativa
  source 1..1806: mol_type = other DNA
atgtttgattgggacgaggaggagctagcaaatataatatgggatgaggctgctgagaatggtgaccatattgttccttatcctgccaaaagtgaagattgtcgcaataagaaggaatggaaacaagaagccaatataaataagcctactgagcagaacaattctaaagttgatttacgtaaagaacaggtggaaagcagtaccaatttgatcaaccatgataaaagttctgattcaagaatgggggtggagtcctggcctgatttgcctttatctaatagtgaaagggaatctatgggtaccaacgtatctaacgccttaagtgagattacgaaatttgactcgtcaacagggactgatcagcttgataaagatgctgaaatatatccaagttcccacgaaggcaaagaacaaggtgattttgactatggctgggctaacattggaagttttgatgattttgagcgtatattcagcaacgatgatgctgtatttggtcaaactagtctgggtaatgctgatgagctttggtgttcctccaaagatattactaacagcccagacaaatccttccccatttcttctgagttgctcagtctgagcccaaaaacagtaagtaatacatctcagcagttggaaataaagacaaaagttgtgcaagaagatgacctgtgtagcctccgctatggggaaacagatgattccacatctcacagtttgcacagtgttaatgcaattctggagcatgcagaagatgcagtaggaaaaagtgagcctcttgccaaggagaagataaatttggacagagtggtgaagacaaccttatcaacatctaatccagctattgaaaatgcttctacccaaaataaaaatgctgagaaggttttctctccaactgtgactagacagaagaagctatttaaatctcgtaaaaagtcggatgtcaagagtgagggtaaacagttacaagaattatatggtgcctggccttcaccaagaaaccattctggtcaactagaaaatcgtatacaacaccctgcaattcaaccatctccctctacagttcagcagatgcagcttcaatgtcctgaaatttttcatttccagcatattccaaatcaatttgtggtgcatcctatgtatggaaacctggcaaatacatttcctgccatgcctgtgctatcgcgtattcagcctgggaagtataagcagcagcctttgctttctagccgtgatgtttcaccagctaacagcaatcttgtaaacaattcggcagaagctcctgtgaaaactacaatcatgacgcctcaggaaaaaatcgagaagttaaggcgacgacaacaatttcaggcgatgcttgctattcagaaacagcagcaacaacttagccaacaagtctcttcttccaaccattccaccactcaaacttgtcctcaacaatttgggggaagtgatgttcgacttgaagatttgagcactttctcttctttggaacctatctctcctatggaagaagatgattcaagttctacatctgcagctatcgatgattactctgtcaatgaatctatacttcacagacttcaagatgtgatatcaaagttggacatgacagtgagaatctgtatacgagatagcttgttccgtctggctcaaagtgcaagagaaaggcagaatctcagtagtacaaacaaaggtagcaaggacgaacgtgaaacacttgcagtagatgtaaccaatacctctaacaggatacaagatttccccaaatataggaacaatacccagaagtggatctccggttag

SEQ ID NO: 47 - DNA, 627 bp, Cannabis sativa
  source 1..627: mol_type = other DNA
atgggtttggctactaacttagaattggatcactttcatgtgttggctgtggatgatagcatcatagatagaaaattgattgaaactctcctcaagacttcttcctatcaagttactgcagtagattctggaactaaagcactagaatttcttgattactttatgcctggaatgactggctatgatctcctcaagaaagttaaggaatcaaaatctcttaaagatataccagttgttatcatgtcatctgagaatgaaccatccagaatcaacaggtgtttggaagatggagctgaggaattcttcttgaaaccagttcagttatcagatgtgaagaaactaagatctcatttggaggctaaagatatccagtcacgttcttcaaagcctagaatacccttagcttctgaggacattcctgagcgtattaaaaggaaaacagcagcttcctacagagaactagaagcaagaaagaaaagggtcaatgagttggagaaaatctacatggatatgtcaatgcagcaggaattgcagaaaaagggtcggaagcgcaaactacgcgaggatgagattgtcacgccaacatccaaaccggttttcaaatggagggcagagcggaagcgttga

SEQ ID NO: 48 - DNA, 963 bp, Cannabis sativa
  source 1..963: mol_type = other DNA
atggctttggaaggagaagttaccactttggagctcataaggcaacatcttcttaatgattttggttcaatggacagttttatcaacagccttaacaactgtacggccaatattaatacccaaaaccaaactaatcatattgatcatgattattctttctcatcgcaaaataaccaatcattcaaagttccaaagaaagaaacaactaataacgatgactattgtgaggaggcaaatcacagttttgagtttcaaaccaaactaaacacatcagttaatacccgcggccactactctaaaccctccagtttaagccagcgccgcccttctattaacgtagccataccaccggcaaattctccggttccggtgaaggttccggccatgtcatcgtcggtttctgacaacgctcctgacagcagacactaccggggtgtaaggaagaggccgtgggggaagtacgccgccgagattcgtgaccccaatcgccgcggctcgcgcgtgtggctaggaacctttgaaaccgccatagaagctgccagagcttacgaccgcgctgccttcaggatgagaggtagcaaagccatactgaattttcctctagaggccggcaggtcgccgatgccggagagcactgaggttcacgcgccggccaattgtactgttttgaaaaggaggagaggaagtgatgaggataaggaggttattagagaagcgaaggagttgaagaaggaagaaacgacgtcgtcgacattaacggagagtactacatcgtcaaatgatcatcaggaggtgaactcatcagggagtagtaacggtaacagtgcagttgtaccgttaacgccgtcaaattggacgtcggtatgggacagtcaagacgttaaggggatttttactgtgcctccgttatcacccttatctcctcacttttctattggatattctcaacttatggttatctga

SEQ ID NO: 49 - DNA, 1515 bp, Cannabis sativa
  source 1..1515: mol_type = other DNA
atgaacaaaacccaattaaaccgaggtcatcatcatcatcgtccatggccaggaggtttcccaacctccaaggccttaggaacctcctttggggacgccaattgcatggagcaacttctagttcattgcgccaacgccattgaaaccaacgacgtcactttggcccaacagatcttgtgggtcctcaacaacatagctcaaccagacggagattcaaaccagagactcacctcagctttcctccgagccctaatcacacgcgccgcacgcatcggcaccagctgcatcaagatgctcgcctcagccgttgcagttcaaacccaaaacgacgataatctcaacgtcgtttttgacgttccacacagattctcagtcattgacttcgctaccttcatcgacttgacaccctggcaccgcttcggcttcacagccgctaacgcagccatcctagaagccgtcgagggcttttcggtaattcacttggtcgatttgagctcaactcactgcatgcagatcccaaccttactcgacgccattgcctcccgacactacgaggtgacacctgtaatcaaactgacggtagtggtggcagcagaagcaaacgacgtcgttccaccaatccttgacatctcatacgacgaactcggtttaaagttggtcaacttcgctcggtcaagaaacataattctagaatttcggttagttcattcaacttactcagacggtttcgcttcactaatcgaacatctccgactccaacaacaacagtacttagttaataataatcataattacgaaataacccccgaagctctcgttattaactgtcacatgatgcttcattacatcccagaagaaaccctcataaacagtaataataataataatgataatgatattaactgttcatcatcgtcatcctctctgagatcaatgtttttgaaatcgattcggagtttggatccaaacgtggtcgttttagtggatgaagacgcagacttaacgtctaataacttggtgtgtagattaaggtctgcgtttaactttctttggataccttatgacacggtggagacttttcttcctaaggggagtaaacaaaggcagtggtacgaagctgatattggttggaagattgagaacgtaattgctaatgaaggagttcagagagtggagaggcttgagccgaagagtaggtgggttcagaggatgagaaatgcgagttttcggagcgttggattcggagaagaagctgtttcggaggttaagactatgcttgatgaacatgctgctggttggggacttaagaaggaagatgaagatcatcttgttcttacatggaaaggacataatgttgtttttgctactctttggatgcctgcttctgttgcactaactactactaattaattaatgcttcttacttaattattaattagatacttaataattaatttaagtagcatattattattattatctaattttatatattataattatctgattggttga

SEQ ID NO: 50 - DNA, 420 bp, Cannabis sativa
  source 1..420: mol_type = other DNA
atgaacacgtttgattccttctctgccgtagaatcgagcatggaagacgaatcgaaccgccggaatcggagtaggatgcgggcccgaagccgatccaaacccgccccagcctccattgccgtctccgatggagaagaggaagatggagatcctgaattgtggaagaacttcgaccagaatttcggacaggtacagacagttctggatcgaaacagagcgttgattcaacaggtgaacgagaatcaccagtctcgaattccagacaacatggtgaagaacgtggctctaatccaggagatcaacggcaacatttcgaaggtccgttcgctctactccgacctcggatccaacttcacaacctacttccacaacaacaaatcgcagaatggcaagcccgataaccttcgtcatcatcattga

SEQ ID NO: 51 - DNA, 1620 bp, Cannabis sativa
  source 1..1620: mol_type = other DNA
atggcgaccgaattggttgtcaacacggagagagtccagaaattcttcaacgacctggaggagcaaaagaccatcctttctacctgcactcagctattcacaaccatatccacccacttcaattctctccaacattccctttcccaaaagtaccaatccctcgaatcaagaatccaagctcttgaatcccattcccgcgacaccctccagtccctcgacacccgcgaggcttccattcccgaacgagaatccgcctccctcgcccgcatccaggagcagaaggaagctgctttgtccgagttcgaaaagcaaattcccgaaaacttggaactttcagaggcccttaaatcattttcccggaaaatggattcggcgggattggtgaaattcatcgtttccaagaggaaggagtccgtatctttgagggcagagatcgttcagtccttggcggaggctgtggacccgcctagactggttcttgattcattggaggattttctgaattccaagtcggccaaagctggggttactgataagcgatgggcttgtggattgcttgttcaggctttgtttcctgagagccattcggagaagataggacctcaatttgcgagaactgtcgtggagagagcagtcatgattttggagcagtggaaggggcagatggatggtggtgaatcagaaggcttggctttaggggctgccgaagctgttatgtttgtgcagatggtagttgggttcgggttgaatgcgaagttcgataaggctttcttgagaaagctaatgctgcagcatgcttcgaggagggatatggcaaagctcgcagctgttttagaagataaagactcaatgcaagagattattgatgagttggtgaagaataacaaagaagtggaagctgtctattttgcttgtgaatctgggttggctgaaagattctctcctgttactttgctcaagtctcatctcaagaacttcaaaaagaatgttgccaacatattaaagaatggaaacaatagcttggttgcaatggaggaatctagtaatgtggagttaaattccatcagagcaataatcaaatgtgtagaagaccacaaattagagtcagaattttctgttgaaagcttaagaaagcgggctactcatctggagaaagccaaggcagagagaaagaagagttcaggagcctccagtaaacctcaaaataagcgaggatacagtgccagcagcggccgaggtggtggtggtggatcatcttcttttcgccaatccaaagctgccaagtattccaacacttattcctcttttagcaggaggaatgcgaatcctcctccgccggttcatcacagcactgctgcaagatattctggagcatacaactaccctgttcaagctgtgtacgacacaccagctccagccccagccccatatccgtcctcatacggggttccccatgctcaaagccctgtcgcaattcctcaacactatctctcggccgacaacttgagtagtactgcctttcggagcagtggctcttacagtggtctgaccaactacagaccatatgattatgccgaagcagctccacatacttaccaaccgtcctcatatccaaaatag

SEQ ID NO: 52 - DNA, 1728 bp, Cannabis sativa
  source 1..1728: mol_type = other DNA
atggagatagcaaaggtgacgagcaagactaataagaaggtggattcaagaaaagcttcttctagtcctagcaatcagccttctagaaagcaaaacaggaagggagaaaatccagtgcgactccctccatctcttgaacaatcttccgaatttggattgtcaaactcgtggatatgtaaaaattctgcatgtagagctgtcctgtcaatagatgacacattttgcaggcggtgctcttgctgtatctgccacttgtttgatgacaacaaggaccctagtctttggctggtttgctcatcggaatctggggagggagactcttgtggattgtcttgccacattgagtgtgcccttcaacgtgaaaaggttggggtggttgatcttgggcagctgatgcagctagatggaagttattgttgtgcttcttgtggtaaagtgtctgggatacttgggtttcgagaggtgcatgatatagttaaagaagccaaggctaaattggaaacagaagttggcccggtgaatggtgtttcttctaaaatggcacgtggaatcgtcagcagactctctattgctggtgacgtgcagaagctctgctcccttgcaattgggaaagcagatgaatggctggccaatgtttctagtctcaatccaaatagcagagaggattcacttccttctgcttgcaagtttctttttgaagaagtaacaccatcatctgttgtgattattttaattgagctgtctaaagcatcttcgaatgagattaagggctacaagctttggtattacaataaaactggtgatttgggtcactctgaggcaaagtgtttcaccaagagcattgagatacttcacaagaatcctaatttacaagctgtgaaaatcgataagaaagagaatacctccactgaggaaagtcctagtgggaagggggaatcgaaaaatgcagtggcagctggttctgaattcaaggttcgagacctgggaaagatactacatctagcatgggctcaagagaaaggctacctagaagggttttgcagtgcggatgtagaaaagtgttgtgcagctgacaaagtagtaaaaacagaaatcatacaagaacgggtgccttctgtctcacgtgggcttgacttgaatgttgtttctgtccctgatctaaatgaagaactaactccaccatttgagtcttccagagatgaagacaatggctgcagcttgcagctaactgttgaagcagatgacgctgcttctcatgacatattgaagaacggtttagcaagatcgcatggtagtggtgactcccagacctggactcatggagtgacaggagatgtgccagccgttgattcaaggaccgatttttgtcggaaaagggcattggtgaacacaattgaggaggcacatgagtgtgacagtacactgattaatggttctccattccgaatatccaatggatctagttccttggatgaaaactttgagtactgtgtgaaaataatccggtggcttgaatgtgaaagtcatataacacaggaatttaggttgaaattgctaacttggtttagccttagatcaactgagcaggagcggagagttgtgaatacgttcattcaaaccatgattgatgatccaagtagcttggctggccagctggtagactctttctcagatattatctccagcaagaggtcaaagaacggtttctgtagtaagctgtggcattaa

SEQ ID NO: 53 - DNA, 1863 bp, Cannabis sativa
  source 1..1863: mol_type = other DNA
atgggaatttttgaagagatgggattctgtggtaattttgactttatttcatgcccttctcgggatggagaggaggttcaagagtgcgaaccagaggcaactgtggaagaggattatagcgatgaagagatggatgtggatgagcttgagaggaggatgtggcgggacaggatgttattgaggcggctgaaagagcaaagcaaggggaagcaaggagttgacaatgcaaagcagcgtcaatcccaggagcaagcgcggaggaagaagatgtctagagcacaagatgggatcttgaagtacatgctaaaaatgatggaggtttgtaaagctcaaggttttgtgtatggtattattcctgagaagggaaagcctgtgactggcgcttctgacaatctccgtgcttggtggaaagaaaaggttagatttgatcggaatggtccagctgcaattgctaggtaccaggcagaacactccatccctggtcagaatgaggactgcagtaccatggcatcgactcctcataccctgcaggagcttcaagacactactcttggctcgctcttgtctgctttgatgcagcactgtgatccaccccaaaggcgtttcccattggaaaagggtattgccccaccatggtggcctactggtagtgaggagtggtggcctcaattgggtatgcctaaggatcaggggcctccaccttataagaaacctcatgacttaaaaaaggcttggaaggttagtgtcctcacagctgtgatcaagcacatgtctcctgacattgcaaagattcgcaagcttgttcgtcaatccaaatgcttgcaggacaaaatgactgctaaggagagtgcaacttggttggccattataaaccaggaggaagctttggcccgaaagctgtatcctgataaatgtccacctatgtcagctactgggaatgggtctcttgtaatcagtgagaccagtgattatgatgttgatggtgtggatgatgaacctaattttgaagtggaggaatgcaagccccgtgatattggtcgcttcaacatcgggactattgttccttcaagggataggattcttatgcctcaagtggcttctcgaattaagggagaaatcattgaaaccaaccctgattttgttcaaaagaggaagcaagtgggtgatgagccacagatgatgatggatcagaagatattcacctgtgagtactctcagtgtccatatcatgattaccgtcttggctttctcgataggacctccagaaacaatcaccagatgaattgtgcataccgtcccaattcttctcaagcttctcagcaaacttttggaatgccaagctttcagattaacaacgataaaccagctgtcttctccttaccattttcccaacctaagccaccattggctcctcaacaaataaaccaatcatctcctttcactggccttgatattccggaagatgggcagaagatgatctctgaccttatgtcactttatgacacaaatgcacagcagaggaacaaaggtttgaatcctgggaaccattctgttcccaccaacatggagaaccatactcaaccacaacaaaaataccaatttcagatggatgatagcttcttcagccagggtgttatgggaggaaacatgtctgagcaagcaaataatatctcatcacaacaagtggttttcccatccgcagaggttcagtttgatcagtgcaaggcgttcgattctccattcgacaataatcccaatgataatattgcagacttaagattttcttctccatttaacatggcaccagttgaatttgctgtggactcactgccaaagcaggatgtctccctctggtacctctga

SEQ ID NO: 54 - protein, 561 aa, Cannabis sativa
MMLDLNVNINNGADSTYEKTKERGAELMIMEVEEKGSTTQIMEDSGSSGSSVVNVEIDALSSTTTITTSNSVFREEDSSINVTNTTSSTFFFDIMKREKDCNNGATAGKETNNISPPGFLTRSFFPVAGEKVGNQFVEAGSGSSSSRPQWLNLSFADSGGGGAAVQPPADVKVLQQKQQIKKSRRDQGPGALSTVALRFTGEPAGGNPIFEDFVDLFVFFFVYSFIYLFILGGFDTAHAAARAYDRAAIKFRGVDADINFNVTDYDEDMMQMKNLSKEEFVQILRRQSTGFSRGSSKYRGVTLHKCGRWEARMGQFLGKKYIYLGLFDSELEAARAYDKAAIKCNGRDAITNFEHSTYQGEIILDTNAQGNDHNLDLNLGISPPCDGPKGREYSFGLGDTRVHWNPREGPHRIRPIMIDGQPSPHILPPNHAASATCSGVYPAFLPKHEEMRAMADDHQKRIEAANSSQGFLNWAWKIHGNGSNTTTTTTTSGCVTAMPTFSIAASSGFSSSTSLAALSATNNINPQANFVQNNICLSPSMPITTTNSVTNNFHNSQIHRG

SEQ ID NO: 55 - protein, 1148 aa, Cannabis sativa
MNYLPMLSHQQLLYLLTMSFMSRVPRSLLPGVRSSRLKDRLKEGREFLVKKAFIEDILNENNTLSIVLQKDSRYRAVLWNPDILTYPSKETLAPTAGAIVSTSSTATENTPLDYRKHDNEQNNLLDEMNLYMENLNDLYLGSDDLSCDLQVDSGALACVACGILGFPFMSVVQPSEKASLELLHDNHLLFQECPGGYLNCFSSLDLDVSNNASITENVSTVSNPPSMDLPVPSVNKFDGGQNAYNKFLRPRSFCLEHAVEIIDILQDYQKIKAHAAAIAEEIGSPFNYKEVPLDIASKDDLNLIDIAIDDEHDECGKDWTSKLGINLQHCIKVRRNSSSKQVRHALTLGGLFSDKPSSQDFSPLKWQLRRSRSKKIYVKPFDSAQVKKVVGETSSTIEKEKKLIQYSRRKFNRKPGGLTGAGKFCGQHAKGEILMNLPKEHLKLTSEATWNNSMKASCGSVENQVLKEMNLDSQACLVASSDASKMQDESSVPTQKVSITDKLVEKLEACPFSHDTKESSGLLGDQLKVGQSNKTDGANVDSEGHSNVKAGKEGVLTDLNSDLSNMTNSHAIESQDDNLMGQIKNAVVENVSMTVEVCDSVSVDKEVTLVVHATRIDDGEPVSCNSTVIIDNIGTSKEECFNCSGEICSVEYPSSNGKALKVESEEEIKTRINKYPLASVEETSKARSETHSTKDFSETIPVLIEETSEFLSDPSSAAKLCCAVASEQILKQKVGTSDGSSEELASSSVTQMEVHHRHCVSIEEHSDVLRESSVEDDLGTDVTSDNGFHKEASMKNGEQRSVSPVTQTVDQPTPISVGESETPWNPSKEHPGDGLTLDDKKRKSETTALPNSISFQKCSRVRRETHTNEDLCNGSEVCSSQDDRALDSESKDIDTCSNPVKGRKRKKEIELLTENQFSCNGFTRSPCEGLRPRTLKDATSCETDTLREVERKPAVKVKKTSNVRGPPKDKKENSRKPHKCDLESCRMSFDTKAELMLHKRNRCPHEGCGKRFSSHKYAMIHHRVHDDQRPLKCPWEGCSMSFKWAWARTEHIRVHTGERPYQCKIDGCGLSFSRGLKGSLRVVAPEFLAGCLDVDNDAPSFYSSGGRAVQWAWMAEKHDRRKWCSQVRAIRGNAACHQNWWSSSIELG

SEQ ID NO: 56 - protein, 175 aa, Cannabis sativa
MAKNLNLDKTESESDLSELTGFIRAYRYPICSEFSKEHPAWGMDSHTDSSVLSILSCEDQVGGLQVLKNDNWLTVEPISNTLIVNLGDMMQAISNDEYKSVKHRVKVNRYKERISICYFVFPKEGSVIRSINYKPFTYTEFQKQVQQDVKALGFKVGLDRFKHTTTAAAAITEAC

SEQ ID NO: 57 - protein, 292 aa, Cannabis sativa
MASVNPFFEDMRTKSEVIDPPQSEDMLDVGEILNDSPQNIGKPNVAVTSSVRDLLECPVCLNAMYPPIHQCSNGHTLCSGCKPRVHNRCPTCRHELGNIRCLALEKVAASLELPCKYQSYGCIGIYPYYSKLKHESQCIYRPYNCPYAGSECTIVGDIPSLVAHLKDDHKVDMHNGCTFNHRYVKSNPQEVENATWMLTVFSCFGQYFCLHFEAFQLGTAPVYIAFLRFMGDDVEAKSYNYSLEVVDMGGRWYGRGFQEALGTVTEKFVTVLMVSLSNEIWLSPSLGETGKS

SEQ ID NO: 58 - protein, 107 aa, Cannabis sativa
MHIFSFLSPIPDRYNTALVCDRWRYLACHPRLWLRVDRSLKESEAGVFPNIDSAVAAARPGDTILIAAGGMHLASNIQIKKPLCLIGGGELPDETTLICSRGSDRFD

SEQ ID NO: 59 - protein, 601 aa, Cannabis sativa
MFDWDEEELANIIWDEAAENGDHIVPYPAKSEDCRNKKEWKQEANINKPTEQNNSKVDLRKEQVESSTNLINHDKSSDSRMGVESWPDLPLSNSERESMGTNVSNALSEITKFDSSTGTDQLDKDAEIYPSSHEGKEQGDFDYGWANIGSFDDFERIFSNDDAVFGQTSLGNADELWCSSKDITNSPDKSFPISSELLSLSPKTVSNTSQQLEIKTKVVQEDDLCSLRYGETDDSTSHSLHSVNAILEHAEDAVGKSEPLAKEKINLDRVVKTTLSTSNPAIENASTQNKNAEKVFSPTVTRQKKLFKSRKKSDVKSEGKQLQELYGAWPSPRNHSGQLENRIQHPAIQPSPSTVQQMQLQCPEIFHFQHIPNQFVVHPMYGNLANTFPAMPVLSRIQPGKYKQQPLLSSRDVSPANSNLVNNSAEAPVKTTIMTPQEKIEKLRRRQQFQAMLAIQKQQQQLSQQVSSSNHSTTQTCPQQFGGSDVRLEDLSTFSSLEPISPMEEDDSSSTSAAIDDYSVNESILHRLQDVISKLDMTVRICIRDSLFRLAQSARERQNLSSTNKGSKDERETLAVDVTNTSNRIQDFPKYRNNTQKWISG

SEQ ID NO: 60 - protein, 208 aa, Cannabis sativa
MGLATNLELDHFHVLAVDDSIIDRKLIETLLKTSSYQVTAVDSGTKALEFLDYFMPGMTGYDLLKKVKESKSLKDIPVVIMSSENEPSRINRCLEDGAEEFFLKPVQLSDVKKLRSHLEAKDIQSRSSKPRIPLASEDIPERIKRKTAASYRELEARKKRVNELEKIYMDMSMQQELQKKGRKRKLREDEIVTPTSKPVFKWRAERKR

SEQ ID NO: 61 - protein, 320 aa, Cannabis sativa
MALEGEVTTLELIRQHLLNDFGSMDSFINSLNNCTANINTQNQTNHIDHDYSFSSQNNQSFKVPKKETTNNDDYCEEANHSFEFQTKLNTSVNTRGHYSKPSSLSQRRPSINVAIPPANSPVPVKVPAMSSSVSDNAPDSRHYRGVRKRPWGKYAAEIRDPNRRGSRVWLGTFETAIEAARAYDRAAFRMRGSKAILNFPLEAGRSPMPESTEVHAPANCTVLKRRRGSDEDKEVIREAKELKKEETTSSTLTESTTSSNDHQEVNSSGSSNGNSAVVPLTPSNWTSVWDSQDVKGIFTVPPLSPLSPHFSIGYSQLMVI

SEQ ID NO: 62 - protein, 469 aa, Cannabis sativa
MNKTQLNRGHHHHRPWPGGFPTSKALGTSFGDANCMEQLLVHCANAIETNDVTLAQQILWVLNNIAQPDGDSNQRLTSAFLRALITRAARIGTSCIKMLASAVAVQTQNDDNLNVVFDVPHRFSVIDFATFIDLTPWHRFGFTAANAAILEAVEGFSVIHLVDLSSTHCMQIPTLLDAIASRHYEVTPVIKLTVVVAAEANDVVPPILDISYDELGLKLVNFARSRNIILEFRLVHSTYSDGFASLIEHLRLQQQQYLVNNNHNYEITPEALVINCHMMLHYIPEETLINSNNNNNDNDINCSSSSSSLRSMFLKSIRSLDPNVVVLVDEDADLTSNNLVCRLRSAFNFLWIPYDTVETFLPKGSKQRQWYEADIGWKIENVIANEGVQRVERLEPKSRWVQRMRNASFRSVGFGEEAVSEVKTMLDEHAAGWGLKKEDEDHLVLTWKGHNVVFATLWMPASVALTTTN

SEQ ID NO: 63 - protein, 5 aa, Cannabis sativa
LMLLT

SEQ ID NO: 64 - protein, 28 aa, Cannabis sativa
LLIRYLIINLSSILLLLSNFIYYNYLIG

SEQ ID NO: 65 - protein, 139 aa, Cannabis sativa
MNTFDSFSAVESSMEDESNRRNRSRMRARSRSKPAPASIAVSDGEEEDGDPELWKNFDQNFGQVQTVLDRNRALIQQVNENHQSRIPDNMVKNVALIQEINGNISKVRSLYSDLGSNFTTYFHNNKSQNGKPDNLRHHH

SEQ ID NO: 66 - protein, 539 aa, Cannabis sativa
MATELVVNTERVQKFFNDLEEQKTILSTCTQLFTTISTHFNSLQHSLSQKYQSLESRIQALESHSRDTLQSLDTREASIPERESASLARIQEQKEAALSEFEKQIPENLELSEALKSFSRKMDSAGLVKFIVSKRKESVSLRAEIVQSLAEAVDPPRLVLDSLEDFLNSKSAKAGVTDKRWACGLLVQALFPESHSEKIGPQFARTVVERAVMILEQWKGQMDGGESEGLALGAAEAVMFVQMVVGFGLNAKFDKAFLRKLMLQHASRRDMAKLAAVLEDKDSMQEIIDELVKNNKEVEAVYFACESGLAERFSPVTLLKSHLKNFKKNVANILKNGNNSLVAMEESSNVELNSIRAIIKCVEDHKLESEFSVESLRKRATHLEKAKAERKKSSGASSKPQNKRGYSASSGRGGGGGSSSFRQSKAAKYSNTYSSFSRRNANPPPPVHHSTAARYSGAYNYPVQAVYDTPAPAPAPYPSSYGVPHAQSPVAIPQHYLSADNLSSTAFRSSGSYSGLTNYRPYDYAEAAPHTYQPSSYPK

SEQ ID NO: 67 - protein, 575 aa, Cannabis sativa
MEIAKVTSKTNKKVDSRKASSSPSNQPSRKQNRKGENPVRLPPSLEQSSEFGLSNSWICKNSACRAVLSIDDTFCRRCSCCICHLFDDNKDPSLWLVCSSESGEGDSCGLSCHIECALQREKVGVVDLGQLMQLDGSYCCASCGKVSGILGFREVHDIVKEAKAKLETEVGPVNGVSSKMARGIVSRLSIAGDVQKLCSLAIGKADEWLANVSSLNPNSREDSLPSACKFLFEEVTPSSVVIILIELSKASSNEIKGYKLWYYNKTGDLGHSEAKCFTKSIEILHKNPNLQAVKIDKKENTSTEESPSGKGESKNAVAAGSEFKVRDLGKILHLAWAQEKGYLEGFCSADVEKCCAADKVVKTEIIQERVPSVSRGLDLNVVSVPDLNEELTPPFESSRDEDNGCSLQLTVEADDAASHDILKNGLARSHGSGDSQTWTHGVTGDVPAVDSRTDFCRKRALVNTIEEAHECDSTLINGSPFRISNGSSSLDENFEYCVKIIRWLECESHITQEFRLKLLTWFSLRSTEQERRVVNTFIQTMIDDPSSLAGQLVDSFSDIISSKRSKNGFCSKLWH

SEQ ID NO: 68 - protein, 620 aa, Cannabis sativa
MGIFEEMGFCGNFDFISCPSRDGEEVQECEPEATVEEDYSDEEMDVDELERRMWRDRMLLRRLKEQSKGKQGVDNAKQRQSQEQARRKKMSRAQDGILKYMLKMMEVCKAQGFVYGIIPEKGKPVTGASDNLRAWWKEKVRFDRNGPAAIARYQAEHSIPGQNEDCSTMASTPHTLQELQDTTLGSLLSALMQHCDPPQRRFPLEKGIAPPWWPTGSEEWWPQLGMPKDQGPPPYKKPHDLKKAWKVSVLTAVIKHMSPDIAKIRKLVRQSKCLQDKMTAKESATWLAIINQEEALARKLYPDKCPPMSATGNGSLVISETSDYDVDGVDDEPNFEVEECKPRDIGRFNIGTIVPSRDRILMPQVASRIKGEIIETNPDFVQKRKQVGDEPQMMMDQKIFTCEYSQCPYHDYRLGFLDRTSRNNHQMNCAYRPNSSQASQQTFGMPSFQINNDKPAVFSLPFSQPKPPLAPQQINQSSPFTGLDIPEDGQKMISDLMSLYDTNAQQRNKGLNPGNHSVPTNMENHTQPQQKYQFQMDDSFFSQGVMGGNMSEQANNISSQQVVFPSAEVQFDQCKAFDSPFDNNPNDNIADLRFSSPFNMAPVEFAVDSLPKQDVSLWYL

SEQ ID NO: 69 - DNA, 201 bp, Cannabis sativa
  misc_feature 101: note = "w" is a or t, wherein t is the predictive variant
  source 1..201: mol_type = genomic DNA
acatcccagacccagaataaaaaacaaacacaaaagaagagtaaagaaaaaaaaaacagagagctttatgagatcaatgtcttcacacacacaccaaaaawatacccataaactaaaacaaaaactcaaaccaaaaaaacgaaccgaccccaaaagatttcccatttacaatcatgttttcaagatgaatatatatatatt

SEQ ID NO: 70 - DNA, 201 bp, Cannabis sativa
  variation 101: note = "y" is t or c, wherein c is the predictive variant
  source 1..201: mol_type = genomic DNA
ttaatataattttccatctgtcctctctttctctcttccatatttatgtctgtgatccatttttcttcagacccaactgccgtttttctcttttcattttytcacacaactttacaggaaaacctctgttttttttttttttttttttcccttcttgggtttttttttttgatctgaactgggtttggattttctagagct

SEQ ID NO: 71 - DNA, 204 bp, Cannabis sativa
  variation 102: note = "n" is t or absent, wherein n is absent in the predictive variant
  variation 103: note = "n" is t or absent, wherein n is absent in the predictive variant
  variation 104: note = "n" is a or absent, wherein n is absent in the predictive variant
  source 1..204: mol_type = genomic DNA
tcccttcttgggtttttttttttgatctgaactgggtttggattttctagagctttttttctgaaaaataatccattcaggaagtgggaattttttattttnnntttttttttcccatttcaacctcttcaaaacaaatttaccatctgaaaattttgataaagagatcgagagagaaaatatataaagaaagaaactgaggaa

SEQ ID NO: 72 - DNA, 203 bp, Cannabis sativa
  variation 102: note = "n" is t or absent, wherein n is t in the predictive variant
  variation 103: note = "n" is a or absent, wherein n is a in the predictive variant
  source 1..203: mol_type = genomic DNA
cccttcttgggtttttttttttgatctgaactgggtttggattttctagagctttttttctgaaaaataatccattcaggaagtgggaattttttatttttnnttttttttcccatttcaacctcttcaaaacaaatttaccatctgaaaattttgataaagagatcgagagagaaaatatataaagaaagaaactgaggaat

SEQ ID NO: 73 - DNA, 201 bp, Cannabis sativa
  variation 101: note = "s" is g or c, wherein g is the predictive variant
  source 1..201: mol_type = genomic DNA
ggaagattctggttcctctggttcttcggttgttaatgttgaaatcgatgctctgagtagtactacaacaattactacctctaatagcgtctttagagaasaagactcttccatcaatgtcacgaacacaacatcttcgacatttttctttgacataatgaagagagaaaaggactgtaacaacggtgccactgccgggaa

SEQ ID NO: 74 - DNA, 201 bp, Cannabis sativa
  variation 101: note = "w" is a or t, wherein t is the predictive variant
  source 1..201: mol_type = genomic DNA
aggactgtaacaacggtgccactgccgggaaagaaactaataacatctctccgccgggatttctaaccaggtcttttttcccggtggccggagaaaaggtwgggaatcagttcgttgaagccggttctgggtcgtcttcctcgcgacctcagtggttgaacttatcttttgcggattccggtggaggaggcgccgccgtcc

SEQ ID NO: 75 - DNA, 201 bp, Cannabis sativa
  variation 101: note = "y" is t or c, wherein c is the predictive variant
  source 1..201: mol_type = genomic DNA
gagaaaaggtagggaatcagttcgttgaagccggttctgggtcgtcttcctcgcgacctcagtggttgaacttatcttttgcggattccggtggaggaggygccgccgtccaaccgccggcggatgttaaagttttgcagcagaaacaacagataaagaaaagccggcgggaccaaggtcccggagctctcagtaccgtgg

SEQ ID NO: 76 - DNA, 201 bp, Cannabis sativa
  variation 101: note = "y" is t or c, wherein c is the predictive variant
  source 1..201: mol_type = genomic DNA
gcaaaaatgcaggcaatgaccacaaccttgatctgaacttgggaatttctccaccatgtgatggtccaaaagggcgtgagtactcttttggtcttggggayactcgtgttcattggaaccctcgtgaaggacctcacagaataagaccgataatggtacattgattttaataataggcacagtttagaaagtgcagttttc

SEQ ID NO: 77 - DNA, 201 bp, Cannabis sativa
  variation 101: note = "m" is a or c, wherein a is the predictive variant
  source 1..201: mol_type = genomic DNA
catccccacatattctacctcccaaccatgctgcttcagctacatgttcaggagtgtatccagccttcttaccaaaacatgaggtactttctttcgcatcmtcacaaacttgccaaatacttaaagttgattatctgaaacacttttttgggagatacaggagatgagagcaatggcagatgatcatcaaaagaggatcga

SEQ ID NO: 78 - DNA, 201 bp, Cannabis sativa
  variation 101: note = "y" is t or c, wherein c is the predictive variant
  source 1..201: mol_type = genomic DNA
tctacctcccaaccatgctgcttcagctacatgttcaggagtgtatccagccttcttaccaaaacatgaggtactttctttcgcatcctcacaaacttgcyaaatacttaaagttgattatctgaaacacttttttgggagatacaggagatgagagcaatggcagatgatcatcaaaagaggatcgaagccgccaattct

SEQ ID NO: 79 - DNA, 201 bp, Cannabis sativa
  variation 101: note = "r" is g or a, wherein a is the predictive variant
  source 1..201: mol_type = genomic DNA
acctcccaaccatgctgcttcagctacatgttcaggagtgtatccagccttcttaccaaaacatgaggtactttctttcgcatcctcacaaacttgccaartacttaaagttgattatctgaaacacttttttgggagatacaggagatgagagcaatggcagatgatcatcaaaagaggatcgaagccgccaattcttcg

SEQ ID NO: 80 - DNA, 201 bp, Cannabis sativa
  variation 101: note = "m" is a or c, wherein a is the predictive variant
  source 1..201: mol_type = genomic DNA
accaaaacatgaggtactttctttcgcatcctcacaaacttgccaaatacttaaagttgattatctgaaacacttttttgggagatacaggagatgagagmaatggcagatgatcatcaaaagaggatcgaagccgccaattcttcgcagggttttttgaactgggcatggaaaattcatggcaatggtagcaacactact

SEQ ID NO: 81 - DNA, 201 bp, Cannabis sativa
  variation 101: note = "r" is g or a, wherein a is the predictive variant
  source 1..201: mol_type = genomic DNA
aaacatgaggtactttctttcgcatcctcacaaacttgccaaatacttaaagttgattatctgaaacacttttttgggagatacaggagatgagagcaatrgcagatgatcatcaaaagaggatcgaagccgccaattcttcgcagggttttttgaactgggcatggaaaattcatggcaatggtagcaacactactacta

SEQ ID NO: 82 - DNA, 201 bp, Cannabis sativa
  variation 101: note = "r" is g or a, wherein g is the predictive variant
  source 1..201: mol_type = genomic DNA
atacttaaagttgattatctgaaacacttttttgggagatacaggagatgagagcaatggcagatgatcatcaaaagaggatcgaagccgccaattcttcrcagggttttttgaactgggcatggaaaattcatggcaatggtagcaacactactactactactactacatctggttgtgtaacagccatgcctactttct

SEQ ID NO: 83 - DNA, 201 bp, Cannabis sativa
  variation 101: note = "r" is g or a, wherein g is the predictive variant
  source 1..201: mol_type = genomic DNA
aaacacttttttgggagatacaggagatgagagcaatggcagatgatcatcaaaagaggatcgaagccgccaattcttcgcagggttttttgaactgggcrtggaaaattcatggcaatggtagcaacactactactactactactacatctggttgtgtaacagccatgcctactttctccattgcagcatcatcaggat

SEQ ID NO: 84 - DNA, 201 bp, Cannabis sativa
  variation 101: note = "s" is g or c, wherein c is the predictive variant
  source 1..201: mol_type = genomic DNA
aacatttcgaaggtccgttcgctctactccgacctcggatccaacttcacaacctacttccacaacaacaaatcgcagaatggcaagcccgataaccttcstcatcatcattgatattcttcgtttaaaaagttttcaaaatcattgcttttagtatcttctttttaaataatcatgttgtcttttaataaagtgaggctt

SEQ ID NO: 85 - DNA, 201 bp, Cannabis sativa
  variation 101: note = "w" is a or t, wherein a is the predictive variant
  source 1..201: mol_type = genomic DNA
tcttcttcatcaccaaaaatagggtttagggtttttcatattcttccattcagtgacctttaaactccaccatttatcatcatcatcaatggcgaccgaawtggttgtcaacacggagagagtccagaaattcttcaacgacctggaggagcaaaagaccatcctttctacctgcactcagctattcacaaccatatccac

SEQ ID NO: 86 - DNA, 201 bp, Cannabis sativa
  variation 101: note = "y" is t or c, wherein c is the predictive variant
  source 1..201: mol_type = genomic DNA
gaaagattctctcctgttactttgctcaagtctcatctcaagaacttcaaaaagaatgttgccaacatattaaagaatggaaacaatagcttggttgcaayggtttgtctatattctgacatattttacaaatgtctttttgcatgtagtcacatgactgcactttttaagattttataatccaagtcgtatcatccacag

SEQ ID NO: 87 - DNA, 201 bp, Cannabis sativa
  variation 101: note = "y" is t or c, wherein t is the predictive variant
  source 1..201: mol_type = genomic DNA
agagaaagaagagttcaggagcctccagtaaacctcaaaataagcgaggatacagtgccagcagcggccgaggtggtggtggtggatcatcttcttttcgycaatccaaagctgccaagtattccaacacttattcctcttttagcaggaggaatgcgaatcctcctccgccggttcatcacagcactgctgcaagatatt

SEQ ID NO: 88 - DNA, 201 bp, Cannabis sativa
  variation 101: note = "r" is g or a, wherein a is the predictive variant
  source 1..201: mol_type = genomic DNA
taaattgctactggaaacctgtctattttctagtgcaatgaagtataatatggacaatgaggcaaggcaatgtaagtaggttaagagataaaagtttcaargcactggaaaaggaatggaaagtataatcatagttaggcagagaaagatttgaactgttatacggttcttttatgggatgggagtttattatgaatttca

SEQ ID NO: 89 - DNA, 201 bp, Cannabis sativa
  variation 102: note = "n" is g or a, wherein a is the predictive variant
  variation 103: note = "n" is g or a, wherein g is the predictive variant
  variation 107: note = "n" is g or a, wherein a is the predictive variant
  source 1..201: mol_type = genomic DNA
gctactggaaacctgtctattttctagtgcaatgaagtataatatggacaatgaggcaaggcaatgtaagtaggttaagagataaaagtttcaaggcactgnnaaangaatggaaagtataatcatagttaggcagagaaagatttgaactgttatacggttcttttatgggatgggagtttattatgaatttcaagtgga

SEQ ID NO: 90 - DNA, 201 bp, Cannabis sativa
  variation 101: note = "r" is g or a, wherein a is the predictive variant
  source 1..201: mol_type = genomic DNA
ttttctctcttctacctcaattttcaaaatcaatctctctctactcaattttcaaaatactcttatatattattacaacttaatggctttggaaggagaarttaccactttggagctcataaggcaacatcttcttaatgattttggttcaatggacagttttatcaacagccttaacaactgtacggccaatattaatac

SEQ ID NO: 91 - DNA, 201 bp, Cannabis sativa
  variation 101: note = "k" is g or t, wherein g is the predictive variant
  source 1..201: mol_type = genomic DNA
agctaggctagctattcacaatggctagaaaaagtcaatgggtagttagggttttatgtgtaaatatacgtatatgtatgtatgagtggtattaattatakgttcaaattaagtagtattttttttagacagcttgcttaagtatgcaaaatggctgatgtactatgatgtaagcaccactatttatatggttgagaattc

SEQ ID NO: 92 - DNA, 201 bp, Cannabis sativa
  variation 101: note = "b" is g or c, wherein c is the predictive variant
  source 1..201: mol_type = genomic DNA
aacatttcgaaggtccgttcgctctactccgacctcggatccaacttcacaacctacttccacaacaacaaatcgcagaatggcaagcccgataaccttcbtcatcatcattgatattcttcgtttaaaaagttttcaaaatcattgcttttagtatcttctttttaaataatcatgttgtcttttaataaagtgaggctt

SEQ ID NO: 93 - DNA, 202 bp, Cannabis sativa
  variation 102: note = "n" is t or absent, wherein n is t in the predicitve variant
  source 1..202: mol_type = genomic DNA
attgcttttagtatcttctttttaaataatcatgttgtcttttaataaagtgaggcttctgggtgtaagtccttgagctaattagagagagcccacatgganttatatatatatatatatatatatatattgtataagtatgaactgtgtttgttgtgatgtctgcttgtcccctgtttgtactaatatgtgtaacacttgt

SEQ ID NO: 94 - DNA, 202 bp, Cannabis sativa
  variation 102: note = "n" is a or absent, wherein n is a in the predictive variant
  source 1..202: mol_type = genomic DNA
tgcttttagtatcttctttttaaataatcatgttgtcttttaataaagtgaggcttctgggtgtaagtccttgagctaattagagagagcccacatggattntatatatatatatatatatatatattgtataagtatgaactgtgtttgttgtgatgtctgcttgtcccctgtttgtactaatatgtgtaacacttgttgt

SEQ ID NO: 95 - DNA, 203 bp, Cannabis sativa
  variation 102: note = "n" is t or absent, wherein n is t in the predictive variant
  variation 103: note = "n" is t or absent, wherein n is t in the predictive variant
  source 1..203: mol_type = genomic DNA
tatcttctttttaaataatcatgttgtcttttaataaagtgaggcttctgggtgtaagtccttgagctaattagagagagcccacatggattatatatatanntatatatatatatatattgtataagtatgaactgtgtttgttgtgatgtctgcttgtcccctgtttgtactaatatgtgtaacacttgttgtgttttaaa

SEQ ID NO: 96 - DNA, 202 bp, Cannabis sativa
  variation 102: note = "n" is t or absent, wherein n is t in the predictive variant
  source 1..202: mol_type = genomic DNA
atctaatgatgagatcgaacatttccctgcaccttcaagagtaactgtaaggaagagaccaactacttctgttatagaattgaaagaaccagaggtacggcncctacttgatgttttagctttaactggtagagtgggattcagttgacttgatatggttttttttttcttgttttaggtttattcaaattcaaggggaagt

SEQ ID NO: 97 - DNA, 206 bp, Cannabis sativa
  variation 102: note = "n" is t or absent, wherein n is t in the predictive variant
  variation 103: note = "n" is a or absent, wherein n is a in the predictive variant
  variation 104: note = "n" is a or absent, wherein n is a in the predictive variant
  variation 105: note = "n" is a or absent, wherein n is a in the predictive variant
  variation 106: note = "n" is t or absent, wherein n is t in the predictive variant
  source 1..206: mol_type = genomic DNA
tctaatgatgagatcgaacatttccctgcaccttcaagagtaactgtaaggaagagaccaactacttctgttatagaattgaaagaaccagaggtacggccnnnnnctacttgatgttttagctttaactggtagagtgggattcagttgacttgatatggttttttttttcttgttttaggtttattcaaattcaaggggaagtc

SEQ ID NO: 98 - DNA, 201 bp, Cannabis sativa
  variation 101: note = "y" is t or c, wherein t is the predictive variant
  source 1..201: mol_type = genomic DNA
aaaccaaataacataagtaggaaactcatccgattttctttttcacattactttgggacatagttattaatttttattaaggtgacacaactgatatggayatttaagaaatgattggacgaataaccagttcaagtgaacttataaaacttaaacctttctttttctttttttcattaaaagaaaaggcagatggttaga

SEQ ID NO: 99 - DNA, 201 bp, Cannabis sativa
  variation 101: note = "b" is g or c, wherein g is the predictive variant
  source 1..201: mol_type = genomic DNA
tagctttcttagtggtgattctgtagaaccggttctaaagcatgtatatcccttagattcattacaataattttagttgtttttcgtttgctttttttttbctctttttctgttttagtcctattttagtgtggcttgggtctcatttagacaaaatcatttaaattgtcatattggccatttttctagtttgattaaatg

SEQ ID NO: 100 - DNA, 201 bp, Cannabis sativa
  variation 101: note = "y" is t or c, wherein t is the predictive variant
  source 1..201: mol_type = genomic DNA
ctactgatgcagtaacgagagttcttcaaattctcaagggtaatgtattatcaaaaataatttataaagctttattttcattttaatttaatacccagttytctgacttactaaatctggaagaagataatgcaatgaatccgacacaattccatcattcactatccaatatatcatggatgattagaaaataagaaaaga

SEQ ID NO: 101 - DNA, 202 bp, Cannabis sativa
  variation 102: note = "n" is a or absent, wherein n is absent in the predictive variant
  source 1..202: mol_type = genomic DNA
ttcttgggttttttatttttgatctgaactgggtttggattttctagagcttttttttctgaaaaataatccattcaggaagtgggaattttttattttatntttttttttcccatttcaacctcttcaaaacaaattcaccatctgaaaattttgatagagatcgagagagaagatagataaagaaaatttttgatatcat

SEQ ID NO: 102 - DNA, 201 bp, Cannabis sativa
  variation 101: note = "w" is a or t, wherein a is the predictive variant
  source 1..201: mol_type = genomic DNA
tcttgggttttttatttttgatctgaactgggtttggattttctagagcttttttttctgaaaaataatccattcaggaagtgggaattttttattttatwttttttttcccatttcaacctcttcaaaacaaattcaccatctgaaaattttgatagagatcgagagagaagatagataaagaaaatttttgatatcatg

SEQ ID NO: 103 - DNA, 201 bp, Cannabis sativa
  variation 101: note = "b" is g or c, wherein g is the predictive variant
  source 1..201: mol_type = genomic DNA
ggaagattctggttcctctggctcttccgttgttaatgttgaaatcgatgctctgagtagtactacaactattactacctctaatggcgtctttagagaabaagactcttccatcaatgtcacgaacacaacatcttcgacatttttctttgacataatgaagagagaaaaggactgtaacaacggtgccactgccgggaa

SEQ ID NO: 104 - DNA, 201 bp, Cannabis sativa; identical to SEQ ID NO: 74
  variation 101: note = "w" is a or t, wherein t is the predictive variant
  source 1..201: mol_type = genomic DNA

SEQ ID NO: 105 - DNA, 201 bp, Cannabis sativa
  variation 101: note = "y" is t or c, wherein c is the predictive variant
  source 1..201: mol_type = genomic DNA
gagaaaaggtagggaatcagttcgttgaagccggttctgggtcgtcttcctcgcgacctcagtggttgaacttatcttttgcggattccggtggaggaggygccgccgtccaaccgccggcggatgttaaagttttgcagcagaaacaacagataaagaaaagccggcggggaccaaggtcccggagctctcagtaccgcg

SEQ ID NO: 106 - DNA, 202 bp, Cannabis sativa
  variation 101: note = "n" is t or absent, wherein n is absent in the predictive variant
  source 1..202: mol_type = genomic DNA
cttggattattcgacagcgaactagaggctgcaaggtcctcatttgttcatgaatatatttgaagaattcaatattgtcctctaatattgacaacattttcntttttaactcgatagaataaaacacattatgcctttggcttggcttttgtttggtggggatttaatccaacacaagttttttcttttatctttgtttaaa

SEQ ID NO: 107 - DNA, 201 bp, Cannabis sativa
  variation 101: note = "y" is t or c, wherein t is the predictive variant
  source 1..201: mol_type = genomic DNA
atattgacaacattttctttttaactcgatagaataaaacacattatgcctttggcttggcttttgtttggtggggatttaatccaacacaagttttttcytttatctttgtttaaattcagggcttatgacaaagcagccatcaaatgtaatggaagggatgcaattaccaactttgagcatagtacttatcagggtgag

SEQ ID NO: 108 - DNA, 201 bp, Cannabis sativa
  variation 101: note = "w" is a or t, wherein a is the predictive variant
  source 1..201: mol_type = genomic DNA
agtactcttttggtcttggggacactcgtgttcattggaaccctcgtgaaggacctcacagaataagaccaataatggtacattgattttaataataggcwcagtttagaaagtgcagttttctttcctttctgatataaaaactaaaattagtatccactactcccaacagattgatgggcagtcatccccacatattct

SEQ ID NO: 109 - DNA, 201 bp, Cannabis sativa
  variation 101: note = "y" is t or c, wherein t is the predictive variant
  source 1..201: mol_type = genomic DNA
ctcgtgttcattggaaccctcgtgaaggacctcacagaataagaccaataatggtacattgattttaataataggcacagtttagaaagtgcagttttctytcctttctgatataaaaactaaaattagtatccactactcccaacagattgatgggcagtcatccccacatattctacctcccaaccatgctgcttcagc

SEQ ID NO: 110 - DNA, 201 bp, Cannabis sativa
  variation 101: note = "w" is a or t, wherein t is the predictive variant
  source 1..201: mol_type = genomic DNA
gtacattgattttaataataggcacagtttagaaagtgcagttttctttcctttctgatataaaaactaaaattagtatccactactcccaacagattgawgggcagtcatccccacatattctacctcccaaccatgctgcttcagctacatgttcaggagtgtatccatccttcttaccaaaacatgaggtactttctt

SEQ ID NO: 111 - DNA, 201 bp, Cannabis sativa
  variation 101: note = "m" is a or c, wherein a is the predictive variant
  source 1..201: mol_type = genomic DNA
accaaaacatgaggtactttctttcgcatcatcacaaacttgccaaatacttaaagttgattatctgaaacacttttttgggagatacaggagatgagagmaatggcagatgatcatcaaaagaggatcgaagccgccaattcttcgcagggttttttgaactgggcatggaaaattcatggcaatggtagcaacactact

SEQ ID NO: 112 - DNA, 201 bp, Cannabis sativa
  variation 101: note = "r" is g or a, wherein a is the predictive variant
  source 1..201: mol_type = genomic DNA
aaacatgaggtactttctttcgcatcatcacaaacttgccaaatacttaaagttgattatctgaaacacttttttgggagatacaggagatgagagcaatrgcagatgatcatcaaaagaggatcgaagccgccaattcttcgcagggttttttgaactgggcatggaaaattcatggcaatggtagcaacactactacta

SEQ ID NO: 113 - DNA, 201 bp, Cannabis sativa
  variation 101: note = "r" is g or a, wherein g is the predictive variant
  source 1..201: mol_type = genomic DNA
atacttaaagttgattatctgaaacacttttttgggagatacaggagatgagagcaatggcagatgatcatcaaaagaggatcgaagccgccaattcttcrcagggttttttgaactgggcatggaaaattcatggcaatggtagcaacactactactactacatctagttgtgtaacagccatgcctactttctccattg

SEQ ID NO: 114 - DNA, 201 bp, Cannabis sativa
  variation 101: note = "r" is g or a, wherein g is the predictive variant
  source 1..201: mol_type = genomic DNA
aaacacttttttgggagatacaggagatgagagcaatggcagatgatcatcaaaagaggatcgaagccgccaattcttcgcagggttttttgaactgggcrtggaaaattcatggcaatggtagcaacactactactactacatctagttgtgtaacagccatgcctactttctccattgcagcatcatcaggattctcat

SEQ ID NO: 115 - DNA, 202 bp, Cannabis sativa
  variation 102: note = "n" is g or absent, wherein n is absent in the predictive variant
  source 1..202: mol_type = genomic DNA
ccaattcttcgcagggttttttgaactgggcatggaaaattcatggcaatggtagcaacactactactactacatctagttgtgtaacagccatgcctactnttctccattgcagcatcatcaggattctcatcttccacatctttggctgctctatcagctaccaataatataaatccccaagccaattttgttcaaaata

SEQ ID NO: 116 - DNA, 24 bp, synthetic construct
  misc_feature 1..24: note = UPF2 Primer 1
  source 1..24: mol_type = other DNA
gtacagtaaactatctcaatttct

SEQ ID NO: 117 - DNA, 24 bp, synthetic construct
  misc_feature 1..24: note = UPF2 Primer 2
  source 1..24: mol_type = other DNA
accacaccttttccaattggactc

SEQ ID NO: 118 - DNA, 26 bp, synthetic construct
  source 1..26: mol_type = other DNA
  misc_feature 1..26: note = qRT-PCR_FT(12434)_Forward Primer
caactgtggggattcatcgatatgtg

SEQ ID NO: 119 - DNA, 26 bp, synthetic construct
  source 1..26: mol_type = other DNA
  misc_feature 1..26: note = qRT-PCR_FT(12434)_Reverse Primer
cgagtagtagtagtcctggttctgcc

SEQ ID NO: 120 - DNA, 20 bp, synthetic construct
  source 1..20: mol_type = other DNA
  misc_feature 1..20: note = qRT-PCR_FT(7085)_Forward Primer
cactggcctggatcctcttg

SEQ ID NO: 121 - DNA, 21 bp, synthetic construct
  source 1..21: mol_type = other DNA
  misc_feature 1..21: note = qRT-PCR_FT(7085)_Reverse Primer
ccaccaatctcaaccctaggt

SEQ ID NO: 122 - DNA, 22 bp, synthetic construct
  source 1..22: mol_type = other DNA
  misc_feature 1..22: note = qRT-PCR_FT(4768)_Forward Primer
ccaagcttcggacaagaggtgg

SEQ ID NO: 123 - DNA, 26 bp, synthetic construct
  source 1..26: mol_type = other DNA
  misc_feature 1..26: note = qRT-PCR_FT(4768)_Reverse Primer
gtctttagtgttgaagttctgacgcc

SEQ ID NO: 124 - DNA, 24 bp, synthetic construct
  source 1..24: mol_type = other DNA
  misc_feature 1..24: note = qRT-PCR_PRR7_Forward Primer
gtggctacgaagttacagctgtag

SEQ ID NO: 125 - DNA, 24 bp, synthetic construct
  source 1..24: mol_type = other DNA
  misc_feature 1..24: note = qRT-PCR_PRR7_Reverse Primer
gctcatgatcttgcctagaagacc

SEQ ID NO: 126 - DNA, 28 bp, synthetic construct
  source 1..28: mol_type = other DNA
  misc_feature 1..28: note = qRT-PCR_TOE1_Forward Primer
gataatggaggtagaagagaaaggttcc

SEQ ID NO: 127 - DNA, 27 bp, synthetic construct
  source 1..27: mol_type = other DNA
  misc_feature 1..27: note = qRT-PCR_TOE1_Reverse Primer
gttcgtgacattgatggaagagtcttc

SEQ ID NO: 128 - DNA, 24 bp, synthetic construct
  source 1..24: mol_type = other DNA
  misc_feature 1..24: note = qRT-PCR_CUL3A_Forward Primer
gaggagctttacagaaatgcctac

SEQ ID NO: 129 - DNA, 22 bp, synthetic construct
  source 1..22: mol_type = other DNA
  misc_feature 1..22: note = qRT-PCR_CUL3A_Reverse Primer
caagaaacaagcctccctgagc

SEQ ID NO: 130 - DNA, 23 bp, synthetic construct
  source 1..23: mol_type = other DNA
  misc_feature 1..23: note = qRT-PCR_AGC_Forward Primer
gcagaggccaagttgaacatgtg

SEQ ID NO: 131 - DNA, 26 bp, synthetic construct
  source 1..26: mol_type = other DNA
  misc_feature 1..26: note = qRT-PCR_AGC_Reverse Primer
catcttcagtcaaagtgtccttccgc

SEQ ID NO: 132 - DNA, 23 bp, synthetic construct
  source 1..23: mol_type = other DNA
  misc_feature 1..23: note = qRT-PCR_PRR7_24hr_Forward Primer
cagcctcaaggacatttggttcg

SEQ ID NO: 133 - DNA, 24 bp, synthetic construct
  source 1..24: mol_type = other DNA
  misc_feature 1..24: note = qRT-PCR_PRR7_24hr_Reverse Primer
ctacagctgtaacttcgtagccac

SEQ ID NO: 134 - DNA, 22 bp, synthetic construct
  source 1..22: mol_type = other DNA
  misc_feature 1..22: note = qRT-PCR_PRR5_24hr_Forward Primer
ctttggtttgggcagttgctgc

SEQ ID NO: 135 - DNA, 30 bp, synthetic construct
  source 1..30: mol_type = other DNA
  misc_feature 1..30: note = qRT-PCR_PRR5_24hr_Reverse Primer
cgaatcttgtgaagacatcattataacagg

SEQ ID NO: 136 - DNA, 24 bp, synthetic construct
  source 1..24: mol_type = other DNA
  misc_feature 1..24: note = qRT-PCR_PRR9_24hr_Forward Primer
ctaagaacatgacagctgctcaac

SEQ ID NO: 137 - DNA, 26 bp, synthetic construct
  source 1..26: mol_type = other DNA
  misc_feature 1..26: note = qRT-PCR_PRR9_24hr_Reverse Primer
cattgtacaagagctttgggaatcac

SEQ ID NO: 138 - DNA, 28 bp, synthetic construct
  source 1..28: mol_type = other DNA
  misc_feature 1..28: note = PRR7-Start-Seq-Forward
atgaataataaagctcccgtaaccaatg

SEQ ID NO: 139 - DNA, 20 bp, synthetic construct
  source 1..20: mol_type = other DNA
  misc_feature 1..20: note = PRR7-EX3-Seq-Forward
atcgaaaggtgccgttgact

SEQ ID NO: 140 - DNA, 24 bp, synthetic construct
  source 1..24: mol_type = other DNA
  misc_feature 1..24: note = PRR7-EX5-Seq-Reverse
gcgtggtgttactgttgcattttc

SEQ ID NO: 141 - DNA, 27 bp, synthetic construct
  source 1..27: mol_type = other DNA
  misc_feature 1..27: note = PRR7-EX8-Seq-Reverse
ctgtgattactaatattgccttccatg

SEQ ID NO: 142 - DNA, 27 bp, synthetic construct
  source 1..27: mol_type = other DNA
  misc_feature 1..27: note = PRR7-End-STOP-Reverse
tcaaagcagcttctctttgtgcaaatc

SEQ ID NO: 143 - DNA, 23 bp, synthetic construct
  source 1..23: mol_type = other DNA
  misc_feature 1..23: note = pJET1.2 Forward
cgactcactatagggagagcggc

SEQ ID NO: 144 - DNA, 24 bp, synthetic construct
  source 1..24: mol_type = other DNA
  misc_feature 1..24: note = pJET1.2 Reverse
aagaacatcgattttccatggcag

SEQ ID NO: 145 - DNA, 1987 bp, Cannabis sativa
  source 1..1987: mol_type = other DNA
atgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattatgatgtcttctcatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtataacattgtttcaactgcaaaccaagctccaacagggaacataggaagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcctcccttcagactattattcaagatgcttcacagtgtggttcatccaatgcattgagagcacccatggaaggcaatattagtaatcacagtctcaacaggagtgggtcaggtagtaaccatggtagcaacggacaaaagagaagcaccaatgcttcaaactccagggggaaaaagacagaaagtgagagtgtggttactggaagagggaaaaccattgaaggaagtgaatcggatggaaatcgatttgcacaaagagaagctgctttga

SEQ ID NO: 146 - DNA, 1905 bp, Cannabis sativa
  source 1..1905: mol_type = other DNA
atgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgagtgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtataacattgtttcaactgcaaatcaagctccaacagggaacatagggagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcttcccttcagactattattcaagatgcttcacagtgtggttcatccaatgcattgagagcacccatggaaggcaatattagtaatcacagtctcaacaggagtgggtcaggtagtaaccatggtagcaacggacaaaagagaagcaccaatgcttcaaactccagagggaaaaagacagaaagtgagagtgtggttactggaagagggaaaaccattgaaggaagtgaatcggatggaaatcgatttgcacaaagagaagctgctttga

SEQ ID NO: 147 - DNA, 1991 bp, Cannabis sativa
  source 1..1991: mol_type = other DNA
atgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatccctgtaattattgatgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtataacattgtttcaactgcaaatcaagctccaacagggaacatagggagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcttcccttcagactattattcaagatgcttcacagtgtggttcatccaatgcattgagagcacccatggaaggcaatattagtaatcacagtctcaacaggagtgggtcaggtagtaaccatggtagcaacggacaaaagagaagcaccaatgcttcaaactccagagggaaaaagacagaaagtgagagtgtggttactggaagagggaaaaccattgaaggaagtgaatcggatggaaatcgatttgcacaaagagaagctgctttga

SEQ ID NO: 148 - DNA, 1966 bp, Cannabis sativa
  source 1..1966: mol_type = other DNA
atgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacattgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtataacattgtttcaactgcaaatcaagctccaacagggaacatagggagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcttcccttcagactattattcaagatgcttcacagtgtggttcatccaatgcattgagagcacccatggaaggcaatattagtaatcacagtctcaacaggagtgggtcaggtagtaaccatggtagcaacggacaaaagagaagcaccaatgcttcaaactccagagggaaaaagacagaaagtgagagtgtggttactggaagagggaaaaccattgaaggaagtgaatcggatggaaatcgatttgcacaaagagaagctgctttga

SEQ ID NO: 149 - DNA, 1980 bp, Cannabis sativa
  source 1..1980: mol_type = other DNA
atgaataataaagctcccgtaaccaatgatgagctaactgagctgaatcaccggattcaagatgggaaaaaagaaagaagggagagggttacgagagaacgccaagggctctcagaggaacatgaatctaggatcaatgaagatgtgcaacaacatgtcagcaatggggagattggaacagtacaggctctggagaggagtcattctggccagcggaggtctcagcaacagcctcaaggacatttggttcgctgggagaggttcctagctttcaggtcgctaaaggttttattggtggaaaatgatgactcaactcgccatattgtcagcgcgctactaagaaattgtggctacgaagttacagctgtagaaaatggcagacaagcttggaaagtcttagaagatcttgtgacagatgttgatctcgttttgactgaggtagtcatgccctgtttatctggtattggtcttctaggcaagatcatgagcaaaaaaacatgcaaggacatcccttgatgtcttcacatgattctaggagtatggtctttaagtgtttatcgaaaggtgccgttgactttttagtgaaacctattcgaaagaatgagctgaaaaacctttggcaacatgtttggagaaaatgccacatttctagtaatagtggaagtgaaagtggtatatggattgaaaagcctttaaagtcaagaactgtggaacattcagacaacaacagtggcagcaatgatgaggatgatactgacagcattggtctaaatttcaggaatgaaagtgacagtggaacacagagctcttggacaaagagagcagaagttgacagccctcaggcagtgtcgtgggagcagtttgctgatcttcctgatagcactaatcatcaggtcaatcatccaaggcaagaagcctttggaaacaactgggtacctgaaaatgcaacagtaacaccacgcccacataatgatgagcttgacaaaaaagtcatgggaaaagacttgaaaataggattacctagccttcttgaagacacaagtgaaaaagggctgaccaacatggaaggtactaataaagataaatgttctgaactgaactcaaagaaagatgatcaggagcaggagaaaagggaattagacctcaacaatgaagaaccgagtgcagaaaagacccaagctgttgatctgatgggtgtctccaattatagtattgatcctcacatggaaagtggagttcttgatgtcccaaacgaactctccaaggctgcctgcatgagagataatgccaaccatgagaataaagaaacacctttttttgagctcattttaaagaggccaagagatatccaagatactggaaccagcgcacacgatcgaaatgttttgagacattccgatatttcagctttttcaaggtataacattgtttcaactgcaaatcaagctccaacagggaacatagggagctgttctcctctagataatagctcagatgcagcaaaaacagaatcaatcccaaatttgcaatctgattcaaatggtacacctcccaaccagggttccaatggtagtagcaacaataatgacatgggatccactacgaataatgcttttaccaaacaagtggcttttgcagagaggcctacaaacaaatccacaatcaaactccaatcaaacactggtttccaacaagtgcaaaatggccaagcttcccttcagactattattcaagatgcttcacagtgtggttcatccaatgcattgagagcacccatggaaggcaatattagtaatcacagtctcaacaggagtgggtcaggtagtaaccatggtagcaacggacaaaagagaagcaccaatgcttcaaactccagagggaaaaagacagaaagtgagagtgtggttactggaagagggaaaaccattgaaggaagtgaatcggatggaaatcgatttgcacaaagagaagctgctttga